(19) United States
(12) Patent Application Publication
(10) Pub. No.: US 2023/0332165 A1
(43) Pub. Date: Oct. 19, 2023

(54) MARKER COMPOSITION FOR SELECTING LIVING MODIFIED ORGANISM, LIVING MODIFIED ORGANISM, AND TRANSFORMATION METHOD

(71) Applicant: INDUSTRY-ACADEMIC COOPERATION FOUNDATION GYEONGSANG NATIONAL UNIVERSITY, GYEONGSANGNAM-DO (KR)

(72) Inventors: Seon Won KIM, Gyeongsangnam-do (KR); Hyo Jung HAN, Gyeongsangnam-do (KR); Sin Young KIM, Gyeongsangnam-do (KR); Ji Bin PARK, Busan (KR)

(21) Appl. No.: 18/076,510

(22) Filed: Dec. 7, 2022

Related U.S. Application Data
(62) Division of application No. 16/612,918, filed on Nov. 12, 2019, now Pat. No. 11,549,117.

(30) Foreign Application Priority Data
May 11, 2017 (KR) 10-2017-0058829
May 11, 2018 (KR) 10-2018-0054080

(51) Int. Cl. C12N 15/70; C12N 1/20; C12N 15/74; C12P 5/00; C12P 7/02; C12N 15/63

(52) U.S. Cl. CPC C12N 15/70; C12N 1/20; C12N 15/746; C12P 5/002; C12P 7/02; C12N 15/63

(57) ABSTRACT

The present invention provides a marker composition for selecting a living modified organism, which allows transformation and the production of a target product without antibiotics or antibiotic resistance genes, a living modified organism, a method of transforming an organism, and a method of producing a target product. The present invention provides a marker composition for selecting a living modified organism, which may basically prevent problems caused by the use of antibiotics and antibiotic resistance genes and produce a target product at a high yield, a living modified organism, a method of transforming an organism, and a method of producing a target product.

### CROSS REFERENCE TO RELATED APPLICATIONS AND CLAIM OF PRIORITY

This application is a divisional application of application Ser. No. 16/612,918, filed on Nov. 12, 2019, which is a National Stage entry from International Application No. PCT/KR2018/005425 filed on May 11, 2018, which claims priority to the benefit of Korean Patent Application Nos. 10-2017-0058829 filed on May 11, 2017 and 10-2018-0054080 filed on May 11, 2018 in the Korean Intellectual Property Office, the entire contents of which are incorporated herein by reference.

### BACKGROUND

### 1. Technical Field

The present invention relates to a marker composition for selecting a living modified organism, a living modified organism and a method of transforming an organism.

### 2. Background Art

Recently, for industrial production of useful materials, LMOs has been mostly used. In this case, in order to enhance expressions of a metabolic pathway and a target product biosynthetic pathway, related genes are induced using various expression vectors (plasmids). In this case, the vectors have antibiotic resistance genes as a selection marker, and antibiotics are added to a culture liquid to stably maintain the plasmids in a host cell during the culture. However, when using antibiotics in the cultural process, there are problems such as an increase in production costs due to the use of expensive antibiotics, environmental pollution due to antibiotic leakage, a risk of a generation of antibiotic resistance mutations in the natural world, a need for additional separation and purification processes due to antibiotics remaining in the final product, difficulties in using antibiotic resistance gene marker-containing strains and acquiring a permission.

Further, if a cultural time is increased in a case of cultivation using antibiotics, a loss of plasmids containing antibiotic resistance genes as a selection marker occurs due to degradation and modification of antibiotics, and thereby causing a drastic decrease in productivity in the second half of the culture. The degradation and modification of the antibiotics are caused by enzymes expressed in antibiotic marker genes and by spontaneous instability of antibiotics, which result in serious problems such as a generation of secondary products in cultural processes requiring a long-term fermentation.

In order to solve these problems, there is a method of inserting foreign genes necessary for the production of the target product into chromosomes of a host organism, but this method has problems such as a decrease in an expression amount of proteins due to a reduction in an amount of genes, difficulties in introducing and expressing a number of genes into the chromosomes compared to the introduction of plasmids having a plurality of copies of the genes.

Due to the above-described reasons, developing antibiotic marker-free organisms has become an issue in the bioprocess industry in recent years. However, to date, there are no or very limited antibiotic marker-free systems that can be stably and usefully used in the industry. Although auxotrophic selection markers may be used in place of the antibiotic markers in auxotrophic selection mutant strains, there is a disadvantage that complex media, which is a commonly used industrial medium, cannot be used.

Another example is StabyExpress™, developed by Delphi Genetics. This uses ccd operons (ccdA and ccdB), which are antidote/poison systems present in bacteria. However, they are operated only in some bacteria, and are not operated frequently if an expression ratio of the ccdA/ccdB is not exactly correct.

### SUMMARY

An object of the present invention is to provide a marker composition for selecting a living modified organism that can replace an antibiotic and an antibiotic resistance marker.

Another object of the present invention is to provide a transformation method that does not require use of antibiotics and antibiotic resistance markers, and a living modified organism.

1. A marker composition for selecting a living modified organism including: a plasmid into which at least one of genes encoding enzymes in an isopentenyl diphosphate or dimethylallyl diphosphate synthetic pathway is introduced.

2. The marker composition for selecting a living modified organism according to the above 1, wherein the organism inherently has the isopentenyl diphosphate or dimethylallyl diphosphate synthetic pathway.

3. The marker composition for selecting a living modified organism according to the above 1, wherein the synthetic pathway is a MEP pathway or an MVA pathway.

4. The marker composition for selecting a living modified organism according to the above 1, wherein the gene encoding enzymes in the synthetic pathway is a gene encoding one or more enzymes selected from the group consisting of 1-dioxy-D-xylulose-5-phosphate (DXP) synthase, DXP reductoisomerase, 2-C-methyl-D-erythritol-4-phosphate (MEP) cytidyltransferase, 4-diphosphocytidyl-2-C-methyl-D-erythritol kinase, 2-C-methyl-D-erythritol-2,4-cyclodiphosphate (MEcPP) synthase, 4-hydroxy-3-methyl-2-butenyl diphosphate (HMBPP) synthase, HMBPP reductase, acetoacetyl-CoA synthase, 3-hydroxyl-3-methylglutary-CoA (HMG-CoA) synthase, HMG-CoA reductase, mevalonate kinase, mevalonate-5-phosphate kinase, mevalonate-5-diphosphate decarboxylase and IPP isomerase.

5. The marker composition for selecting a living modified organism according to the above 1, wherein the composition is transformed into an organism in which a gene encoding the same enzyme as said gene or a complementary gene thereof is attenuated or deleted.

6. The marker composition for selecting a living modified organism according to the above 5, wherein the gene is a gene encoding enzymes in the MEP pathway, and the complementary gene is a gene encoding at least one of enzymes in the MVA pathway.

7. The marker composition for selecting a living modified organism according to the above 6, wherein the complementary gene is a gene encoding acetoacetyl-CoA synthase, 3-hydroxyl-3-methylglutary-CoA (HMG-CoA) synthase, HMG-CoA reductase, mevalonate kinase, mevalonate-5-phosphate kinase, mevalonate-5-diphosphate decarboxylase and IPP isomerase.

8. The marker composition for selecting a living modified organism according to the above 5, wherein the gene is a gene encoding enzymes in the MVA pathway, and the complementary gene is a gene encoding at least one of enzymes in the MEP pathway.

9. The marker composition for selecting a living modified organism according to the above 1, including at least two of the genes, and these genes are introduced into a separate plasmid, respectively.

10. The marker composition for selecting a living modified organism according to the above 1, wherein the plasmid further includes a gene introduced therein to encode enzymes in a pathway selected from the group consisting of isoprenoid, santalene, bisabolol and retinol synthetic pathways.

11. A living modified organism transformed with a plasmid in which at least one of genes encoding enzymes in an isopentenyl diphosphate or dimethylallyl diphosphate synthetic pathway is attenuated or deleted, wherein a gene encoding the same enzyme as the attenuated or deleted gene or a complementary gene thereof is introduced therein.

12. The organism according to the above 11, wherein the organism inherently has the isopentenyl diphosphate or dimethylallyl diphosphate synthetic pathway.

13. The organism according to the above 11, wherein the synthetic pathway is a MEP pathway or an MVA pathway.

14. The organism according to the above 11, wherein the gene encoding enzymes in the synthetic pathway is a gene encoding one or more enzymes selected from the group consisting of 1-dioxy-D-xylulose-5-phosphate (DXP) synthase, DXP reductoisomerase, 2-C-methyl-D-erythritol-4-phosphate (MEP) cytidyltransferase, 4-diphosphocytidyl-2-C-methyl-D-erythritol kinase, 2-C-methyl-D-erythritol-2,4-cyclodiphosphate (MEcPP) synthase, 4-hydroxy-3-methyl-2-butenyl diphosphate (HMBPP) synthase, HMBPP reductase, acetoacetyl-CoA synthase, 3-hydroxyl-3-methylglutary-CoA (HMG-CoA) synthase, HMG-CoA reductase, mevalonate kinase, mevalonate-5-phosphate kinase, mevalonate-5-diphosphate decarboxylase and IPP isomerase.

15. The organism according to the above 11, wherein the gene to be attenuated or deleted is a gene encoding enzymes in the MEP pathway.

16. The organism according to the above 15, wherein the gene is a gene encoding at least one of DXP synthase and DXP reductoisomerase.

17. The organism according to the above 11, wherein the gene to be attenuated or deleted is a gene encoding enzymes in the MEP pathway, and the complementary gene is a gene encoding at least one of enzymes in the MVA pathway.

18. The organism according to the above 17, wherein the complementary gene is a gene encoding acetoacetyl-CoA synthase, 3-hydroxyl-3-methylglutary-CoA (HMG-CoA) synthase, HMG-CoA reductase, mevalonate kinase, mevalonate-5-phosphate kinase, mevalonate-5-diphosphate decarboxylase and IPP isomerase.

19. The organism according to the above 11, wherein the attenuated or deleted gene is a gene encoding enzymes in the MVA pathway.

20. The organism according to the above 11, wherein the plasmid further includes a gene introduced therein to encode enzymes in a pathway selected from the group consisting of isoprenoid, santalene, bisabolol and retinol synthetic pathways.

21. A method of transforming an organism including: attenuating or deleting at least one of genes encoding enzymes in an isopentenyl diphosphate or dimethylallyl diphosphate synthetic pathway of an organism to be transformed; and
transforming the organism with a recombinant plasmid into which a gene encoding the same enzyme as the attenuated or deleted gene or a complementary gene thereof is introduced.

22. The method of transforming an organism according to the above 21, wherein the organism inherently has the isopentenyl diphosphate or dimethylallyl diphosphate synthetic pathway.

23. The method of transforming an organism according to the above 21, wherein the transformation is performed without antibiotics.

24. The method of transforming an organism according to the above 21, wherein the synthetic pathway is a MEP pathway or an MVA pathway.

25. The method of transforming an organism according to the above 21, wherein the gene encoding enzymes in the synthetic pathway is a gene encoding one or more enzymes selected from the group consisting of 1-dioxy-D-xylulose-5-phosphate (DXP) synthase, DXP reductoisomerase, 2-C-methyl-D-erythritol-4-phosphate (MEP) cytidyltransferase, 4-diphosphocytidyl-2-C-methyl-D-erythritol kinase (IspE), 2-C-methyl-D-erythritol-2,4-cyclodiphosphate (MEcPP) synthase, 4-hydroxy-3-methyl-2-butenyl diphosphate (HMBPP) synthase, HMBPP reductase, acetoacetyl-CoA synthase, 3-hydroxyl-3-methylglutary-CoA (HMG-CoA) synthase, HMG-CoA reductase, mevalonate kinase, mevalonate-5-phosphate kinase, mevalonate-5-diphosphate decarboxylase and IPP isomerase.

26. The method of transforming an organism according to the above 21, wherein the attenuated or deleted gene is a gene encoding enzymes in the MEP pathway.

27. The method of transforming an organism according to the above 26, wherein the gene is a gene encoding at least one of DXP synthase and DXP reductoisomerase.

28. The method of transforming an organism according to the above 21, wherein the gene to be attenuated or deleted is a gene encoding enzymes in the MEP pathway, and the complementary gene is a gene encoding at least one of enzymes in the MVA pathway.

29. The method of transforming an organism according to the above 28, wherein the complementary gene is a gene encoding acetoacetyl-CoA synthase, 3-hydroxyl-3-methylglutary-CoA (HMG-CoA) synthase, HMG-CoA reductase, mevalonate kinase, mevalonate-5-phosphate kinase, mevalonate-5-diphosphate decarboxylase and IPP isomerase.

30. The method of transforming an organism according to the above 21, wherein the gene to be attenuated or deleted is a gene encoding at least one of enzymes in the MVA pathway.

31. The method of transforming an organism according to the above 21, wherein the plasmid further includes a gene encoding enzymes in a pathway selected from the group consisting of isoprenoid, santalene, bisabolol and retinol synthetic pathways.

32. The method of transforming an organism according to the above 21, wherein at least two genes are attenuated or deleted, and a strain is transformed with two plasmid including a gene encoding the same enzyme as the attenuated or deleted gene, respectively.

33. A method of producing a target product including: culturing the organism according to any one of the above 11 to 20 in a medium including a substrate.

34. The method of producing a target product according to the above 33, wherein the medium does not include antibiotics.

The marker composition for selecting a living modified organism of the present invention does not use antibiotic resistance genes. Thus, transformation is possible without the use of antibiotics and antibiotic resistance genes, thereby basically preventing many problems caused by the use of antibiotics and antibiotic resistance genes.

The marker composition for selecting a living modified organism of the present invention is less likely to disappear even when culturing the living organism (briefly, ‘organism’) for a long period time.

The organism of the present invention is capable of transforming and producing a target product without antibiotics and antibiotic resistance genes, thereby basically preventing many problems caused by the use of antibiotics and antibiotic resistance genes.

The transformation method of the present invention can transform an organism without antibiotics or antibiotic resistance genes.

The production method of a target product of the present invention can produce the target product in a high yield.

### BRIEF DESCRIPTION OF THE DRAWINGS

FIG. 1 is a view showing IPP and DMAPP biosynthetic pathways.

FIG. 2 is a graph showing a HPLC analysis profile of retinoid.

FIG. 3 is graphs showing standard curves for quantitative analysis of retinoid.

FIG. 4 is a graph showing a GC analysis profile of santalene.

FIG. 5 is a graph showing a GC analysis profile of bisabolol.

FIG. 6 is a graph showing a standard curve for quantitative analysis of bisabolol.

FIG. 7 is a view showing a fermentation by-product generation pathway.

FIG. 8 is a graph showing culture results according to the presence or absence of antibiotics of santalene producing strains that do not require antibiotics (briefly, ‘antibiotic-free’) constructed by complementing a foreign MVA pathway in MEP pathway-defective strains.

FIG. 9 is a graph showing culture results according to the presence or absence of antibiotics in the antibiotic-free retinoid producing strains constructed by dividing the foreign MVA pathway into two plasmids in the MEP pathway-defective strains.

FIG. 10 is graphs showing culture results according to the presence or absence of antibiotics of the retinoid producing strains from which the antibiotic marker is removed.

FIG. 11 is a graph showing culture results according to the presence or absence of antibiotics of the santalene producing strains using a deleted MEP pathway gene as a selection marker.

FIG. 12 is a graph showing culture results of antibiotic-free isoprenoid (santalene, and bisabolol) producing strains constructed by dividing the deleted MEP pathway gene into a plurality of plasmids.

FIG. 13 is graphs showing culture results of the retinoid producing strains from which antibiotic markers having the foreign MV pathway additionally introduced together with the deleted MEP pathway gene are removed.

FIG. 14 is photographs showing results of fluorescent protein expression of antibiotic-free strain using the deleted MEP pathway gene as a selection marker.

FIG. 15 is a schematic view of pCIN-mvaA-EGFP recombinant plasmid.

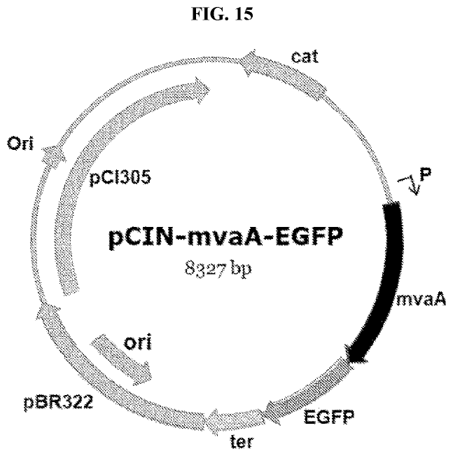

FIG. 16 is photographs showing the results of fluorescent protein expression of antibiotic-free strain using the deleted MVA pathway genes as a selection marker.

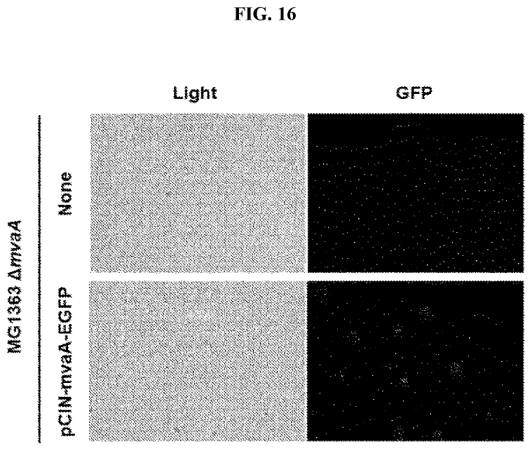

### DETAILED DESCRIPTION

Hereinafter, the present invention will be described in detail.

The present invention provides a marker composition for selecting a living modified organism including a plasmid into which at least one of genes encoding enzymes in an isopentenyl diphosphate or dimethylallyl diphosphate synthetic pathway is introduced.

The isopentenyl diphosphate (IPP) or dimethylallyl diphosphate (DMAPP) synthetic pathway is a biosynthetic pathway which is essentially included in all living organisms. The IPP and DMAPP are metabolites in cells, and the cells cannot survive upon lacking the same. In addition, these substances are strongly negatively charged phosphorylated substances, and cannot be introduced into the cells even when they are present in a medium, such that it is necessary to be generated in the cells.

Thus, when including the plasmid into which at least one of genes encoding enzymes in the isopentenyl diphosphate or dimethylallyl diphosphate synthetic pathway introduced, it is possible to be used as a marker composition for selecting a living modified organism.

Types of the organisms according to the present invention are not limited so long as they inherently have the isopentenyl diphosphate (IPP) or dimethylallyl diphosphate (DMAPP) synthetic pathway, and may include animals, plants, and microorganisms, and specifically, the microorganisms.

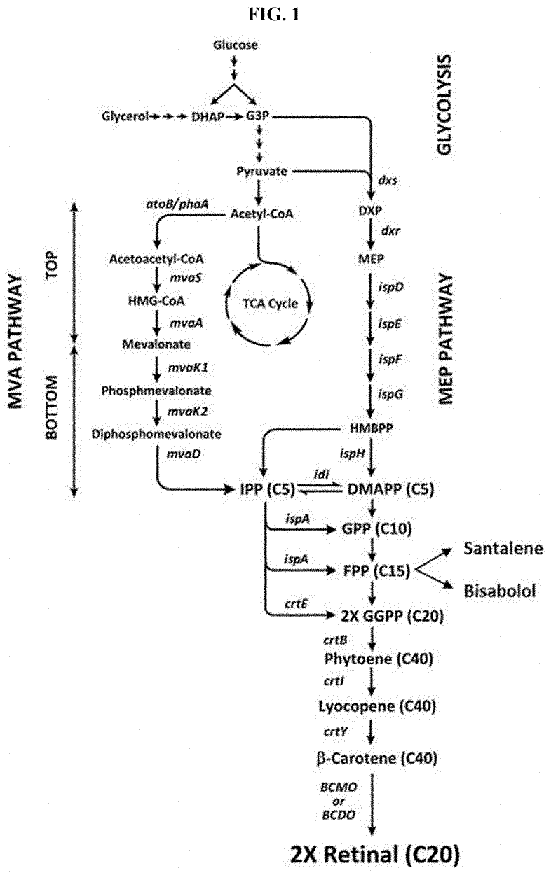

Specifically, the isopentenyl diphosphate or dimethylallyl diphosphate synthetic pathway may be an MEP pathway or MVA pathway shown in FIG. 1.

The gene encoding enzymes in the isopentenyl diphosphate or dimethylallyl diphosphate synthetic pathway may include a gene such as, for example, 1-dioxy-D-xylulose-5-phosphate (DXP) synthase of the MEP pathway, DXP reductoisomerase, 2-C-methyl-D-erythritol-4-phosphate (MEP) cytidyltransferase, 4-diphosphocytidyl-2-C-methyl-D-erythritol kinase (IspE), 2-C-methyl-D-erythritol-2,4-cyclodiphosphate (MEcPP) synthase, 4-hydroxy-3-methyl-2-butenyl diphosphate (HMBPP) synthase, HMBPP reductase, acetoacetyl-CoA synthase of the MVA pathway, 3-hydroxyl-3-methylglutary-CoA (HMG-CoA) synthase, HMG-CoA reductase, mevalonate kinase, mevalonate-5-phosphate kinase, mevalonate-5-diphosphate decarboxylase, IPP isomerase and the like.

The marker composition for selecting a living modified organism of the present invention may be transformed into an organism in which a gene encoding the same enzyme as said gene or a complementary gene thereof is attenuated or deleted.

As described above, when the isopentenyl diphosphate and dimethylallyl diphosphate synthetic pathways are inactivated, the cells cannot survive, whereas the marker composition of the present invention includes the plasmid into which at least one of genes encoding enzymes in an isopentenyl diphosphate or dimethylallyl diphosphate synthetic pathway is introduced, thereby the organisms modified therewith can survive by activating the defective IPP/DMAPP pathways again. Therefore, it is possible to confirm the modified organism by the survival of the organism.

The organism to be transformed may be one in which a gene encoding the same enzyme as the gene of the marker composition or the complementary gene thereof is attenuated or deleted.

In a case of the gene encoding the same enzyme, the gene may be the same gene as the attenuated or deleted gene, or may be a gene encoding the same enzyme derived from other species.

As a particular example, the organism to be transformed may be one in which a gene encoding DXP reductoisomerase is attenuated or deleted, and the plasmid according to the present invention may be one into which the same gene as said gene or a gene encoding the same enzyme derived from other species (with different genes) is introduced.

The complementary gene means a gene that may activate a pathway other than the pathways inactivated by attenuation or deletion of the gene, thereby producing isopentenyl diphosphate or dimethylallyl diphosphate. For example, in a case of an organism whose gene of the MEP pathway is attenuated or deleted, the plasmid of the marker composition may be one in which a gene encoding enzymes into the entire MVA pathway is introduced, and in a case of an organism whose gene of the MVA pathway is attenuated or deleted, the plasmid of the marker composition may be one into which a gene encoding the enzymes in the entire MEP pathway is introduced.

When the composition of the present invention includes a plasmid into which at least two or more of genes encoding enzymes in the isopentenyl diphosphate or dimethylallyl diphosphate synthetic pathway is introduced, two or more genes may be introduced into one plasmid, or each gene may be introduced into separate plasmids, respectively.

When the genes are respectively introduced into the separate plasmids, the organism should be transformed with all the plasmids to survive, therefore, the organisms may be selected as an organism transformed with all the plasmids.

A particular example of the organism to be transformed may be an organism in which at least one of a gene encoding DXP synthase and a gene encoding DXP reductoisomerase is attenuated or deleted, and the composition of the present invention may include two plasmids into which the same gene as said gene or a gene encoding the same enzyme derived from other species (with different genes) is introduced

In general, transformation of the organisms is performed in a culture liquid in which antibiotics are present, such that antibiotic resistance genes are used as a selection marker. However, in this case, there are problems such as an increase in production costs due to the use of expensive antibiotics, environmental pollution due to antibiotic leakage, a risk of a generation of antibiotic resistance mutant organisms and the like.

In addition, since the antibiotic resistance gene does not completely protect host cells, a damage to the host cells due to the antibiotics may occur. Further, setting and maintaining an optimal concentration of the used antibiotic to minimize the damage is recognized as a very difficult work in the industry, which may vary whenever the host cell is changed.

Furthermore, if a cultural time is increased in a case of cultivation using antibiotics, a loss of plasmids containing antibiotic resistance genes as a selection marker occurs due to degradation and modification of antibiotics, and thereby causing a drastic decrease in productivity in the second half of the cultivation. The degradation and modification of the antibiotics are caused by enzymes expressed in antibiotic marker genes and by spontaneous instability of antibiotics, which result in serious problems such as a generation of secondary products in cultural processes requiring a long-term fermentation.

However, by using the marker composition for selecting a living modified organism of the present invention, an occurrence of the above-described problems may be basically prevented, and the plasmid is safely maintained in the host organism without using antibiotics, as well as the living modified organisms may be selected.

Further, by additionally introducing genes encoding enzymes in a pathway for producing a target product into such a plasmid, it is possible to be used in stably mass-producing the target product in the host organism.

In addition, the present invention provides an organism transformed with a plasmid in which at least one of genes encoding enzymes in the isopentenyl diphosphate or dimethylallyl diphosphate synthetic pathway is attenuated or deleted, wherein a gene encoding the same enzyme as the attenuated or deleted gene or a complementary gene thereof is introduced therein.

The isopentenyl diphosphate or dimethylallyl diphosphate synthetic pathway may be the MEP pathway or the MVA pathway, and these enzymes are as described above.

The gene attenuated or deleted in the organism of the present invention may be at least one of genes encoding enzymes in the isopentenyl diphosphate or dimethylallyl diphosphate synthetic pathway, and specifically, at least one of genes encoding enzymes in the MEP pathway or the MVA pathway.

The organism of the present invention may be transformed with a plasmid into which the same gene as the attenuated or deleted gene or a gene encoding the same enzyme derived from other species is introduced.

As a particular example, it may be an organism in which at least one of genes encoding DXP synthase and DXP reductoisomerase is attenuated or deleted, or may be an organism transformed with a plasmid into which the same gene as said gene or a gene encoding the same enzyme derived from other species (with different genes) is introduced

In addition, the organism of the present invention may be transformed with a plasmid into which a complementary gene of the attenuated or deleted gene is introduced.

The complementary gene means a gene that may activate a pathway other than the pathways inactivated by attenuation or deletion of the gene, thereby producing isopentenyl diphosphate or dimethylallyl diphosphate. For example, in a case of an organism whose gene of the MEP pathway is attenuated or deleted, it may be transformed with a plasmid into which a gene encoding enzymes in the entire MVA pathway is introduced, and in a case of an organism whose gene of the MVA pathway is attenuated or deleted, it may be transformed with a plasmid in which a gene encoding the enzymes in the entire MEP pathway is introduced.

The organism of the present invention may be further transformed with a gene encoding enzymes in a target product synthetic pathway for producing a target product.

The genes may be variously selected according to the target product, and examples of the target product synthetic pathway may include an isoprenoid synthetic pathway, a santalene synthetic pathway, a retinol synthetic pathway, and a bisabolol synthetic pathway, but it is not limited thereto. All of the respective synthetic pathways are known pathways, and may be transformed with genes encoding enzymes in the known pathways.

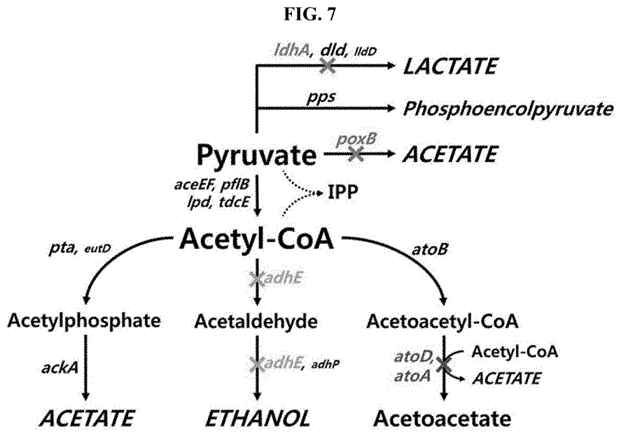

In addition, the organism of the present invention may be one in which a gene encoding enzymes in a by-product generation pathway of the target product is attenuated or deleted in the target product synthetic pathway. As a result, a yield of the target product may be further improved. An example thereof may include an enzyme for converting acetyl-CoA, which is a starting material of the MVA pathway, into acetate, lactate and ethanol of fermentation by-products, and specifically, acetaldehyde dehydrogenase (adhE), paruvate oxidase (PoxB), lactate dehydrogenase (ldhA), acetyl-COA, acetoacetyl-CoA (atoDA) synthase, and the like, but it is not limited thereto (see FIG. 7).

The organism of the present invention may not include an antibiotic resistance gene. Since the antibiotic resistance gene is not used as a marker, there is no need to include the same. Of course, after the above-described transformation, the organism may include the antibiotic resistance gene by further transforming with a plasmid into which the antibiotic resistance gene is subsequently introduced.

The present invention provides a method of transforming an organism including: attenuating or deleting at least one of genes encoding enzymes in an isopentenyl diphosphate or dimethylallyl diphosphate synthetic pathway of an organism to be transformed; and transforming the organism with a recombinant plasmid into which a gene encoding the same enzyme as the attenuated or deleted gene or a complementary gene thereof is introduced.

The gene encoding enzymes in the isopentenyl diphosphate or dimethylallyl diphosphate synthetic pathway may be a gene within the above-described range.

As described above, the transformation of organisms is usually performed in a culture liquid containing antibiotics, but since the transformation method of the present invention does not use the antibiotic resistance marker, the transformation of the present invention may be performed without antibiotics.

The attenuated or deleted gene may be at least one of genes encoding enzymes in the isopentenyl diphosphate or dimethylallyl diphosphate synthetic pathway. Specifically, it may be a gene encoding enzymes in the MEP pathway or the MVA pathway.

During transformation, the organism may be transformed with a plasmid into which the same gene as the attenuated or deleted gene or a gene encoding the same enzyme derived from other species is introduced.

As a specific example, the organism may be transformed with a plasmid into which a gene encoding at least one of DXP synthase and DXP reductoisomerase is attenuated or deleted, and may be transformed with the plasmid into which the same gene as said gene or a gene encoding the same enzyme derived from other species (with different genes) is introduced.

In addition, the organism may be transformed with a plasmid into which the complementary gene of the attenuated or deleted gene is introduced.

The complementary gene means a gene that may activate a pathway other than the pathways inactivated by attenuation or deletion of the gene, thereby producing isopentenyl diphosphate or dimethylallyl diphosphate. For example, in a case of an organism whose gene of the MEP pathway is attenuated or deleted, it may be transformed with a plasmid into which a gene encoding enzymes in the entire MVA pathway is introduced, and in a case of an organism whose gene of the MVA pathway is attenuated or deleted, it may be transformed with a plasmid in which a gene encoding the enzymes in the entire MEP pathway is introduced.

When introducing two or more genes, these genes may be introduced into a single plasmid or may be introduced into a plurality of plasmids, respectively.

As more specific examples of the deletion and transformation methods without limitation, as described above, the organism cannot survive without the IPP and DMAPP. Therefore, by adding 2-C-methyl-D-erythritol in the medium in the absence of dxs or ispC of the MEP pathway to produce 2-C-methyl-D-erythritol-4-phosphate (MEP) which is a metabolite of ispC, the organism may be transformed with a gene encoding the same enzyme as said gene or the complementary gene thereof while maintaining the growth of the organism. In addition, the MVA lower pathway gene may be first introduced, and then attenuation or deletion of the MEP pathway gene may be performed in a medium to which mevalonic acid is added.

When deleting the MVA pathway gene, a specific method may vary according to upper or lower pathway attenuation or deletion.

As a specific example of the upper pathway deletion, the growth of organism is maintained only by the lower pathway by adding mevalonic acid to the medium, and in this state, the upper pathway gene is attenuated or deleted, then the organism may be transformed with a gene encoding the same enzyme as said gene or the complementary gene thereof.

As a specific example of the lower pathway deletion, the growth of organism is maintained by introducing all the MEP pathway genes, and in this state, the upper pathway gene is attenuated or deleted, then the organism may be transformed with a gene encoding the same enzyme as said gene or the complementary gene thereof.

The plasmid according to the present invention may further include a gene encoding enzymes in the target product generation pathway for producing a target product.

The genes may be variously selected according to the target product, and examples of the target product synthetic pathway may include an isoprenoid synthetic pathway, a santalene synthetic pathway, a retinol synthetic pathway, and a bisabolol synthetic pathway, but it is not limited thereto. All of the respective synthetic pathways are known pathways, and genes encoding enzymes of the known pathways may be further introduced.

In addition, the organism of the present invention may be one in which a gene encoding enzymes in a by-product generation pathway of the target product is attenuated or deleted in the target product synthetic pathway. As a result, a yield of the target product may be further improved. An example thereof may include an enzyme for converting acetyl-CoA, which is a starting material of the MVA pathway, into acetate, lactate and ethanol of fermentation by-products, and specifically, acetaldehyde dehydrogenase (adhE), paruvate oxidase (PoxB), lactate dehydrogenase (ldhA), acetyl-COA, acetoacetyl-CoA (atoDA) synthase, and the like, but it is not limited thereto (see FIG. 7).

In addition, the present invention provides a method of producing a target product using the organism or including the transformation method.

The method of producing a target product of the present invention includes transforming the organism with a gene encoding enzymes in the target product generation pathway to produce the target product.

The gene encoding the enzymes in the target product generation pathway may be introduced into the above-described plasmid to be transformed into the organism.

The target product may be produced by culturing the organism in a medium containing a substrate, and the cultivation may be performed under a culture condition without antibiotics.

The methods of the present invention is capable of transforming the organism and producing the target product without antibiotics and antibiotic resistance genes, thereby basically preventing the problems caused by the use of antibiotics, as well as, a loss of plasmids due to degradation and modification of the antibiotics not occur, thus it is possible to produce the target product in a higher yield than the prior art.

Hereinafter, the present invention will be described in detail with reference to examples.

### Example

1. Material and Method

1) Experimental Strain and Material

Microorganisms used in experiments were purchased from the American Type Culture Collection (ATCC), the Korea Collection for Type Cultures (KCTC), and the Korea Culture Center of Microorganisms (KCCM), which are summarized in Table 1 below.

TABLE 1

| Experimental | | |
|---|---|---|
| strain | Description | Source & Reference |
| DH5a | F⁻, λ⁻, endA1, glnV44, thi-1, | NEB C2987 |
| | recA1, relA1, gyrA96, deoR, Φ | |
| | 80, dlacZ Δ15, ΔlacZYA-argF) | |
| | U169, hsdR17(r_{K}⁻ | |
| | m_{K}⁺), supE44 | |
| MG1655(DE3) | F⁻ λ⁻ IlvG rfb-50 rph-1 (DE3) | KCCM 41810 |
| MG1655(DE3) | DadhE (::P_{cTrc-}SN12Didi-ter) | — |
| DadhE::MVA | | |
| bottom | | |
| MG1655(DE3) | DatoDA (::P_{cTrc-}SN12Didi-ter) | — |
| DatoDA::MVA | | |
| bottom | | |
| MG1655(DE3) | DldhA (::P_{cTrc-}SN12Didi-ter) | — |
| DldhA::MVA | | |
| bottom | | |
| (MG1655(DE3) | DpoxB (::P_{cTrc-}SN12Didi-ter) | — |
| DpoxB::MVA | | |
| bottom | | |
| MG1655(DE3) | Δ dxr DadhE | — |
| Δdxr | (::P_{cTrc-}SN12Didi-ter) | |
| DadhE::MVA | | |
| bottom | | |
| MG1655(DE3) | Δ dxr, Δdxs, DadhE | — |
| Δdxr/s | (::P_{cTrc-}SN12Didi-ter) | |
| DadhE::MVA | | |
| bottom | | |
| E. coli EC1000 | RepA+ MC1000, KmR, | Leenhouts et al Mol |
| | carrying a single copy of the | Gen Genet. 1996 |
| | pWV01 repA gene in the glgB | Nov. 27:253(1- |
| | gene; host for pOR128-based | 2):217-24 |
| | plasmids | |
| L. lactis subsp. | Plasmid-free derivative of L. | Plasmid complement |
| cremoris | lactis subsp. Cremoris | of Streptococcus |
| MG1363 | NCD0712 | lactis NCD0712 and |
| | | other lactic |
| | | streptococci after |
| | | protoplast-induced |
| | | curing. J. Bacteriol. |
| | | 154:1-9. |
| L. lactis subsp. | ΔmvaA | — |
| cremoris | | |
| MG1383 | | |
| ΔmvaA | | |

DH5α(F-f80dlacZDM15D(lacZYA-argF)U169 deoR recA1 endA1 hsdR17(r_{K-}, m_{K+}) phoA supE44λ⁻thi-¹ gyrA96 relA1) was used in gene cloning, and MG1655(DE3)(F⁻λ⁻ilvG rfb-50 rph-1(DE3)) was used in production of a target product. pTrc99A and pSTV28 were used as an expression vector (Table 2). Products of New England Biolabs (U.S.) were used as a restriction enzyme and other enzymes. In order to perform PCR, products of Solgent (Korea) and Thermo Scientific (U.S.) were used as Pfu-X DNA polymerase and Phusion DNA polymerase, respectively. A product of Invitrogen (U.S.) was used as a DNA size maker. Products of Promega (U.S.); Sigma (U.S.); Merck (U.S.), and Amresco (U.S.) were used as IPTG; L(+)-arabinose, glucose and lactose; acetone; and glycerol, respectively. Other products of Sigma (U.S.) were used as other reagents.

Preparation of a medium for culturing microorganisms was conducted in accordance with the recommended medium composition and Difco manual (11th edition, Difco; BD Science, U.S.) of each strain distribution institution. Reagents used to prepare the media were purchased from BD Science (U.S.) and Sigma (U.S.). Cell amounts in the culture were represented as results measured by a spectrophotometer (Shimadzu UV-1601, Japan) at an optical density (OD) of 600 nm, and pH was measured by a pH meter B-212 (HORIBA, Japan).

Ampicillin and kanamycin were used as antibiotics for maintain plasmids in gene cloning at concentrations of 100 μg/ml and 50 μg/mL, respectively.

2) Extraction and Analysis of Retinoid

Retinoids were analyzed by the following method. 50-100 μl of culture liquid was taken, and cells were recovered by centrifugation at 14,000 rpm for 40 seconds. The cells were resuspended by adding 400 μl of acetone to the recovered cells, followed by extraction at 55° C. for 15 minutes in a dark place, and again adding 600 μl of acetone thereto for 15 minutes. The extract was centrifuged at 14,000 rpm for 10 minutes, and then only a supernatant was taken for HPLC quantitative analysis. In a case of adding a heavy mineral oil layer to the retinoid culture liquid, only the heavy mineral oil layer was centrifuged at 14,000 rpm for 10 minutes, and then 5-50 μl of the heavy mineral oil layer containing the retinoid was resuspended in 1 mL of acetone. The extract was left at room temperature for 15 minutes, while vortexing the same at an interval of 5 minutes. The extract was centrifuged at 14,000 rpm for 10 minutes, and then subjected to HPLC quantitative analysis by taking only the acetone layer.

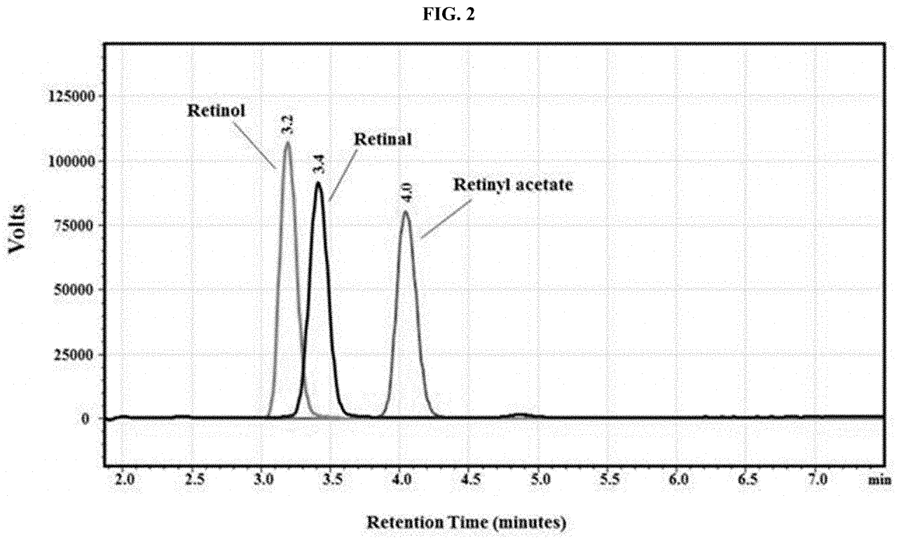

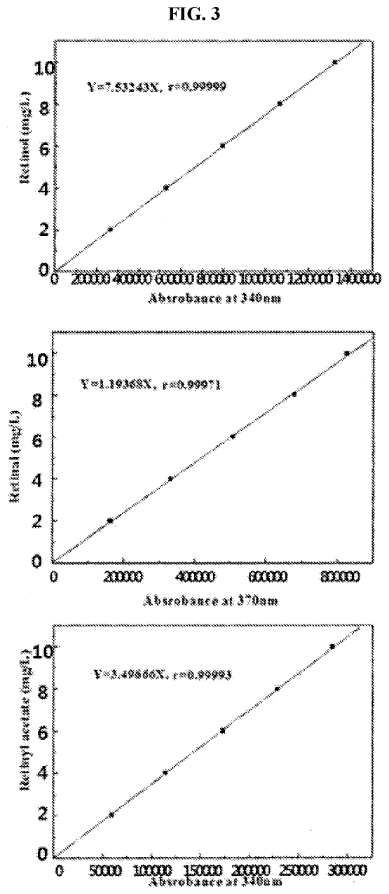

SHIMADZU LC-20A series with UV/Vis detector (Shimadzu, Kyoto, Japan) was used as a retinoid analysis system, and Symmetry C18 (250×4.6, 5 μm) with Symmetry guard C18 (15×4.6, 5 μm) was used as an analysis column. A mobile phase was analyzed in a mixture solution of methanol:acetonitrile (95:5, v/v) for 15 minutes. A flow rate was set to be 1.5 mL/min, and detection wavelengths of retinal; and retinol and retinyl acetate were measured at 370 nm; and 340 nm, respectively, followed by analyzing 20 μl of sample injection amount and 40° C. of oven temperature. Retinoid standard samples were used by dissolving in ethanol. Peak retention times of the standard samples were about 3.2 minutes for retinol, about 3.4 minutes for retinal, and about 4.0 minutes for retinyl acetate (FIG. 2). Calculation was performed in such a way that the analyzed results are put into a calibration curve calculated by an area of the analyzed peak as a peak area of the standard samples and converted into a dilution factor (FIG. 3).

3) Extraction and Analysis of Santalene

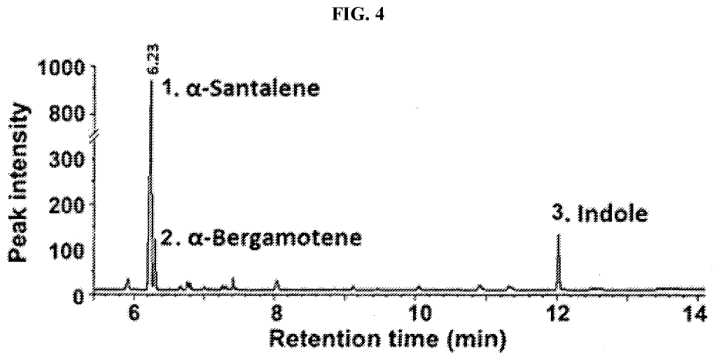

For analysis of santalene produced in a two-phase culture of applying decane to a medium, a decane layer was subjected to gas chromatography (GC) and gas chromatography-mass spectrometry (GC-MS) analyses (FIG. 4). The GC analysis was performed using GC/FID (Agilent Technologies7890A) equipped with 19091N-133 HP-Innowax column (30 m; internal diameter, 0.25 mm; film thickness, 250 nm). A temperature of a column oven was raised to 250° C. at a rate of 10° C./min from an initial temperature of 80° C. for 1 minute, and held for 1 minute. Nitrogen flowed as a mobile phase gas at a pressure of 39 psi, and a detector was maintained at a temperature of 260° C. The GC-MS analysis was performed using GCMS-QP2010 Ultra (SHIMADU, Tokyo, Japan), and helium was used as the mobile phase gas.

4) Extraction and Analysis of Bisabolol

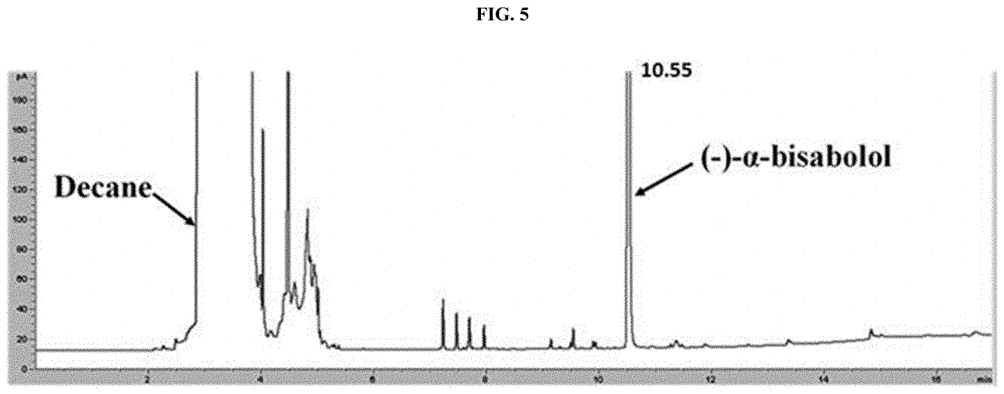

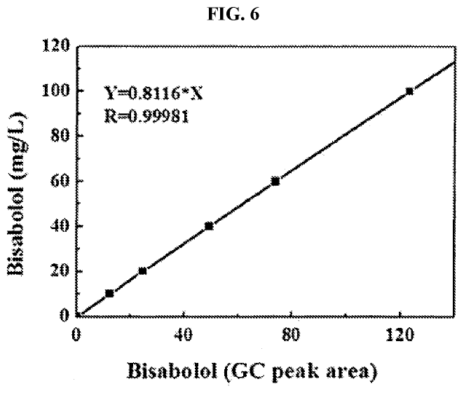

After the culture is completed, the decane layer was recovered by centrifugation (14,000 rpm, 10 min), and was subjected to the gas chromatography (GC) and gas chromatography-mass spectrometry (GC-MS) analyses (FIG. 5). The GC analysis was performed using GC/FID (Agilent Technologies 7890A) equipped with 19091N-133 HP-Innowax column (30 m; internal diameter, 0.25 mm; film thickness, 250 nm). The temperature of a column oven was increased at a rate of 20° C./min from an initial temperature of 50° C., and the temperature was increased at a rate of 15° C./min after reaching 90° C. After reaching 150° C., the temperature was increased to 190° C. at a rate of 20° C./min, and then allowed the temperature to reach 260° C. at a rate of 10° C./min, and maintained for 2 minutes. Nitrogen was supplied as the mobile phase gas at a pressure of 30 psi, and the detector was maintained at a temperature of 280° C. The GC-MS analysis was performed using GCMS-QP2010 Ultra (SHIMADU, Japan) and helium was used as the mobile phase gas. A peak area value of the measured bisabolol was calculated as following Equation 1 according to the calibration curve (FIG. 6) prepared in advance.

α-Bisabolol (mg/L)=0.8116×GC Peak area×Dilution factor [Equation 1]

2. Process of Constructing MEP Pathway-Defective Strain
Example of introducing MVA lower pathway gene into chromosome when constructing MEP pathway-defective strain, and performing a construction work in a mevalonic acid-added medium.
Deletion of MEP upper pathway gene

1) Insertion of Foreign MVA Lower Pathway into E. coli Chromosome

Generally, there are two methods for inserting a foreign pathway into E. coli chromosomes: PCR-based homologous recombination method using k-Red recombinase; and P1 transduction. In this experiment, by using the PCR-based homologous recombination method using k-Red recombinase, a foreign MVA lower pathway was inserted into E. coli MG1655(DE3). In the MVA pathway, a pathway from mevalonate to DMAPP was referred to as a lower pathway (see FIG. 1). Currently, the MVA lower pathway includes: mvaK1, mvaK2, and mvaD of Streptococcus pneumoniae; and idi genes of E. coli. The gene was amplified by using primers of SEQ ID NO: 1 and SEQ ID NO: 2, and cloned into a pTFCC(DPB) vector to construct pTFCC(DPB)-SN12Didi. The MVA lower pathways were inserted into four genes including adhE, poxB, ldhA and atoDA, respectively at insertion positions. They are genes that convert acetyl-CoA, which is a starting material of the MVA pathway, into acetate, lactate and ethanol of fermentation by-products, and it is possible to improve fermentation productivity and use efficiency of carbon source by blocking the same (FIG. 7).

The experimental method is as follows. First, vectors including a promoter, a multi-cloning site, a terminator, an FRT site, and an antibiotic marker were constructed so that a pathway to be inserted into the E. coli chromosome is expressed in the cell, which is shown in Table 2 below.

TABLE 2

| | | Genbank | | SEQ ID |
|---|---|---|---|---|
| Plasmid | Description | Grant NO. | Product | NO. |
| pSTV28 | p15A origin, lac promoter, | M22744 | Takara Korea | 71 |
| | lacZ, and cat | | (Korea) | |
| pTrc99A | pMB1 origin, trc promoter, | U13872.1 | Pharmacia | 72 |
| | lacI^{q}, and bla | | (U.S.) | |
| pTFKC(DPB) | ColE1 origin, trc promoter | — | | 73 |
| | without operator region, | | | |
| | kanamycin cassette with FRT, | | | |
| | and bla | | | |
| pTFCC(DPB) | ColE1 origin, trc promoter | — | | 74 |
| | without operator region, | | | |
| | chloramphenicol cassette with | | | |
| | FRT, and bla | | | |
| pCP20 | cI857λts, FLP, cat, and bla | — | | 75 |
| pKD46 | repA101ts, oriR101, exo, bet, | AY048746 | | 76 |
| | gam, tL3, P_{BAD}, araC, and bla | | | |
| pT-ispA-STS | pTrc99A vector; lacIq; Ampr; | — | | 77 |
| | E. coli-derived FPP Synthase, | | | |
| | ispA; Ptrc expression vector | | | |
| | including Clausena lansium- | | | |
| | derived santalene synthase, | | | |
| | STS | | | |
| pTAS-NA | pTrc99A vector; lacIq; Ampr; | — | In this study | 78 |
| | IspA and idi, which are E. | | | |
| | coli-derived FPP synthase; | | | |
| | Clausena lansium-derived | | | |
| | santalene synthase, STS; E. | | | |
| | faecalis-derived mvaE and | | | |
| | mvaS; Ptrc expression vector | | | |
| | including S. pneumonia- | | | |
| | derived mvaK1, mvaK2 and | | | |
| | mvaD | | | |
| pSNAK | pSTV28 vector; E. faecalis- | — | | 79 |
| | derived lacZ; Km^{r}; mvaE and | | | |
| | mvaS; S. pneumonia-d | | | |
| | mvaK1, mvaK2 and mvaD; | | | |
| | Plac expression vector | | | |
| | including E. coli-derived idi | | | |
| pSNAK(-E) | pSTV28 vector; lacZ; Km^{r}; E. | — | In this study | 80 |
| | faecalis-derived mvaS; S. | | | |
| | pneumonia-derived mvaK1, | | | |
| | mvaK2 and mvaD; Plac | | | |
| | expression vector including | | | |
| | E. coli-derived idi | | | |
| pSNA(-E)free | pSTV28 vector; lacZ; E. | — | In this study | 81 |
| | faecalis-derived mvaS; S. | | | |
| | pneumonia-derived mvaK1, | | | |
| | mvaK2 and mvaD; Vector | | | |
| | including E. coli-derived idi | | | |
| | without antibiotic markers | | | |
| pTEFAmvaE | pTrc99A vector; lacIq; Amp^{r}; | — | | 82 |
| | Ptrc expression vector | | | |
| | including E. faecalis-derived | | | |
| | mvaE | | | |
| pT- | pTrc99A vector; lacIq; Amp^{r}; | — | | 83 |
| DHBSRYbbO | P. agglomerans-derived crtE, | | | |
| | crtB and crtI; P.ananatis- | | | |
| | derived vrtY; codon- | | | |
| | optimized uncultured marine | | | |
| | bacterium 66A03-derived SR; | | | |
| | dxs and YbbO of E.coli | | | |
| pT- | pTrc99A vector; lacIq; Amp^{r}; | — | In this study | 84 |
| HBSRYbbO | P. agglomerans-derived crtE, | | | |
| | crtB and crtI; P. ananatis- | | | |
| | derived crtY; codon- | | | |
| | optimized uncultured marine | | | |
| | bacterium 66A03-derived SR; | | | |
| | YbbO of E.coli | | | |
| pT- | pTrc99A vector; lacIq; Amp^{r}; | — | In this study | 85 |
| HBSREYbbO | P. agglomerans-derived crtE, | | | |
| | crtB and crtI; P. ananatis- | | | |
| | derived crtY; codon- | | | |
| | optimized uncultured marine | | | |
| | bacterium 66A03-derived SR; | | | |
| | YbbO of E. coli; E. faecalis- | | | |
| | derived mvaE | | | |
| pT- | pTrc99A vector; lacIq; | — | In this study | 86 |
| HBSREYbbOfree | P. agglomerans-derived crtE, | | | |
| | crtB and crtl; P. ananatis- | | | |
| | derived crtY; codon- | | | |
| | optimized uncultured marine | | | |
| | bacterium 66A03-derived SR; | | | |
| | YbbO of E. coli; Vector | | | |
| | including E. faecalis-derived | | | |
| | mvaE without antibiotic | | | |
| | markers | | | |
| pT-dxr | pTrc99A vector; lacIq; Amp^{r}; | — | | 87 |
| | Ptrc expression vector | | | |
| | including E. coli-derived dxr | | | |
| pTAS-dxr | pTrc99A vector; lacIq; Amp^{r}; | — | In this study | 88 |
| | E. coli-derived FPP | | | |
| | Synthases, ispA and dxr; Ptrc | | | |
| | expression vector including | | | |
| | STS, Clausena lansium- | | | |
| | derived santalene synthase | | | |
| pT-dxr/s | pTrc99A vector; lacIq; Ampr; | — | | 89 |
| | Ptrc expression vector | | | |
| | including E. coli-derived dxr | | | |
| | and dxs | | | |
| pT-ispA- | pTrc99A vector; lacIq; Ampr; | — | In this study | 90 |
| MrBBS | E. coli-derived FPP Synthase, | | | |
| | ispA; Ptrc expression vector | | | |
| | including codon-optimized | | | |
| | Matricaria recutita-derived | | | |
| | α-bisabolol synthase, MrBBS | | | |
| pTAS-dxs | pTrc99A vector; lacIq; Ampr; | — | In this study | 91 |
| | E. coli-derived FPP synthase, | | | |
| | ispA and dxs; Ptrc expression | | | |
| | vector including Clausena | | | |
| | lansium-derived santalene | | | |
| | synthase, STS | | | |
| pTAB-idi-dxr | pTrc99A vector; lacIq; Ampr; | — | In this study | 92 |
| | E. coli-derived FPP | | | |
| | Synthases, ispA and dxr; Ptrc | | | |
| | expression vector including | | | |
| | codon-optimized Matricaria | | | |
| | recutita-derived α-bisabolol | | | |
| | synthase, MrBBS | | | |
| pSNAK(-E)- | pSTV28 vector; lacZ; Km^{r}; E. | — | In this study | 93 |
| dxs | faecalis-derived mvaS; S. | | | |
| | pneumonia-derived mvaK1, | | | |
| | mvaK2 and mvaD; Plac | | | |
| | expression vector including | | | |
| | E. coli-derived idi and dxs | | | |
| pSNA(-E)- | pSTV28 vector; lacZ; E. | — | In this study | 94 |
| dxsfree | faecalis-derived MvaS; S. | | | |
| | pneumonia-derived mvaK1, | | | |
| | mvaK2 and mvaD; Vector | | | |
| | including E. coli-derived idi | | | |
| | and dxs without antibiotic | | | |
| | markers | | | |
| PT- | pTrc99A vector; lacIq; | — | In this study | 95 |
| HBSREYbbO | P. agglomerans-derived crtE, | | | |
| dxrfree | crtB and crtl; P. ananatis- | | | |
| | derived crtY; codon- | | | |
| | optimized uncultured marine | | | |
| | bacterium 66A03-derived SR; | | | |
| | YbbO and E. coli-derived | | | |
| | dxr; Vector including E. | | | |
| | faecalis-derived mvaE | | | |
| | without antibiotic markers | | | |
| pEGFP | pUC origin, lac promoter; | — | Clontech | 96 |
| | EGFP, and AmpR | | (U.S.) | |
| pEGFP-dxr | pUC origin, lac promoter; | — | In this study | 97 |
| | EGFP, E.coli dxr, and AmpR | | | |
| PORI19 | Em^{r} Ori⁺ RepA⁻ lacZ′ | A System To | — | 98 |
| | derivative of pORI28 | Generate | | |
| | | Chromosomal | | |
| | | Mutations in | | |
| | | Lactococcus | | |
| | | lactis Which | | |
| | | Allows Fast | | |
| | | Analysis of | | |
| | | Targeted | | |
| | | Genes | | |
| pORI19-mvaA | PORI19-mvaA | — | In this study | 99 |
| pCI372 | E. coli/L. lactis shuttle vector, | Identification | — | 100 |
| | CamR | of the minimal | | |
| | | replicon of | | |
| | | lactococcus | | |
| | | lactis subsp. | | |
| | | lactis UC317 | | |
| | | plasmid | | |
| | | pCI305 | | |
| pCIN | pCI372-FP promoter, MCS, | — | In this study | 101 |
| | rrnp terminator | | | |
| pCIN-mvaA | pCIN-mvaA | — | In this study | 102 |
| pCIN-mvaA- | pCIN-mvaA,EGFP | — | In this study | 103 |
| EGFP | | | | |

All the constructed vectors were used with trc promoters or used by modifying so as to express the trc promoters at all times. Therefore, the constructed pBFKC has the trc promoter, and the pTFKC (DPB) and pTFCC (DPB) have the modified trc promoters, respectively, so as to be expressed at all times. A desired pathway was inserted using a multi-cloning site of the constructed vector, and PCR was performed using each of the constructed plasmids as a template, and a primer having a homology of 50 bp with a portion into which E. coli is inserted. Each primer information is represented in SEQ ID NO: 3 to SEQ ID NO: 10 in Table 4. The obtained PCR product was purified, followed by performing electro-transformation to 1.8 kV through a cuvette having an interval of 1 mm on E. coli MG1655(DE3) competent cells containing pKD46. Thereafter, immediately adding 1 mL of SOC medium (2% of Bacto Tryptone, 0.5% of yeast extract, 10 mM of NaCl, 2.5 mM of KCl, 10 mM of MgCl₂, 10 mM of MgSO₄, and 20 mM of glucose) thereto, followed by shaking culture at 37° C. for 1 hour. After smearing it on a plate medium to which antibiotics are added to culture at 37° C., single colonies were obtained. Herein, the obtained single colonies were suspended in a small amount of distilled water, heated for 10 minutes and centrifuged, then the supernatant was used as a template to perform PCR using each primer from SEQ ID NO: 11 to SEQ ID NO: 18 to confirm whether the pathway is inserted or not. In order to remove the antibiotic marker, single colonies were obtained by transforming pCP20 plasmids into strains confirmed the foreign pathway insertion, followed by culturing at 30° C. PCR was performed through the above primers for confirmation using the obtained single colonies as a template, and consequently, it was confirmed that the antibiotic marker was removed. The confirmed colonies were cultured at 43° C. to remove the pCP20 plasmid, which is a temperature sensitive plasmid, thereby obtaining a foreign pathway-inserted strain after completion.

2) Deletion of dxr and dxs Genes in MG1655(DE3) ΔadhE::MVAbottom Strain

Deletion of gene was also performed using Datsenko and Wanner's methods to delet dxr gene in E. coli chromosome.

In order to replace the dxr gene with kanamycin gene which is a selection marker gene by homologous recombination, PCR primers having base sequences at upstream and downstream ends of the dxr gene while binding to the kanamycin gene of pTFKC (DPB) plasmid were prepared. A forward primer consists of a 50 bp base sequence at the upstream end of the dxr gene, followed by a 15 bp kanamycin gene binding base sequence, and a reward primer consists of a 50 bp base sequence at the downstream end of the dxr gene, followed by a 20 bp kanamycin gene binding base sequence. The primers used herein are shown in Table 7 below. PCR reaction was performed using oligonucleotides of SEQ ID NO: 19 and SEQ ID NO: 20 as a primer, and pTFKC (DPB) as a template.

The purified PCR reaction product was transformed into MG1655(DE3) ΔadhE::MVAbottom competent cells containing pKD46 (Datsenko K A and Wanner B L, 2000 Proc Natl Acad Sci U.S.A., 97(12):6640-6645). Colonies were obtained from a plate medium containing 3.3 mM mevalonate and kanamycin, and the obtained colonies were confirmed by PCR to confirm that the kanamycin gene replaced the dxr gene by the homologous recombination. In addition, it was confirmed that the MEP pathway was blocked due to the deletion of the dxr gene based on the fact that colonies could not grow by smearing on a mevalonate-free plate medium. Primers used for colony of PCR were prepared so as to be bound to a region immediately adjacent to the dxr gene on the E. coli MG1655(DE3) chromosome. The used primer for confirmation is a primer of oligonucleotides of SEQ ID NOs: 21, 22 and 23.

In order to remove the antibiotic marker on the chromosome, single colonies were obtained by transforming pCP20 plasmids into strains confirmed the foreign pathway insertion, followed by culturing at 30° C. PCR was performed through the above primers for confirmation using the obtained single colonies as a template, and consequently, it was confirmed that the antibiotic marker was removed. The confirmed colonies were cultured at 43° C. to remove the pCP20 plasmid, which is a temperature sensitive plasmid, thereby obtaining MG1655(DE3) Δdxr ΔadhE::MVAbottom strain after completion.

In the same manner as in the above method, the MG1655(DE3) Δdxr ΔadhE::MVAbottom strain was subjected to deletion of the dxs gene to obtain MG1655(DE3) Δdxr/s ΔadhE::MVAbottom strain. For the homologous recombination, PCR reaction was performed using primers of SEQ ID NO: 24 and SEQ ID NO: 25, and pTFKC (DPB) as a template. Thereafter, oligonucleotides of SEQ ID NOs: 26, 27 and 28 were used as a primer for confirmation.

3. Methods of Constructing and Culturing Plasmid (MEP Pathway-Defective Strain)
Use of a strain in which a foreign MVA lower pathway is inserted into a chromosome and an MEP upper pathway gene is deleted
The selection marker of the plasmid used in the MEP pathway-defective strain may be an activated MVA pathway or a deleted MEP pathway gene of the chromosome, which may complement the deleted MEP pathway. Single or plurality of plasmids may be introduced into the defective strain, and the number thereof is not particularly limited. In a single plasmid method, it is necessary to compensate for the deletion of the host's MEP pathway by the single plasmid to be introduced, and in a plurality of plasmids method, only when all the plurality of plasmids to be introduced are present, it is necessary to compensate for the deletion of the host's MEP pathway.

1) Isoprenoid Biosynthetic Plasmid Based on Foreign MVA Pathway

A. Single Plasmid Method (Example of Producing Santalene)
This is a method in which all the activated MVA pathways that complement for the deletion of the host's MEP pathway are present in the single plasmid, and it is constructed so that the MVA pathway and the biosynthetic pathway of the target product are coexisted in this plasmid.

i. Construction of Plasmid

In order to use the foreign MVA pathway as a selection marker to replace antibiotic markers in the MEP pathway-defective strain, a recombinant plasmid is constructed by introducing an MVA pathway into a plasmid for producing santalene (‘santalene producing plasmid’). The defective strain may be grown when introducing recombinant plasmids to produce santalene.

Santalene producing plasmid pT-ispA-STS is formed by introducing an ispA gene which is FPP synthase of E. coli and STS which is a santalene synthase gene of Clausena lansium into pTrc99A vector. A foreign MVA pathway operon in a pSNAK plasmid was cloned using restriction enzyme sites BglII and SbfI located behind an STS gene in the plasmid. The pSNAK plasmid includes mvaK1, mvaD and mvaK2 of Streptococcus pneumoniae, mvaE and mvaS of Enterococcus faecalis, and an idi gene of E. coli, which are introduced therein. An MVA operon was introduced into a pT-ispA-STS vector by cleaving restriction enzymes BamHI and SbfI at both ends thereof. Restriction enzymes BamHI and BglII have the same cohesive end as each other. Finally, pTAS-NA was constructed in which a santalene producing operon and the MVA pathway operon were introduced together.

In the cloning process of the plasmid above, for selection of transformants, E. coli DH5u or E. coli MG1655(DE3) Δdxr ΔadhE::MVAbottom may be used. When using E. coli DH5u, it is possible to select on an ampicillin plate by using an antibiotic marker present in the plasmid to be constructed. When using E. coli MG1655(DE3) Δdxr ΔadhE::MVAbottom, the transformants may be selected without antibiotics, because the strain is capable of growing when the MEP pathway of host E. coli is inactivated and a plasmid with activated MVA pathway is introduced.

ii. Introduction of Plasmid

The defective strain is capable of growing when introducing recombinant plasmids to produce santalene. The constructed plasmid pTAS-NA may be introduced into MG1655(DE3) Δdxr ΔadhE::MVAbottom strain, in which the MEP pathway is deleted, to obtain a transformant on a mevalonate-free plate, thereby selecting the recombinant strains using the foreign MVA pathway of the plasmid. Thereafter, by culturing the recombinant strain in a non-antibiotic medium to confirm the productivity of santalene, it is possible to confirm maintenance and activation abilities of the plasmid having the selection marker based on the MVA pathway.

Recombinant strain having a plasmid related to antibiotic-free santalene production was spawn cultured under a non-antibiotic condition, followed by culturing in a mixed medium of 4 mL of 2YT medium (16 g of trypton per liter, 10 g of yeast extract, and 5 g of NaCl) containing 2% (v/v) of glycerol and 0.2 mM of IPTG and 1 mL of decane, which is a production medium. For cultivation, the mixed medium is put into a tube having a groove of 15 cm in length and 25 mm in diameter and is inoculated with each of the strains, followed by culturing in a shaking incubator at 30° C. while stirring at a speed of about 250 rpm.

iii. Culture Result

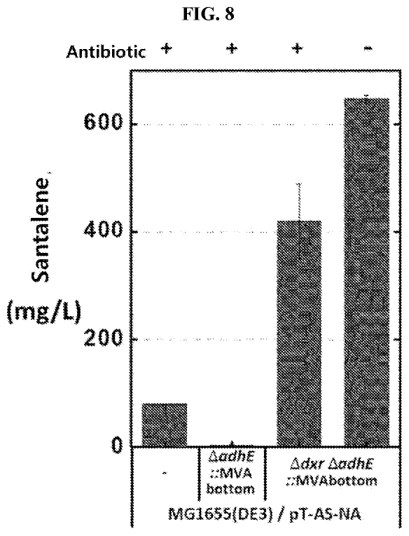

The santalene productions were compared in MG1655(DE3) Δdxr ΔadhE::MVAbottom, a newly constructed recombinant strain using wild type E. coli MG155 (DE3) strain as a control group. The culture results are shown in FIG. 8 and Table 3 below.

More specifically, the cultivation was performed in such a way that MG1655(DE3) and MG1655(DE3) ΔadhE::MVAbottom strains were cultured by adding antibiotics thereto, and MG1655(DE3) Δdxr ΔadhE::MVAbottom, to which an antibiotic-free system can be applied, was cultured by changing with or without the addition of the antibiotics. After 48 hours from the cultivation, the decane layer was recovered and the santalene production was analyzed through GC. As a result, the new recombinant strain MG1655(DE3) ΔadhE::MVAbottom strain exhibited 5 times or higher santalene production (420.2 mg/L) than strain MG1655(DE3) which produced 79.8 mg/L of santalene. In addition, the non-antibiotic culture exhibited higher santalene production (646.7 mg/L) than the case of adding the antibiotics. The same experiment as the above was performed in the MG1655(DE3) ΔadhE::MVAbottom strain to confirm the possibility that such an increase in the santalene production was caused by the MVA lower pathway additionally introduced into the chromosome. However, in this case, it exhibited an aspect that the santalene could not be produced.

TABLE 3

| | Strain (48 h) | | | |
|---|---|---|---|---|
| | MG1655 | MG1655(DE3) | MG1655(DE3) Δdxr | |
| | (DE3) | ΔadhE::MVAbottom | ΔadhE::MVAbottom | |
| | pTAS-NA | pTAS-NA | pTAS-NA | |
| | Antibiotic addition | | | |
| | − | + | + | − |
| Cell growth | 10.9 ± 0.5 | 9.1 ± 1.0 | 10.7 ± 0.3 | 12.7 ± 0.3 |
| (OD _{600 nm}) | | | | |
| Santalene | 79.8 ± 0.9 | 2.9 ± 1.3 | 420.2 ± 69.3 | 646.7 ± 6.6 |
| (mg/L) | | | | |

B. Multiple Plasmid Method (Example of Producing Retinoid)

This is a method in which the MVA pathways that compensate for the MEP pathway deletion of the host are distributed and present in a plurality of plasmids, so as to activate the MVA pathway only when all the plurality of plasmids are present. That is, it is constructed so that the MVA pathway and the biosynthetic pathway of the target product are efficiently distributed and arranged in these plasmids in consideration of a size of each plasmid and a required expression amount of constitutive genes.

i. Construction of Plasmid

As a foreign MVA pathway-based plasmid selection marker that can be used in a defective strain having the MVA lower pathway introduced therein into the chromosome, there are mvaE gene and mvaS gene, which are MVA upper pathway genes. The mvaE gene is a gene in which an atoB gene and the mvaA gene are fused, and if necessary, it may be divided into the two genes to be used as a selection marker. In this experiment, a plasmid system to maintain both plasmids under a non-antibiotic condition was constructed using pSNAK plasmid which expresses the foreign MVA pathway and pT-DHBSRYbbO which produces retinoid. The selection markers mvaE gene and mvaS gene are distributed and arranged in two plasmids so that both plasmids are introduced together to compensate for the deletion of the host's MEP pathway. Since one MVA pathway selection marker gene has to be moved from a pSNAK plasmid with a relatively low copy number and lac promoter to the pT-DHBSRYbbO plasmid with a high copy number and trc promoter, the mvaE gene, which requires higher expression amount, was transferred. That is, the mvaS gene is used as a selection marker of a plasmid expressing the MVA pathway except for mvaE, and the mvaE gene serves as a selection marker of a retinoid producing plasmid.

The restriction enzyme site HpaI of the pSNAK plasmid was used to construct a pSNAK(-E) plasmid from which the mvaE gene is removed by self-ligation after cleaving the mvaE gene. In the pSNAK(-E) plasmid, a Kanamycin antibiotic marker gene was removed by PCR using primers of SEQ ID NO: 29 and SEQ ID NO: 30 having a phosphate group at 5′ end, and then pSNA(-E) free was constructed by self-ligation.

pT-DHBSRYbbO is a plasmid in which Pantoea agglomerans-derived crtE, crtB and crtI, Pantoea ananatis-derived crtY, dxs and YbbO of E. coli, and codon-optimized uncultured marine bacterium 66A03-derived SR gene is introduced into pTrc99A vector. In the above plasmid, the dxs gene was removed by PCR using primers of SEQ ID NOs: 31 and 32 having a phosphate group at 5′ end to construct pT-HBSRYbbO.

The pTEFAmvaE plasmid is a plasmid prepared by introducing mvaE gene of Enterococcus faecalis into pTrc99A vector, and serves to amplify the trc promoter and mvaE gene together using the above plasmid as a template by PCR using primers of SEQ ID NOs: 33 and 34. This plasmid was cleaved with restriction enzymes NotI and SalI and inserted into the same restriction enzyme site of the pT-HBSRYbbO vector to prepare a plasmid pT-HBSREYbbO positioned between the idi gene (ipiHP1) and the crtY gene. pT-HBSREYbbOfree was constructed by introducing HindIII restriction enzyme sites after removing the ampicillin antibiotic marker through two PCRs using primers of SEQ ID NOs: 35 and 36 and SEQ ID NOs: 37 and 38 in pT-HBSREYbbO.

ii. Introduction and Culture Results of Plasmid

(1) New recombinant strain that maintains the plasmid in a non-antibiotic medium was constructed by introducing new two plasmids pSNAK(-E) and pT-HBSREYbbO, from which the antibiotic marker is not removed, and pT-HBSREYbbO together into MG1655(DE3) Δdxr ΔadhE::MVAbottom strain. Retinol production of new the recombinant strain with or without the addition of the antibiotics was confirmed using MG1655(DE3) strains containing the existing pSNAK and pT-DHBSRYbbO plasmids as a control group.

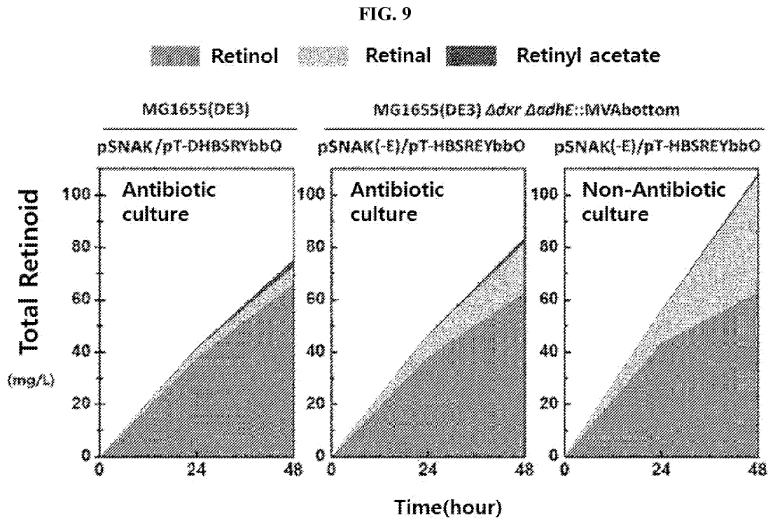

The culture results are shown in Table 4 below and FIG. 9. More specifically, it can be seen that the production of the retinoid was increased in the new recombinant strain MG1655(DE3) Δdxr ΔadhE::MVAbottom than the existing MG1655(DE3) strain. Comparing the culture results with or without antibiotics of the new strain, cell growth in a non-antibiotice condition was slightly lower, but the production of retinoid was higher than those of the case in an antibiotice condition for 48 hours of cultivation. Front these results, it can be seen that the plasmid is well maintained and functioned with no loss thereof without a selection pressure for antibiotics, and that metabolic flow is also more directed toward the retinoid production from the cell growth. In addition, it can be seen that the difference in the production of retinoid with or without antibiotics is caused by the fact that the antibiotic resistance gene expressed in the plasmid does not completely compensate for the toxicity of the antibiotics. The antibiotics used in this cultivation are ampicillin and kanamycin, wherein the ampicillin serves to inhibit the synthesis of cell walls, and the kanamycin serves to inhibit 30s subunits of ribosome, thereby reducing overall production of proteins. Therefore, it is determined that, in the cultivation under the non-antibiotic condition, the protein synthesis is better performed due to an absence of a factor that adversely affects the cells, thus to increase the production.

TABLE 4

| | Strain (48 h) | | |
|---|---|---|---|
| | MG1655(DE3) | MG1655(DE3) Δdxr | |
| | pSANK/pT- | ΔadhE::MVAbottom | |
| | DHBSRYbbO | pSNAK(-E)/pT-HBSREYbbO | |
| | Antibiotic addition | | |
| | + | + | − |
| Cell growth | 18.3 ± 0.5 | 11.4 ± 0.1 | 11.5 ± 0.2 |
| (OD _{600 nm}) | | | |
| Total retinoid | 75.4 ± 0.1 | 83.7 ± 2 | 108 ± 1 |
| (mg/L) | | | |

(2) Further, cultivation was performed under a non-antibiotic condition by transforming plasmids from which antibiotic resistance genes were removed, pSNA(-E)free and pT-HBSREYbbOfree together into MG1655(DE3) Δdxr ΔadhE::MVAbottom strain. The cultivation was performed under a retinoid culture condition up to 72 hours which is a maximum time for test tube culture of E. coli.

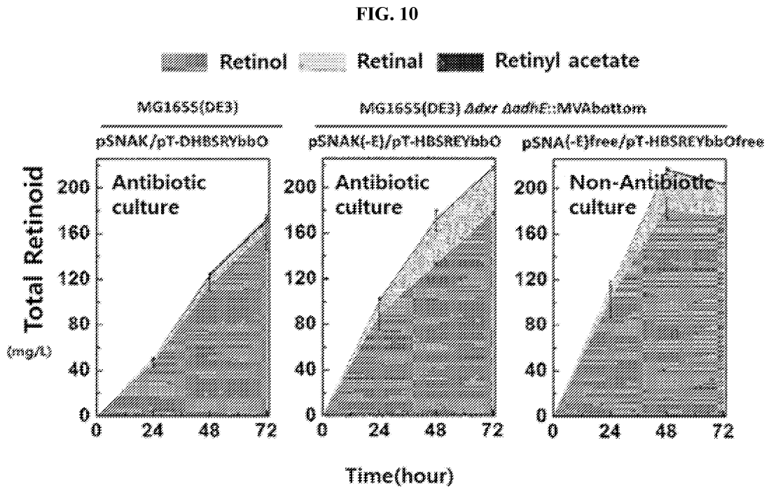

The culture results are shown in Table 5 below and FIG. 10. More specifically, it can be seen that the recombinant strain including the plasmid introduced therein, from which the antibiotic marker is completely removed, exhibits the fastest production rate of retinoid, and reaches the maximum production at 48 hours, while slightly decreasing at 72 hours due to depletion of the carbon source. Therefore, it is estimated that the metabolic burden on the host cell is reduced by removing the antibiotic marker from the plasmid.

TABLE 5

| | Strain (72 h) | | |
|---|---|---|---|
| | | MG1655(DE3) Δdxr | |
| | MG1655(DE3) | ΔadhE::MVAbottom | |
| | pSANK/pT- | pSNAK(-E)/pT- | pSNA(-E)free/pT- |
| | DHBSRYbbO | HBSREYbbO | HBSREYbbOfree |
| | Antibiotic addition | | |
| | + | − | − |
| Cell growth | 22.1 ± 0.03 | 12.9 ± 0.08 | 13.0 ± 0.4 |
| (OD _{600 nm}) | | | |
| Total retinoid | 173.3 ± 6 | 218.1 ± 1 | 204.2 ± 3 |
| (mg/L) | | | |

2) MEP Pathway-Based Isoprenoid Biosynthetic Plasmid Using Deleted MEP Pathway Gene of Chromosome as a Selection Marker

A. Single Plasmid Method (Example of Producing Santalene)

This is a method of constructing a single plasmid having the deleted MEP pathway gene of a chromosome as a selection marker and introducing it into the host, and in this case, the host's MEP pathway deletion is complemented by the introduced plasmid.

i. Construction of Plasmid

pTAS-dxr was constructed by introducing a dxr, which is the deleted MEP pathway gene in the defective strain, behind a santalene biosynthetic operon of pT-ispA-STS, which is the santalene producing plasmid. More specifically, restriction enzyme sites BglII and XhoI were introduced into both ends of the dxr gene and amplified using a pT-dxr plasmid having a dxr gene of E. coli introduced into pTrc99A plasmid as a template, by PCR using the primers of SEQ ID NO: 39 and SEQ ID NO: 40. Using this, a santalene producing plasmid pTAS-dxr having the deleted dxr gene was constructed by cloning with the same restriction enzyme sites BglII and SalI located behind the STS gene of the pT-ispA-STS vector. Restriction enzymes SalI and XhoI have the same cohesive end as each other.

In the cloning process of the plasmid above, for selection of transformants, E. coli DH5α or E. coli MG1655(DE3) Δdxr ΔadhE::MVAbottom may be used. When using E. coli DH5u, it is possible to select on an ampicillin plate by using an antibiotic marker present in the plasmid to be constructed. When using E. coli MG1655(DE3) Δdxr ΔadhE::MVAbottom, the transformants may be selected without antibiotics, because the strain is capable of growing when the MEP pathway of host E. coli is inactivated and a plasmid with activated MVA pathway is introduced.

ii. Introduction of Plasmid

The constructed plasmid pTAS-dxr was may be introduced into MG1655(DE3) Δdxr ΔadhE::MVAbottom strain, in which the MEP pathway was blocked, to obtain a strain from a mevalonate-free plate, thereby selecting an inherent MEP pathway using the dxr gene of the plasmid. Thereafter, by performing cultivation of the strain in anon-antibiotic medium to confirm the production of santalene, it is possible to confirm maintenance and activation abilities of the plasmid using the deleted MEP pathway gene.

Recombinant strain having a plasmid related to antibiotic-free santalene production was spawn cultured under a non-antibiotic condition, followed by culturing in a mixed medium of 4 mL of 2YT medium (16 g of trypton per liter, 10 g of yeast extract, and 5 g of NaCl) containing 2% (v/v) of glycerol and 0.2 mM of IPTG and 1 mL of decane, which is a production medium. For cultivation, the mixed medium is put into a tube having a groove of 15 cm in length and 25 mm in diameter and is inoculated with each of the strains, followed by culturing in a shaking incubator at 30° C. while stirring at a speed of about 250 rpm.

iii. Culture Result

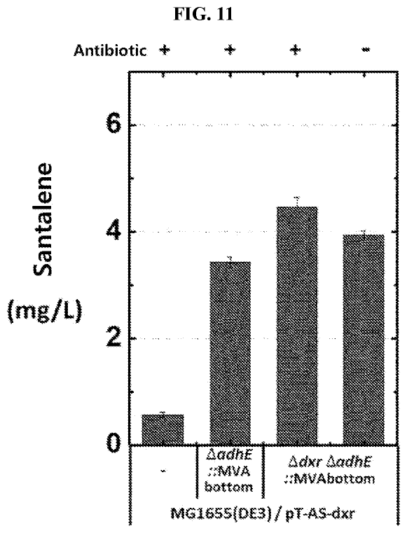

More specifically, the santalene productions were compared in MG1655(DE3) Δdxr ΔadhE::MVAbottom, a newly constructed recombinant strain using wild type E. coli MG155 (DE3) strain as a control group. The culture results are shown in FIG. 11 and Table 6 below. More specifically, the cultivation was performed in such a way that MG1655(DE3) and MG1655(DE3) ΔadhE::MVAbottom strains were cultured by adding antibiotics thereto, and MG1655(DE3) Δdxr ΔadhE::MVAbottom, to which an antibiotic-free system can be applied, was cultured by changing with or without the addition of the antibiotics. After 48 hours from the cultivation, the decane layer was recovered and the santalene production was analyzed through GC. As a result, MG1655(DE3) produced 0.6 mg/L of santalene, whereas the newly constructed MG1655(DE3) Δdxr ΔadhE::MVAbottom strain produced 4.5 mg/L of santalene. The production of santalene in the MG1655(DE3) ΔadhE::MVAbottom strain was also 3.4 mg/L, which is a greater amount of the produced santalene than that of the wild type strain. The increased amount of santalene may be due to the additional introduction of the MVA lower pathway, but it is difficult to see as an increase in IPP and DMAPP through the MVA lower pathway because no mevalonate was additionally introduced. Depletion of adhE causes Acetyl-CoA to accumulate without producing ethanol of a by-product, which may alter the flow of glycolysis in the corresponding process and cause an increase in the amount of precursor G3P and Pyruvate in the MEP pathway. Therefore, the production of santalene might be increased.

TABLE 6

| | Strain (48 h) | | | |
|---|---|---|---|---|
| | | MG1655(DE3) | MG1655(DE3) Δdxr | |
| | MG1655(DE3) | ΔadhE::MVAbottom | ΔadhE::MVAbottom | |
| | pTAS-dxr | pTAS-dxr | pTAS-dxr | |
| | Antibiotic addition | | | |
| | + | + | + | − |
| Cell growth | 4.9 ± 0.1 | 5.5 ± 0.2 | 5.0 ± 0.2 | 4.8 ± 0.2 |
| (OD _{600 nm}) | | | | |
| Santalene | 0.6 ± 0.0 | 3.4 ± 0.1 | 4.5 ± 0.2 | 3.9 ± 0.1 |
| (mg/L) | | | | |

B. Multiple Plasmid Method (Example of Producing Santalene+Bisabolol)

This is a method of constructing a plurality of plasmids having selection markers of the deleted MEP pathway genes of the chromosome and introducing into the host. In this case, to compensate for the host's MEP pathway deletion only when all the plurality of plasmids are present, it is necessary for the deleted MEP pathway genes to be evenly distributed in each plasmid. In particular, in a defective strain used as a host strain, the MEP pathway genes of the chromosome should be deleted by more than the number of plasmids to be introduced. In other words, when using two plasmids, at least two chromosome MEP pathway genes should be deleted and these genes are distributed and arranged in each plasmid having a selection marker.

i. Construction of Plasmid

pTAS-dxs were constructed by introducing a dxs gene behind a santalene biosynthetic operon of pT-ispA-STS, which is the santalene producing plasmid. More specifically, restriction enzyme sites BglII and XhoI were introduced into both ends of the dxs gene and amplified using a pT-dxs/r plasmid having dxs gene and dxr gene of E. coli introduced into pTrc99A vector as a template, by PCR using the primers of SEQ ID NO: 41 and SEQ ID NO: 42. Using this, a santalene producing plasmid pTAS-dxs having the deleted dxr gene was constructed by cloning with the same restriction enzyme sites BglII and SalI located behind the STS gene of the pT-ispA-STS vector. Restriction enzymes SalI and XhoI have the same cohesive end as each other.

Similarly, pTAB-idi-dxr was constructed by introducing dxr gene and idi gene (b2889) behind a bisabolol biosynthetic operon of pT-ispA-MrBBS, which is the bisabolol producing plasmid including an ispA gene that is FPP Synthase of E. coli and MrBBS that is α-bisabolol synthase of Matricaria recutita into pTrc99A vector. The idi gene is able to improve the production of isoprenoid by balancing IPP and DMAPP, when the MEP pathway is enhanced by the plasmid constructed with IPP isomerase. More specifically, in the construction process, by using restriction enzyme sites BglII and SalI located behind the MrBBS gene of the pT-ispA-MrBBS vector, the PTAB-idi-dxr was constructed by introducing the idi gene amplified while introducing restriction enzyme sites BamHI and SalI by PCR using primers of SEQ ID NO: 43 and SEQ ID NO: 44 and the dxr gene amplified while introducing restriction enzyme sites SalI and HindIII by PCR using primers of SEQ ID NO: 45 and SEQ ID NO: 46 into the bisabolol producing plasmid.

In the cloning process of the plasmid above, for selection of transformants, E. coli MG1655(DE3) Δdxr/s ΔadhE::MVAbottom may be used. When using E. coli MG1655(DE3) Δdxr/s ΔadhE::MVAbottom, the transformants may be selected without antibiotics, because the strain is capable of growing when the MEP pathway of host E. coli is inactivated and two plasmids respectively having the dxr gene and the dxs gene introduced therein are introduced together.

ii. Introduction of Plasmid

A recombinant strain was constructed by transforming plasmid pTAS-dxs having the constructed dxs gene as the selection marker and the plasmid pTAB-idi-dxr having the dxr gene as the selection marker together into MG1655(DE3) Δdxr/s ΔadhE::MVAbottom strain in which both the dxr and dxs genes are deleted. Thereafter, by culturing the recombinant strain in a non-antibiotic medium environment to confirm the productions of santalene and bisabolol, it can be confirmed that each of the deleted MEP genes are operated as a selection marker of a plurality of plasmids.

The recombinant strain having the plurality of plasmids was spawn cultured under a non-antibiotic condition, followed by culturing in a mixed medium of 4 mL of 2YT medium (16 g of trypton per liter, 10 g of yeast extract, and 5 g of NaCl) containing 2% (v/v) of glycerol and 0.2 mM of IPTG and 1 mL of decane, which is a production medium. For cultivation, the mixed medium is put into a tube having a groove of 15 cm in length and 25 mm in diameter and is inoculated with each of the strains, followed by culturing in a shaking incubator at 30° C. while stirring at a speed of about 250 rpm.

iii. Culture Result

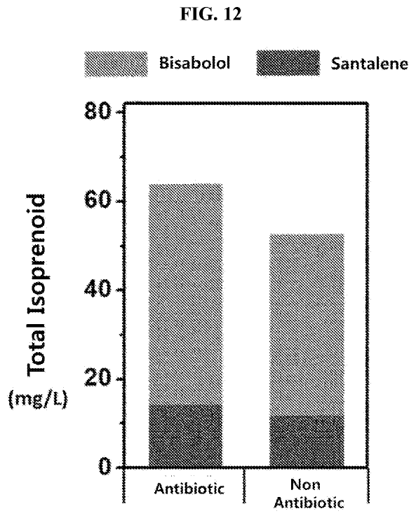

The constructed recombinant strain was cultured using the medium with or without the addition of antibiotics. After 58 hours from the cultivation, the decane layer was recovered and productions of the santalene and bisabolol were analyzed through GC. The culture results are shown in FIG. 12 and Table 7 below. More specifically, santalene and bisabolol were produced in amounts of 7.7 mg/L and 40.7 mg/L, respectively, even in without the addition of antibiotics. Through this, it can be seen that both plasmids respectively having a santalene biosynthetic gene and a bisabolol biosynthetic gene are well maintained to express genes.

TABLE 7

| | Strain (58 h) | |
|---|---|---|
| | MG1655(DE3) Δdxs/r ΔadhE::MVAbottom | |
| | pTAS-dxs/pTAB-idi-dxr | |
| | Antibiotic addition | |
| | + | − |
| Cell growth (OD _{600 nm}) | 10.1 ± 0.8 | 10.7 ± 0.7 |
| Santalene (mg/L) | 14.3 ± 1.6 | 7.7 ± 2.5 |
| Bisabolor (mg/L) | 49.7 ± 5.0 | 40.7 ± 7.1 |

C. Method of Adding Foreign MVA Pathway (Example of Producing Retinoid)
Based on the preceding results that the introduction of foreign MVA pathways into isoprenoid production is advantageous, the present invention provides a method of using MEM pathway-defective genes as a selection marker while introducing the foreign MVA pathways into plasmids. In this case, since both the MEP and MVA pathways are activated due to the host strain, it may be expected to achieve high production of isoprenoid. However, when using a plurality of plasmids, it is necessary for the foreign MVA pathway to be additionally introduced to be evenly distributed in the used plasmids without being concentrated on any one side, so as to compensate for the host's MEP pathway deletion only when all the plurality of plasmids are present.

i. Construction of Plasmid

By using a non-antibiotic retinoid producing strain that separately expresses the constructed MVA pathway gene in two plasmids, a recombinant plasmid using the foreign MVA pathway is also constructed while using the defective gene of the MEP pathway as a selection marker. Herein, a dxr gene with a relatively small size was introduced into retinoid producing plasmid pT-HBSREYbbOfree into which the mvaE gene with the large plasmid size is introduced, and a dxs gene was introduced into plasmid pSNAK(-E) that expresses the MVA pathway except for the mvaE gene having a size margin.

BglII and XhoI were introduced into both ends of the dxs, which is an MEP upper pathway gene, and amplified, using pT-dxs/r plasmid as a template by PCR using primers of SEQ ID NO: 47 and SEQ ID NO: 48. Further, restriction enzyme sequences BglII and XhoI were introduced into both ends thereof to amplify the vector using pSNAK(-E) as a template by PCR using primers of SEQ ID NO: 49 and SEQ ID NO: 50. Two PCR products were cleaved with restriction enzymes BglII and XhoI, then the dxs gene is inserted between the idi gene (b2889) and the mvaS gene of the vector to construct pSNAK(-E)-dxs. Then, pSNA(-E)-dxs_{free} was constructed by removing kanamycin antibiotic resistance gene by PCR using the antibiotic removal primer.

Restriction enzymes NheI and ScaI were introduced into both ends of dxr, which is the upper MEP pathway gene, and amplified using pT-dxr plasmid as a template by PCR using primers of SEQ ID NO: 51 and SEQ ID NO: 52. Then, pT-HBSREYbbOfree plasmid was amplified into two fragments, 7.1 kb and 5.6 kb each, using the restriction enzyme XhoI site as a starting point, through two PCRs using primers of SEQ ID NO: 36 and SEQ ID NO: 53, and SEQ ID NO: 37 and SEQ ID NO: 54. The two fragments were linked again by xhoI, and the dxr gene is cleaved with restriction enzymes ScaI and NheI and inserted into an end of the retinoid operon to construct pT-HBSREYbbOdxr_{free}.

ii. Introduction of Plasmid

pS-HBSREYbbOdxrfree plasmid having dxr gene of MEP pathway and mvaE gene of the MVA pathway, and pSNA(-E)-dxs_{free} having all MVA pathway genes except for the dxs gene of the MEP pathway and mvaE gene were introduced together into E. coli MG1655(DE3) Δdxr/s ΔadhE::MVAbottom, to confirm retinol production of non-antibiotic retinol producing recombinant strain simultaneously using the MVA and MEP pathways.

iii. Culture Result

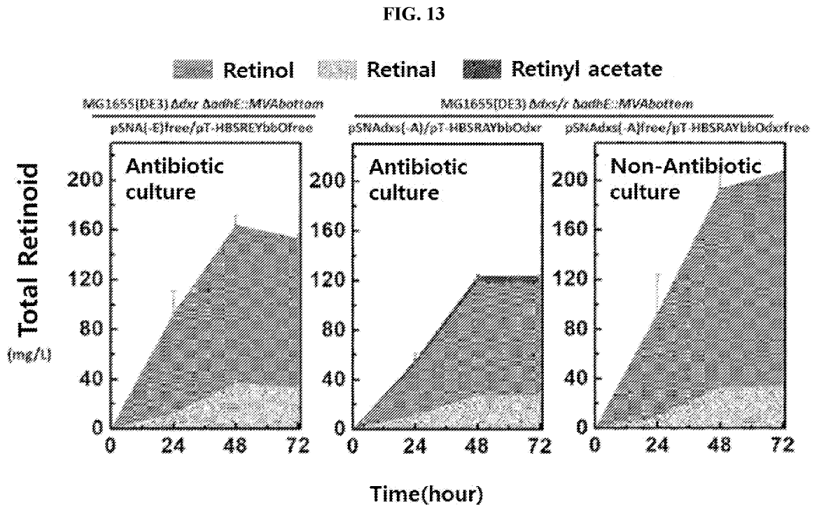

An antibiotic-free retinol producing recombinant strain simultaneously using the MVA pathway and the MEP pathway together with a non-antibiotic retinol producing recombinant strain using only the foreign MVA pathway in the MEP pathway-defective strain were cultured in a non-antibiotic condition, to compare the retinol production. The culture results are shown in FIG. 13 and Table 8 below. More specifically, when removing the antibiotic resistance gene by the non-antibiotic resistance retinol producing recombinant strain simultaneously using the constructed MVA and MEP pathways, a higher retinol production was shown than the retinoid producing strain in which the deletion of the MEP pathway was compensated only by the foreign MVA pathway. As a result, it was confirmed that the deleted MEP pathway gene could be used as a selection marker while additionally compensating the foreign MVA pathway.

TABLE 8

| | Strain (72 h) | | |
|---|---|---|---|
| | MG1655(DE3) Δdxr | MG1655(DE3) Δdxs/r | |
| | ΔadhE::MVAbottom | ΔadhE::MVAbottom | |
| | pSNA(-E)free/pT- | pSNAdxs(-A)/pT- | pSNAdxs(-A)free/pT- |
| | HBSREYbbOfree | HBSRAYbbOdxr | HBSRAYbbOdxrfree |
| | Antibiotic addition | | |
| | − | − | − |
| Cell | 12.7 ± 0.24 | 12.6 ± 0.28 | 12.1 ± 0.12 |
| growth | | | |
| (OD _{600 nm}) | | | |
| Total | 153.2 ± 2 | 124.0 ± 3 | 207.2 ± 1 |
| retinoid | | | |
| (mg/L) | | | |

3) Plasmid for Expression of Protein Using Deleted MEP Pathway Gene in Chromosome as Selection Marker
Confirmation of stability in expression of the protein using GFP protein
It is not limited to the expression of a single protein but may also be used to express a variety of metabolite biosynthetic pathways.

i. Construction of Plasmid

PEGFP-dxr was constructed by introducing dxr, which is the deleted MEP pathway gene of defective strain, behind EGFP gene of vector pEGFP expressing green fluorescence. More specifically, by using restriction enzyme sites StuI and SpeI behind the EGFP gene of the vector, the dxr gene amplified by PCR using primers of SEQ ID NO: 51 and SEQ ID NO: 52 was introduced. Restriction enzyme sites ScaI and NheI are introduced into both ends of the amplified PCR product. Restriction enzymes StuI and ScaI have a blunt end, respectively, and restriction enzymes SpeI and NheI have the same cohesive end as each other. Finally, pEGFP-dxr, which is a plasmid having a dxr gene expressing green fluorescence, was constructed.

ii. Introduction of Plasmid

The constructed pEGFP-dxr plasmid is transformed into MG1655(DE3) Δdxr ΔadhE::MVAbottom strain. By smearing it on an LB plate medium without antibiotics, fluorescence was observed.

In order to ensure the expression of a target protein of the constructed recombinant strain, 1 mM of IPTG as an inducer is added and cultured. More specifically, the strain was spawn cultured under a non-antibiotic condition, and inoculated in 5 ml of LB medium which is the production medium, and cultured so as to be OD_{600 nm} 0.1. After the cultivation, 1 mM of IPTG was added thereto at the time to be OD_{600 nm} 0.6. For cultivation, the mixed medium was put into a tube having a groove of 15 cm in length and 25 mm in diameter and is inoculated with each of the strains, followed by culturing in a shaking incubator at 30° C. while stirring at a speed of about 250 rpm. The culture liquid was collected, and the stability in expression of the genes contained in the plasmid was confirmed under the non-antibiotic medium condition by SDS-PAGE and fluorescence measurement.

iii. Culture Result

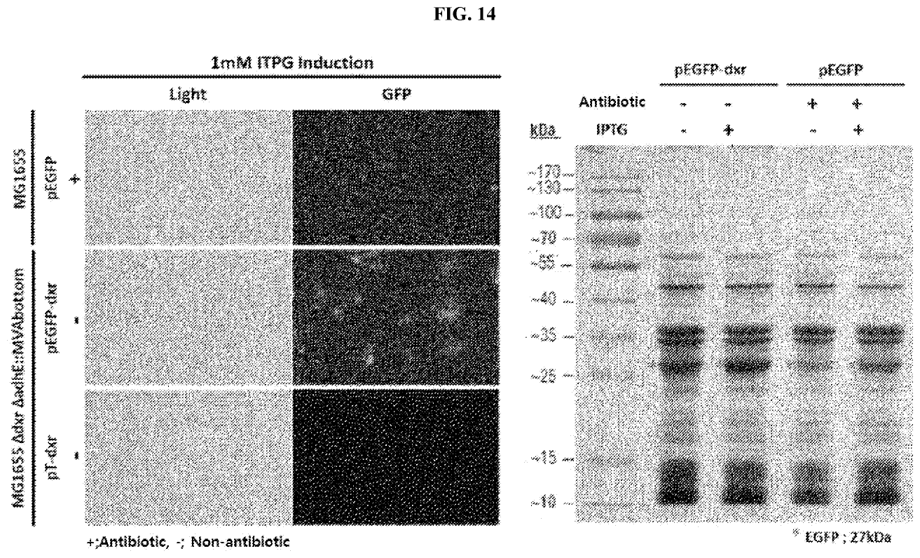

SDS-PAGE and fluorescence results are shown in FIG. 14. More specifically, as a result of observing green fluorescence by a fluorescence microscope, it was confirmed that clearer fluorescence was obtained in a case of using the deleted MEP pathway gene as the selection marker of the plasmid than the control group including the antibiotics as the selection marker. In addition, comparing the amount of protein expression through the SDS-PAGE, a larger amount of protein expression exhibited in the non-antibiotic culture of the newly constructed strain than the control group. It was confirmed that, even without treatment with an inducer such as IPTG, the plasmid was sufficiently maintained by the selection pressure of the deleted MEP pathway gene of the strain to express the gene.

4. Method of Constructing MVA Pathway Mutant

1) Inactivation of Gene Encoding Lactobacillus HMG-coA Reductase (mvaA)

A rate-limiting enzyme, Hydroxymethylglutaryl-CoA reductase (mvaA) gene, was inactivated using a homologous recombination system in an inherent MVA pathway of lactobacillus. For mvaA gene mutation of Lactococcus lactis MG1363, a front 1Kb portion of the mvaA gene in MG1363 was amplified by polymerase chain reaction (PCR) using primers of SEQ ID NO: 55 and SEQ ID NO: 56, and a rear 1Kb portion after the mvaA gene was amplified using primers of SEQ ID NO: 57 and SEQ ID NO: 58, then two PCR products were amplified using primers of SEQ ID NO: 59 and SEQ ID NO: 60 through splicing by overhang extension (SOE) PCR. In addition, pORI19 plasmid was amplified by PCR using primers of SEQ ID NO: 61 and SEQ ID NO: 62, followed by ligation of the two PCR products, then electroporation was performed on 100 μl of EC1000 competent cell through a cuvette at an interval of 2 mm under conditions of 25 μF, 200Ω and 2,500 V. After performing the electroporation, 1 ml of LB medium was added thereto, followed by culturing at 37° C. for 30 minutes, and then 100 μl thereof was smeared on an LB solid plate medium containing 300 μg/ml erythromycin antibiotic. Finally, pORI19-mvaA plasmid was obtained by culturing at 37° C. for 12 hours (Table 2).

In order to prepare a competent cell of lactobacillus, L. lactis MG1363 (pVE6007) strain containing plasmid pVE6007 was inoculated in 5 ml of M17 medium (5.0 g of Pancreatic Digest of Casein, 5.0 g of soy peptone, 5.0 g of beef extract, 2.5 g of yeast extract, 0.5 g of ascorbic acid, 0.25 g of magnesium sulfate, and 10.0 g of disodium-β-glycerophosphate per liter) containing 0.5% (v/v) glucose and 5 μg/ml chloramphenicol antibiotic added thereto, followed by spawn culturing at 30° C. for 16-24 hours. 1 ml of a spawn culture liquid was inoculated in 9 ml of M17 medium containing 0.5% (v/v) of glucose, 0.5 M of sucrose, 1.5% (w/v) of glycine and 5 μg/ml of chloramphenicol, followed by culturing at 30° C. for 16-24 hours. 5 ml of the spawn culture liquid was inoculated in 35 ml of the same fresh medium, followed by culturing again at 30° C. to OD_{600 nm}, 0.25. After cooling the spawn culture liquid on ice for 5 minutes to form the competent cell, a supernatant was removed by performing centrifugation at 4° C., 5,000 rpm for 15 minutes, and washed twice with 40 ml of wash buffer (0.5 M of sucrose, and 10% of glycerol) in the same amount as the culture liquid. The washed cells were suspended in 0.4 ml of wash buffer to obtain the competent cells.

3-5 μg of pORI19-mvaA plasmid was put into 100 ul of the obtained competent cells, and was subjected to electroporation through a cuvette at an interval of 2 mm under conditions of 25 μF, 200Ω and 2,500 V. After the electroporation, 1 ml of GM17 medium added with 5 μg/ml of chloramphenicol was added thereto, followed by culturing at 30° C. for 2 hours, and then 100 μl thereof was smeared on a GM17 solid plate medium containing 5 μg/ml of chloramphenicol and 5 μg/ml of erythromycin added thereto, to obtain MG1363 (pORI19-mvaA, pVE6007) strain.

In order to remove pVE6007 plasmid, the strains obtained above were inoculated in 5 ml of GM17 medium containing 5 μg/ml of chloramphenicol and 5 μg/ml of erythromycin added thereto, followed by culturing at 30° C. for 16-24 hours and performing cell down on 1 ml of culture liquid to wash twice with 1 ml of GM17 medium, then 10 ml of GM17 medium containing 5 μg/ml of erythromycin added thereto was inoculated in an amount of 0.1% (v/v), followed by culturing at 30° C. for 16-24 hours. After performing subculture 3 times by inoculating 0.10% (v/v) thereof in the same medium at 37° C. with an interval of 12 hours, and then diluted in 10⁵⁻⁷ and smeared on a GM17 solid plate medium containing 5 μg/ml of erythromycin, followed by culturing at 30° C. for 16-24 hours.

Strains in which single cross over (SCO) occurred were selected by PCR. PCR was performed using a total of three primers in which 200 colonies were additionally added with a primer of SEQ ID NO: 65 for upstream confirmation and a primer of SEQ ID NO: 66 for downstream confirmation together with primers of SEQ ID NO: 63 and SEQ ID NO: 64, respectively. When the SCO occurs in the upstream, it can be confirmed by the primers of SEQ ID NO: 63 and SEQ ID NO: 65, and when the SCO occurs in the downstream, it can be confirmed by the primers of SEQ ID NO: 64 and SEQ ID NO: 66. In addition, in a case of the wild type without SCO occurred therein, it can be confirmed by the primers of SEQ ID NO: 63 and SEQ ID NO: 64. The strains in which the SCO occurred were inoculated in 5 ml of GM17 medium containing 5 μg/ml of erythromycin, followed by culturing at 30° C. for 16-24 hours and performing cell down with 1 ml of culture liquid to wash twice with 1 ml of GM17 medium, then 10 ml of GM17 medium containing 3.3 mM of mevalonate added thereto was inoculated in an amount of 0.1% (v/v), followed by culturing at 30° C. for 16-24 hours. Mevalonate was added to the medium for growth of the mutant of gene mvaA. After performing subculture 3 times by inoculating 0.1% (v/v) thereof in the same medium at 30° C. with an interval of 12 hours, and then diluted in 10⁵⁻⁷ and smeared on a GM17 solid plate medium containing 3.3 mM of mevalonate, followed by culturing at 30° C. for 16-24 hours.

Strains in which single cross over (SCO) occurred were selected by PCR. 400 colonies were subjected to PCR using primers of SEQ ID NO: 63 and SEQ ID NO: 64 to construct Lactococcus lactis MG1363ΔmvaA strain.

5. Methods of Constructing and Culturing Plasmid (MVA Pathway Mutant Strain)
Use of strains with mutated MVA upstream pathway gene in host chromosome

1) Construction of E. coli-lactobacillus recombinant shuttle vector The existing E. coli-lactobacillus shuttle vector pCI372 was cleaved with restriction enzymes AgeI and NheI, and a pCIN vector, into which a promoter, a multi-cloning site and a terminator were introduced, was constructed (Table 2).

2) Plasmid for Protein Expression Using Mutated MVA Pathway Gene of Chromosome as Selection Marker

i. Construction of Plasmid

The mvaA gene for use as a selection marker was amplified by polymerase chain reaction (PCR) using primers of SEQ ID NO: 67 and SEQ ID NO: 68 from MG1363 strain, and cleaved with restriction enzymes BamHI and XbaI, then pCIN-mvaA vector was constructed by introducing it into the same restriction enzyme site of pCIN vector. EGFP gene for confirmation of green fluorescent protein expression was amplified by polymerase chain reaction (PCR) using primers of SEQ ID NO: 69 and SEQ ID NO: 70 from pEGFP vector and cleaved with restriction enzymes SalI and SphI, then PCIN-mvaA-EGFP was constructed by introducing it into the same restriction enzyme site of pCIN-mvaA vector (FIG. 10). The above constructed vectors are shown in Table 2.

ii. Introduction of Plasmid

The pCIN-mvaA-EGFP recombinant plasmid was transformed into L. lactis MG1363ΔmvaA strain to prepare a lactobacillus transformant having mvaA as a selection marker.

A competent cell was prepared to transform the pCIN-mvaA-EGFP recombinant shuttle vector into L. lactis MG1363ΔmvaA strain. MG1363ΔmvaA strains were inoculated in 5 ml of M17 medium containing 0.5% (v/v) of glucose and 3.3 mM of mevalonate added thereto, followed by culturing at 30° C. for 16-24 hours. 1 ml of spawn culture liquid was inoculated in 9 ml of M17 medium containing 0.5% (v/v) of glucose, 0.5M of sucrose, 1.5% (w/v) of glycine and 3.3 mM of mevalonate added thereto, followed by culturing at 30° C. for 16-24 hours. 5 ml of the spawn culture liquid was inoculated in 35 ml of the same fresh medium, followed by culturing again at 30° C. to OD_{600 nm}, 0.25. After cooling the spawn culture liquid on ice for 5 minutes to form the competent cell, a supernatant was removed by performing centrifugation at 4° C., 5,000 rpm for 15 minutes, and washed twice with 40 ml of wash buffer (0.5 M of sucrose, and 10% of glycerol) in the same amount as the culture liquid. The washed cells were suspended in 0.4 ml of wash buffer to obtain the competent cells.

5 μl of pCIN-mvaA-EGFP plasmid was put into 100 μl of the obtained competent cells, and was subjected to electroporation through an electroporation cuvette (at an interval of 2 mm) under conditions of 25 μF, 200Ω and 2,500 V. After electroporation, 1 ml of M17 medium added with 0.5% (v/v) of glucose was added thereto, followed by culturing at 30° C. for 1 hour. Cell samples into which pCIN-mvaA-EGFP plasmid was introduced by electroporation were diluted in 10¹-10³ and smeared on a M17 solid plate medium containing 0.5% (v/v) glucose added thereto to obtain MG1363ΔmvaA (pCIN-mvaA-EGFP) transformant.

iii. Confirmation of Stability in Expression of Protein Using GFP Protein

The constructed MG1363ΔmvaA (pCIN-mvaA-EGFP) strains were smeared on a M17 solid plate medium containing 0.5% (v/v) of glucose added thereto without antibiotics to observe fluorescence.

In order to ensure the expression of the target protein of the constructed recombinant strain, cultivation thereof was performed. More specifically, strains were spawn cultured under a non-antibiotic condition, and inoculated in 5 ml of M17 medium containing 0.5% (v/v) of glucose added thereto, which is a production medium, to culture so as to be OD_{600 nm} 0.1, followed by culturing at 30° C. This culture liquid is collected and the stability in expression of the gene contained in the plasmid under the non-antibiotic medium condition was confirmed by fluorescence measurement.

TABLE 9

| SEQ ID NO. | Primer name |
|---|---|
| 1 | SN12Didi-F |
| 2 | SN12Didi-R |
| 3 | IS poxB-Ptrc-F |
| 4 | KO poxB -R |
| 5 | IS ldhA-Ptrc-F |
| 6 | KO ldhA-R |
| 7 | IS adhE-Ptrc-F |
| 8 | KO adhE-R |
| 9 | IS atoDA-Ptrc-F |
| 10 | KO atoDA-R |
| 11 | KOpoxBCF-F |
| 12 | KOpoxBCF-R |
| 13 | KOldhACF-F |
| 14 | KOldhACF-R |
| 15 | KOadhECF-F |
| 16 | KOadhECF-R |
| 17 | KOatoDACF-F |
| 18 | KOatoDACF-R |
| 19 | KO dxr-F |
| 20 | KO dxr-R |
| 21 | KOdxrCF-F |
| 22 | KOdxrCF-R |
| 23 | dxrCF-R |
| 24 | KO dxs-F |
| 25 | KO dxs-R |
| 26 | KOdxsCF-F |
| 27 | KOdxsCF-R |
| 28 | KmCF-F |
| 29 | dCmp(NA)-F |
| 30 | dCmp(NA)-R |
| 31 | ddxs(RET)-F |
| 32 | ddxs(RET)-R |
| 33 | P1mvaE-F |
| 34 | P1mvaE-R |
| 35 | dAmp(RET)-F |
| 36 | RET-R |
| 37 | RET-F |
| 38 | dAmp(RET)-R |
| 39 | dxr_1-F |
| 40 | dxr_1-R |
| 41 | dxs_1-F |
| 42 | dxs_1-R |
| 43 | idi-F |
| 44 | idi-R |
| 45 | dxr_3-F |
| 46 | dxr_3-R |
| 47 | dxs_2-F |
| 48 | dxs_2-R |
| 49 | dmvaE-F |
| 50 | dmvaE-R |
| 51 | dxr_2-F |
| 52 | dxr_2-R |
| 53 | RET_2-F |
| 54 | RET_2-R |
| 55 | mvaA u/s-Fwd |
| 56 | mvaA u/s-Rev |
| 57 | mvaA d/s-Fwd |
| 58 | mvaA d/s-Rev |
| 59 | mvaA ex-Fwd |
| 60 | mvaA ex-Rev |
| 61 | PORI19-Rev |
| 62 | PORI19-Fwd |
| 63 | DCO-Fwd |
| 64 | DCO-Rev |
| 65 | SCO u/s-Rev |
| 66 | SCO u/s-Fwd |
| 67 | mvaA-Fwd |
| 68 | mvaA-Rev |
| 69 | EGFP-Fwd |
| 70 | EGFP-Rev |

TABLE 10

| | | | | | Amino |
|---|---|---|---|---|---|
| | | | | Base | acid |
| | | | | sequence | sequence |
| Gene | | | Genbank | SEQ ID | SEQ ID |
| name | Enzyme | Origin | Grant NO. | NO. | NO. |
| mvaE | Acetyl-CoA | Enterococcus faecalis | AF290092 | 104 | 124 |
| | Acetyltransferase/ | | | | |
| | hydroxymethylglutaryl | | | | |
| | (HMG)-COA Reductase | | | | |
| mvaS | HMG-COA Synthase | Enterococcus faecalis | AF290092 | 105 | 125 |
| mvaK1 | Mevalonate kinase | Streptococcus | AF290099 | 106 | 126 |
| | | pneumoniae | | | |
| mvaK2 | Phosphomevalonate | Streptococcus | AF290099 | 107 | 127 |
| | Kinase | pneumoniae | | | |
| mvaD | Mevalonate diphosphate | Streptococcus | AF290099 | 108 | 128 |
| | decarboxylase | pneumoniae | | | |
| Idi | IPP Isomerase | Escherichia coli | U00096 | 109 | 129 |
| ipiHp1 | IPP Isoformerase | Haematococcuspluvialis | AF082325 | 110 | 130 |
| crtE | Geranylgeranyl | pantoea agglomerans | M87280 | 111 | 131 |
| | pyrophosphate (GGPP) | | | | |
| | synthase | | | | |
| crtB | Phytoen synthase | pantoea agglomerans | M87280 | 112 | 132 |
| crtI | Phytoene dehydrogenase | pantoea agglomerans | M87280 | 113 | 133 |
| crtY | Lycopene-β-cyclase | pantoea ananatis | D90087 | 114 | 134 |
| SR | β-carotene | unculturedmarine | E. coli codon | 115 | 135 |
| | monooxygenase | bacterium 66A03 | optimization | | |
| | | | sequence of | | |
| | | | blh | | |
| YbbO | Oxidoreductase | wild type Escherichia | 1786701 | 116 | 136 |
| | | coli MG1655; taxid | | | |
| | | 511145 | | | |
| dxs | 1-deoxyxylulose-5- | Escherichia coli | AF035440.1 | 117 | 137 |
| | phosphate (DXP) | | | | |
| | synthase | | | | |
| dxr | 1-deoxy-D-xylulose | Escherichia coli | AB013300.1 | 118 | 138 |
| | 5-phosphate | | | | |
| | reductoisomerase | | | | |
| ispA | Panesil Pyrophosphate | Escherichia coli | | 119 | 139 |
| | Synthase | | | | |
| STS | Santalene Synthase | Clausena lansium | | 120 | 140 |
| MrB | α-bisabolol synthase | Matricaria recutita | E. coli codon | 121 | 141 |
| BS | | | optimization | | |
| | | | sequence of | | |
| | | | KM259907.1 | | |
| EGFP | enhanced green | Synthetic construct | KX130867.1 | 122 | 142 |
| | fluorescent protein | | | | |
| mvaA | Hydroxymethylglutaryl- | Lactococcus lactis | (WP_ | 123 | 143 |
| | COA reductase | subsp. cremoris | 011834877.1) | | |
| | | MG1363 | | | |

### REFERENCE TO AN ELECTRONIC SEQUENCE LISTING

A sequence listing electronically submitted on May 24, 2023 as a XML file named 20230524_LC00319-V_TU_SEQ.XML, created on May 24, 2023 and having a size of 459,168 bytes, is incorporated herein by reference in its entirety.

What is claimed is:

1. A non-human organism transformed with a plasmid,
wherein, in the non-human organism, at least one of genes encoding enzymes in an isopentenyl diphosphate or dimethylallyl diphosphate synthetic pathway is attenuated or deleted;
a selection marker gene and a target product gene are introduced into the plasmid;
the selection marker gene comprises at least one of genes encoding the enzymes or a complimentary gene thereof; and
the target product gene comprises a gene encoding a target protein other than the enzymes in the isopentenyl diphosphate or the dimethylallyl diphosphate synthetic pathway.

2. The non-human organism according to claim 1, wherein the non-human organism inherently has the isopentenyl diphosphate or dimethylallyl diphosphate synthetic pathway.

3. The non-human organism according to claim 1, wherein the isopentenyl diphosphate or dimethylallyl diphosphate synthetic pathway is a methylerythritol 4-phosphate (MEP) pathway or a mevalonate (MVA) pathway.

4. The non-human organism according to claim 1, wherein the at least one of genes encoding the enzymes in the isopentenyl diphosphate or dimethylallyl diphosphate synthetic pathway is a gene encoding one or more enzymes selected from the group consisting of 1-dioxy-D-xylulose-5-phosphate (DXP) synthase, DXP reductoisomerase, 2-C-methyl-D-erythritol-4-phosphate (MEP) cytidyltransferase, 4-diphosphocytidyl-2-C-methyl-D-erythritol kinase, 2-C-methyl-D-erythritol-2,4-cyclodiphosphate (MEcPP) synthase, 4-hydroxy-3-methyl-2-butenyl diphosphate (HMBPP) synthase, HMBPP reductase, acetoacetyl-CoA synthase, 3-hydroxyl-3-methylglutary-CoA (HMG-CoA) synthase, HMG-CoA reductase, mevalonate kinase, mevalonate-5-phosphate kinase, mevalonate-5-diphosphate decarboxylase and isopentenyl pyrophosphate (IPP) isomerase.

5. The non-human organism according to claim 1, wherein the at least one of genes encoding the enzymes in the isopentenyl diphosphate or dimethylallyl diphosphate synthetic pathway is a gene encoding enzymes in the methylerythritol 4-phosphate (MEP) pathway.

6. The non-human organism according to claim 5, wherein the at least one of genes encoding the enzymes in the isopentenyl diphosphate or dimethylallyl diphosphate synthetic pathway is a gene encoding at least one of 1-dioxy-D-xylulose-5-phosphate (DXP) synthase and DXP reductoisomerase.

7. The non-human organism according to claim 1, wherein the at least one of genes encoding enzymes in an isopentenyl diphosphate or dimethylallyl diphosphate synthetic pathway is a gene encoding enzymes in the methylerythritol 4-phosphate (MEP) pathway, and the complementary gene is a gene encoding at least one of enzymes in the mevalonate (MVA) pathway.

8. The non-human organism according to claim 7, wherein the complementary gene is a gene encoding acetoacetyl-CoA synthase, 3-hydroxyl-3-methylglutary-CoA (HMG-CoA) synthase, HMG-CoA reductase, mevalonate kinase, mevalonate-5-phosphate kinase, mevalonate-5-diphosphate decarboxylase and isopentenyl pyrophosphate (IPP) isomerase.

9. The non-human organism according to claim 1, wherein the at least one of genes encoding the enzymes in the isopentenyl diphosphate or dimethylallyl diphosphate synthetic pathway is a gene encoding enzymes in the mevalonate (MVA) pathway.

10. The non-human organism according to claim 1, wherein the target product comprises an enzyme in a pathway selected from the group consisting of isoprenoid, santalene, bisabolol and retinol synthetic pathways.

11. The non-human organism according to claim 1, wherein the target protein is an enzyme in a target product synthetic pathway synthesizing the target product.

12. The non-human organism according to claim 1, wherein the target protein is green fluorescent protein (GFP).

13. The non-human organism according to claim 1, wherein the non-human organism is a microorganism.

14. The non-human organism according to claim 1, wherein the non-human organism is Lactococcus lactis or Escherichia coli.


SEQUENCE LISTING (143 sequences)

SEQ ID NO: 1 - DNA, 46 bp, synthetic construct
  misc_feature 1..46: note = SN12Didi-F
  source 1..46: mol_type = other DNA
gcgaattcaggaggtaataatatgacaaaaaaagttggtgtcggtc

SEQ ID NO: 2 - DNA, 39 bp, synthetic construct
  misc_feature 1..39: note = SN12Didi-R
  source 1..39: mol_type = other DNA
cgcctgcaggttatttaagctgggtaaatgcagataatc

SEQ ID NO: 3 - DNA, 74 bp, synthetic construct
  misc_feature 1..74: note = IS poxB-Ptrc-F
  source 1..74: mol_type = other DNA
gatgaactaaacttgttaccgttatcacattcaggagatggagaaccatggtttgacagcttatcatcgactgc

SEQ ID NO: 4 - DNA, 70 bp, synthetic construct
  misc_feature 1..70: note = KO poxB -R
  source 1..70: mol_type = other DNA
ccttattatgacgggaaatgccaccctttttaccttagccagtttgtttttgtaggctggagctgcttcg

SEQ ID NO: 5 - DNA, 74 bp, synthetic construct
  misc_feature 1..74: note = IS ldhA-Ptrc-F
  source 1..74: mol_type = other DNA
atttttagtagcttaaatgtgattcaacatcactggagaaagtcttatgagtttgacagcttatcatcgactgc

SEQ ID NO: 6 - DNA, 70 bp, synthetic construct
  misc_feature 1..70: note = KO ldhA-R
  source 1..70: mol_type = other DNA
ctcccctggaatgcaggggagcggcaagattaaaccagttcgttcgggcatgtaggctggagctgcttcg

SEQ ID NO: 7 - DNA, 74 bp, synthetic construct
  misc_feature 1..74: note = IS adhE-Ptrc-F
  source 1..74: mol_type = other DNA
cgagcagatgatttactaaaaaagtttaacattatcaggagagcattatggtttgacagcttatcatcgactgc

SEQ ID NO: 8 - DNA, 70 bp, synthetic construct
  misc_feature 1..70: note = KO adhE-R
  source 1..70: mol_type = other DNA
ccgtttatgttgccagacagcgctactgattaagcggattttttcgcttttgtaggctggagctgcttcg

SEQ ID NO: 9 - DNA, 74 bp, synthetic construct
  misc_feature 1..74: note = IS atoDA-Ptrc-F
  source 1..74: mol_type = other DNA
ctattgcctgactgtacccacaacggtgtatgcaagagggataaaaaatggtttgacagcttatcatcgactgc

SEQ ID NO: 10 - DNA, 70 bp, synthetic construct
  misc_feature 1..70: note = KO atoDA-R
  source 1..70: mol_type = other DNA
acgcgtcataaaacgcgatatgcgaccaatcataaatcaccccgttgcgttgtaggctggagctgcttcg

SEQ ID NO: 11 - DNA, 22 bp, synthetic construct
  misc_feature 1..22: note = KOpoxBCF-F
  source 1..22: mol_type = other DNA
ttacgtactggcctgctcctgc

SEQ ID NO: 12 - DNA, 22 bp, synthetic construct
  misc_feature 1..22: note = KOpoxBCF-R
  source 1..22: mol_type = other DNA
gtcgggtaacggtatcactgcg

SEQ ID NO: 13 - DNA, 20 bp, synthetic construct
  misc_feature 1..20: note = KOldhACF-F
  source 1..20: mol_type = other DNA
tcatcagcagcgtcaacggc

SEQ ID NO: 14 - DNA, 20 bp, synthetic construct
  misc_feature 1..20: note = KOldhACF-R
  source 1..20: mol_type = other DNA
cgctggtcacgggcttaccg

SEQ ID NO: 15 - DNA, 20 bp, synthetic construct
  misc_feature 1..20: note = KOadhECF-F
  source 1..20: mol_type = other DNA
ccgcactgactatactctcg

SEQ ID NO: 16 - DNA, 20 bp, synthetic construct
  misc_feature 1..20: note = KOadhECF-R
  source 1..20: mol_type = other DNA
tgatcggcattgcccagaag

SEQ ID NO: 17 - DNA, 22 bp, synthetic construct
  misc_feature 1..22: note = KOatoDACF-F
  source 1..22: mol_type = other DNA
ctggcgaggtaaaaacagcccc

SEQ ID NO: 18 - DNA, 22 bp, synthetic construct
  misc_feature 1..22: note = KOatoDACF-R
  source 1..22: mol_type = other DNA
aagcgcgatcacgaatgttagc

SEQ ID NO: 19 - DNA, 65 bp, synthetic construct
  misc_feature 1..65: note = KO dxr-F
  source 1..65: mol_type = other DNA
ggctggcggcgttttgctttttattctgtctcaactctggatgtttcatgggggatccgtcgacc

SEQ ID NO: 20 - DNA, 70 bp, synthetic construct
  misc_feature 1..70: note = KO dxr-R
  source 1..70: mol_type = other DNA
tagcgcgactctctgtagccggattatcctcagcttgcgagacgcatcactgtaggctggagctgcttcg

SEQ ID NO: 21 - DNA, 24 bp, synthetic construct
  misc_feature 1..24: note = KOdxrCF-F
  source 1..24: mol_type = other DNA
ctgtgtgtgactgtctggtctgac

SEQ ID NO: 22 - DNA, 27 bp, synthetic construct
  misc_feature 1..27: note = KOdxrCF-R
  source 1..27: mol_type = other DNA
catctgtaaagtaaagttcggcatagg

SEQ ID NO: 23 - DNA, 20 bp, synthetic construct
  misc_feature 1..20: note = dxrCF-R
  source 1..20: mol_type = other DNA
ccattttgctcaaggtcagc

SEQ ID NO: 24 - DNA, 70 bp, synthetic construct
  misc_feature 1..70: note = KO dxs-F
  source 1..70: mol_type = other DNA
tttcttaagcatagcaggagtggagtagggattatgccagccaggccttgcaattcgatggggatccgtc

SEQ ID NO: 25 - DNA, 70 bp, synthetic construct
  misc_feature 1..70: note = KO dxs-R
  source 1..70: mol_type = other DNA
gtattaataggcccctgatgagttttgatattgccaaatacccgaccctgtgtaggctggagctgcttcg

SEQ ID NO: 26 - DNA, 17 bp, synthetic construct
  misc_feature 1..17: note = KOdxsCF-F
  source 1..17: mol_type = other DNA
ggaaacgcgaaggtcgg

SEQ ID NO: 27 - DNA, 20 bp, synthetic construct
  misc_feature 1..20: note = KOdxsCF-R
  source 1..20: mol_type = other DNA
gcggactacatcatccagcg

SEQ ID NO: 28 - DNA, 20 bp, synthetic construct
  misc_feature 1..20: note = KmCF-F
  source 1..20: mol_type = other DNA
ccttgctcctgccgagaaag

SEQ ID NO: 29 - DNA, 23 bp, synthetic construct
  misc_feature 1..23: note = dCmp(NA)-F
  source 1..23: mol_type = other DNA
tttttttaaggcagttattggtg

SEQ ID NO: 30 - DNA, 20 bp, synthetic construct
  misc_feature 1..20: note = dCmp(NA)-R
  source 1..20: mol_type = other DNA
agcttccttagctcctgaaa

SEQ ID NO: 31 - DNA, 20 bp, synthetic construct
  misc_feature 1..20: note = ddxs(RET)-F
  source 1..20: mol_type = other DNA
gtacccagcttggctgtttt

SEQ ID NO: 32 - DNA, 20 bp, synthetic construct
  misc_feature 1..20: note = ddxs(RET)-R
  source 1..20: mol_type = other DNA
catggtgaattcctcctgct

SEQ ID NO: 33 - DNA, 32 bp, synthetic construct
  misc_feature 1..32: note = P1mvaE-F
  source 1..32: mol_type = other DNA
ttgcggccgcgctgttgacaattaatcatccg

SEQ ID NO: 34 - DNA, 30 bp, synthetic construct
  misc_feature 1..30: note = P1mvaE-R
  source 1..30: mol_type = other DNA
ttctcgagcctgaaacggctacctaatgtg

SEQ ID NO: 35 - DNA, 29 bp, synthetic construct
  misc_feature 1..29: note = dAmp(RET)-F
  source 1..29: mol_type = other DNA
cccaagcttcgtcagaccccgtagaaaag

SEQ ID NO: 36 - DNA, 23 bp, synthetic construct
  misc_feature 1..23: note = RET-R
  source 1..23: mol_type = other DNA
cctcgagttatcaggcgattttc

SEQ ID NO: 37 - DNA, 21 bp, synthetic construct
  misc_feature 1..21: note = RET-F
  source 1..21: mol_type = other DNA
gcctgataactcgaggaggta

SEQ ID NO: 38 - DNA, 31 bp, synthetic construct
  misc_feature 1..31: note = dAmp(RET)-R
  source 1..31: mol_type = other DNA
cccaagctttgagcggatacatatttgaatg

SEQ ID NO: 39 - DNA, 32 bp, synthetic construct
  misc_feature 1..32: note = dxr_1-F
  source 1..32: mol_type = other DNA
aaaagatctgtgtggaattgtgagcggataac

SEQ ID NO: 40 - DNA, 30 bp, synthetic construct
  misc_feature 1..30: note = dxr_1-R
  source 1..30: mol_type = other DNA
aaaactcgagtagatcagcttgcgagacgc

SEQ ID NO: 41 - DNA, 32 bp, synthetic construct; identical to SEQ ID NO: 39
  misc_feature 1..32: note = dxs_1-F
  source 1..32: mol_type = other DNA

SEQ ID NO: 42 - DNA, 29 bp, synthetic construct
  misc_feature 1..29: note = dxs_1-R
  source 1..29: mol_type = other DNA
aaaactcgagttatgccagccaggccttg

SEQ ID NO: 43 - DNA, 46 bp, synthetic construct
  misc_feature 1..46: note = idi-f
  source 1..46: mol_type = other DNA
acggatcctgaggaggtaacgtatgcaaacggaacacgtcatttta

SEQ ID NO: 44 - DNA, 42 bp, synthetic construct
  misc_feature 1..42: note = idi-r
  source 1..42: mol_type = other DNA
tatcgtcgactctaagatcttatttaagctgggtaaatgcag

SEQ ID NO: 45 - DNA, 32 bp, synthetic construct
  misc_feature 1..32: note = dxr_3-F
  source 1..32: mol_type = other DNA
aaagtcgacgtgtggaattgtgagcggataac

SEQ ID NO: 46 - DNA, 30 bp, synthetic construct
  misc_feature 1..30: note = dxr_3-R
  source 1..30: mol_type = other DNA
ttaaaagctttagatcagcttgcgagacgc

SEQ ID NO: 47 - DNA, 32 bp, synthetic construct
  misc_feature 1..32: note = dxs_2-F
  source 1..32: mol_type = other DNA
aaaactcgagtgtggaattgtgagcggataac

SEQ ID NO: 48 - DNA, 28 bp, synthetic construct
  misc_feature 1..28: note = dxs_2-R
  source 1..28: mol_type = other DNA
aaaagatctttatgccagccaggccttg

SEQ ID NO: 49 - DNA, 30 bp, synthetic construct
  misc_feature 1..30: note = dmvaE-F
  source 1..30: mol_type = other DNA
aaaagatctcattaggtagccgtttcaggc

SEQ ID NO: 50 - DNA, 38 bp, synthetic construct
  misc_feature 1..38: note = dmvaE-R
  source 1..38: mol_type = other DNA
aaaagatctttctcgagactccatggtctgtttcctcg

SEQ ID NO: 51 - DNA, 32 bp, synthetic construct
  misc_feature 1..32: note = dxr_2-F
  source 1..32: mol_type = other DNA
cccagtactgtgtggaattgtgagcggataac

SEQ ID NO: 52 - DNA, 29 bp, synthetic construct
  misc_feature 1..29: note = dxr_2-R
  source 1..29: mol_type = other DNA
cccgctagctagatcagcttgcgagacgc

SEQ ID NO: 53 - DNA, 27 bp, synthetic construct
  misc_feature 1..27: note = RET_2-F
  source 1..27: mol_type = other DNA
cccgctagcacagaatttgcctggcgg

SEQ ID NO: 54 - DNA, 31 bp, synthetic construct
  misc_feature 1..31: note = RET_2-R
  source 1..31: mol_type = other DNA
cccagtactcatgcctgcagttaacgatgag

SEQ ID NO: 55 - DNA, 22 bp, synthetic construct
  misc_feature 1..22: note = mvaA u/s-Fwd
  source 1..22: mol_type = other DNA
ccagcttcaaccattaatgaag

SEQ ID NO: 56 - DNA, 53 bp, synthetic construct
  misc_feature 1..53: note = mvaA u/s-Rev
  source 1..53: mol_type = other DNA
caggctattattttctcaaattttttagttaaaatttttttctcatatttccc

SEQ ID NO: 57 - DNA, 51 bp, synthetic construct
  misc_feature 1..51: note = mvaA d/s-Fwd
  source 1..51: mol_type = other DNA
gggaaatatgagaaaaaaattttaactaaaaaatttgagaaaataatagcc

SEQ ID NO: 58 - DNA, 19 bp, synthetic construct
  misc_feature 1..19: note = mvaA d/s-Rev
  source 1..19: mol_type = other DNA
gaatgacgagtggatcacc

SEQ ID NO: 59 - DNA, 24 bp, synthetic construct
  misc_feature 1..24: note = mvaA ex-Fwd
  source 1..24: mol_type = other DNA
ccattaatgaagtttgtggttcag

SEQ ID NO: 60 - DNA, 21 bp, synthetic construct
  misc_feature 1..21: note = mvaA ex-Rev
  source 1..21: mol_type = other DNA
cccttacatcctctgtctcac

SEQ ID NO: 61 - DNA, 21 bp, synthetic construct
  misc_feature 1..21: note = pORI19-Rev
  source 1..21: mol_type = other DNA
taccgagctcgaattcactgg

SEQ ID NO: 62 - DNA, 20 bp, synthetic construct
  misc_feature 1..20: note = pORI19-Fwd
  source 1..20: mol_type = other DNA
atcctctagagtcgacctgc

SEQ ID NO: 63 - DNA, 21 bp, synthetic construct
  misc_feature 1..21: note = DCO-Fwd
  source 1..21: mol_type = other DNA
cgccttaagaacaccagtcgg

SEQ ID NO: 64 - DNA, 19 bp, synthetic construct
  misc_feature 1..19: note = DCO-Rev
  source 1..19: mol_type = other DNA
gctctccgtaggtcatggc

SEQ ID NO: 65 - DNA, 22 bp, synthetic construct
  misc_feature 1..22: note = SCO u/s-Rev
  source 1..22: mol_type = other DNA
acaatttcacacaggaaacagc

SEQ ID NO: 66 - DNA, 21 bp, synthetic construct
  misc_feature 1..21: note = SCO u/s-Fwd
  source 1..21: mol_type = other DNA
cccccattaagtgccgagtgc

SEQ ID NO: 67 - DNA, 52 bp, synthetic construct
  misc_feature 1..52: note = mvaA-Fwd
  source 1..52: mol_type = other DNA
cgcggatccaggaggtaataaatatgagaaaaaaattttatcaaatgtcgcc

SEQ ID NO: 68 - DNA, 39 bp, synthetic construct
  misc_feature 1..39: note = mvaA-Rev
  source 1..39: mol_type = other DNA
gtctagattattttctcaaattttttagtaaattttggg

SEQ ID NO: 69 - DNA, 42 bp, synthetic construct
  misc_feature 1..42: note = EGFP-Fwd
  source 1..42: mol_type = other DNA
cgcgtcgacaggaggtaataaatatggtgagcaagggcgagg

SEQ ID NO: 70 - DNA, 31 bp, synthetic construct
  misc_feature 1..31: note = EGFP-Rev
  source 1..31: mol_type = other DNA
ggcatgcttacttgtacagctcgtccatgcc

SEQ ID NO: 71 - DNA, 2999 bp, synthetic construct
  misc_feature 1..2999: note = pSTV28
  source 1..2999: mol_type = other DNA
cgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggtgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagagcctgataaaaacggttagcgcttcgttaatacagatgtaggtgttccacagggtagccagcagcatcctgcgatgcagatccggaacataatggtgcagggcgcttgtttcggcgtgggtatggtggcaggccccgtggccgggggactgttgggcgctgccggcacctgtcctacgagttgcatgataaagaagacagtcataagtgcggcgacgatagtcatgccccgcgcccaccggaaggagctaccggacagcggtgcggactgttgtaactcagaataagaaatgaggccgctcatggcgttccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatcctctagagtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgagcttatcgatgataagctgtcaaacatgagaattacaacttatatcgtatggggctgacttcaggtgctacatttgaagagataaattgcactgaaatctagaaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccaggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattt

SEQ ID NO: 72 - DNA, 4176 bp, synthetic construct
  misc_feature 1..4176: note = pTrc99A
  source 1..4176: mol_type = other DNA
gtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatggaattcgagctcggtacccggggatcctctagagtcgacctgcaggcatgcaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctacagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctg

SEQ ID NO: 73 - DNA, 4620 bp, synthetic construct
  misc_feature 1..4620: note = pTFKC(DPB)
  source 1..4620: mol_type = other DNA
atcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggtcacacaggaaacagaccatggaattcgagctcggtacccggggatcctctagagtcgacctgcaggcatgcaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataacccgataattcgatggggatccgtcgacctgcagttcgaagttcctattctctagaaagtataggaacttcagagcgcttttgaagctcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctaataaggggatcttgaagttcctattccgaagttcctattctctagaaagtataggaacttcgaagcagctccagcctacaaggaggtaataaatatgatcaagcttatcgataccgtcgacctcgagggggggcccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgt

SEQ ID NO: 74 - DNA, 4335 bp, synthetic construct
  misc_feature 1..4335: note = pTFCC(DPB)
  source 1..4335: mol_type = other DNA
gtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggtcacacaggaaacagaccatggaattcgagctcggtacccggggatcctctagagtcgacctgcaggcatgcaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataacccgataattcgatcatatgaatatcctccttagttcctattccgaagttcctattctctagaaagtataggaacttcggcgcgcctacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattacgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagatgcttaatgaatacaacagtactgcgatgagtggcagggcggggcgtaaggcgcgccatttaaatgaagttcctattccgaagttcctattctctagaaagtataggaacttcgaagcagctccagcctacaaggaggtaataaatatgatcaagcttatcgataccgtcgacctcgagggggggcccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttag

SEQ ID NO: 75 - DNA, 9497 bp, synthetic construct
  misc_feature 1..9497: note = pCP20
  source 1..9497: mol_type = other DNA
gaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgcctgtagtgccatttacccccattcactgccagagccgtgagcgcagcgaactgaatgtcacgaaaaagacagcgactcaggtgcctgatggtcggagacaaaaggaatattcagcgatttgcccgagcttgcgagggtgctacttaagcctttagggttttaaggtctgttttgtagaggagcaaacagcgtttgcgacatccttttgtaatactgcggaactgactaaagtagtgagttatacacagggctgggatctattctttttatctttttttattctttctttattctataaattataaccacttgaatataaacaaaaaaaacacacaaaggtctagcggaatttacagagggtctagcagaatttacaagttttccagcaaaggtctagcagaatttacagatacccacaactcaaaggaaaaggactagtaattatcattgactagcccatctcaattggtatagtgattaaaatcacctagaccaattgagatgtatgtctgaattagttgttttcaaagcaaatgaactagcgattagtcgctatgacttaacggagcatgaaaccaagctaattttatgctgtgtggcactactcaaccccacgattgaaaaccctacaaggaaagaacggacggtatcgttcacttataaccaatacgttcagatgatgaacatcagtagggaaaatgcttatggtgtattagctaaagcaaccagagagctgatgacgagaactgtggaaatcaggaatcctttggttaaaggctttgagattttccagtggacaaactatgccaagttctcaagcgaaaaattagaattagtttttagtgaagagatattgccttatcttttccagttaaaaaaattcataaaatataatctggaacatgttaagtcttttgaaaacaaatactctatgaggatttatgagtggttattaaaagaactaacacaaaagaaaactcacaaggcaaatatagagattagccttgatgaatttaagttcatgttaatgcttgaaaataactaccatgagtttaaaaggcttaaccaatgggttttgaaaccaataagtaaagatttaaacacttacagcaatatgaaattggtggttgataagcgaggccgcccgactgatacgttgattttccaagttgaactagatagacaaatggatctcgtaaccgaacttgagaacaaccagataaaaatgaatggtgacaaaataccaacaaccattacatcagattcctacctacgtaacggactaagaaaaacactacacgatgctttaactgcaaaaattcagctcaccagttttgaggcaaaatttttgagtgacatgcaaagtaagcatgatctcaatggttcgttctcatggctcacgcaaaaacaacgaaccacactagagaacatactggctaaatacggaaggatctgaggttcttatggctcttgtatctatcagtgaagcatcaagactaacaaacaaaagtagaacaactgttcaccgttagatatcaaagggaaaactgtccatatgcacagatgaaaacggtgtaaaaaagatagatacatcagagcttttacgagtttttggtgcatttaaagctgttcaccatgaacagatcgacaatgtaacagatgaacagcatgtaacacctaatagaacaggtgaaaccagtaaaacaaagcaactagaacatgaaattgaacacctgagacaacttgttacagctcaacagtcacacatagacagcctgaaacaggcgatgctgcttatcgaatcaaagctgccgacaacacgggagccagtgacgcctcccgtggggaaaaaatcatggcaattctggaagaaaatagcgcctgtttcgtttcaggcaggttatcagggagtgtcagcgtcctgcggttctccggggcgttcgggtcatgcagcccgtaatggtgatttaacagcgtctgccaagcatcaattctaggcctgtctgcgcggtcgtagtacggctggaggcgttttccggtctgtagctccatgttcggaatgacaaaattcagctcaagccgtcccttgtcctggtgctccacccacaggatgctgtactgatttttttcgagaccgggcatcagtacacgctcaaagctcgccatcactttttcacgtcctcccggcggcagctccttctccgcgaacgacagaacaccggacgtgtatttcttcgcaaatggcgtggcatcgatgagttcccggacttcttccggattaccctgaagcaccgttgcgccttcgcggttacgctccctccccagcaggtaatcaaccggaccactgccaccaccttttcccctggcatgaaatttaactatcatcccgcgccccctgttccctgacagccagacgcagccggcgcagctcatccccgatggccatcagtgcggccaccacctgaacccggtcaccggaagaccactgcccgctgttcaccttacgggctgtctgattcaggttatttccgatggcggccagctgacgcagtaacggcggtgccagtgtcggcagttttccggaacgggcaaccggctcccccaggcagacccgccgcatccataccgccagttgtttaccctcacagcgttcaagtaaccgggcatgttcatcatcagtaacccgtattgtgagcatcctctcgcgtttcatcggtatcattaccccatgaacagaaatcccccttacacggaggcatcagtgactaaacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattttataaaccgtggagcgggcaatactgagctgatgagcaatttccgttgcaccagtgcccttctgatgaagcgtcagcacgacgttcctgtccacggtacgcctgcggccaaatttgattcctttcagctttgcttcctgtcggccctcattcgtgcgctctaggatcctctacgccggacgcatcgtggccggcatcaccggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttctagaggatccccctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatatgcatagtaccgagaaactagtgcgaagtagtgatcaggtattgctgttatctgatgagtatacgttgtcctggccacggcagaagcacgcttatcgctccaatttcccacaacattagtcaactccgttaggcccttcattgaaagaaatgaggtcatcaaatgtcttccaatgtgagattttgggccattttttatagcaaagattgaataaggcgcatttttcttcaaagctttattgtacgatctgactaagttatcttttaataattggtattcctgtttattgcttgaagaattgccggtcctatttactcgttttaggactggttcagaattcctcaaaaattcatccaaatatacaagtggatcgatcctaccccttgcgctaaagaagtatatgtgcctactaacgcttgtctttgtctctgtcactaaacactggattattactcccagatacttattttggactaatttaaatgatttcggatcaacgttcttaatatcgctgaatcttccacaattgatgaaagtagctaggaagaggaattggtataaagtttttgtttttgtaaatctcgaagtatactcaaacgaatttagtattttctcagtgatctcccagatgctttcaccctcacttagaagtgctttaagcatttttttactgtggctatttcccttatctgcttcttccgatgattcgaactgtaattgcaaactacttacaatatcagtgatatcagattgatgtttttgtccatagtaaggaataattgtaaattcccaagcaggaatcaatttctttaatgaggcttccagaattgttgctttttgcgtcttgtatttaaactggagtgatttattgacaatatcgaaactcagcgaattgcttatgatagtattatagctcatgaatgtggctctcttgattgctgttccgttatgtgtaatcatccaacataaataggttagttcagcagcacataatgctattttctcacctgaaggtctttcaaacctttccacaaactgacgaacaagcaccttaggtggtgttttacataatataccaaattgtggcatacaacctccttagtacatgcaaccattatcaccgccagaggtaaaatagtcaacacgcacggtgttagatatttatcccttgcggtgatagatttaacgtatgagcacaaaaaagaaaccattaacacaagagcagcttgaggacgcacgtcgccttaaagcaatttatgaaaaaaagaaaaatgaacttggcttatcccaggaatctgtcgcagacaagatggggatggggcagtcaggcgttggtgctttatttaatggcatcaatgcattaaatgcttataacgccgcattgcttacaaaaattctcaaagttagcgttgaagaatttagcccttcaatcgccagagaaatctacgagatgtatgaagcggttagtatgcagccgtcacttagaagtgagtatgagtaccctgttttttctcatgttcaggcagggatgttctcacctaagcttagaacctttaccaaaggtgatgcggagagatgggtaagcacaaccaaaaaagccagtgattctgcattctggcttgaggttgaaggtaattccatgaccgcaccaacaggctccaagccaagctttcctgacggaatgttaattctcgttgaccctgagcaggctgttgagccaggtgatttctgcatagccagacttgggggtgatgagtttaccttcaagaaactgatcagggatagcggtcaggtgtttttacaaccactaaacccacagtacccaatgatcccatgcaatgagagttgttccgttgtggggaaagttatcgctagtcagtggcctgaagagacgtttggctgatcggcaaggtgttctggtcggcgcatagctgataacaattgagcaagaatcttcatcgaattaggggaattttcactcccctcagaacataacatagtaaatggattgaattatgaagaatggtttttatgcgacttaccgcagcaaaaataaagggaaagataagcgctcaataaacctgtctgttttccttaattctctgctggctgataatcatcacctgcaggttggctccaattatttgtatattcataaaatcgagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacg

SEQ ID NO: 76 - DNA, 6329 bp, synthetic construct
  misc_feature 1..6329: note = pKD46
  source 1..6329: mol_type = other DNA
catcgatttattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggaattcgagctctaaggaggttataaaaaatggatattaatactgaaactgagatcaagcaaaagcattcactaaccccctttcctgttttcctaatcagcccggcatttcgcgggcgatattttcacagctatttcaggagttcagccatgaacgcttattacattcaggatcgtcttgaggctcagagctgggcgcgtcactaccagcagctcgcccgtgaagagaaagaggcagaactggcagacgacatggaaaaaggcctgccccagcacctgtttgaatcgctatgcatcgatcatttgcaacgccacggggccagcaaaaaatccattacccgtgcgtttgatgacgatgttgagtttcaggagcgcatggcagaacacatccggtacatggttgaaaccattgctcaccaccaggttgatattgattcagaggtataaaacgaatgagtactgcactcgcaacgctggctgggaagctggctgaacgtgtcggcatggattctgtcgacccacaggaactgatcaccactcttcgccagacggcatttaaaggtgatgccagcgatgcgcagttcatcgcattactgatcgttgccaaccagtacggccttaatccgtggacgaaagaaatttacgcctttcctgataagcagaatggcatcgttccggtggtgggcgttgatggctggtcccgcatcatcaatgaaaaccagcagtttgatggcatggactttgagcaggacaatgaatcctgtacatgccggatttaccgcaaggaccgtaatcatccgatctgcgttaccgaatggatggatgaatgccgccgcgaaccattcaaaactcgcgaaggcagagaaatcacggggccgtggcagtcgcatcccaaacggatgttacgtcataaagccatgattcagtgtgcccgtctggccttcggatttgctggtatctatgacaaggatgaagccgagcgcattgtcgaaaatactgcatacactgcagaacgtcagccggaacgcgacatcactccggttaacgatgaaaccatgcaggagattaacactctgctgatcgccctggataaaacatgggatgacgacttattgccgctctgttcccagatatttcgccgcgacattcgtgcatcgtcagaactgacacaggccgaagcagtaaaagctcttggattcctgaaacagaaagccgcagagcagaaggtggcagcatgacaccggacattatcctgcagcgtaccgggatcgatgtgagagctgtcgaacagggggatgatgcgtggcacaaattacggctcggcgtcatcaccgcttcagaagttcacaacgtgatagcaaaaccccgctccggaaagaagtggcctgacatgaaaatgtcctacttccacaccctgcttgctgaggtttgcaccggtgtggctccggaagttaacgctaaagcactggcctggggaaaacagtacgagaacgacgccagaaccctgtttgaattcacttccggcgtgaatgttactgaatccccgatcatctatcgcgacgaaagtatgcgtaccgcctgctctcccgatggtttatgcagtgacggcaacggccttgaactgaaatgcccgtttacctcccgggatttcatgaagttccggctcggtggtttcgaggccataaagtcagcttacatggcccaggtgcagtacagcatgtgggtgacgcgaaaaaatgcctggtactttgccaactatgacccgcgtatgaagcgtgaaggcctgcattatgtcgtgattgagcgggatgaaaagtacatggcgagttttgacgagatcgtgccggagttcatcgaaaaaatggacgaggcactggctgaaattggttttgtatttggggagcaatggcgatgacgcatcctcacgataatatccgggtaggcgcaatcactttcgtctactccgttacaaagcgaggctgggtatttcccggcctttctgttatccgaaatccactgaaagcacagcggctggctgaggagataaataataaacgaggggctgtatgcacaaagcatcttctgttgagttaagaacgagtatcgagatggcacatagccttgctcaaattggaatcaggtttgtgccaataccagtagaaacagacgaagaatccatgggtatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcggttgcggccgcaatcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 77 - DNA, 6738 bp, synthetic construct
  misc_feature 1..6738: note = pT-ispA-STS
  source 1..6738: mol_type = other DNA
gtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaggtaataaatatggaattcatggactttccgcagcaactcgaagcctgcgttaagcaggccaaccaggcgctgagccgttttatcgccccactgccctttcagaacactcccgtggtcgaaaccatgcagtatggcgcattattaggtggtaagcgcctgcgacctttcctggtttatgccaccggtcatatgttcggcgttagcacaaacacgctggacgcacccgctgccgccgttgagtgtatccacgcttactcattaattcatgatgatttaccggcaatggatgatgacgatctgcgtcgcggtttgccaacctgccatgtgaagtttggcgaagcaaacgcgattctcgctggcgacgctttacaaacgctggcgttctcgattttaagcgatgccgatatgccggaagtgtcggaccgcgacagaatttcgatgatttctgaactggcgagcgccagtggtattgccggaatgtgcggtggtcaggcattagatttagacgcggaaggcaaacacgtacctctggacgcgcttgagcgtattcatcgtcataaaaccggcgcattgattcgcgccgccgttcgccttggtgcattaagcgccggagataaaggacgtcgtgctctgccggtactcgacaagtatgcagagagcatcggccttgccttccaggttcaggatgacatcctggatgtggtgggagatactgcaacgttgggaaaacgccagggtgccgaccagcaacttggtaaaagtacctaccctgcacttctgggtcttgagcaagcccggaagaaagcccgggatctgatcgacgatgcccgtcagtcgctgaaacaactggctgaacagtcactcgatacctcggcactggaagcgctagcggactacatcatccagcgtaataaataaggatccaaaaaggaggtaataaaccatgtcaactcaacaagtttcatcagagaacattgttcgtaacgctgcgaatttccatcctaatatatggggaaaccatttcctcacatgtccttctcagacgattgatagttggactcaacagcaccacaaagaactgaaagaagaggtgaggaaaatgatggtgtctgatgcaaataaacctgcccagagattgcgcttgattgatactgtccaaaggctaggtgtggcttaccactttgaaaaggagattgatgatgcattggagaaaataggtcatgacccttttgatgataaagatgatctctacattgtctctctttgttttcgattgctgaggcagcatggaattaagatatcatgtgatgtgtttgagaagtttaaagatgacgatggaaaattcaaggcatcattgatgaatgatgttcaaggcatgctaagtttatatgaggcagcacacctagccattcacggagaagatattttagatgaagcaattgttttcacgaccactcaccttaagtcaacggtatctaattctcctgtaaactctacttttgctgaacaaatacgtcattctctcagagttcctctccgtaaagctgtacctaggttagagtcgaggtatttcttggatatctattcaagagatgatttgcacgataaaactttgctcaatttcgcaaagttagactttaatatactacaagcaatgcaccagaaggaagcaagtgagatgaccaggtggtggagagattttgacttccttaaaaagctgccttatataagagacagagtcgtggagctatatttttggattctggtgggagtgtcttatcagcccaaattcagcactggtagaatttttttgtccaaaataatatgccttgagaccctcgtagatgatacatttgacgcctacggtacttttgacgagctcgcaatctttactgaagcagttacaagatgggaccttggccacagagatgcactaccagaatacatgaaattcattttcaagacactcattgatgtctacagtgaagctgagcaagaactggcaaaggaagggagatcatacagcatacactatgcaatacgatcgttccaagaactagttatgaagtacttctgcgaagccaagtggttaaataaaggttatgttccgagcctggacgattataaatcagtttcattaagaagtatcggttttttaccgatagcggtagcttccttcgttttcatgggtgatattgcaactaaggaggtctttgaatgggaaatgaataaccctaagatcataatagccgcagaaacgattttcagattcctggatgacatagcaggccataggtttgagcaaaagagagaacatagtccatcagctattgaatgctacaagaatcaacatggagtgtctgaggaagaggcagttaaagcgttgtcgttagaagttgctaatagttggaaagatataaatgaggagctgcttctcaacccaatggctattcctttacctctgcttcaggtgattcttgatctctcacgttcggccgattttatgtacggtaatgctcaagatcgcttcacgcattcaacgatgatgaaagaccaagttgatttggtgctgaaggaccccgttaagcttgacgattaaagatctgtcgacctgcaggcatgcaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctacagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctg

SEQ ID NO: 78 - DNA, 13899 bp, synthetic construct
  misc_feature 1..13899: note = pTAS-NA
  source 1..13899: mol_type = other DNA
gatcctacctgacgctttttatcgcaactctctactgtttctccatacccgtttttttgggctagcaggaggaattcaccatggtaccaatgacaaaaaaagttggtgtcggtcaggcacatagtaagataattttaataggggaacatgcggtcgtttacggttatcctgccatttccctgcctcttttggaggtggaggtgacctgtaaggtagttcctgcagagagtccttggcgcctttatgaggaggataccttgtccatggcggtttatgcctcactggagtatttgaatatcacagaagcctgcattcgttgtgagattgactcggctatccctgagaaacgggggatgggttcgtcagcggctatcagcatagcggccattcgtgcagtatttgactactatcaggctgatctgcctcatgatgtactagaaatcttggtcaatcgagctgaaatgattgcccatatgaatcctagtggtttggatgctaagacctgtcttagtgaccaacctattcgctttatcaagaacgtaggatttacagaacttgagatggatttatccgcctatttggtgattgccgatacgggtgtttatggtcatactcgtgaagccatccaagtggttcaaaataagggcaaggatgccctaccgtttttgcatgccttgggagaattaacccagcaagcagaagttgcgatttcacaaaaagatgctgaaggactgggacaaatcctcagtcaagcgcatttacatttaaaagaaattggagtcagtagccctgaggcagactttttggttgaaacgactcttagccatggtgctctgggtgccaagatgagcggtggtgggctaggaggttgtatcatagccttggtaaccaatttgacacacgcacaagaactagcagaaagattagaagagaaaggagctgttcagacatggatagagagcctgtaacagtacgttcctacgcaaatattgctattatcaaatattggggaaagaaaaaagaaaaagagatggtgcctgctactagcagtatttctctaactttggaaaatatgtatacagagacgaccttgtcgcctttaccagccaatgtaacagctgacgaattttacatcaatggtcagctacaaaatgaggtcgagcatgccaagatgagtaagattattgaccgttatcgtccagctggtgagggctttgtccgtatcgatactcaaaacaatatgcctacggcagcgggcctgtcctcaagttctagtggtttgtccgccctggtcaaggcttgtaatgcttatttcaagcttggattggatagaagtcagttggcacaggaagccaaatttgcctcaggctcttcttctcggagtttttatggaccactaggagcctgggataaggatagtggagaaatttaccctgtagagacagacttgaaactagctatgattatgttggtgctagaggacaagaaaaaaccaatctctagccgtgacgggatgaaactttgtgtggaaacctcgacgacttttgacgactgggttcgtcagtctgagaaggactatcaggatatgctgatttatctcaaggaaaatgattttgccaagattggagaattaacggagaaaaatgctctggctatgcatgctacgacaaagactgctagtccagccttttcttatctgacggatgcctcttatgaggctatggcctttgttcgccagcttcgtgagaaaggagaggcctgctactttaccatggatgctggtcccaatgttaaggtcttctgtcaggagaaagacttggagcatttgtcagaaattttcggtcagcgttatcgcttgattgtgtcaaaaacaaaggatttgagtcaagatgattgctgttaaaacttgcggaaaactctattgggcaggtgaatatgctattttagagccagggcagttagctttgataaaggatattcccatctatatgagggctgagattgctttttctgacagctaccgtatctattcagatatgtttgatttcgcagtggacttaaggcccaatcctgactacagcttgattcaagaaacgattgctttgatgggagacttcctcgctgttcgcggtcagaatttaagacctttttccctaaaaatctgtggcaaaatggaacgagaagggaaaaagtttggtctaggttctagtggcagcgtcgttgtcttggttgtcaaggctttactggctctctataatctttcggttgatcagaatctcttgttcaagctgactagcgctgtcttgctcaagcgaggagacaatggttccatgggcgaccttgcctgtattgtggcagaggatttggttctttaccagtcatttgatcgccagaaggcggctgcttggttagaagaagaaaacttggcgacagttctggagcgtgattggggattttttatctcacaagtgaaaccaactttagaatgtgatttcttagtgggatggaccaaggaagtggctgtatcgagtcacatggtccagcaaatcaagcaaaatatcaatcaaaattttttaagttcctcaaaagaaacggtggtttctttggtcgaagccttggagcaggggaaagccgaaaaagttatcgagcaagtagaagtagccagcaagcttttagaaggcttgagtacagatatttacacgcctttgcttagacagttgaaagaagccagtcaagatttgcaggccgttgccaagagtagtggtgctggtggtggtgactgtggcatcgccctgagttttgatgcgcaatcttctcgaaacactttaaaaaatcgttgggccgatctggggattgagctcttatatcaagaaaggataggacatgacgacaaatcgtaatctagccccgggaggagagaaattatgcaaacggaacacgtcattttattgaatgcacagggagttcccacgggtacgctggaaaagtatgccgcacacacggcagacacccgcttacatctcgcgttctccagttggctgtttaatgccaaaggacaattattagttacccgccgcgcactgagcaaaaaagcatggcctggcgtgtggactaactcggtttgtgggcacccacaactgggagaaagcaacgaagacgcagtgatccgccgttgccgttatgagcttggcgtggaaattacgcctcctgaatctatctatcctgactttcgctaccgcgccaccgatccgagtggcattgtggaaaatgaagtgtgtccggtatttgccgcacgcaccactagtgcgttacagatcaatgatgatgaagtgatggattatcaatggtgtgatttagcagatgtattacacggtattgatgccacgccgtgggcgttcagtccgtggatggtgatgcaggcgacaaatcgcgaagccagaaaacgattatctgcatttacccagcttaaataagcatgcactagagtcgaggaaacagaccatggagttgaaaacagtagttattattgatgcattacgaacaccaattggaaaatataaaggcagcttaagtcaagtaagtgccgtagacttaggaacacatgttacaacacaacttttaaaaagacattccactatttctgaagaaattgatcaagtaatctttggaaatgttttacaagctggaaatggccaaaatcccgcacgacaaatagcaataaacagcggtttatctcatgaaattcccgcaatgacagttaatgaggtctgcggatcaggaatgaaggccgttattttggcgaaacaattgattcaattaggagaagcggaagttttaattgctggcgggattgagaatatgtcccaagcacctaaattacaacgatttaattacgaaacagaaagctatgatgcgcctttttctagtatgatgtacgatgggttaacggatgcctttagtggtcaagcaatgggcttaactgctgaaaatgtggccgaaaagtatcatgtaactagagaagagcaagatcaattttctgtacattcacaattaaaagcagctcaagcacaagcagaagggatattcgctgacgaaatagccccattagaagtatcaggaacgcttgtggagaaagatgaagggattcgccctaattcgagcgttgagaagctaggaacgcttaaaacagtttttaaagaagacggtactgtaacagcagggaatgcatcaaccattaatgatggggcttctgctttgattattgcttcacaagaatatgccgaagcacacggtcttccttatttagctattattcgagacagtgtggaagtcggtattgatccagcctatatgggaatttcgccgattaaagccattcaaaaactgttagcgcgcaatcaacttactacggaagaaattgatctgtatgaaatcaacgaagcatttgcagcaacttcaatcgtggtccaaagagaactggctttaccagaggaaaaggtcaacatttatggtggcggtatttcattaggtcatgcgattggtgccacaggtgctcgtttattaacgagtttaagttatcaattaaatcaaaaagaaaagaaatatggagtggcttctttatgtatcggcggtggcttaggactcgctatgctactagagagacctcagcaaaaaaaaaacagccgattttatcaaatgagtcctgaggaacgcctggcttctcttcttaatgaaggccagatttctgctgatacaaaaaaagaatttgaaaatacggctttatcttcgcagattgccaatcatatgattgaaaatcaaatcagtgaaacagaagtgccgatgggcgttggcttacatttaacagtggacgaaactgattatttggtaccaatggcgacagaagagccctcagtgattgcggctttgagtaatggtgcaaaaatagcacaaggatttaaaacagtgaatcaacaacgtttaatgcgtggacaaatcgttttttacgatgttgcagacgccgagtcattgattgatgaactacaagtaagagaaacggaaatttttcaacaagcagagttaagttatccatctatcgttaaacgcggcggcggcttaagagatttgcaatatcgtgcttttgatgaatcatttgtatctgtcgactttttagtagatgttaaggatgcaatgggggcaaatatcgttaacgctatgttggaaggtgtggccgagttgttccgtgaatggtttgcggagcaaaagattttattcagtattttaagtaattatgccacggagtcggttgttacgatgaaaacggctattccagtttcacgtttaagtaaggggagcaatggccgggaaattgctgaaaaaattgttttagcttcacgctatgcttcattagatccttatcgggcagtcacgcataacaaagggatcatgaatggcattgaagctgtcgttttagctacaggaaatgatacacgcgctgttagcgcttcttgtcatgcttttgcggtgaaggaaggtcgctaccaaggtttgactagttggacgctggatggcgaacaactaattggtgaaatttcagttccgcttgcgttagccacggttggcggtgccacaaaagtcttacctaaatctcaagcagctgctgatttgttagcagtgacggatgcaaaagaactaagtcgagtagtagcggctgttggtttggcacaaaatttagcggcgttacgggccttagtctctgaaggaattcaaaaaggacacatggctctacaagcacgttctttagcgatgacggtcggagctactggtaaagaagttgaggcagtcgctcaacaattaaaacgtcaaaaaacgatgaaccaagaccgagccttggctattttaaatgatttaagaaaacaataaaaaaacagttcagcagaaattattctgctgaactgttttttttcacattaggtagccgtttcaggccacgagctcaggagttaaagaaatgacaattgggattgataaaattagtttttttgtgcccccttattatattgatatgacggcactggctgaagccagaaatgtagaccctggaaaatttcatattggtattgggcaagaccaaatggcggtgaacccaatcagccaagatattgtgacatttgcagccaatgccgcagaagcgatcttgaccaaagaagataaagaggccattgatatggtgattgtcgggactgagtccagtatcgatgagtcaaaagcggccgcagttgtcttacatcgtttaatggggattcaacctttcgctcgctctttcgaaatcaaggaagcttgttacggagcaacagcaggcttacagttagctaagaatcacgtagccttacatccagataaaaaagtcttggttgtagcagcagatattgcaaaatatggattaaattctggcggtgagcctacacaaggagctggggcggttgcaatgttagttgctagtgaaccgcgcatcttggctttaaaagaggataatgtgatgctgacgcaagatatctatgacttttggcgtccaacaggccatccgtatcctatggtcgatggtcctttgtcaaacgaaacctacatccaatcttttgcccaagtctgggatgaacataaaaaaagaaccggtcttgattttgcagattatgatgctttagcgttccatattccttacacaaaaatgggcaaaaaagccttattagcaaaaatctccgaccaaactgaagcagaacaggaacgaattttagcccgttatgaagaaagcatcatctatagtcgtcgcgtaggaaacttgtatacgggttcactttatctgggactcatttcccttttagaaaatgcaacgactttaaccgcaggcaatcaaattgggttattcagttatggttctggtgctgtcgctgaatttttcactggtgaattagtagctggttatcaaaatcatttacaaaaagaaactcatttagcactgctagataatcggacagaactttctatcgctgaatatgaagccatgtttgcagaaactttagacacagatattgatcaaacgttagaagatgaattaaaatatagtatttctgctattaataataccgttcgctcttatcgaaactaactgcagcctcgacctgcaggcatgcaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctacagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctggtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaggtaataaatatggaattcatggactttccgcagcaactcgaagcctgcgttaagcaggccaaccaggcgctgagccgttttatcgccccactgccctttcagaacactcccgtggtcgaaaccatgcagtatggcgcattattaggtggtaagcgcctgcgacctttcctggtttatgccaccggtcatatgttcggcgttagcacaaacacgctggacgcacccgctgccgccgttgagtgtatccacgcttactcattaattcatgatgatttaccggcaatggatgatgacgatctgcgtcgcggtttgccaacctgccatgtgaagtttggcgaagcaaacgcgattctcgctggcgacgctttacaaacgctggcgttctcgattttaagcgatgccgatatgccggaagtgtcggaccgcgacagaatttcgatgatttctgaactggcgagcgccagtggtattgccggaatgtgcggtggtcaggcattagatttagacgcggaaggcaaacacgtacctctggacgcgcttgagcgtattcatcgtcataaaaccggcgcattgattcgcgccgccgttcgccttggtgcattaagcgccggagataaaggacgtcgtgctctgccggtactcgacaagtatgcagagagcatcggccttgccttccaggttcaggatgacatcctggatgtggtgggagatactgcaacgttgggaaaacgccagggtgccgaccagcaacttggtaaaagtacctaccctgcacttctgggtcttgagcaagcccggaagaaagcccgggatctgatcgacgatgcccgtcagtcgctgaaacaactggctgaacagtcactcgatacctcggcactggaagcgctagcggactacatcatccagcgtaataaataaggatccaaaaaggaggtaataaaccatgtcaactcaacaagtttcatcagagaacattgttcgtaacgctgcgaatttccatcctaatatatggggaaaccatttcctcacatgtccttctcagacgattgatagttggactcaacagcaccacaaagaactgaaagaagaggtgaggaaaatgatggtgtctgatgcaaataaacctgcccagagattgcgcttgattgatactgtccaaaggctaggtgtggcttaccactttgaaaaggagattgatgatgcattggagaaaataggtcatgacccttttgatgataaagatgatctctacattgtctctctttgttttcgattgctgaggcagcatggaattaagatatcatgtgatgtgtttgagaagtttaaagatgacgatggaaaattcaaggcatcattgatgaatgatgttcaaggcatgctaagtttatatgaggcagcacacctagccattcacggagaagatattttagatgaagcaattgttttcacgaccactcaccttaagtcaacggtatctaattctcctgtaaactctacttttgctgaacaaatacgtcattctctcagagttcctctccgtaaagctgtacctaggttagagtcgaggtatttcttggatatctattcaagagatgatttgcacgataaaactttgctcaatttcgcaaagttagactttaatatactacaagcaatgcaccagaaggaagcaagtgagatgaccaggtggtggagagattttgacttccttaaaaagctgccttatataagagacagagtcgtggagctatatttttggattctggtgggagtgtcttatcagcccaaattcagcactggtagaatttttttgtccaaaataatatgccttgagaccctcgtagatgatacatttgacgcctacggtacttttgacgagctcgcaatctttactgaagcagttacaagatgggaccttggccacagagatgcactaccagaatacatgaaattcattttcaagacactcattgatgtctacagtgaagctgagcaagaactggcaaaggaagggagatcatacagcatacactatgcaatacgatcgttccaagaactagttatgaagtacttctgcgaagccaagtggttaaataaaggttatgttccgagcctggacgattataaatcagtttcattaagaagtatcggttttttaccgatagcggtagcttccttcgttttcatgggtgatattgcaactaaggaggtctttgaatgggaaatgaataaccctaagatcataatagccgcagaaacgattttcagattcctggatgacatagcaggccataggtttgagcaaaagagagaacatagtccatcagctattgaatgctacaagaatcaacatggagtgtctgaggaagaggcagttaaagcgttgtcgttagaagttgctaatagttggaaagatataaatgaggagctgcttctcaacccaatggctattcctttacctctgcttcaggtgattcttgatctctcacgttcggccgattttatgtacggtaatgctcaagatcgcttcacgcattcaacgatgatgaaagaccaagttgatttggtgctgaaggaccccgttaagcttgacgattaaa

SEQ ID NO: 79 - DNA, 10342 bp, synthetic construct
  misc_feature 1..10342: note = pSNAK
  source 1..10342: mol_type = other DNA
gcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatcctacctgacgctttttatcgcaactctctactgtttctccatacccgtttttttgggctagcaggaggaattcaccatggtaccaatgacaaaaaaagttggtgtcggtcaggcacatagtaagataattttaataggggaacatgcggtcgtttacggttatcctgccatttccctgcctcttttggaggtggaggtgacctgtaaggtagttcctgcagagagtccttggcgcctttatgaggaggataccttgtccatggcggtttatgcctcactggagtatttgaatatcacagaagcctgcattcgttgtgagattgactcggctatccctgagaaacgggggatgggttcgtcagcggctatcagcatagcggccattcgtgcagtatttgactactatcaggctgatctgcctcatgatgtactagaaatcttggtcaatcgagctgaaatgattgcccatatgaatcctagtggtttggatgctaagacctgtcttagtgaccaacctattcgctttatcaagaacgtaggatttacagaacttgagatggatttatccgcctatttggtgattgccgatacgggtgtttatggtcatactcgtgaagccatccaagtggttcaaaataagggcaaggatgccctaccgtttttgcatgccttgggagaattaacccagcaagcagaagttgcgatttcacaaaaagatgctgaaggactgggacaaatcctcagtcaagcgcatttacatttaaaagaaattggagtcagtagccctgaggcagactttttggttgaaacgactcttagccatggtgctctgggtgccaagatgagcggtggtgggctaggaggttgtatcatagccttggtaaccaatttgacacacgcacaagaactagcagaaagattagaagagaaaggagctgttcagacatggatagagagcctgtaacagtacgttcctacgcaaatattgctattatcaaatattggggaaagaaaaaagaaaaagagatggtgcctgctactagcagtatttctctaactttggaaaatatgtatacagagacgaccttgtcgcctttaccagccaatgtaacagctgacgaattttacatcaatggtcagctacaaaatgaggtcgagcatgccaagatgagtaagattattgaccgttatcgtccagctggtgagggctttgtccgtatcgatactcaaaacaatatgcctacggcagcgggcctgtcctcaagttctagtggtttgtccgccctggtcaaggcttgtaatgcttatttcaagcttggattggatagaagtcagttggcacaggaagccaaatttgcctcaggctcttcttctcggagtttttatggaccactaggagcctgggataaggatagtggagaaatttaccctgtagagacagacttgaaactagctatgattatgttggtgctagaggacaagaaaaaaccaatctctagccgtgacgggatgaaactttgtgtggaaacctcgacgacttttgacgactgggttcgtcagtctgagaaggactatcaggatatgctgatttatctcaaggaaaatgattttgccaagattggagaattaacggagaaaaatgctctggctatgcatgctacgacaaagactgctagtccagccttttcttatctgacggatgcctcttatgaggctatggcctttgttcgccagcttcgtgagaaaggagaggcctgctactttaccatggatgctggtcccaatgttaaggtcttctgtcaggagaaagacttggagcatttgtcagaaattttcggtcagcgttatcgcttgattgtgtcaaaaacaaaggatttgagtcaagatgattgctgttaaaacttgcggaaaactctattgggcaggtgaatatgctattttagagccagggcagttagctttgataaaggatattcccatctatatgagggctgagattgctttttctgacagctaccgtatctattcagatatgtttgatttcgcagtggacttaaggcccaatcctgactacagcttgattcaagaaacgattgctttgatgggagacttcctcgctgttcgcggtcagaatttaagacctttttccctaaaaatctgtggcaaaatggaacgagaagggaaaaagtttggtctaggttctagtggcagcgtcgttgtcttggttgtcaaggctttactggctctctataatctttcggttgatcagaatctcttgttcaagctgactagcgctgtcttgctcaagcgaggagacaatggttccatgggcgaccttgcctgtattgtggcagaggatttggttctttaccagtcatttgatcgccagaaggcggctgcttggttagaagaagaaaacttggcgacagttctggagcgtgattggggattttttatctcacaagtgaaaccaactttagaatgtgatttcttagtgggatggaccaaggaagtggctgtatcgagtcacatggtccagcaaatcaagcaaaatatcaatcaaaattttttaagttcctcaaaagaaacggtggtttctttggtcgaagccttggagcaggggaaagccgaaaaagttatcgagcaagtagaagtagccagcaagcttttagaaggcttgagtacagatatttacacgcctttgcttagacagttgaaagaagccagtcaagatttgcaggccgttgccaagagtagtggtgctggtggtggtgactgtggcatcgccctgagttttgatgcgcaatcttctcgaaacactttaaaaaatcgttgggccgatctggggattgagctcttatatcaagaaaggataggacatgacgacaaatcgtaatctagccccgggaggagagaaattatgcaaacggaacacgtcattttattgaatgcacagggagttcccacgggtacgctggaaaagtatgccgcacacacggcagacacccgcttacatctcgcgttctccagttggctgtttaatgccaaaggacaattattagttacccgccgcgcactgagcaaaaaagcatggcctggcgtgtggactaactcggtttgtgggcacccacaactgggagaaagcaacgaagacgcagtgatccgccgttgccgttatgagcttggcgtggaaattacgcctcctgaatctatctatcctgactttcgctaccgcgccaccgatccgagtggcattgtggaaaatgaagtgtgtccggtatttgccgcacgcaccactagtgcgttacagatcaatgatgatgaagtgatggattatcaatggtgtgatttagcagatgtattacacggtattgatgccacgccgtgggcgttcagtccgtggatggtgatgcaggcgacaaatcgcgaagccagaaaacgattatctgcatttacccagcttaaataagcatgcactagagtcgaggaaacagaccatggagttgaaaacagtagttattattgatgcattacgaacaccaattggaaaatataaaggcagcttaagtcaagtaagtgccgtagacttaggaacacatgttacaacacaacttttaaaaagacattccactatttctgaagaaattgatcaagtaatctttggaaatgttttacaagctggaaatggccaaaatcccgcacgacaaatagcaataaacagcggtttatctcatgaaattcccgcaatgacagttaatgaggtctgcggatcaggaatgaaggccgttattttggcgaaacaattgattcaattaggagaagcggaagttttaattgctggcgggattgagaatatgtcccaagcacctaaattacaacgatttaattacgaaacagaaagctatgatgcgcctttttctagtatgatgtacgatgggttaacggatgcctttagtggtcaagcaatgggcttaactgctgaaaatgtggccgaaaagtatcatgtaactagagaagagcaagatcaattttctgtacattcacaattaaaagcagctcaagcacaagcagaagggatattcgctgacgaaatagccccattagaagtatcaggaacgcttgtggagaaagatgaagggattcgccctaattcgagcgttgagaagctaggaacgcttaaaacagtttttaaagaagacggtactgtaacagcagggaatgcatcaaccattaatgatggggcttctgctttgattattgcttcacaagaatatgccgaagcacacggtcttccttatttagctattattcgagacagtgtggaagtcggtattgatccagcctatatgggaatttcgccgattaaagccattcaaaaactgttagcgcgcaatcaacttactacggaagaaattgatctgtatgaaatcaacgaagcatttgcagcaacttcaatcgtggtccaaagagaactggctttaccagaggaaaaggtcaacatttatggtggcggtatttcattaggtcatgcgattggtgccacaggtgctcgtttattaacgagtttaagttatcaattaaatcaaaaagaaaagaaatatggagtggcttctttatgtatcggcggtggcttaggactcgctatgctactagagagacctcagcaaaaaaaaaacagccgattttatcaaatgagtcctgaggaacgcctggcttctcttcttaatgaaggccagatttctgctgatacaaaaaaagaatttgaaaatacggctttatcttcgcagattgccaatcatatgattgaaaatcaaatcagtgaaacagaagtgccgatgggcgttggcttacatttaacagtggacgaaactgattatttggtaccaatggcgacagaagagccctcagtgattgcggctttgagtaatggtgcaaaaatagcacaaggatttaaaacagtgaatcaacaacgtttaatgcgtggacaaatcgttttttacgatgttgcagacgccgagtcattgattgatgaactacaagtaagagaaacggaaatttttcaacaagcagagttaagttatccatctatcgttaaacgcggcggcggcttaagagatttgcaatatcgtgcttttgatgaatcatttgtatctgtcgactttttagtagatgttaaggatgcaatgggggcaaatatcgttaacgctatgttggaaggtgtggccgagttgttccgtgaatggtttgcggagcaaaagattttattcagtattttaagtaattatgccacggagtcggttgttacgatgaaaacggctattccagtttcacgtttaagtaaggggagcaatggccgggaaattgctgaaaaaattgttttagcttcacgctatgcttcattagatccttatcgggcagtcacgcataacaaagggatcatgaatggcattgaagctgtcgttttagctacaggaaatgatacacgcgctgttagcgcttcttgtcatgcttttgcggtgaaggaaggtcgctaccaaggtttgactagttggacgctggatggcgaacaactaattggtgaaatttcagttccgcttgcgttagccacggttggcggtgccacaaaagtcttacctaaatctcaagcagctgctgatttgttagcagtgacggatgcaaaagaactaagtcgagtagtagcggctgttggtttggcacaaaatttagcggcgttacgggccttagtctctgaaggaattcaaaaaggacacatggctctacaagcacgttctttagcgatgacggtcggagctactggtaaagaagttgaggcagtcgctcaacaattaaaacgtcaaaaaacgatgaaccaagaccgagccttggctattttaaatgatttaagaaaacaataaaaaaacagttcagcagaaattattctgctgaactgttttttttcacattaggtagccgtttcaggccacgagctcaggagttaaagaaatgacaattgggattgataaaattagtttttttgtgcccccttattatattgatatgacggcactggctgaagccagaaatgtagaccctggaaaatttcatattggtattgggcaagaccaaatggcggtgaacccaatcagccaagatattgtgacatttgcagccaatgccgcagaagcgatcttgaccaaagaagataaagaggccattgatatggtgattgtcgggactgagtccagtatcgatgagtcaaaagcggccgcagttgtcttacatcgtttaatggggattcaacctttcgctcgctctttcgaaatcaaggaagcttgttacggagcaacagcaggcttacagttagctaagaatcacgtagccttacatccagataaaaaagtcttggttgtagcagcagatattgcaaaatatggattaaattctggcggtgagcctacacaaggagctggggcggttgcaatgttagttgctagtgaaccgcgcatcttggctttaaaagaggataatgtgatgctgacgcaagatatctatgacttttggcgtccaacaggccatccgtatcctatggtcgatggtcctttgtcaaacgaaacctacatccaatcttttgcccaagtctgggatgaacataaaaaaagaaccggtcttgattttgcagattatgatgctttagcgttccatattccttacacaaaaatgggcaaaaaagccttattagcaaaaatctccgaccaaactgaagcagaacaggaacgaattttagcccgttatgaagaaagcatcatctatagtcgtcgcgtaggaaacttgtatacgggttcactttatctgggactcatttcccttttagaaaatgcaacgactttaaccgcaggcaatcaaattgggttattcagttatggttctggtgctgtcgctgaatttttcactggtgaattagtagctggttatcaaaatcatttacaaaaagaaactcatttagcactgctagataatcggacagaactttctatcgctgaatatgaagccatgtttgcagaaactttagacacagatattgatcaaacgttagaagatgaattaaaatatagtatttctgctattaataataccgttcgctcttatcgaaactaactgcagcctcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgagcttatcgatgataagctgtcaaacatgagaattacaacttatatcgtatggggctgacttcaggtgctacatttgaagagataaattgcactgaaatctagaaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccaggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaaagtactgcgatgagtggcagggcggggcgtaatttttttaaggcagttattggtgcccttaaacgcctggtgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagagcctgataaaaacggttagcgcttcgttaatacagatgtaggtgttccacagggtagccagcagcatcctgcgatgcagatccggaacataatggtgcagggcgcttgtttcggcgtgggtatggtggcaggccccgtggccgggggactgttgggcgctgccggcacctgtcctacgagttgcatgataaagaagacagtcataagtgcggcgacgatagtcatgccccgcgcccaccggaaggagctaccggacagcggtgcggactgttgtaactcagaataagaaatgaggccgctcatggcgttccaatacgcaaaccgcctctccccgcgcgttggccgattcattaatgcagctggcacgacaggtttcccgactggaaagcgggcagtga

SEQ ID NO: 80 - DNA, 9081 bp, synthetic construct
  misc_feature 1..9081: note = pSNAK(-E)
  source 1..9081: mol_type = other DNA
aacgctatgttggaaggtgtggccgagttgttccgtgaatggtttgcggagcaaaagattttattcagtattttaagtaattatgccacggagtcggttgttacgatgaaaacggctattccagtttcacgtttaagtaaggggagcaatggccgggaaattgctgaaaaaattgttttagcttcacgctatgcttcattagatccttatcgggcagtcacgcataacaaagggatcatgaatggcattgaagctgtcgttttagctacaggaaatgatacacgcgctgttagcgcttcttgtcatgcttttgcggtgaaggaaggtcgctaccaaggtttgactagttggacgctggatggcgaacaactaattggtgaaatttcagttccgcttgcgttagccacggttggcggtgccacaaaagtcttacctaaatctcaagcagctgctgatttgttagcagtgacggatgcaaaagaactaagtcgagtagtagcggctgttggtttggcacaaaatttagcggcgttacgggccttagtctctgaaggaattcaaaaaggacacatggctctacaagcacgttctttagcgatgacggtcggagctactggtaaagaagttgaggcagtcgctcaacaattaaaacgtcaaaaaacgatgaaccaagaccgagccttggctattttaaatgatttaagaaaacaataaaaaaacagttcagcagaaattattctgctgaactgttttttttcacattaggtagccgtttcaggccacgagctcaggagttaaagaaatgacaattgggattgataaaattagtttttttgtgcccccttattatattgatatgacggcactggctgaagccagaaatgtagaccctggaaaatttcatattggtattgggcaagaccaaatggcggtgaacccaatcagccaagatattgtgacatttgcagccaatgccgcagaagcgatcttgaccaaagaagataaagaggccattgatatggtgattgtcgggactgagtccagtatcgatgagtcaaaagcggccgcagttgtcttacatcgtttaatggggattcaacctttcgctcgctctttcgaaatcaaggaagcttgttacggagcaacagcaggcttacagttagctaagaatcacgtagccttacatccagataaaaaagtcttggttgtagcagcagatattgcaaaatatggattaaattctggcggtgagcctacacaaggagctggggcggttgcaatgttagttgctagtgaaccgcgcatcttggctttaaaagaggataatgtgatgctgacgcaagatatctatgacttttggcgtccaacaggccatccgtatcctatggtcgatggtcctttgtcaaacgaaacctacatccaatcttttgcccaagtctgggatgaacataaaaaaagaaccggtcttgattttgcagattatgatgctttagcgttccatattccttacacaaaaatgggcaaaaaagccttattagcaaaaatctccgaccaaactgaagcagaacaggaacgaattttagcccgttatgaagaaagcatcatctatagtcgtcgcgtaggaaacttgtatacgggttcactttatctgggactcatttcccttttagaaaatgcaacgactttaaccgcaggcaatcaaattgggttattcagttatggttctggtgctgtcgctgaatttttcactggtgaattagtagctggttatcaaaatcatttacaaaaagaaactcatttagcactgctagataatcggacagaactttctatcgctgaatatgaagccatgtttgcagaaactttagacacagatattgatcaaacgttagaagatgaattaaaatatagtatttctgctattaataataccgttcgctcttatcgaaactaactgcagcctcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgagcttatcgatgataagctgtcaaacatgagaattacaacttatatcgtatggggctgacttcaggtgctacatttgaagagataaattgcactgaaatctagaaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccaggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaaagtactgcgatgagtggcagggcggggcgtaatttttttaaggcagttattggtgcccttaaacgcctggtgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagagcctgataaaaacggttagcgcttcgttaatacagatgtaggtgttccacagggtagccagcagcatcctgcgatgcagatccggaacataatggtgcagggcgcttgtttcggcgtgggtatggtggcaggccccgtggccgggggactgttgggcgctgccggcacctgtcctacgagttgcatgataaagaagacagtcataagtgcggcgacgatagtcatgccccgcgcccaccggaaggagctaccggacagcggtgcggactgttgtaactcagaataagaaatgaggccgctcatggcgttccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatcctacctgacgctttttatcgcaactctctactgtttctccatacccgtttttttgggctagcaggaggaattcaccatggtaccaatgacaaaaaaagttggtgtcggtcaggcacatagtaagataattttaataggggaacatgcggtcgtttacggttatcctgccatttccctgcctcttttggaggtggaggtgacctgtaaggtagttcctgcagagagtccttggcgcctttatgaggaggataccttgtccatggcggtttatgcctcactggagtatttgaatatcacagaagcctgcattcgttgtgagattgactcggctatccctgagaaacgggggatgggttcgtcagcggctatcagcatagcggccattcgtgcagtatttgactactatcaggctgatctgcctcatgatgtactagaaatcttggtcaatcgagctgaaatgattgcccatatgaatcctagtggtttggatgctaagacctgtcttagtgaccaacctattcgctttatcaagaacgtaggatttacagaacttgagatggatttatccgcctatttggtgattgccgatacgggtgtttatggtcatactcgtgaagccatccaagtggttcaaaataagggcaaggatgccctaccgtttttgcatgccttgggagaattaacccagcaagcagaagttgcgatttcacaaaaagatgctgaaggactgggacaaatcctcagtcaagcgcatttacatttaaaagaaattggagtcagtagccctgaggcagactttttggttgaaacgactcttagccatggtgctctgggtgccaagatgagcggtggtgggctaggaggttgtatcatagccttggtaaccaatttgacacacgcacaagaactagcagaaagattagaagagaaaggagctgttcagacatggatagagagcctgtaacagtacgttcctacgcaaatattgctattatcaaatattggggaaagaaaaaagaaaaagagatggtgcctgctactagcagtatttctctaactttggaaaatatgtatacagagacgaccttgtcgcctttaccagccaatgtaacagctgacgaattttacatcaatggtcagctacaaaatgaggtcgagcatgccaagatgagtaagattattgaccgttatcgtccagctggtgagggctttgtccgtatcgatactcaaaacaatatgcctacggcagcgggcctgtcctcaagttctagtggtttgtccgccctggtcaaggcttgtaatgcttatttcaagcttggattggatagaagtcagttggcacaggaagccaaatttgcctcaggctcttcttctcggagtttttatggaccactaggagcctgggataaggatagtggagaaatttaccctgtagagacagacttgaaactagctatgattatgttggtgctagaggacaagaaaaaaccaatctctagccgtgacgggatgaaactttgtgtggaaacctcgacgacttttgacgactgggttcgtcagtctgagaaggactatcaggatatgctgatttatctcaaggaaaatgattttgccaagattggagaattaacggagaaaaatgctctggctatgcatgctacgacaaagactgctagtccagccttttcttatctgacggatgcctcttatgaggctatggcctttgttcgccagcttcgtgagaaaggagaggcctgctactttaccatggatgctggtcccaatgttaaggtcttctgtcaggagaaagacttggagcatttgtcagaaattttcggtcagcgttatcgcttgattgtgtcaaaaacaaaggatttgagtcaagatgattgctgttaaaacttgcggaaaactctattgggcaggtgaatatgctattttagagccagggcagttagctttgataaaggatattcccatctatatgagggctgagattgctttttctgacagctaccgtatctattcagatatgtttgatttcgcagtggacttaaggcccaatcctgactacagcttgattcaagaaacgattgctttgatgggagacttcctcgctgttcgcggtcagaatttaagacctttttccctaaaaatctgtggcaaaatggaacgagaagggaaaaagtttggtctaggttctagtggcagcgtcgttgtcttggttgtcaaggctttactggctctctataatctttcggttgatcagaatctcttgttcaagctgactagcgctgtcttgctcaagcgaggagacaatggttccatgggcgaccttgcctgtattgtggcagaggatttggttctttaccagtcatttgatcgccagaaggcggctgcttggttagaagaagaaaacttggcgacagttctggagcgtgattggggattttttatctcacaagtgaaaccaactttagaatgtgatttcttagtgggatggaccaaggaagtggctgtatcgagtcacatggtccagcaaatcaagcaaaatatcaatcaaaattttttaagttcctcaaaagaaacggtggtttctttggtcgaagccttggagcaggggaaagccgaaaaagttatcgagcaagtagaagtagccagcaagcttttagaaggcttgagtacagatatttacacgcctttgcttagacagttgaaagaagccagtcaagatttgcaggccgttgccaagagtagtggtgctggtggtggtgactgtggcatcgccctgagttttgatgcgcaatcttctcgaaacactttaaaaaatcgttgggccgatctggggattgagctcttatatcaagaaaggataggacatgacgacaaatcgtaatctagccccgggaggagagaaattatgcaaacggaacacgtcattttattgaatgcacagggagttcccacgggtacgctggaaaagtatgccgcacacacggcagacacccgcttacatctcgcgttctccagttggctgtttaatgccaaaggacaattattagttacccgccgcgcactgagcaaaaaagcatggcctggcgtgtggactaactcggtttgtgggcacccacaactgggagaaagcaacgaagacgcagtgatccgccgttgccgttatgagcttggcgtggaaattacgcctcctgaatctatctatcctgactttcgctaccgcgccaccgatccgagtggcattgtggaaaatgaagtgtgtccggtatttgccgcacgcaccactagtgcgttacagatcaatgatgatgaagtgatggattatcaatggtgtgatttagcagatgtattacacggtattgatgccacgccgtgggcgttcagtccgtggatggtgatgcaggcgacaaatcgcgaagccagaaaacgattatctgcatttacccagcttaaataagcatgcactagagtcgaggaaacagaccatggagttgaaaacagtagttattattgatgcattacgaacaccaattggaaaatataaaggcagcttaagtcaagtaagtgccgtagacttaggaacacatgttacaacacaacttttaaaaagacattccactatttctgaagaaattgatcaagtaatctttggaaatgttttacaagctggaaatggccaaaatcccgcacgacaaatagcaataaacagcggtttatctcatgaaattcccgcaatgacagttaatgaggtctgcggatcaggaatgaaggccgttattttggcgaaacaattgattcaattaggagaagcggaagttttaattgctggcgggattgagaatatgtcccaagcacctaaattacaacgatttaattacgaaacagaaagctatgatgcgcctttttctagtatgatgtacgatgggtt

SEQ ID NO: 81 - DNA, 8230 bp, synthetic construct
  misc_feature 1..8230: note = pSNA(-E)free
  source 1..8230: mol_type = other DNA
tttttttaaggcagttattggtgcccttaaacgcctggtgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagagcctgataaaaacggttagcgcttcgttaatacagatgtaggtgttccacagggtagccagcagcatcctgcgatgcagatccggaacataatggtgcagggcgcttgtttcggcgtgggtatggtggcaggccccgtggccgggggactgttgggcgctgccggcacctgtcctacgagttgcatgataaagaagacagtcataagtgcggcgacgatagtcatgccccgcgcccaccggaaggagctaccggacagcggtgcggactgttgtaactcagaataagaaatgaggccgctcatggcgttccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatcctacctgacgctttttatcgcaactctctactgtttctccatacccgtttttttgggctagcaggaggaattcaccatggtaccaatgacaaaaaaagttggtgtcggtcaggcacatagtaagataattttaataggggaacatgcggtcgtttacggttatcctgccatttccctgcctcttttggaggtggaggtgacctgtaaggtagttcctgcagagagtccttggcgcctttatgaggaggataccttgtccatggcggtttatgcctcactggagtatttgaatatcacagaagcctgcattcgttgtgagattgactcggctatccctgagaaacgggggatgggttcgtcagcggctatcagcatagcggccattcgtgcagtatttgactactatcaggctgatctgcctcatgatgtactagaaatcttggtcaatcgagctgaaatgattgcccatatgaatcctagtggtttggatgctaagacctgtcttagtgaccaacctattcgctttatcaagaacgtaggatttacagaacttgagatggatttatccgcctatttggtgattgccgatacgggtgtttatggtcatactcgtgaagccatccaagtggttcaaaataagggcaaggatgccctaccgtttttgcatgccttgggagaattaacccagcaagcagaagttgcgatttcacaaaaagatgctgaaggactgggacaaatcctcagtcaagcgcatttacatttaaaagaaattggagtcagtagccctgaggcagactttttggttgaaacgactcttagccatggtgctctgggtgccaagatgagcggtggtgggctaggaggttgtatcatagccttggtaaccaatttgacacacgcacaagaactagcagaaagattagaagagaaaggagctgttcagacatggatagagagcctgtaacagtacgttcctacgcaaatattgctattatcaaatattggggaaagaaaaaagaaaaagagatggtgcctgctactagcagtatttctctaactttggaaaatatgtatacagagacgaccttgtcgcctttaccagccaatgtaacagctgacgaattttacatcaatggtcagctacaaaatgaggtcgagcatgccaagatgagtaagattattgaccgttatcgtccagctggtgagggctttgtccgtatcgatactcaaaacaatatgcctacggcagcgggcctgtcctcaagttctagtggtttgtccgccctggtcaaggcttgtaatgcttatttcaagcttggattggatagaagtcagttggcacaggaagccaaatttgcctcaggctcttcttctcggagtttttatggaccactaggagcctgggataaggatagtggagaaatttaccctgtagagacagacttgaaactagctatgattatgttggtgctagaggacaagaaaaaaccaatctctagccgtgacgggatgaaactttgtgtggaaacctcgacgacttttgacgactgggttcgtcagtctgagaaggactatcaggatatgctgatttatctcaaggaaaatgattttgccaagattggagaattaacggagaaaaatgctctggctatgcatgctacgacaaagactgctagtccagccttttcttatctgacggatgcctcttatgaggctatggcctttgttcgccagcttcgtgagaaaggagaggcctgctactttaccatggatgctggtcccaatgttaaggtcttctgtcaggagaaagacttggagcatttgtcagaaattttcggtcagcgttatcgcttgattgtgtcaaaaacaaaggatttgagtcaagatgattgctgttaaaacttgcggaaaactctattgggcaggtgaatatgctattttagagccagggcagttagctttgataaaggatattcccatctatatgagggctgagattgctttttctgacagctaccgtatctattcagatatgtttgatttcgcagtggacttaaggcccaatcctgactacagcttgattcaagaaacgattgctttgatgggagacttcctcgctgttcgcggtcagaatttaagacctttttccctaaaaatctgtggcaaaatggaacgagaagggaaaaagtttggtctaggttctagtggcagcgtcgttgtcttggttgtcaaggctttactggctctctataatctttcggttgatcagaatctcttgttcaagctgactagcgctgtcttgctcaagcgaggagacaatggttccatgggcgaccttgcctgtattgtggcagaggatttggttctttaccagtcatttgatcgccagaaggcggctgcttggttagaagaagaaaacttggcgacagttctggagcgtgattggggattttttatctcacaagtgaaaccaactttagaatgtgatttcttagtgggatggaccaaggaagtggctgtatcgagtcacatggtccagcaaatcaagcaaaatatcaatcaaaattttttaagttcctcaaaagaaacggtggtttctttggtcgaagccttggagcaggggaaagccgaaaaagttatcgagcaagtagaagtagccagcaagcttttagaaggcttgagtacagatatttacacgcctttgcttagacagttgaaagaagccagtcaagatttgcaggccgttgccaagagtagtggtgctggtggtggtgactgtggcatcgccctgagttttgatgcgcaatcttctcgaaacactttaaaaaatcgttgggccgatctggggattgagctcttatatcaagaaaggataggacatgacgacaaatcgtaatctagccccgggaggagagaaattatgcaaacggaacacgtcattttattgaatgcacagggagttcccacgggtacgctggaaaagtatgccgcacacacggcagacacccgcttacatctcgcgttctccagttggctgtttaatgccaaaggacaattattagttacccgccgcgcactgagcaaaaaagcatggcctggcgtgtggactaactcggtttgtgggcacccacaactgggagaaagcaacgaagacgcagtgatccgccgttgccgttatgagcttggcgtggaaattacgcctcctgaatctatctatcctgactttcgctaccgcgccaccgatccgagtggcattgtggaaaatgaagtgtgtccggtatttgccgcacgcaccactagtgcgttacagatcaatgatgatgaagtgatggattatcaatggtgtgatttagcagatgtattacacggtattgatgccacgccgtgggcgttcagtccgtggatggtgatgcaggcgacaaatcgcgaagccagaaaacgattatctgcatttacccagcttaaataagcatgcactagagtcgaggaaacagaccatggagttgaaaacagtagttattattgatgcattacgaacaccaattggaaaatataaaggcagcttaagtcaagtaagtgccgtagacttaggaacacatgttacaacacaacttttaaaaagacattccactatttctgaagaaattgatcaagtaatctttggaaatgttttacaagctggaaatggccaaaatcccgcacgacaaatagcaataaacagcggtttatctcatgaaattcccgcaatgacagttaatgaggtctgcggatcaggaatgaaggccgttattttggcgaaacaattgattcaattaggagaagcggaagttttaattgctggcgggattgagaatatgtcccaagcacctaaattacaacgatttaattacgaaacagaaagctatgatgcgcctttttctagtatgatgtacgatgggttaacgctatgttggaaggtgtggccgagttgttccgtgaatggtttgcggagcaaaagattttattcagtattttaagtaattatgccacggagtcggttgttacgatgaaaacggctattccagtttcacgtttaagtaaggggagcaatggccgggaaattgctgaaaaaattgttttagcttcacgctatgcttcattagatccttatcgggcagtcacgcataacaaagggatcatgaatggcattgaagctgtcgttttagctacaggaaatgatacacgcgctgttagcgcttcttgtcatgcttttgcggtgaaggaaggtcgctaccaaggtttgactagttggacgctggatggcgaacaactaattggtgaaatttcagttccgcttgcgttagccacggttggcggtgccacaaaagtcttacctaaatctcaagcagctgctgatttgttagcagtgacggatgcaaaagaactaagtcgagtagtagcggctgttggtttggcacaaaatttagcggcgttacgggccttagtctctgaaggaattcaaaaaggacacatggctctacaagcacgttctttagcgatgacggtcggagctactggtaaagaagttgaggcagtcgctcaacaattaaaacgtcaaaaaacgatgaaccaagaccgagccttggctattttaaatgatttaagaaaacaataaaaaaacagttcagcagaaattattctgctgaactgttttttttcacattaggtagccgtttcaggccacgagctcaggagttaaagaaatgacaattgggattgataaaattagtttttttgtgcccccttattatattgatatgacggcactggctgaagccagaaatgtagaccctggaaaatttcatattggtattgggcaagaccaaatggcggtgaacccaatcagccaagatattgtgacatttgcagccaatgccgcagaagcgatcttgaccaaagaagataaagaggccattgatatggtgattgtcgggactgagtccagtatcgatgagtcaaaagcggccgcagttgtcttacatcgtttaatggggattcaacctttcgctcgctctttcgaaatcaaggaagcttgttacggagcaacagcaggcttacagttagctaagaatcacgtagccttacatccagataaaaaagtcttggttgtagcagcagatattgcaaaatatggattaaattctggcggtgagcctacacaaggagctggggcggttgcaatgttagttgctagtgaaccgcgcatcttggctttaaaagaggataatgtgatgctgacgcaagatatctatgacttttggcgtccaacaggccatccgtatcctatggtcgatggtcctttgtcaaacgaaacctacatccaatcttttgcccaagtctgggatgaacataaaaaaagaaccggtcttgattttgcagattatgatgctttagcgttccatattccttacacaaaaatgggcaaaaaagccttattagcaaaaatctccgaccaaactgaagcagaacaggaacgaattttagcccgttatgaagaaagcatcatctatagtcgtcgcgtaggaaacttgtatacgggttcactttatctgggactcatttcccttttagaaaatgcaacgactttaaccgcaggcaatcaaattgggttattcagttatggttctggtgctgtcgctgaatttttcactggtgaattagtagctggttatcaaaatcatttacaaaaagaaactcatttagcactgctagataatcggacagaactttctatcgctgaatatgaagccatgtttgcagaaactttagacacagatattgatcaaacgttagaagatgaattaaaatatagtatttctgctattaataataccgttcgctcttatcgaaactaactgcagcctcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgagcttatcgatgataagctgtcaaacatgagaattacaacttatatcgtatggggctgacttcaggtgctacatttgaagagataaattgcactgaaatctagaaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccaggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagct

SEQ ID NO: 82 - DNA, 6678 bp, synthetic construct
  misc_feature 1..6678: note = pTEFAmvaE
  source 1..6678: mol_type = other DNA
gtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatggggctgcaggaattcgatgccatggagttgaaaacagtagttattattgatgcattacgaacaccaattggaaaatataaaggcagcttaagtcaagtaagtgccgtagacttaggaacacatgttacaacacaacttttaaaaagacattccactatttctgaagaaattgatcaagtaatctttggaaatgttttacaagctggaaatggccaaaatcccgcacgacaaatagcaataaacagcggtttgtctcatgaaattcccgcaatgacggttaatgaggtctgcggatcaggaatgaaggccgttattttggcgaaacaattgattcaattaggagaagcggaagttttaattgctggcgggattgagaatatgtcccaagcacctaaattacaacgttttaattacgaaacagaaagctacgatgcgcctttttctagtatgatgtatgatggattaacggatgcctttagtggtcaggcaatgggcttaactgctgaaaatgtggccgaaaagtatcatgtaactagagaagagcaagatcaattttctgtacattcacaattaaaagcagctcaagcacaagcagaagggatattcgctgacgaaatagccccattagaagtatcaggaacgcttgtggagaaagatgaagggattcgccctaattcgagcgttgagaagctaggaacgcttaaaacagtttttaaagaagacggtactgtaacagcagggaatgcatcaaccattaatgatggggcttctgctttgattattgcttcacaagaatatgccgaagcacacggtcttccttatttagctattattcgagacagtgtggaagtcggtattgatccagcctatatgggaatttcgccgattaaagccattcaaaaactgttagcgcgcaatcaacttactacggaagaaattgatctgtatgaaatcaacgaagcatttgcagcaacttcaatcgtggtccaaagagaactggctttaccagaggaaaaggtcaacatttatggtggcggtatttcattaggtcatgcgattggtgccacaggtgctcgtttattaacgagtttaagttatcaattaaatcaaaaagaaaagaaatatggagtggcttctttatgtatcggcggtggcttaggactcgctatgctactagagagacctcagcaaaaaaaaaacagccgattttatcaaatgagtcctgaggaacgcctggcttctcttcttaatgaaggccagatttctgctgatacaaaaaaagaatttgaaaatacggctttatcttcgcagattgccaatcatatgattgaaaatcaaatcagtgaaacagaagtgccgatgggcgttggcttacatttaacagtggacgaaactgattatttggtaccaatggcgacagaagagccctcagttattgcggctttgagtaatggtgcaaaaatagcacaaggatttaaaacagtgaatcaacaacgcttaatgcgtggacaaatcgttttttacgatgttgcagatcccgagtcattgattgataaactacaagtaagagaagcggaagtttttcaacaagcagagttaagttatccatctatcgttaaacggggcggcggcttaagagatttgcaatatcgtacttttgatgaatcatttgtatctgtcgactttttagtagatgttaaggatgcaatgggggcaaatatcgttaacgctatgttggaaggtgtggccgagttgttccgtgaatggtttgcggagcaaaagattttattcagtattttaagtaattatgccacggagtcggttgttacgatgaaaacggctattccagtttcacgtttaagtaaggggagcaatggccgggaaattgctgaaaaaattgttttagcttcacgctatgcttcattagatccttatcgggcagtcacgcataacaaaggaatcatgaatggcattgaagctgtagttttagctacaggaaatgatacacgcgctgttagcgcttcttgtcatgcttttgcggtgaaggaaggtcgctaccaaggcttgactagttggacgctggatggcgaacaactaattggtgaaatttcagttccgcttgctttagccacggttggcggtgccacaaaagtcttacctaaatctcaagcagctgctgatttgttagcagtgacggatgcaaaagaactaagtcgagtagtagcggctgttggtttggcacaaaatttagcggcgttacgggccttagtctctgaaggaattcaaaaaggacacatggctctacaagcacgttctttagcgatgacggtcggagctactggtaaagaagttgaggcagtcgctcaacaattaaaacgtcaaaaaacgatgaaccaagaccgagccatggctattttaaatgatttaagaaaacaataaaaaaacagttcagcagaaattattctgctgaactattttttttcacattaggtagccgtttcaggccacgagctcggtacccggggatcctctagagtcgacctgcaggcatgcaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctacagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctg

SEQ ID NO: 83 - DNA, 13288 bp, synthetic construct
  misc_feature 1..13288: note = pT-DHBSRYbbO
  source 1..13288: mol_type = other DNA
gtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatggaattctacgtactagtgaattcaggaggtaataaatatgggtctgatgctgattgattggtgtgcactggctctggttgttttcattggcctgccgcacggcgcgctggatgctgccatttctttttctatgatctcttctgcaaaacgcattgctcgtctggctggtattctgctgatctatctgctgctggcgaccgcgttcttcctgatctggtatcagctgccagcgtttagcctgctgatcttcctgctgatctccattatccactttggtatggcagacttcaacgcgtccccaagcaaactgaaatggccgcatatcatcgcccacggcggtgttgttactgtttggctgccgctgatccagaaaaacgaagtaactaaactgtttagcatcctgactaacggtccgactccgatcctgtgggacatcctgctgattttcttcctgtgttggtctattggcgtgtgtctgcacacgtacgaaaccctgcgctctaaacattacaacatcgcctttgaactgatcggtctgattttcctggcgtggtatgcgccgcctctggttacgtttgccacttacttctgcttcattcattcccgtcgccacttctcctttgtgtggaagcagctgcaacacatgtcttccaaaaagatgatgattggcagcgcgattatcctgtcctgtacctcttggctgatcggcggtggtatctatttcttcctgaactccaaaatgatcgcctctgaggctgcgctgcagactgtgttcatcggtctggcggcactgaccgtgccgcacatgattctgatcgacttcatcttccgtccgcactcttcccgtatcaaaatcaaaaactaattaattaaaggaggtaataatatgactcataaagcaacggagatcctgacaggtaaagttatgcaaaaatcggtcttaattaccggatgttccagtggaattggcctggaaagcgcgctcgaattaaaacgccagggttttcatgtgctggcaggttgccggaaaccggatgatgttgagcgcatgaacagcatgggatttaccggcgtgttgatcgatctggattcaccagaaagtgttgatcgcgcagccgacgaggtgatcgccctgaccgataattgtctgtatgggatctttaacaatgccggattcggcatgtatggccccctttccaccatcagccgtgcgcagatggaacagcagttttccgccaactttttcggcgcacaccagctcaccatgcgcctgttacccgcgatgttaccgcacggtgaagggcgtattgtgatgacatcatcggtgatgggattaatctccacgccgggtcgtggcgcttacgcggccagtaaatatgcgctggaggcgtggtcagatgcactgcgcatggagctgcgccacagcggaattaaagtcagcctgatcgaacccggtcccattcgtactcgcttcaccgacaacgtcaaccagacgcaaagtgataaaccagtcgaaaatcccggcatcgccgcccgctttacgttgggaccggaagcggtggtggacaaagtacgccatgcttttattagcgagaagccgaagatgcgctatccggtgacgctggtgacctgggcggtaatggtgcttaagcgcctgctgccggggcgcgtgatggacaaaatattgcaggggtaactagtaggaggtaataaatatggtgagtggcagtaaagcgggcgtttcgcctcatcgcgaaatagaagtaatgagacaatccattgacgatcacctggctggcctgttacctgaaaccgacagccaggatatcgtcagccttgcgatgcgtgaaggcgtcatggcacccggtaaacggatccgtccgctgctgatgctgctggccgcccgcgacctccgctaccagggcagtatgcctacgctgctcgatctcgcctgcgccgttgaactgacccataccgcgtcgctgatgctcgacgacatgccctgcatggacaacgccgagctgcgccgcggtcagcccactacccacaaaaaatttggtgagagcgtggcgatccttgcctccgttgggctgctctctaaagcctttggtctgatcgccgccaccggcgatctgccgggggagaggcgtgcccaggcggtcaacgagctctctaccgccgtgggcgtgcagggcctggtactggggcagtttcgcgatcttaacgatgccgccctcgaccgtacccctgacgctatcctcagcaccaaccacctcaagaccggcattctgttcagcgcgatgctgcagatcgtcgccattgcttccgcctcgtcgccgagcacgcgagagacgctgcacgccttcgccctcgacttcggccaggcgtttcaactgctggacgatctgcgtgacgatcacccggaaaccggtaaagatcgcaataaggacgcgggaaaatcgacgctggtcaaccggctgggcgcagacgcggcccggcaaaagctgcgcgagcatattgattccgccgacaaacacctcacttttgcctgtccgcagggcggcgccatccgacagtttatgcatctgtggtttggccatcaccttgccgactggtcaccggtcatgaaaatcgcctgataactcgaggaggtataaaggatgaaaaaaaccgttgtgattggcgcaggctttggtggcctggcgctggcgattcgcctgcaggcggcagggatcccaaccgtactgctggagcagcgggacaagcccggcggtcgggcctacgtctggcatgaccagggctttacctttgacgccgggccgacggtgatcaccgatcctaccgcgcttgaggcgctgttcaccctggccggcaggcgcatggaggattacgtcaggctgctgccggtaaaacccttctaccgactctgctgggagtccgggaagaccctcgactatgctaacgacagcgccgagcttgaggcgcagattacccagttcaacccccgcgacgtcgagggctaccggcgctttctggcttactcccaggcggtattccaggagggatatttgcgcctcggcagcgtgccgttcctctcttttcgcgacatgctgcgcgccgggccgcagctgcttaagctccaggcgtggcagagcgtctaccagtcggtttcgcgctttattgaggatgagcatctgcggcaggccttctcgttccactccctgctggtaggcggcaaccccttcaccacctcgtccatctacaccctgatccacgcccttgagcgggagtggggggtctggttccctgagggcggcaccggggcgctggtgaacggcatggtgaagctgtttaccgatctgggcggggagatcgaactcaacgcccgggtcgaagagctggtggtggccgataaccgcgtaagccaggtccggctggcggatggtcggatctttgacaccgacgccgtagcctcgaacgctgacgtggtgaacacctataaaaagctgctcggccaccatccggtggggcagaagcgggcggcagcgctggagcgcaagagcatgagcaactcgctgtttgtgctctacttcggcctgaaccagcctcattcccagctggcgcaccataccatctgttttggtccccgctaccgggagctgatcgacgagatctttaccggcagcgcgctggcggatgacttctcgctctacctgcactcgccctgcgtgaccgatccctcgctcgcgcctcccggctgcgccagcttctacgtgctggccccggtgccgcatcttggcaacgcgccgctggactgggcgcaggaggggccgaagctgcgcgaccgcatctttgactaccttgaagagcgctatatgcccggcctgcgtagccagctggtgacccagcggatctttaccccggcagacttccacgacacgctggatgcgcatctgggatcggccttctccatcgagccgctgctgacccaaagcgcctggttccgcccgcacaaccgcgacagcgacattgccaacctctacctggtgggcgcaggtactcaccctggggcgggcattcctggcgtagtggcctcggcgaaagccaccgccagcctgatgattgaggatctgcaatgagccaaccgccgctgcttgaccacgccacgcagaccatggccaacggctcgaaaagttttgccaccgctgcgaagctgttcgacccggccacccgccgtagcgtgctgatgctctacacctggtgccgccactgcgatgacgtcattgacgaccagacccacggcttcgccagcgaggccgcggcggaggaggaggccacccagcgcctggcccggctgcgcacgctgaccctggcggcgtttgaaggggccgagatgcaggatccggccttcgctgcctttcaggaggtggcgctgacccacggtattacgccccgcatggcgctcgatcacctcgacggctttgcgatggacgtggctcagacccgctatgtcacctttgaggatacgctgcgctactgctatcacgtggcgggcgtggtgggtctgatgatggccagggtgatgggcgtgcgggatgagcgggtgctggatcgcgcctgcgatctggggctggccttccagctgacgaatatcgcccgggatattattgacgatgcggctattgaccgctgctatctgcccgccgagtggctgcaggatgccgggctgaccccggagaactatgccgcgcgggagaatcgggccgcgctggcgcgggtggcggagcggcttattgatgccgcagagccgtactacatctcctcccaggccgggctacacgatctgccgccgcgctgcgcctgggcgatcgccaccgcccgcagcgtctaccgggagatcggtattaaggtaaaagcggcgggaggcagcgcctgggatcgccgccagcacaccagcaaaggtgaaaaaattgccatgctgatggcggcaccggggcaggttattcgggcgaagacgacgagggtgacgccgcgtccggccggtctttggcagcgtcccgtttagtaatctagaggaggtaataaaatatgcttcgttcgttgctcagaggcctcacgcatatcccccgcgtgaactccgcccagcagcccagctgtgcacacgcgcgactccagtttaagctcaggagcatgcagatgacgctcatgcagcccagcatctcagccaatctgtcgcgcgccgaggaccgcacagaccacatgaggggtgcaagcacctgggcaggcgggcagtcgcaggatgagctgatgctgaaggacgagtgcatcttggtggatgttgaggacaacatcacaggccatgccagcaagctggagtgtcacaagttcctaccacatcagcctgcaggcctgctgcaccgggccttctctgtgttcctgtttgacgatcaggggcgactgctgctgcaacagcgtgcacgctcaaaaatcaccttcccaagtgtgtggacgaacacctgctgcagccaccctttacatgggcagaccccagatgaggtggaccaactaagccaggtggccgacggaacagtacctggcgcaaaggctgctgccatccgcaagttggagcacgagctggggataccagcgcaccagctgccggcaagcgcgtttcgcttcctcacgcgtttgcactactgtgccgcggacgtgcagccagctgcgacacaatcagcgctctggggcgagcacgaaatggactacatcttgttcatccgggccaacgtcaccttggcgcccaaccctgacgaggtggacgaagtcaggtacgtgacgcaagaggagctgcggcagatgatgcagccggacaacgggctgcaatggtcgccgtggtttcgcatcatcgccgcgcgcttccttgagcgttggtgggctgacctggacgcggccctaaacactgacaaacacgaggattggggaacggtgcatcacatcaacgaagcgtgataagcggccgcgtcgacaggaggagcggctatgcaaccgcattatgatctgattctcgtgggggctggactcgcgaatggccttatcgccctgcgtcttcagcagcagcaacctgatatgcgtattttgcttatcgacgccgcaccccaggcgggcgggaatcatacgtggtcatttcaccacgatgatttgactgagagccaacatcgttggatagctccgctggtggttcatcactggcccgactatcaggtacgctttcccacacgccgtcgtaagctgaacagcggctacttttgtattacttctcagcgtttcgctgaggttttacagcgacagtttggcccgcacttgtggatggataccgcggtcgcagaggttaatgcggaatctgttcggttgaaaaagggtcaggttatcggtgcccgcgcggtgattgacgggcggggttatgcggcaaattcagcactgagcgtgggcttccaggcgtttattggccaggaatggcgattgagccacccgcatggtttatcgtctcccattatcatggatgccacggtcgatcagcaaaatggttatcgcttcgtgtacagcctgccgctctcgccgaccagattgttaattgaagacacgcactatattgataatgcgacattagatcctgaatgcgcgcggcaaaatatttgcgactatgccgcgcaacagggttggcagcttcagacactgctgcgagaagaacagggcgccttacccattactctgtcgggcaatgccgacgcattctggcagcagcgccccctggcctgtagtggattacgtgccggtctgttccatcctaccaccggctattcactgccgctggcggttgccgtggccgaccgcctgagtgcacttgatgtctttacgtcggcctcaattcaccatgccattacgcattttgcccgcgagcgctggcagcagcagggctttttccgcatgctgaatcgcatgctgtttttagccggacccgccgattcacgctggcgggttatgcagcgtttttatggtttacctgaagatttaattgcccgtttttatgcgggaaaactcacgctgaccgatcggctacgtattctgagcggcaagccgcctgttccggtattagcagcattgcaagccattatgacgactcatcgttaactgcaggcatgcaagctggggatcctacctgacgctttttatcgcaactctctactgtttctccatacccgtttttttgggctagcaggaggaattcaccatggaattcatgagttttgatattgccaaatacccgaccctggcactggtcgactccacccaggagttacgactgttgccgaaagagagtttaccgaaactctgcgacgaactgcgccgctatttactcgacagcgtgagccgttccagcgggcacttcgcctccgggctgggcacggtcgaactgaccgtggcgctgcactatgtctacaacaccccgtttgaccaattgatttgggatgtggggcatcaggcttatccgcataaaattttgaccggacgccgcgacaaaatcggcaccatccgtcagaaaggcggtctgcacccgttcccgtggcgcggcgaaagcgaatatgacgtattaagcgtcgggcattcatcaacctccatcagtgccggaattggtattgcggttgctgccgaaaaagaaggcaaaaatcgccgcaccgtctgtgtcattggcgatggcgcgattaccgcaggcatggcgtttgaagcgatgaatcacgcgggcgatatccgtcctgatatgctggtgattctcaacgacaatgaaatgtcgatttccgaaaatgtcggcgcgctcaacaaccatctggcacagctgctttccggtaagctttactcttcactgcgcgaaggcgggaaaaaagttttctctggcgtgccgccaattaaagagctgctcaaacgcaccgaagaacatattaaaggcatggtagtgcctggcacgttgtttgaagagctgggctttaactacatcggcccggtggacggtcacgatgtgctggggcttatcaccacgctaaagaacatgcgcgacctgaaaggcccgcagttcctgcatatcatgaccaaaaaaggtcgtggttatgaaccggcagaaaaagacccgatcactttccacgccgtgcctaaatttgatccctccagcggttgtttgccgaaaagtagcggcggtttgccgagctattcaaaaatctttggcgactggttgtgcgaaacggcagcgaaagacaacaagctgatggcgattactccggcgatgcgtgaaggttccggcatggtcgagttttcacgtaaattcccggatcgctacttcgacgtggcaattgccgagcaacacgcggtgacctttgctgcgggtctggcgattggtgggtacaaacccattgtcgcgatttactccactttcctgcaacgcgcctatgatcaggtgctgcatgacgtggcgattcaaaagcttccggtcctgttcgccatcgaccgcgcgggcattgttggtgctgacggtcaaacccatcagggtgcttttgatctctcttacctgcgctgcataccggaaatggtcattatgaccccgagcgatgaaaacgaatgtcgccagatgctctataccggctatcactataacgatggcccgtcagcggtgcgctacccgcgtggcaacgcggtcggcgtggaactgacgccgctggaaaaactaccaattggcaaaggcattgtgaagcgtcgtggcgagaaactggcgatccttaactttggtacgctgatgccagaagcggcgaaagtcgccgaatcgctgaacgccacgctggtcgatatgcgttttgtgaaaccgcttgatgaagcgttaattctggaaatggccgccagccatgaagcgctggtcaccgtagaagaaaacgccattatgggcggcgcaggcagcggcgtgaacgaagtgctgatggcccatcgtaaaccagtacccgtgctgaacattggcctgccggacttctttattccgcaaggaactcaggaagaaatgcgcgccgaactcggcctcgatgccgctggtatggaagccaaaatcaaggcctggctggcataaggtacccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctacagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctg

SEQ ID NO: 84 - DNA, 11378 bp, synthetic construct
  misc_feature 1..11378: note = pT-HBSRYbbO
  source 1..11378: mol_type = other DNA
gtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatggaattctacgtactagtgaattcaggaggtaataaatatgggtctgatgctgattgattggtgtgcactggctctggttgttttcattggcctgccgcacggcgcgctggatgctgccatttctttttctatgatctcttctgcaaaacgcattgctcgtctggctggtattctgctgatctatctgctgctggcgaccgcgttcttcctgatctggtatcagctgccagcgtttagcctgctgatcttcctgctgatctccattatccactttggtatggcagacttcaacgcgtccccaagcaaactgaaatggccgcatatcatcgcccacggcggtgttgttactgtttggctgccgctgatccagaaaaacgaagtaactaaactgtttagcatcctgactaacggtccgactccgatcctgtgggacatcctgctgattttcttcctgtgttggtctattggcgtgtgtctgcacacgtacgaaaccctgcgctctaaacattacaacatcgcctttgaactgatcggtctgattttcctggcgtggtatgcgccgcctctggttacgtttgccacttacttctgcttcattcattcccgtcgccacttctcctttgtgtggaagcagctgcaacacatgtcttccaaaaagatgatgattggcagcgcgattatcctgtcctgtacctcttggctgatcggcggtggtatctatttcttcctgaactccaaaatgatcgcctctgaggctgcgctgcagactgtgttcatcggtctggcggcactgaccgtgccgcacatgattctgatcgacttcatcttccgtccgcactcttcccgtatcaaaatcaaaaactaattaattaaaggaggtaataatatgactcataaagcaacggagatcctgacaggtaaagttatgcaaaaatcggtcttaattaccggatgttccagtggaattggcctggaaagcgcgctcgaattaaaacgccagggttttcatgtgctggcaggttgccggaaaccggatgatgttgagcgcatgaacagcatgggatttaccggcgtgttgatcgatctggattcaccagaaagtgttgatcgcgcagccgacgaggtgatcgccctgaccgataattgtctgtatgggatctttaacaatgccggattcggcatgtatggccccctttccaccatcagccgtgcgcagatggaacagcagttttccgccaactttttcggcgcacaccagctcaccatgcgcctgttacccgcgatgttaccgcacggtgaagggcgtattgtgatgacatcatcggtgatgggattaatctccacgccgggtcgtggcgcttacgcggccagtaaatatgcgctggaggcgtggtcagatgcactgcgcatggagctgcgccacagcggaattaaagtcagcctgatcgaacccggtcccattcgtactcgcttcaccgacaacgtcaaccagacgcaaagtgataaaccagtcgaaaatcccggcatcgccgcccgctttacgttgggaccggaagcggtggtggacaaagtacgccatgcttttattagcgagaagccgaagatgcgctatccggtgacgctggtgacctgggcggtaatggtgcttaagcgcctgctgccggggcgcgtgatggacaaaatattgcaggggtaactagtaggaggtaataaatatggtgagtggcagtaaagcgggcgtttcgcctcatcgcgaaatagaagtaatgagacaatccattgacgatcacctggctggcctgttacctgaaaccgacagccaggatatcgtcagccttgcgatgcgtgaaggcgtcatggcacccggtaaacggatccgtccgctgctgatgctgctggccgcccgcgacctccgctaccagggcagtatgcctacgctgctcgatctcgcctgcgccgttgaactgacccataccgcgtcgctgatgctcgacgacatgccctgcatggacaacgccgagctgcgccgcggtcagcccactacccacaaaaaatttggtgagagcgtggcgatccttgcctccgttgggctgctctctaaagcctttggtctgatcgccgccaccggcgatctgccgggggagaggcgtgcccaggcggtcaacgagctctctaccgccgtgggcgtgcagggcctggtactggggcagtttcgcgatcttaacgatgccgccctcgaccgtacccctgacgctatcctcagcaccaaccacctcaagaccggcattctgttcagcgcgatgctgcagatcgtcgccattgcttccgcctcgtcgccgagcacgcgagagacgctgcacgccttcgccctcgacttcggccaggcgtttcaactgctggacgatctgcgtgacgatcacccggaaaccggtaaagatcgcaataaggacgcgggaaaatcgacgctggtcaaccggctgggcgcagacgcggcccggcaaaagctgcgcgagcatattgattccgccgacaaacacctcacttttgcctgtccgcagggcggcgccatccgacagtttatgcatctgtggtttggccatcaccttgccgactggtcaccggtcatgaaaatcgcctgataactcgaggaggtataaaggatgaaaaaaaccgttgtgattggcgcaggctttggtggcctggcgctggcgattcgcctgcaggcggcagggatcccaaccgtactgctggagcagcgggacaagcccggcggtcgggcctacgtctggcatgaccagggctttacctttgacgccgggccgacggtgatcaccgatcctaccgcgcttgaggcgctgttcaccctggccggcaggcgcatggaggattacgtcaggctgctgccggtaaaacccttctaccgactctgctgggagtccgggaagaccctcgactatgctaacgacagcgccgagcttgaggcgcagattacccagttcaacccccgcgacgtcgagggctaccggcgctttctggcttactcccaggcggtattccaggagggatatttgcgcctcggcagcgtgccgttcctctcttttcgcgacatgctgcgcgccgggccgcagctgcttaagctccaggcgtggcagagcgtctaccagtcggtttcgcgctttattgaggatgagcatctgcggcaggccttctcgttccactccctgctggtaggcggcaaccccttcaccacctcgtccatctacaccctgatccacgcccttgagcgggagtggggggtctggttccctgagggcggcaccggggcgctggtgaacggcatggtgaagctgtttaccgatctgggcggggagatcgaactcaacgcccgggtcgaagagctggtggtggccgataaccgcgtaagccaggtccggctggcggatggtcggatctttgacaccgacgccgtagcctcgaacgctgacgtggtgaacacctataaaaagctgctcggccaccatccggtggggcagaagcgggcggcagcgctggagcgcaagagcatgagcaactcgctgtttgtgctctacttcggcctgaaccagcctcattcccagctggcgcaccataccatctgttttggtccccgctaccgggagctgatcgacgagatctttaccggcagcgcgctggcggatgacttctcgctctacctgcactcgccctgcgtgaccgatccctcgctcgcgcctcccggctgcgccagcttctacgtgctggccccggtgccgcatcttggcaacgcgccgctggactgggcgcaggaggggccgaagctgcgcgaccgcatctttgactaccttgaagagcgctatatgcccggcctgcgtagccagctggtgacccagcggatctttaccccggcagacttccacgacacgctggatgcgcatctgggatcggccttctccatcgagccgctgctgacccaaagcgcctggttccgcccgcacaaccgcgacagcgacattgccaacctctacctggtgggcgcaggtactcaccctggggcgggcattcctggcgtagtggcctcggcgaaagccaccgccagcctgatgattgaggatctgcaatgagccaaccgccgctgcttgaccacgccacgcagaccatggccaacggctcgaaaagttttgccaccgctgcgaagctgttcgacccggccacccgccgtagcgtgctgatgctctacacctggtgccgccactgcgatgacgtcattgacgaccagacccacggcttcgccagcgaggccgcggcggaggaggaggccacccagcgcctggcccggctgcgcacgctgaccctggcggcgtttgaaggggccgagatgcaggatccggccttcgctgcctttcaggaggtggcgctgacccacggtattacgccccgcatggcgctcgatcacctcgacggctttgcgatggacgtggctcagacccgctatgtcacctttgaggatacgctgcgctactgctatcacgtggcgggcgtggtgggtctgatgatggccagggtgatgggcgtgcgggatgagcgggtgctggatcgcgcctgcgatctggggctggccttccagctgacgaatatcgcccgggatattattgacgatgcggctattgaccgctgctatctgcccgccgagtggctgcaggatgccgggctgaccccggagaactatgccgcgcgggagaatcgggccgcgctggcgcgggtggcggagcggcttattgatgccgcagagccgtactacatctcctcccaggccgggctacacgatctgccgccgcgctgcgcctgggcgatcgccaccgcccgcagcgtctaccgggagatcggtattaaggtaaaagcggcgggaggcagcgcctgggatcgccgccagcacaccagcaaaggtgaaaaaattgccatgctgatggcggcaccggggcaggttattcgggcgaagacgacgagggtgacgccgcgtccggccggtctttggcagcgtcccgtttagtaatctagaggaggtaataaaatatgcttcgttcgttgctcagaggcctcacgcatatcccccgcgtgaactccgcccagcagcccagctgtgcacacgcgcgactccagtttaagctcaggagcatgcagatgacgctcatgcagcccagcatctcagccaatctgtcgcgcgccgaggaccgcacagaccacatgaggggtgcaagcacctgggcaggcgggcagtcgcaggatgagctgatgctgaaggacgagtgcatcttggtggatgttgaggacaacatcacaggccatgccagcaagctggagtgtcacaagttcctaccacatcagcctgcaggcctgctgcaccgggccttctctgtgttcctgtttgacgatcaggggcgactgctgctgcaacagcgtgcacgctcaaaaatcaccttcccaagtgtgtggacgaacacctgctgcagccaccctttacatgggcagaccccagatgaggtggaccaactaagccaggtggccgacggaacagtacctggcgcaaaggctgctgccatccgcaagttggagcacgagctggggataccagcgcaccagctgccggcaagcgcgtttcgcttcctcacgcgtttgcactactgtgccgcggacgtgcagccagctgcgacacaatcagcgctctggggcgagcacgaaatggactacatcttgttcatccgggccaacgtcaccttggcgcccaaccctgacgaggtggacgaagtcaggtacgtgacgcaagaggagctgcggcagatgatgcagccggacaacgggctgcaatggtcgccgtggtttcgcatcatcgccgcgcgcttccttgagcgttggtgggctgacctggacgcggccctaaacactgacaaacacgaggattggggaacggtgcatcacatcaacgaagcgtgataagcggccgcgtcgacaggaggagcggctatgcaaccgcattatgatctgattctcgtgggggctggactcgcgaatggccttatcgccctgcgtcttcagcagcagcaacctgatatgcgtattttgcttatcgacgccgcaccccaggcgggcgggaatcatacgtggtcatttcaccacgatgatttgactgagagccaacatcgttggatagctccgctggtggttcatcactggcccgactatcaggtacgctttcccacacgccgtcgtaagctgaacagcggctacttttgtattacttctcagcgtttcgctgaggttttacagcgacagtttggcccgcacttgtggatggataccgcggtcgcagaggttaatgcggaatctgttcggttgaaaaagggtcaggttatcggtgcccgcgcggtgattgacgggcggggttatgcggcaaattcagcactgagcgtgggcttccaggcgtttattggccaggaatggcgattgagccacccgcatggtttatcgtctcccattatcatggatgccacggtcgatcagcaaaatggttatcgcttcgtgtacagcctgccgctctcgccgaccagattgttaattgaagacacgcactatattgataatgcgacattagatcctgaatgcgcgcggcaaaatatttgcgactatgccgcgcaacagggttggcagcttcagacactgctgcgagaagaacagggcgccttacccattactctgtcgggcaatgccgacgcattctggcagcagcgccccctggcctgtagtggattacgtgccggtctgttccatcctaccaccggctattcactgccgctggcggttgccgtggccgaccgcctgagtgcacttgatgtctttacgtcggcctcaattcaccatgccattacgcattttgcccgcgagcgctggcagcagcagggctttttccgcatgctgaatcgcatgctgtttttagccggacccgccgattcacgctggcgggttatgcagcgtttttatggtttacctgaagatttaattgcccgtttttatgcgggaaaactcacgctgaccgatcggctacgtattctgagcggcaagccgcctgttccggtattagcagcattgcaagccattatgacgactcatcgttaactgcaggcatgcaagctggggatcctacctgacgctttttatcgcaactctctactgtttctccatacccgtttttttgggctggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctacagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctg

SEQ ID NO: 85 - DNA, 13938 bp, synthetic construct
  misc_feature 1..13938: note = pT-HBSREYbbO
  source 1..13938: mol_type = other DNA
tcgacaggaggagcggctatgcaaccgcattatgatctgattctcgtgggggctggactcgcgaatggccttatcgccctgcgtcttcagcagcagcaacctgatatgcgtattttgcttatcgacgccgcaccccaggcgggcgggaatcatacgtggtcatttcaccacgatgatttgactgagagccaacatcgttggatagctccgctggtggttcatcactggcccgactatcaggtacgctttcccacacgccgtcgtaagctgaacagcggctacttttgtattacttctcagcgtttcgctgaggttttacagcgacagtttggcccgcacttgtggatggataccgcggtcgcagaggttaatgcggaatctgttcggttgaaaaagggtcaggttatcggtgcccgcgcggtgattgacgggcggggttatgcggcaaattcagcactgagcgtgggcttccaggcgtttattggccaggaatggcgattgagccacccgcatggtttatcgtctcccattatcatggatgccacggtcgatcagcaaaatggttatcgcttcgtgtacagcctgccgctctcgccgaccagattgttaattgaagacacgcactatattgataatgcgacattagatcctgaatgcgcgcggcaaaatatttgcgactatgccgcgcaacagggttggcagcttcagacactgctgcgagaagaacagggcgccttacccattactctgtcgggcaatgccgacgcattctggcagcagcgccccctggcctgtagtggattacgtgccggtctgttccatcctaccaccggctattcactgccgctggcggttgccgtggccgaccgcctgagtgcacttgatgtctttacgtcggcctcaattcaccatgccattacgcattttgcccgcgagcgctggcagcagcagggctttttccgcatgctgaatcgcatgctgtttttagccggacccgccgattcacgctggcgggttatgcagcgtttttatggtttacctgaagatttaattgcccgtttttatgcgggaaaactcacgctgaccgatcggctacgtattctgagcggcaagccgcctgttccggtattagcagcattgcaagccattatgacgactcatcgttaactgcaggcatgcaagctggggatcctacctgacgctttttatcgcaactctctactgtttctccatacccgtttttttgggctggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctacagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctggtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatggaattctacgtactagtgaattcaggaggtaataaatatgggtctgatgctgattgattggtgtgcactggctctggttgttttcattggcctgccgcacggcgcgctggatgctgccatttctttttctatgatctcttctgcaaaacgcattgctcgtctggctggtattctgctgatctatctgctgctggcgaccgcgttcttcctgatctggtatcagctgccagcgtttagcctgctgatcttcctgctgatctccattatccactttggtatggcagacttcaacgcgtccccaagcaaactgaaatggccgcatatcatcgcccacggcggtgttgttactgtttggctgccgctgatccagaaaaacgaagtaactaaactgtttagcatcctgactaacggtccgactccgatcctgtgggacatcctgctgattttcttcctgtgttggtctattggcgtgtgtctgcacacgtacgaaaccctgcgctctaaacattacaacatcgcctttgaactgatcggtctgattttcctggcgtggtatgcgccgcctctggttacgtttgccacttacttctgcttcattcattcccgtcgccacttctcctttgtgtggaagcagctgcaacacatgtcttccaaaaagatgatgattggcagcgcgattatcctgtcctgtacctcttggctgatcggcggtggtatctatttcttcctgaactccaaaatgatcgcctctgaggctgcgctgcagactgtgttcatcggtctggcggcactgaccgtgccgcacatgattctgatcgacttcatcttccgtccgcactcttcccgtatcaaaatcaaaaactaattaattaaaggaggtaataatatgactcataaagcaacggagatcctgacaggtaaagttatgcaaaaatcggtcttaattaccggatgttccagtggaattggcctggaaagcgcgctcgaattaaaacgccagggttttcatgtgctggcaggttgccggaaaccggatgatgttgagcgcatgaacagcatgggatttaccggcgtgttgatcgatctggattcaccagaaagtgttgatcgcgcagccgacgaggtgatcgccctgaccgataattgtctgtatgggatctttaacaatgccggattcggcatgtatggccccctttccaccatcagccgtgcgcagatggaacagcagttttccgccaactttttcggcgcacaccagctcaccatgcgcctgttacccgcgatgttaccgcacggtgaagggcgtattgtgatgacatcatcggtgatgggattaatctccacgccgggtcgtggcgcttacgcggccagtaaatatgcgctggaggcgtggtcagatgcactgcgcatggagctgcgccacagcggaattaaagtcagcctgatcgaacccggtcccattcgtactcgcttcaccgacaacgtcaaccagacgcaaagtgataaaccagtcgaaaatcccggcatcgccgcccgctttacgttgggaccggaagcggtggtggacaaagtacgccatgcttttattagcgagaagccgaagatgcgctatccggtgacgctggtgacctgggcggtaatggtgcttaagcgcctgctgccggggcgcgtgatggacaaaatattgcaggggtaactagtaggaggtaataaatatggtgagtggcagtaaagcgggcgtttcgcctcatcgcgaaatagaagtaatgagacaatccattgacgatcacctggctggcctgttacctgaaaccgacagccaggatatcgtcagccttgcgatgcgtgaaggcgtcatggcacccggtaaacggatccgtccgctgctgatgctgctggccgcccgcgacctccgctaccagggcagtatgcctacgctgctcgatctcgcctgcgccgttgaactgacccataccgcgtcgctgatgctcgacgacatgccctgcatggacaacgccgagctgcgccgcggtcagcccactacccacaaaaaatttggtgagagcgtggcgatccttgcctccgttgggctgctctctaaagcctttggtctgatcgccgccaccggcgatctgccgggggagaggcgtgcccaggcggtcaacgagctctctaccgccgtgggcgtgcagggcctggtactggggcagtttcgcgatcttaacgatgccgccctcgaccgtacccctgacgctatcctcagcaccaaccacctcaagaccggcattctgttcagcgcgatgctgcagatcgtcgccattgcttccgcctcgtcgccgagcacgcgagagacgctgcacgccttcgccctcgacttcggccaggcgtttcaactgctggacgatctgcgtgacgatcacccggaaaccggtaaagatcgcaataaggacgcgggaaaatcgacgctggtcaaccggctgggcgcagacgcggcccggcaaaagctgcgcgagcatattgattccgccgacaaacacctcacttttgcctgtccgcagggcggcgccatccgacagtttatgcatctgtggtttggccatcaccttgccgactggtcaccggtcatgaaaatcgcctgataactcgaggaggtataaaggatgaaaaaaaccgttgtgattggcgcaggctttggtggcctggcgctggcgattcgcctgcaggcggcagggatcccaaccgtactgctggagcagcgggacaagcccggcggtcgggcctacgtctggcatgaccagggctttacctttgacgccgggccgacggtgatcaccgatcctaccgcgcttgaggcgctgttcaccctggccggcaggcgcatggaggattacgtcaggctgctgccggtaaaacccttctaccgactctgctgggagtccgggaagaccctcgactatgctaacgacagcgccgagcttgaggcgcagattacccagttcaacccccgcgacgtcgagggctaccggcgctttctggcttactcccaggcggtattccaggagggatatttgcgcctcggcagcgtgccgttcctctcttttcgcgacatgctgcgcgccgggccgcagctgcttaagctccaggcgtggcagagcgtctaccagtcggtttcgcgctttattgaggatgagcatctgcggcaggccttctcgttccactccctgctggtaggcggcaaccccttcaccacctcgtccatctacaccctgatccacgcccttgagcgggagtggggggtctggttccctgagggcggcaccggggcgctggtgaacggcatggtgaagctgtttaccgatctgggcggggagatcgaactcaacgcccgggtcgaagagctggtggtggccgataaccgcgtaagccaggtccggctggcggatggtcggatctttgacaccgacgccgtagcctcgaacgctgacgtggtgaacacctataaaaagctgctcggccaccatccggtggggcagaagcgggcggcagcgctggagcgcaagagcatgagcaactcgctgtttgtgctctacttcggcctgaaccagcctcattcccagctggcgcaccataccatctgttttggtccccgctaccgggagctgatcgacgagatctttaccggcagcgcgctggcggatgacttctcgctctacctgcactcgccctgcgtgaccgatccctcgctcgcgcctcccggctgcgccagcttctacgtgctggccccggtgccgcatcttggcaacgcgccgctggactgggcgcaggaggggccgaagctgcgcgaccgcatctttgactaccttgaagagcgctatatgcccggcctgcgtagccagctggtgacccagcggatctttaccccggcagacttccacgacacgctggatgcgcatctgggatcggccttctccatcgagccgctgctgacccaaagcgcctggttccgcccgcacaaccgcgacagcgacattgccaacctctacctggtgggcgcaggtactcaccctggggcgggcattcctggcgtagtggcctcggcgaaagccaccgccagcctgatgattgaggatctgcaatgagccaaccgccgctgcttgaccacgccacgcagaccatggccaacggctcgaaaagttttgccaccgctgcgaagctgttcgacccggccacccgccgtagcgtgctgatgctctacacctggtgccgccactgcgatgacgtcattgacgaccagacccacggcttcgccagcgaggccgcggcggaggaggaggccacccagcgcctggcccggctgcgcacgctgaccctggcggcgtttgaaggggccgagatgcaggatccggccttcgctgcctttcaggaggtggcgctgacccacggtattacgccccgcatggcgctcgatcacctcgacggctttgcgatggacgtggctcagacccgctatgtcacctttgaggatacgctgcgctactgctatcacgtggcgggcgtggtgggtctgatgatggccagggtgatgggcgtgcgggatgagcgggtgctggatcgcgcctgcgatctggggctggccttccagctgacgaatatcgcccgggatattattgacgatgcggctattgaccgctgctatctgcccgccgagtggctgcaggatgccgggctgaccccggagaactatgccgcgcgggagaatcgggccgcgctggcgcgggtggcggagcggcttattgatgccgcagagccgtactacatctcctcccaggccgggctacacgatctgccgccgcgctgcgcctgggcgatcgccaccgcccgcagcgtctaccgggagatcggtattaaggtaaaagcggcgggaggcagcgcctgggatcgccgccagcacaccagcaaaggtgaaaaaattgccatgctgatggcggcaccggggcaggttattcgggcgaagacgacgagggtgacgccgcgtccggccggtctttggcagcgtcccgtttagtaatctagaggaggtaataaaatatgcttcgttcgttgctcagaggcctcacgcatatcccccgcgtgaactccgcccagcagcccagctgtgcacacgcgcgactccagtttaagctcaggagcatgcagatgacgctcatgcagcccagcatctcagccaatctgtcgcgcgccgaggaccgcacagaccacatgaggggtgcaagcacctgggcaggcgggcagtcgcaggatgagctgatgctgaaggacgagtgcatcttggtggatgttgaggacaacatcacaggccatgccagcaagctggagtgtcacaagttcctaccacatcagcctgcaggcctgctgcaccgggccttctctgtgttcctgtttgacgatcaggggcgactgctgctgcaacagcgtgcacgctcaaaaatcaccttcccaagtgtgtggacgaacacctgctgcagccaccctttacatgggcagaccccagatgaggtggaccaactaagccaggtggccgacggaacagtacctggcgcaaaggctgctgccatccgcaagttggagcacgagctggggataccagcgcaccagctgccggcaagcgcgtttcgcttcctcacgcgtttgcactactgtgccgcggacgtgcagccagctgcgacacaatcagcgctctggggcgagcacgaaatggactacatcttgttcatccgggccaacgtcaccttggcgcccaaccctgacgaggtggacgaagtcaggtacgtgacgcaagaggagctgcggcagatgatgcagccggacaacgggctgcaatggtcgccgtggtttcgcatcatcgccgcgcgcttccttgagcgttggtgggctgacctggacgcggccctaaacactgacaaacacgaggattggggaacggtgcatcacatcaacgaagcgtgataagcggccgcgctgttgacaattaatcatccggctcgtataatgtgtggaattgtgagcggataacaatttcacacaggaaacagaccatggagttgaaaacagtagttattattgatgcattacgaacaccaattggaaaatataaaggcagcttaagtcaagtaagtgccgtagacttaggaacacatgttacaacacaacttttaaaaagacattccactatttctgaagaaattgatcaagtaatctttggaaatgttttacaagctggaaatggccaaaatcccgcacgacaaatagcaataaacagcggtttatctcatgaaattcccgcaatgacagttaatgaggtctgcggatcaggaatgaaggccgttattttggcgaaacaattgattcaattaggagaagcggaagttttaattgctggcgggattgagaatatgtcccaagcacctaaattacaacgatttaattacgaaacagaaagctatgatgcgcctttttctagtatgatgtacgatgggttaacggatgcctttagtggtcaagcaatgggcttaactgctgaaaatgtggccgaaaagtatcatgtaactagagaagagcaagatcaattttctgtacattcacaattaaaagcagctcaagcacaagcagaagggatattcgctgacgaaatagccccattagaagtatcaggaacgcttgtggagaaagatgaagggattcgccctaattcgagcgttgagaagctaggaacgcttaaaacagtttttaaagaagacggtactgtaacagcagggaatgcatcaaccattaatgatggggcttctgctttgattattgcttcacaagaatatgccgaagcacacggtcttccttatttagctattattcgagacagtgtggaagtcggtattgatccagcctatatgggaatttcgccgattaaagccattcaaaaactgttagcgcgcaatcaacttactacggaagaaattgatctgtatgaaatcaacgaagcatttgcagcaacttcaatcgtggtccaaagagaactggctttaccagaggaaaaggtcaacatttatggtggcggtatttcattaggtcatgcgattggtgccacaggtgctcgtttattaacgagtttaagttatcaattaaatcaaaaagaaaagaaatatggagtggcttctttatgtatcggcggtggcttaggactcgctatgctactagagagacctcagcaaaaaaaaaacagccgattttatcaaatgagtcctgaggaacgcctggcttctcttcttaatgaaggccagatttctgctgatacaaaaaaagaatttgaaaatacggctttatcttcgcagattgccaatcatatgattgaaaatcaaatcagtgaaacagaagtgccgatgggcgttggcttacatttaacagtggacgaaactgattatttggtaccaatggcgacagaagagccctcagtgattgcggctttgagtaatggtgcaaaaatagcacaaggatttaaaacagtgaatcaacaacgtttaatgcgtggacaaatcgttttttacgatgttgcagacgccgagtcattgattgatgaactacaagtaagagaaacggaaatttttcaacaagcagagttaagttatccatctatcgttaaacgcggcggcggcttaagagatttgcaatatcgtgcttttgatgaatcatttgtatctgtcgactttttagtagatgttaaggatgcaatgggggcaaatatcgttaacgctatgttggaaggtgtggccgagttgttccgtgaatggtttgcggagcaaaagattttattcagtattttaagtaattatgccacggagtcggttgttacgatgaaaacggctattccagtttcacgtttaagtaaggggagcaatggccgggaaattgctgaaaaaattgttttagcttcacgctatgcttcattagatccttatcgggcagtcacgcataacaaagggatcatgaatggcattgaagctgtcgttttagctacaggaaatgatacacgcgctgttagcgcttcttgtcatgcttttgcggtgaaggaaggtcgctaccaaggtttgactagttggacgctggatggcgaacaactaattggtgaaatttcagttccgcttgcgttagccacggttggcggtgccacaaaagtcttacctaaatctcaagcagctgctgatttgttagcagtgacggatgcaaaagaactaagtcgagtagtagcggctgttggtttggcacaaaatttagcggcgttacgggccttagtctctgaaggaattcaaaaaggacacatggctctacaagcacgttctttagcgatgacggtcggagctactggtaaagaagttgaggcagtcgctcaacaattaaaacgtcaaaaaacgatgaaccaagaccgagccttggctattttaaatgatttaagaaaacaataaaaaaacagttcagcagaaattattctgctgaactgttttttttcacattaggtagccgtttcagc

SEQ ID NO: 86 - DNA, 12898 bp, synthetic construct
  misc_feature 1..12898: note = pT-HBSREYbbOfree
  source 1..12898: mol_type = other DNA
agcttgcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctggtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatggaattctacgtactagtgaattcaggaggtaataaatatgggtctgatgctgattgattggtgtgcactggctctggttgttttcattggcctgccgcacggcgcgctggatgctgccatttctttttctatgatctcttctgcaaaacgcattgctcgtctggctggtattctgctgatctatctgctgctggcgaccgcgttcttcctgatctggtatcagctgccagcgtttagcctgctgatcttcctgctgatctccattatccactttggtatggcagacttcaacgcgtccccaagcaaactgaaatggccgcatatcatcgcccacggcggtgttgttactgtttggctgccgctgatccagaaaaacgaagtaactaaactgtttagcatcctgactaacggtccgactccgatcctgtgggacatcctgctgattttcttcctgtgttggtctattggcgtgtgtctgcacacgtacgaaaccctgcgctctaaacattacaacatcgcctttgaactgatcggtctgattttcctggcgtggtatgcgccgcctctggttacgtttgccacttacttctgcttcattcattcccgtcgccacttctcctttgtgtggaagcagctgcaacacatgtcttccaaaaagatgatgattggcagcgcgattatcctgtcctgtacctcttggctgatcggcggtggtatctatttcttcctgaactccaaaatgatcgcctctgaggctgcgctgcagactgtgttcatcggtctggcggcactgaccgtgccgcacatgattctgatcgacttcatcttccgtccgcactcttcccgtatcaaaatcaaaaactaattaattaaaggaggtaataatatgactcataaagcaacggagatcctgacaggtaaagttatgcaaaaatcggtcttaattaccggatgttccagtggaattggcctggaaagcgcgctcgaattaaaacgccagggttttcatgtgctggcaggttgccggaaaccggatgatgttgagcgcatgaacagcatgggatttaccggcgtgttgatcgatctggattcaccagaaagtgttgatcgcgcagccgacgaggtgatcgccctgaccgataattgtctgtatgggatctttaacaatgccggattcggcatgtatggccccctttccaccatcagccgtgcgcagatggaacagcagttttccgccaactttttcggcgcacaccagctcaccatgcgcctgttacccgcgatgttaccgcacggtgaagggcgtattgtgatgacatcatcggtgatgggattaatctccacgccgggtcgtggcgcttacgcggccagtaaatatgcgctggaggcgtggtcagatgcactgcgcatggagctgcgccacagcggaattaaagtcagcctgatcgaacccggtcccattcgtactcgcttcaccgacaacgtcaaccagacgcaaagtgataaaccagtcgaaaatcccggcatcgccgcccgctttacgttgggaccggaagcggtggtggacaaagtacgccatgcttttattagcgagaagccgaagatgcgctatccggtgacgctggtgacctgggcggtaatggtgcttaagcgcctgctgccggggcgcgtgatggacaaaatattgcaggggtaactagtaggaggtaataaatatggtgagtggcagtaaagcgggcgtttcgcctcatcgcgaaatagaagtaatgagacaatccattgacgatcacctggctggcctgttacctgaaaccgacagccaggatatcgtcagccttgcgatgcgtgaaggcgtcatggcacccggtaaacggatccgtccgctgctgatgctgctggccgcccgcgacctccgctaccagggcagtatgcctacgctgctcgatctcgcctgcgccgttgaactgacccataccgcgtcgctgatgctcgacgacatgccctgcatggacaacgccgagctgcgccgcggtcagcccactacccacaaaaaatttggtgagagcgtggcgatccttgcctccgttgggctgctctctaaagcctttggtctgatcgccgccaccggcgatctgccgggggagaggcgtgcccaggcggtcaacgagctctctaccgccgtgggcgtgcagggcctggtactggggcagtttcgcgatcttaacgatgccgccctcgaccgtacccctgacgctatcctcagcaccaaccacctcaagaccggcattctgttcagcgcgatgctgcagatcgtcgccattgcttccgcctcgtcgccgagcacgcgagagacgctgcacgccttcgccctcgacttcggccaggcgtttcaactgctggacgatctgcgtgacgatcacccggaaaccggtaaagatcgcaataaggacgcgggaaaatcgacgctggtcaaccggctgggcgcagacgcggcccggcaaaagctgcgcgagcatattgattccgccgacaaacacctcacttttgcctgtccgcagggcggcgccatccgacagtttatgcatctgtggtttggccatcaccttgccgactggtcaccggtcatgaaaatcgcctgataactcgaggaggtataaaggatgaaaaaaaccgttgtgattggcgcaggctttggtggcctggcgctggcgattcgcctgcaggcggcagggatcccaaccgtactgctggagcagcgggacaagcccggcggtcgggcctacgtctggcatgaccagggctttacctttgacgccgggccgacggtgatcaccgatcctaccgcgcttgaggcgctgttcaccctggccggcaggcgcatggaggattacgtcaggctgctgccggtaaaacccttctaccgactctgctgggagtccgggaagaccctcgactatgctaacgacagcgccgagcttgaggcgcagattacccagttcaacccccgcgacgtcgagggctaccggcgctttctggcttactcccaggcggtattccaggagggatatttgcgcctcggcagcgtgccgttcctctcttttcgcgacatgctgcgcgccgggccgcagctgcttaagctccaggcgtggcagagcgtctaccagtcggtttcgcgctttattgaggatgagcatctgcggcaggccttctcgttccactccctgctggtaggcggcaaccccttcaccacctcgtccatctacaccctgatccacgcccttgagcgggagtggggggtctggttccctgagggcggcaccggggcgctggtgaacggcatggtgaagctgtttaccgatctgggcggggagatcgaactcaacgcccgggtcgaagagctggtggtggccgataaccgcgtaagccaggtccggctggcggatggtcggatctttgacaccgacgccgtagcctcgaacgctgacgtggtgaacacctataaaaagctgctcggccaccatccggtggggcagaagcgggcggcagcgctggagcgcaagagcatgagcaactcgctgtttgtgctctacttcggcctgaaccagcctcattcccagctggcgcaccataccatctgttttggtccccgctaccgggagctgatcgacgagatctttaccggcagcgcgctggcggatgacttctcgctctacctgcactcgccctgcgtgaccgatccctcgctcgcgcctcccggctgcgccagcttctacgtgctggccccggtgccgcatcttggcaacgcgccgctggactgggcgcaggaggggccgaagctgcgcgaccgcatctttgactaccttgaagagcgctatatgcccggcctgcgtagccagctggtgacccagcggatctttaccccggcagacttccacgacacgctggatgcgcatctgggatcggccttctccatcgagccgctgctgacccaaagcgcctggttccgcccgcacaaccgcgacagcgacattgccaacctctacctggtgggcgcaggtactcaccctggggcgggcattcctggcgtagtggcctcggcgaaagccaccgccagcctgatgattgaggatctgcaatgagccaaccgccgctgcttgaccacgccacgcagaccatggccaacggctcgaaaagttttgccaccgctgcgaagctgttcgacccggccacccgccgtagcgtgctgatgctctacacctggtgccgccactgcgatgacgtcattgacgaccagacccacggcttcgccagcgaggccgcggcggaggaggaggccacccagcgcctggcccggctgcgcacgctgaccctggcggcgtttgaaggggccgagatgcaggatccggccttcgctgcctttcaggaggtggcgctgacccacggtattacgccccgcatggcgctcgatcacctcgacggctttgcgatggacgtggctcagacccgctatgtcacctttgaggatacgctgcgctactgctatcacgtggcgggcgtggtgggtctgatgatggccagggtgatgggcgtgcgggatgagcgggtgctggatcgcgcctgcgatctggggctggccttccagctgacgaatatcgcccgggatattattgacgatgcggctattgaccgctgctatctgcccgccgagtggctgcaggatgccgggctgaccccggagaactatgccgcgcgggagaatcgggccgcgctggcgcgggtggcggagcggcttattgatgccgcagagccgtactacatctcctcccaggccgggctacacgatctgccgccgcgctgcgcctgggcgatcgccaccgcccgcagcgtctaccgggagatcggtattaaggtaaaagcggcgggaggcagcgcctgggatcgccgccagcacaccagcaaaggtgaaaaaattgccatgctgatggcggcaccggggcaggttattcgggcgaagacgacgagggtgacgccgcgtccggccggtctttggcagcgtcccgtttagtaatctagaggaggtaataaaatatgcttcgttcgttgctcagaggcctcacgcatatcccccgcgtgaactccgcccagcagcccagctgtgcacacgcgcgactccagtttaagctcaggagcatgcagatgacgctcatgcagcccagcatctcagccaatctgtcgcgcgccgaggaccgcacagaccacatgaggggtgcaagcacctgggcaggcgggcagtcgcaggatgagctgatgctgaaggacgagtgcatcttggtggatgttgaggacaacatcacaggccatgccagcaagctggagtgtcacaagttcctaccacatcagcctgcaggcctgctgcaccgggccttctctgtgttcctgtttgacgatcaggggcgactgctgctgcaacagcgtgcacgctcaaaaatcaccttcccaagtgtgtggacgaacacctgctgcagccaccctttacatgggcagaccccagatgaggtggaccaactaagccaggtggccgacggaacagtacctggcgcaaaggctgctgccatccgcaagttggagcacgagctggggataccagcgcaccagctgccggcaagcgcgtttcgcttcctcacgcgtttgcactactgtgccgcggacgtgcagccagctgcgacacaatcagcgctctggggcgagcacgaaatggactacatcttgttcatccgggccaacgtcaccttggcgcccaaccctgacgaggtggacgaagtcaggtacgtgacgcaagaggagctgcggcagatgatgcagccggacaacgggctgcaatggtcgccgtggtttcgcatcatcgccgcgcgcttccttgagcgttggtgggctgacctggacgcggccctaaacactgacaaacacgaggattggggaacggtgcatcacatcaacgaagcgtgataagcggccgcgctgttgacaattaatcatccggctcgtataatgtgtggaattgtgagcggataacaatttcacacaggaaacagaccatggagttgaaaacagtagttattattgatgcattacgaacaccaattggaaaatataaaggcagcttaagtcaagtaagtgccgtagacttaggaacacatgttacaacacaacttttaaaaagacattccactatttctgaagaaattgatcaagtaatctttggaaatgttttacaagctggaaatggccaaaatcccgcacgacaaatagcaataaacagcggtttatctcatgaaattcccgcaatgacagttaatgaggtctgcggatcaggaatgaaggccgttattttggcgaaacaattgattcaattaggagaagcggaagttttaattgctggcgggattgagaatatgtcccaagcacctaaattacaacgatttaattacgaaacagaaagctatgatgcgcctttttctagtatgatgtacgatgggttaacggatgcctttagtggtcaagcaatgggcttaactgctgaaaatgtggccgaaaagtatcatgtaactagagaagagcaagatcaattttctgtacattcacaattaaaagcagctcaagcacaagcagaagggatattcgctgacgaaatagccccattagaagtatcaggaacgcttgtggagaaagatgaagggattcgccctaattcgagcgttgagaagctaggaacgcttaaaacagtttttaaagaagacggtactgtaacagcagggaatgcatcaaccattaatgatggggcttctgctttgattattgcttcacaagaatatgccgaagcacacggtcttccttatttagctattattcgagacagtgtggaagtcggtattgatccagcctatatgggaatttcgccgattaaagccattcaaaaactgttagcgcgcaatcaacttactacggaagaaattgatctgtatgaaatcaacgaagcatttgcagcaacttcaatcgtggtccaaagagaactggctttaccagaggaaaaggtcaacatttatggtggcggtatttcattaggtcatgcgattggtgccacaggtgctcgtttattaacgagtttaagttatcaattaaatcaaaaagaaaagaaatatggagtggcttctttatgtatcggcggtggcttaggactcgctatgctactagagagacctcagcaaaaaaaaaacagccgattttatcaaatgagtcctgaggaacgcctggcttctcttcttaatgaaggccagatttctgctgatacaaaaaaagaatttgaaaatacggctttatcttcgcagattgccaatcatatgattgaaaatcaaatcagtgaaacagaagtgccgatgggcgttggcttacatttaacagtggacgaaactgattatttggtaccaatggcgacagaagagccctcagtgattgcggctttgagtaatggtgcaaaaatagcacaaggatttaaaacagtgaatcaacaacgtttaatgcgtggacaaatcgttttttacgatgttgcagacgccgagtcattgattgatgaactacaagtaagagaaacggaaatttttcaacaagcagagttaagttatccatctatcgttaaacgcggcggcggcttaagagatttgcaatatcgtgcttttgatgaatcatttgtatctgtcgactttttagtagatgttaaggatgcaatgggggcaaatatcgttaacgctatgttggaaggtgtggccgagttgttccgtgaatggtttgcggagcaaaagattttattcagtattttaagtaattatgccacggagtcggttgttacgatgaaaacggctattccagtttcacgtttaagtaaggggagcaatggccgggaaattgctgaaaaaattgttttagcttcacgctatgcttcattagatccttatcgggcagtcacgcataacaaagggatcatgaatggcattgaagctgtcgttttagctacaggaaatgatacacgcgctgttagcgcttcttgtcatgcttttgcggtgaaggaaggtcgctaccaaggtttgactagttggacgctggatggcgaacaactaattggtgaaatttcagttccgcttgcgttagccacggttggcggtgccacaaaagtcttacctaaatctcaagcagctgctgatttgttagcagtgacggatgcaaaagaactaagtcgagtagtagcggctgttggtttggcacaaaatttagcggcgttacgggccttagtctctgaaggaattcaaaaaggacacatggctctacaagcacgttctttagcgatgacggtcggagctactggtaaagaagttgaggcagtcgctcaacaattaaaacgtcaaaaaacgatgaaccaagaccgagccttggctattttaaatgatttaagaaaacaataaaaaaacagttcagcagaaattattctgctgaactgttttttttcacattaggtagccgtttcagctcgacaggaggagcggctatgcaaccgcattatgatctgattctcgtgggggctggactcgcgaatggccttatcgccctgcgtcttcagcagcagcaacctgatatgcgtattttgcttatcgacgccgcaccccaggcgggcgggaatcatacgtggtcatttcaccacgatgatttgactgagagccaacatcgttggatagctccgctggtggttcatcactggcccgactatcaggtacgctttcccacacgccgtcgtaagctgaacagcggctacttttgtattacttctcagcgtttcgctgaggttttacagcgacagtttggcccgcacttgtggatggataccgcggtcgcagaggttaatgcggaatctgttcggttgaaaaagggtcaggttatcggtgcccgcgcggtgattgacgggcggggttatgcggcaaattcagcactgagcgtgggcttccaggcgtttattggccaggaatggcgattgagccacccgcatggtttatcgtctcccattatcatggatgccacggtcgatcagcaaaatggttatcgcttcgtgtacagcctgccgctctcgccgaccagattgttaattgaagacacgcactatattgataatgcgacattagatcctgaatgcgcgcggcaaaatatttgcgactatgccgcgcaacagggttggcagcttcagacactgctgcgagaagaacagggcgccttacccattactctgtcgggcaatgccgacgcattctggcagcagcgccccctggcctgtagtggattacgtgccggtctgttccatcctaccaccggctattcactgccgctggcggttgccgtggccgaccgcctgagtgcacttgatgtctttacgtcggcctcaattcaccatgccattacgcattttgcccgcgagcgctggcagcagcagggctttttccgcatgctgaatcgcatgctgtttttagccggacccgccgattcacgctggcgggttatgcagcgtttttatggtttacctgaagatttaattgcccgtttttatgcgggaaaactcacgctgaccgatcggctacgtattctgagcggcaagccgcctgttccggtattagcagcattgcaagccattatgacgactcatcgttaactgcaggcatgcaagctggggatcctacctgacgctttttatcgcaactctctactgtttctccatacccgtttttttgggctggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcaa

SEQ ID NO: 87 - DNA, 5381 bp, synthetic construct
  misc_feature 1..5381: note = pT-dxr
  source 1..5381: mol_type = other DNA
aacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctggtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatggaattcgagctcatgaagcaactcaccattctgggctcgaccggctcgattggttgcagcacgctggacgtggtgcgccataatcccgaacacttccgcgtagttgcgctggtggcaggcaaaaatgtcactcgcatggtagaacagtgcctggaattctctccccgctatgccgtaatggacgatgaagcgagtgcgaaacttcttaaaacgatgctacagcaacagggtagccgcaccgaagtcttaagtgggcaacaagccgcttgcgatatggcagcgcttgaggatgttgatcaggtgatggcagccattgttggcgctgctgggctgttacctacgcttgctgcgatccgcgcgggtaaaaccattttgctggccaataaagaatcactggttacctgcggacgtctgtttatggacgccgtaaagcagagcaaagcgcaattgttaccggtcgatagcgaacataacgccatttttcagagtttaccgcaacctatccagcataatctgggatacgctgaccttgagcaaaatggcgtggtgtccattttacttaccgggtctggtggccctttccgtgagacgccattgcgcgatttggcaacaatgacgccggatcaagcctgccgtcatccgaactggtcgatggggcgtaaaatttctgtcgattcggctaccatgatgaacaaaggtctggaatacattgaagcgcgttggctgtttaacgccagcgccagccagatggaagtgctgattcacccgcagtcagtgattcactcaatggtgcgctatcaggacggcagtgttctggcgcagctgggggaaccggatatgcgtacgccaattgcccacaccatggcatggccgaatcgcgtgaactctggcgtgaagccgctcgatttttgcaaactaagtgcgttgacatttgccgcaccggattatgatcgttatccatgcctgaaactggcgatggaggcgttcgaacaaggccaggcagcgacgacagcattgaatgccgcaaacgaaatcaccgttgctgcttttcttgcgcaacaaatccgctttacggatatcgctgcgttgaatttatccgtactggaaaaaatggatatgcgcgaaccacaatgtgtggacgatgtgttatctgttgatgcgaacgcgcgtgaagtcgccagaaaagaggtgatgcgtctcgcaagctgatctagagcgggggatccactagttctagagtcgacctgcaggcatgcaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctacagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttc

SEQ ID NO: 88 - DNA, 7999 bp, synthetic construct
  misc_feature 1..7999: note = pTAS-dxr
  source 1..7999: mol_type = other DNA
tcgacctgcaggcatgcaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctacagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctggtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaggtaataaatatggaattcatggactttccgcagcaactcgaagcctgcgttaagcaggccaaccaggcgctgagccgttttatcgccccactgccctttcagaacactcccgtggtcgaaaccatgcagtatggcgcattattaggtggtaagcgcctgcgacctttcctggtttatgccaccggtcatatgttcggcgttagcacaaacacgctggacgcacccgctgccgccgttgagtgtatccacgcttactcattaattcatgatgatttaccggcaatggatgatgacgatctgcgtcgcggtttgccaacctgccatgtgaagtttggcgaagcaaacgcgattctcgctggcgacgctttacaaacgctggcgttctcgattttaagcgatgccgatatgccggaagtgtcggaccgcgacagaatttcgatgatttctgaactggcgagcgccagtggtattgccggaatgtgcggtggtcaggcattagatttagacgcggaaggcaaacacgtacctctggacgcgcttgagcgtattcatcgtcataaaaccggcgcattgattcgcgccgccgttcgccttggtgcattaagcgccggagataaaggacgtcgtgctctgccggtactcgacaagtatgcagagagcatcggccttgccttccaggttcaggatgacatcctggatgtggtgggagatactgcaacgttgggaaaacgccagggtgccgaccagcaacttggtaaaagtacctaccctgcacttctgggtcttgagcaagcccggaagaaagcccgggatctgatcgacgatgcccgtcagtcgctgaaacaactggctgaacagtcactcgatacctcggcactggaagcgctagcggactacatcatccagcgtaataaataaggatccaaaaaggaggtaataaaccatgtcaactcaacaagtttcatcagagaacattgttcgtaacgctgcgaatttccatcctaatatatggggaaaccatttcctcacatgtccttctcagacgattgatagttggactcaacagcaccacaaagaactgaaagaagaggtgaggaaaatgatggtgtctgatgcaaataaacctgcccagagattgcgcttgattgatactgtccaaaggctaggtgtggcttaccactttgaaaaggagattgatgatgcattggagaaaataggtcatgacccttttgatgataaagatgatctctacattgtctctctttgttttcgattgctgaggcagcatggaattaagatatcatgtgatgtgtttgagaagtttaaagatgacgatggaaaattcaaggcatcattgatgaatgatgttcaaggcatgctaagtttatatgaggcagcacacctagccattcacggagaagatattttagatgaagcaattgttttcacgaccactcaccttaagtcaacggtatctaattctcctgtaaactctacttttgctgaacaaatacgtcattctctcagagttcctctccgtaaagctgtacctaggttagagtcgaggtatttcttggatatctattcaagagatgatttgcacgataaaactttgctcaatttcgcaaagttagactttaatatactacaagcaatgcaccagaaggaagcaagtgagatgaccaggtggtggagagattttgacttccttaaaaagctgccttatataagagacagagtcgtggagctatatttttggattctggtgggagtgtcttatcagcccaaattcagcactggtagaatttttttgtccaaaataatatgccttgagaccctcgtagatgatacatttgacgcctacggtacttttgacgagctcgcaatctttactgaagcagttacaagatgggaccttggccacagagatgcactaccagaatacatgaaattcattttcaagacactcattgatgtctacagtgaagctgagcaagaactggcaaaggaagggagatcatacagcatacactatgcaatacgatcgttccaagaactagttatgaagtacttctgcgaagccaagtggttaaataaaggttatgttccgagcctggacgattataaatcagtttcattaagaagtatcggttttttaccgatagcggtagcttccttcgttttcatgggtgatattgcaactaaggaggtctttgaatgggaaatgaataaccctaagatcataatagccgcagaaacgattttcagattcctggatgacatagcaggccataggtttgagcaaaagagagaacatagtccatcagctattgaatgctacaagaatcaacatggagtgtctgaggaagaggcagttaaagcgttgtcgttagaagttgctaatagttggaaagatataaatgaggagctgcttctcaacccaatggctattcctttacctctgcttcaggtgattcttgatctctcacgttcggccgattttatgtacggtaatgctcaagatcgcttcacgcattcaacgatgatgaaagaccaagttgatttggtgctgaaggaccccgttaagcttgacgattaaagatctgtgtggaattgtgagcggataacaatttcacacaggaaacagaccatggaattcgagctcatgaagcaactcaccattctgggctcgaccggctcgattggttgcagcacgctggacgtggtgcgccataatcccgaacacttccgcgtagttgcgctggtggcaggcaaaaatgtcactcgcatggtagaacagtgcctggaattctctccccgctatgccgtaatggacgatgaagcgagtgcgaaacttcttaaaacgatgctacagcaacagggtagccgcaccgaagtcttaagtgggcaacaagccgcttgcgatatggcagcgcttgaggatgttgatcaggtgatggcagccattgttggcgctgctgggctgttacctacgcttgctgcgatccgcgcgggtaaaaccattttgctggccaataaagaatcactggttacctgcggacgtctgtttatggacgccgtaaagcagagcaaagcgcaattgttaccggtcgatagcgaacataacgccatttttcagagtttaccgcaacctatccagcataatctgggatacgctgaccttgagcaaaatggcgtggtgtccattttacttaccgggtctggtggccctttccgtgagacgccattgcgcgatttggcaacaatgacgccggatcaagcctgccgtcatccgaactggtcgatggggcgtaaaatttctgtcgattcggctaccatgatgaacaaaggtctggaatacattgaagcgcgttggctgtttaacgccagcgccagccagatggaagtgctgattcacccgcagtcagtgattcactcaatggtgcgctatcaggacggcagtgttctggcgcagctgggggaaccggatatgcgtacgccaattgcccacaccatggcatggccgaatcgcgtgaactctggcgtgaagccgctcgatttttgcaaactaagtgcgttgacatttgccgcaccggattatgatcgttatccatgcctgaaactggcgatggaggcgttcgaacaaggccaggcagcgacgacagcattgaatgccgcaaacgaaatcaccgttgctgcttttcttgcgcaacaaatccgctttacggatatcgctgcgttgaatttatccgtactggaaaaaatggatatgcgcgaaccacaatgtgtggacgatgtgttatctgttgatgcgaacgcgcgtgaagtcgccagaaaagaggtgatgcgtctcgcaagctgatctac

SEQ ID NO: 89 - DNA, 7275 bp, synthetic construct
  misc_feature 1..7275: note = pT-dxr/s
  source 1..7275: mol_type = other DNA
gtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatggaattcatgagttttgatattgccaaatacccgaccctggcactggtcgactccacccaggagttacgactgttgccgaaagagagtttaccgaaactctgcgacgaactgcgccgctatttactcgacagcgtgagccgttccagcgggcacttcgcctccgggctgggcacggtcgaactgaccgtggcgctgcactatgtctacaacaccccgtttgaccaattgatttgggatgtggggcatcaggcttatccgcataaaattttgaccggacgccgcgacaaaatcggcaccatccgtcagaaaggcggtctgcacccgttcccgtggcgcggcgaaagcgaatatgacgtattaagcgtcgggcattcatcaacctccatcagtgccggaattggtattgcggttgctgccgaaaaagaaggcaaaaatcgccgcaccgtctgtgtcattggcgatggcgcgattaccgcaggcatggcgtttgaagcgatgaatcacgcgggcgatatccgtcctgatatgctggtgattctcaacgacaatgaaatgtcgatttccgaaaatgtcggcgcgctcaacaaccatctggcacagctgctttccggtaagctttactcttcactgcgcgaaggcgggaaaaaagttttctctggcgtgccgccaattaaagagctgctcaaacgcaccgaagaacatattaaaggcatggtagtgcctggcacgttgtttgaagagctgggctttaactacatcggcccggtggacggtcacgatgtgctggggcttatcaccacgctaaagaacatgcgcgacctgaaaggcccgcagttcctgcatatcatgaccaaaaaaggtcgtggttatgaaccggcagaaaaagacccgatcactttccacgccgtgcctaaatttgatccctccagcggttgtttgccgaaaagtagcggcggtttgccgagctattcaaaaatctttggcgactggttgtgcgaaacggcagcgaaagacaacaagctgatggcgattactccggcgatgcgtgaaggttccggcatggtcgagttttcacgtaaattcccggatcgctacttcgacgtggcaattgccgagcaacacgcggtgacctttgctgcgggtctggcgattggtgggtacaaacccattgtcgcgatttactccactttcctgcaacgcgcctatgatcaggtgctgcatgacgtggcgattcaaaagcttccggtcctgttcgccatcgaccgcgcgggcattgttggtgctgacggtcaaacccatcagggtgcttttgatctctcttacctgcgctgcataccggaaatggtcattatgaccccgagcgatgaaaacgaatgtcgccagatgctctataccggctatcactataacgatggcccgtcagcggtgcgctacccgcgtggcaacgcggtcggcgtggaactgacgccgctggaaaaactaccaattggcaaaggcattgtgaagcgtcgtggcgagaaactggcgatccttaactttggtacgctgatgccagaagcggcgaaagtcgccgaatcgctgaacgccacgctggtcgatatgcgttttgtgaaaccgcttgatgaagcgttaattctggaaatggccgccagccatgaagcgctggtcaccgtagaagaaaacgccattatgggcggcgcaggcagcggcgtgaacgaagtgctgatggcccatcgtaaaccagtacccgtgctgaacattggcctgccggacttctttattccgcaaggaactcaggaagaaatgcgcgccgaactcggcctcgatgccgctggtatggaagccaaaatcaaggcctggctggcataaggtaccacacaggaaacagaccatggaattcgagctcatgaagcaactcaccattctgggctcgaccggctcgattggttgcagcacgctggacgtggtgcgccataatcccgaacacttccgcgtagttgcgctggtggcaggcaaaaatgtcactcgcatggtagaacagtgcctggaattctctccccgctatgccgtaatggacgatgaagcgagtgcgaaacttcttaaaacgatgctacagcaacagggtagccgcaccgaagtcttaagtgggcaacaagccgcttgcgatatggcagcgcttgaggatgttgatcaggtgatggcagccattgttggcgctgctgggctgttacctacgcttgctgcgatccgcgcgggtaaaaccattttgctggccaataaagaatcactggttacctgcggacgtctgtttatggacgccgtaaagcagagcaaagcgcaattgttaccggtcgatagcgaacataacgccatttttcagagtttaccgcaacctatccagcataatctgggatacgctgaccttgagcaaaatggcgtggtgtccattttacttaccgggtctggtggccctttccgtgagacgccattgcgcgatttggcaacaatgacgccggatcaagcctgccgtcatccgaactggtcgatggggcgtaaaatttctgtcgattcggctaccatgatgaacaaaggtctggaatacattgaagcgcgttggctgtttaacgccagcgccagccagatggaagtgctgattcacccgcagtcagtgattcactcaatggtgcgctatcaggacggcagtgttctggcgcagctgggggaaccggatatgcgtacgccaattgcccacaccatggcatggccgaatcgcgtgaactctggcgtgaagccgctcgatttttgcaaactaagtgcgttgacatttgccgcaccggattatgatcgttatccatgcctgaaactggcgatggaggcgttcgaacaaggccaggcagcgacgacagcattgaatgccgcaaacgaaatcaccgttgctgcttttcttgcgcaacaaatccgctttacggatatcgctgcgttgaatttatccgtactggaaaaaatggatatgcgcgaaccacaatgtgtggacgatgtgttatctgttgatgcgaacgcgcgtgaagtcgccagaaaagaggtgatgcgtctcgcaagctgatctagagcgggggatccactagttctagagtcgacctgcaggcatgcaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctacagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctg

SEQ ID NO: 90 - DNA, 6798 bp, synthetic construct
  misc_feature 1..6798: note = pT-ispA-MrBBS
  source 1..6798: mol_type = other DNA
gtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatggaattcatggactttccgcagcaactcgaagcctgcgttaagcaggccaaccaggcgctgagccgttttatcgccccactgccctttcagaacactcccgtggtcgaaaccatgcagtatggcgcattattaggtggtaagcgcctgcgacctttcctggtttatgccaccggtcatatgttcggcgttagcacaaacacgctggacgcacccgctgccgccgttgagtgtatccacgcttactcattaattcatgatgatttaccggcaatggatgatgacgatctgcgtcgcggtttgccaacctgccatgtgaagtttggcgaagcaaacgcgattctcgctggcgacgctttacaaacgctggcgttctcgattttaagcgatgccgatatgccggaagtgtcggaccgcgacagaatttcgatgatttctgaactggcgagcgccagtggtattgccggaatgtgcggtggtcaggcattagatttagacgcggaaggcaaacacgtacctctggacgcgcttgagcgtattcatcgtcataaaaccggcgcattgattcgcgccgccgttcgccttggtgcattaagcgccggagataaaggacgtcgtgctctgccggtactcgacaagtatgcagagagcatcggccttgccttccaggttcaggatgacatcctggatgtggtgggagatactgcaacgttgggaaaacgccagggtgccgaccagcaacttggtaaaagtacctaccctgcacttctgggtcttgagcaagcccggaagaaagcccgggatctgatcgacgatgcccgtcagtcgctgaaacaactggctgaacagtcactcgatacctcggcactggaagcgctagcggactacatcatccagcgtaataaataaggatccaaggagatatatcaaatgtcaaccctgtcagtctccacgccgtccttctcatcgtcgccgctgtcctcagtgaacaaaaatagcacgaaacaacacgttacgcgcaatagtgtgatctttcatgattccatttggggcgaccagttcctggaatacaaagaaaaattcaacgttgcaaccgaaaaacaactgattgaagaactgaaagaagaagtccgcaatgaactgatgatccgtgcgtgcaacgaagccagtcgctatattaaactgatccagctgattgatgtggttgaacgtctgggcctggcctaccactttgaaaaagaaatcgaagaatccctgcaacatatttatgttacctacggtcacaaatggacgaactacaacaacatcgaaagcctgtctctgtggtttcgcctgctgcgtcagaacggttttaatgtcagctctgatatcttcgaaaaccatattgacgaaaaaggcaatttccaagaaagtctgtgcaacgatccgcaaggcatgctggcactgtatgaagcggcctacatgcgcgtggaaggcgaaattatcctggacaaagctctggaatttaccaaactgcacctgggtattatcagcaacgatccgtcttgtgacagttccctgcgtacggaaattaaacaggcgctgaaacagccgctgcgtcgccgtctgccgcgtctggaagcagttcgttatattgctatctaccagcaaaaagcgagtcattccgaagtcctgctgaaactggccaaactggatttcaatgtgctgcaggaaatgcacaaagacgaactgtcacaaatttgtaaatggtggaaagacctggacatccgtaacaaactgccgtatgttcgcgatcgtctgattgaaggctatttttggattctgggtatttacttcgaaccgcagcattcgcgcacccgtatgtttctgatgaaaacgtgcatgtggctgatcgtcctggatgacacctttgataattatggcacgtacgaagaactggaaattttcacccaggcggtggaacgctggagcatcacgtgtctggatgaactgccggaatacatgaaactgatctaccatgaacagttccgtgtgcaccaagaaatggaagaatctctggaaaaagaaggtaaagcataccagatccattacatcaaagaaatggctaaagaaggcacccgctctctgctgctggaagcgaaatggctgaaagaaggttatatgccgacgctggatgaatacctgtcaaactcgctggttacctgcggctatgcactgatgacggctcgctcatacgttgcccgtgatgacggtattgtcaccgaagatgcgtttaaatgggtggccacgcacccgccgatcgttaaagcagcttgtaaaattctgcgtctgatggatgacatcgcgacccataaagaagaacaggaacgcggccacatcgcctcatcgattgaatgctatcgtaaagaaaccggtgcatctgaagaagaagcgtgtatggattttctgaaacaggtcgaagacggctggaaagtgatcaatcaagaatcactgatgccgaccgatgtgccgttcccgctgctgatcccggcgattaacctggcccgcgtttcggacacgctgtataaagataacgacggttacaatcatgctgataaagaagtgattggttacattaaaagtctgtttgtccacccgatgattgtgtgaagatcttaggtcgacctgcaggcatgcaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctacagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctg

SEQ ID NO: 91 - DNA, 8655 bp, synthetic construct
  misc_feature 1..8655: note = pTAS-dxs
  source 1..8655: mol_type = other DNA
tcgacctgcaggcatgcaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctacagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctggtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaggtaataaatatggaattcatggactttccgcagcaactcgaagcctgcgttaagcaggccaaccaggcgctgagccgttttatcgccccactgccctttcagaacactcccgtggtcgaaaccatgcagtatggcgcattattaggtggtaagcgcctgcgacctttcctggtttatgccaccggtcatatgttcggcgttagcacaaacacgctggacgcacccgctgccgccgttgagtgtatccacgcttactcattaattcatgatgatttaccggcaatggatgatgacgatctgcgtcgcggtttgccaacctgccatgtgaagtttggcgaagcaaacgcgattctcgctggcgacgctttacaaacgctggcgttctcgattttaagcgatgccgatatgccggaagtgtcggaccgcgacagaatttcgatgatttctgaactggcgagcgccagtggtattgccggaatgtgcggtggtcaggcattagatttagacgcggaaggcaaacacgtacctctggacgcgcttgagcgtattcatcgtcataaaaccggcgcattgattcgcgccgccgttcgccttggtgcattaagcgccggagataaaggacgtcgtgctctgccggtactcgacaagtatgcagagagcatcggccttgccttccaggttcaggatgacatcctggatgtggtgggagatactgcaacgttgggaaaacgccagggtgccgaccagcaacttggtaaaagtacctaccctgcacttctgggtcttgagcaagcccggaagaaagcccgggatctgatcgacgatgcccgtcagtcgctgaaacaactggctgaacagtcactcgatacctcggcactggaagcgctagcggactacatcatccagcgtaataaataaggatccaaaaaggaggtaataaaccatgtcaactcaacaagtttcatcagagaacattgttcgtaacgctgcgaatttccatcctaatatatggggaaaccatttcctcacatgtccttctcagacgattgatagttggactcaacagcaccacaaagaactgaaagaagaggtgaggaaaatgatggtgtctgatgcaaataaacctgcccagagattgcgcttgattgatactgtccaaaggctaggtgtggcttaccactttgaaaaggagattgatgatgcattggagaaaataggtcatgacccttttgatgataaagatgatctctacattgtctctctttgttttcgattgctgaggcagcatggaattaagatatcatgtgatgtgtttgagaagtttaaagatgacgatggaaaattcaaggcatcattgatgaatgatgttcaaggcatgctaagtttatatgaggcagcacacctagccattcacggagaagatattttagatgaagcaattgttttcacgaccactcaccttaagtcaacggtatctaattctcctgtaaactctacttttgctgaacaaatacgtcattctctcagagttcctctccgtaaagctgtacctaggttagagtcgaggtatttcttggatatctattcaagagatgatttgcacgataaaactttgctcaatttcgcaaagttagactttaatatactacaagcaatgcaccagaaggaagcaagtgagatgaccaggtggtggagagattttgacttccttaaaaagctgccttatataagagacagagtcgtggagctatatttttggattctggtgggagtgtcttatcagcccaaattcagcactggtagaatttttttgtccaaaataatatgccttgagaccctcgtagatgatacatttgacgcctacggtacttttgacgagctcgcaatctttactgaagcagttacaagatgggaccttggccacagagatgcactaccagaatacatgaaattcattttcaagacactcattgatgtctacagtgaagctgagcaagaactggcaaaggaagggagatcatacagcatacactatgcaatacgatcgttccaagaactagttatgaagtacttctgcgaagccaagtggttaaataaaggttatgttccgagcctggacgattataaatcagtttcattaagaagtatcggttttttaccgatagcggtagcttccttcgttttcatgggtgatattgcaactaaggaggtctttgaatgggaaatgaataaccctaagatcataatagccgcagaaacgattttcagattcctggatgacatagcaggccataggtttgagcaaaagagagaacatagtccatcagctattgaatgctacaagaatcaacatggagtgtctgaggaagaggcagttaaagcgttgtcgttagaagttgctaatagttggaaagatataaatgaggagctgcttctcaacccaatggctattcctttacctctgcttcaggtgattcttgatctctcacgttcggccgattttatgtacggtaatgctcaagatcgcttcacgcattcaacgatgatgaaagaccaagttgatttggtgctgaaggaccccgttaagcttgacgattaaagatctgtgtggaattgtgagcggataacaatttcacacaggaaacagaccatggaattcatgagttttgatattgccaaatacccgaccctggcactggtcgactccacccaggagttacgactgttgccgaaagagagtttaccgaaactctgcgacgaactgcgccgctatttactcgacagcgtgagccgttccagcgggcacttcgcctccgggctgggcacggtcgaactgaccgtggcgctgcactatgtctacaacaccccgtttgaccaattgatttgggatgtggggcatcaggcttatccgcataaaattttgaccggacgccgcgacaaaatcggcaccatccgtcagaaaggcggtctgcacccgttcccgtggcgcggcgaaagcgaatatgacgtattaagcgtcgggcattcatcaacctccatcagtgccggaattggtattgcggttgctgccgaaaaagaaggcaaaaatcgccgcaccgtctgtgtcattggcgatggcgcgattaccgcaggcatggcgtttgaagcgatgaatcacgcgggcgatatccgtcctgatatgctggtgattctcaacgacaatgaaatgtcgatttccgaaaatgtcggcgcgctcaacaaccatctggcacagctgctttccggtaagctttactcttcactgcgcgaaggcgggaaaaaagttttctctggcgtgccgccaattaaagagctgctcaaacgcaccgaagaacatattaaaggcatggtagtgcctggcacgttgtttgaagagctgggctttaactacatcggcccggtggacggtcacgatgtgctggggcttatcaccacgctaaagaacatgcgcgacctgaaaggcccgcagttcctgcatatcatgaccaaaaaaggtcgtggttatgaaccggcagaaaaagacccgatcactttccacgccgtgcctaaatttgatccctccagcggttgtttgccgaaaagtagcggcggtttgccgagctattcaaaaatctttggcgactggttgtgcgaaacggcagcgaaagacaacaagctgatggcgattactccggcgatgcgtgaaggttccggcatggtcgagttttcacgtaaattcccggatcgctacttcgacgtggcaattgccgagcaacacgcggtgacctttgctgcgggtctggcgattggtgggtacaaacccattgtcgcgatttactccactttcctgcaacgcgcctatgatcaggtgctgcatgacgtggcgattcaaaagcttccggtcctgttcgccatcgaccgcgcgggcattgttggtgctgacggtcaaacccatcagggtgcttttgatctctcttacctgcgctgcataccggaaatggtcattatgaccccgagcgatgaaaacgaatgtcgccagatgctctataccggctatcactataacgatggcccgtcagcggtgcgctacccgcgtggcaacgcggtcggcgtggaactgacgccgctggaaaaactaccaattggcaaaggcattgtgaagcgtcgtggcgagaaactggcgatccttaactttggtacgctgatgccagaagcggcgaaagtcgccgaatcgctgaacgccacgctggtcgatatgcgttttgtgaaaccgcttgatgaagcgttaattctggaaatggccgccagccatgaagcgctggtcaccgtagaagaaaacgccattatgggcggcgcaggcagcggcgtgaacgaagtgctgatggcccatcgtaaaccagtacccgtgctgaacattggcctgccggacttctttattccgcaaggaactcaggaagaaatgcgcgccgaactcggcctcgatgccgctggtatggaagccaaaatcaaggcctggctggcataac

SEQ ID NO: 92 - DNA, 8656 bp, synthetic construct
  misc_feature 1..8656: note = pTAB-idi-dxr
  source 1..8656: mol_type = other DNA
gtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatggaattcgagctcggtacccggggatccgatccgggaggttttcagtaatggactttccgcagcaactcgaagcctgcgttaagcaggccaaccaggcgctgagccgttttatcgccccactgccctttcagaacactcccgtggtcgaaaccatgcagtatggcgcattattaggtggtaagcgcctgcgacctttcctggtttatgccaccggtcatatgttcggcgttagcacaaacacgctggacgcacccgctgccgccgttgagtgtatccacgcttactcattaattcatgatgatttaccggcaatggatgatgacgatctgcgtcgcggtttgccaacctgccatgtgaagtttggcgaagcaaacgcgattctcgctggcgacgctttacaaacgctggcgttctcgattttaagcgatgccgatatgccggaagtgtcggaccgcgacagaatttcgatgatttctgaactggcgagcgccagtggtattgccggaatgtgcggtggtcaggcattagatttagacgcggaaggcaaacacgtacctctggacgcgcttgagcgtattcatcgtcataaaaccggcgcattgattcgcgccgccgttcgccttggtgcattaagcgccggagataaaggacgtcgtgctctgccggtactcgacaagtatgcagagagcatcggccttgccttccaggttcaggatgacatcctggatgtggtgggagatactgcaacgttgggaaaacgccagggtgccgaccagcaacttggtaaaagtacctaccctgcacttctgggtcttgagcaagcccggaagaaagcccgggatctgatcgacgatgcccgtcagtcgctgaaacaactggctgaacagtcactcgatacctcggcactggaagcgctagcggactacatcatccagcgtaataaataagatccataaaggaggtaagaatgtcaaccctgtcagtctccacgccgtccttctcatcgtcgccgctgtcctcagtgaacaaaaatagcacgaaacaacacgttacgcgcaatagtgtgatctttcatgattccatttggggcgaccagttcctggaatacaaagaaaaattcaacgttgcaaccgaaaaacaactgattgaagaactgaaagaagaagtccgcaatgaactgatgatccgtgcgtgcaacgaagccagtcgctatattaaactgatccagctgattgatgtggttgaacgtctgggcctggcctaccactttgaaaaagaaatcgaagaatccctgcaacatatttatgttacctacggtcacaaatggacgaactacaacaacatcgaaagcctgtctctgtggtttcgcctgctgcgtcagaacggttttaatgtcagctctgatatcttcgaaaaccatattgacgaaaaaggcaatttccaagaaagtctgtgcaacgatccgcaaggcatgctggcactgtatgaagcggcctacatgcgcgtggaaggcgaaattatcctggacaaagctctggaatttaccaaactgcacctgggtattatcagcaacgatccgtcttgtgacagttccctgcgtacggaaattaaacaggcgctgaaacagccgctgcgtcgccgtctgccgcgtctggaagcagttcgttatattgctatctaccagcaaaaagcgagtcattccgaagtcctgctgaaactggccaaactggatttcaatgtgctgcaggaaatgcacaaagacgaactgtcacaaatttgtaaatggtggaaagacctggacatccgtaacaaactgccgtatgttcgcgatcgtctgattgaaggctatttttggattctgggtatttacttcgaaccgcagcattcgcgcacccgtatgtttctgatgaaaacgtgcatgtggctgatcgtcctggatgacacctttgataattatggcacgtacgaagaactggaaattttcacccaggcggtggaacgctggagcatcacgtgtctggatgaactgccggaatacatgaaactgatctaccatgaacagttccgtgtgcaccaagaaatggaagaatctctggaaaaagaaggtaaagcataccagatccattacatcaaagaaatggctaaagaaggcacccgctctctgctgctggaagcgaaatggctgaaagaaggttatatgccgacgctggatgaatacctgtcaaactcgctggttacctgcggctatgcactgatgacggctcgctcatacgttgcccgtgatgacggtattgtcaccgaagatgcgtttaaatgggtggccacgcacccgccgatcgttaaagcagcttgtaaaattctgcgtctgatggatgacatcgcgacccataaagaagaacaggaacgcggccacatcgcctcatcgattgaatgctatcgtaaagaaaccggtgcatctgaagaagaagcgtgtatggattttctgaaacaggtcgaagacggctggaaagtgatcaatcaagaatcactgatgccgaccgatgtgccgttcccgctgctgatcccggcgattaacctggcccgcgtttcggacacgctgtataaagataacgacggttacaatcatgctgataaagaagtgattggttacattaaaagtctgtttgtccacccgatgattgtgtgaagatcctgaggaggtaacgtatgcaaacggaacacgtcattttattgaatgcacagggagttcccacgggtacgctggaaaagtatgccgcacacacggcagacacccgcttacatctcgcgttctccagttggctgtttaatgccaaaggacaattattagttacccgccgcgcactgagcaaaaaagcatggcctggcgtgtggactaactcggtttgtgggcacccacaactgggagaaagcaacgaagacgcagtgatccgccgttgccgttatgagcttggcgtggaaattacgcctcctgaatctatctatcctgactttcgctaccgcgccaccgatccgagtggcattgtggaaaatgaagtgtgtccggtatttgccgcacgcaccactagtgcgttacagatcaatgatgatgaagtgatggattatcaatggtgtgatttagcagatgtattacacggtattgatgccacgccgtgggcgttcagtccgtggatggtgatgcaggcgacaaatcgcgaagccagaaaacgattatctgcatttacccagcttaaataagatcttagagtcgacgtgtggaattgtgagcggataacaatttcacacaggaaacagaccatggaattcgagctcatgaagcaactcaccattctgggctcgaccggctcgattggttgcagcacgctggacgtggtgcgccataatcccgaacacttccgcgtagttgcgctggtggcaggcaaaaatgtcactcgcatggtagaacagtgcctggaattctctccccgctatgccgtaatggacgatgaagcgagtgcgaaacttcttaaaacgatgctacagcaacagggtagccgcaccgaagtcttaagtgggcaacaagccgcttgcgatatggcagcgcttgaggatgttgatcaggtgatggcagccattgttggcgctgctgggctgttacctacgcttgctgcgatccgcgcgggtaaaaccattttgctggccaataaagaatcactggttacctgcggacgtctgtttatggacgccgtaaagcagagcaaagcgcaattgttaccggtcgatagcgaacataacgccatttttcagagtttaccgcaacctatccagcataatctgggatacgctgaccttgagcaaaatggcgtggtgtccattttacttaccgggtctggtggccctttccgtgagacgccattgcgcgatttggcaacaatgacgccggatcaagcctgccgtcatccgaactggtcgatggggcgtaaaatttctgtcgattcggctaccatgatgaacaaaggtctggaatacattgaagcgcgttggctgtttaacgccagcgccagccagatggaagtgctgattcacccgcagtcagtgattcactcaatggtgcgctatcaggacggcagtgttctggcgcagctgggggaaccggatatgcgtacgccaattgcccacaccatggcatggccgaatcgcgtgaactctggcgtgaagccgctcgatttttgcaaactaagtgcgttgacatttgccgcaccggattatgatcgttatccatgcctgaaactggcgatggaggcgttcgaacaaggccaggcagcgacgacagcattgaatgccgcaaacgaaatcaccgttgctgcttttcttgcgcaacaaatccgctttacggatatcgctgcgttgaatttatccgtactggaaaaaatggatatgcgcgaaccacaatgtgtggacgatgtgttatctgttgatgcgaacgcgcgtgaagtcgccagaaaagaggtgatgcgtctcgcaagctgatctaaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctacagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctg

SEQ ID NO: 93 - DNA, 9789 bp, synthetic construct
  misc_feature 1..9789: note = pSNAK(-E)-dxs
  source 1..9789: mol_type = other DNA
gatctcattaggtagccgtttcaggcacaattgggattgataaaattagtttttttgtgcccccttattatattgatatgacggcactggctgaagccagaaatgtagaccctggaaaatttcatattggtattgggcaagaccaaatggcggtgaacccaatcagccaagatattgtgacatttgcagccaatgccgcagaagcgatcttgaccaaagaagataaagaggccattgatatggtgattgtcgggactgagtccagtatcgatgagtcaaaagcggccgcagttgtcttacatcgtttaatggggattcaacctttcgctcgctctttcgaaatcaaggaagcttgttacggagcaacagcaggcttacagttagctaagaatcacgtagccttacatccagataaaaaagtcttggttgtagcagcagatattgcaaaatatggattaaattctggcggtgagcctacacaaggagctggggcggttgcaatgttagttgctagtgaaccgcgcatcttggctttaaaagaggataatgtgatgctgacgcaagatatctatgacttttggcgtccaacaggccatccgtatcctatggtcgatggtcctttgtcaaacgaaacctacatccaatcttttgcccaagtctgggatgaacataaaaaaagaaccggtcttgattttgcagattatgatgctttagcgttccatattccttacacaaaaatgggcaaaaaagccttattagcaaaaatctccgaccaaactgaagcagaacaggaacgaattttagcccgttatgaagaaagcatcatctatagtcgtcgcgtaggaaacttgtatacgggttcactttatctgggactcatttcccttttagaaaatgcaacgactttaaccgcaggcaatcaaattgggttattcagttatggttctggtgctgtcgctgaatttttcactggtgaattagtagctggttatcaaaatcatttacaaaaagaaactcatttagcactgctagataatcggacagaactttctatcgctgaatatgaagccatgtttgcagaaactttagacacagatattgatcaaacgttagaagatgaattaaaatatagtatttctgctattaataataccgttcgctcttatcgaaactaactgcagcctcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgagcttatcgatgataagctgtcaaacatgagaattacaacttatatcgtatggggctgacttcaggtgctacatttgaagagataaattgcactgaaatctagaaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccaggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaaagtactgcgatgagtggcagggcggggcgtaatttttttaaggcagttattggtgcccttaaacgcctggtgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagagcctgataaaaacggttagcgcttcgttaatacagatgtaggtgttccacagggtagccagcagcatcctgcgatgcagatccggaacataatggtgcagggcgcttgtttcggcgtgggtatggtggcaggccccgtggccgggggactgttgggcgctgccggcacctgtcctacgagttgcatgataaagaagacagtcataagtgcggcgacgatagtcatgccccgcgcccaccggaaggagctaccggacagcggtgcggactgttgtaactcagaataagaaatgaggccgctcatggcgttccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatcctacctgacgctttttatcgcaactctctactgtttctccatacccgtttttttgggctagcaggaggaattcaccatggtaccaatgacaaaaaaagttggtgtcggtcaggcacatagtaagataattttaataggggaacatgcggtcgtttacggttatcctgccatttccctgcctcttttggaggtggaggtgacctgtaaggtagttcctgcagagagtccttggcgcctttatgaggaggataccttgtccatggcggtttatgcctcactggagtatttgaatatcacagaagcctgcattcgttgtgagattgactcggctatccctgagaaacgggggatgggttcgtcagcggctatcagcatagcggccattcgtgcagtatttgactactatcaggctgatctgcctcatgatgtactagaaatcttggtcaatcgagctgaaatgattgcccatatgaatcctagtggtttggatgctaagacctgtcttagtgaccaacctattcgctttatcaagaacgtaggatttacagaacttgagatggatttatccgcctatttggtgattgccgatacgggtgtttatggtcatactcgtgaagccatccaagtggttcaaaataagggcaaggatgccctaccgtttttgcatgccttgggagaattaacccagcaagcagaagttgcgatttcacaaaaagatgctgaaggactgggacaaatcctcagtcaagcgcatttacatttaaaagaaattggagtcagtagccctgaggcagactttttggttgaaacgactcttagccatggtgctctgggtgccaagatgagcggtggtgggctaggaggttgtatcatagccttggtaaccaatttgacacacgcacaagaactagcagaaagattagaagagaaaggagctgttcagacatggatagagagcctgtaacagtacgttcctacgcaaatattgctattatcaaatattggggaaagaaaaaagaaaaagagatggtgcctgctactagcagtatttctctaactttggaaaatatgtatacagagacgaccttgtcgcctttaccagccaatgtaacagctgacgaattttacatcaatggtcagctacaaaatgaggtcgagcatgccaagatgagtaagattattgaccgttatcgtccagctggtgagggctttgtccgtatcgatactcaaaacaatatgcctacggcagcgggcctgtcctcaagttctagtggtttgtccgccctggtcaaggcttgtaatgcttatttcaagcttggattggatagaagtcagttggcacaggaagccaaatttgcctcaggctcttcttctcggagtttttatggaccactaggagcctgggataaggatagtggagaaatttaccctgtagagacagacttgaaactagctatgattatgttggtgctagaggacaagaaaaaaccaatctctagccgtgacgggatgaaactttgtgtggaaacctcgacgacttttgacgactgggttcgtcagtctgagaaggactatcaggatatgctgatttatctcaaggaaaatgattttgccaagattggagaattaacggagaaaaatgctctggctatgcatgctacgacaaagactgctagtccagccttttcttatctgacggatgcctcttatgaggctatggcctttgttcgccagcttcgtgagaaaggagaggcctgctactttaccatggatgctggtcccaatgttaaggtcttctgtcaggagaaagacttggagcatttgtcagaaattttcggtcagcgttatcgcttgattgtgtcaaaaacaaaggatttgagtcaagatgattgctgttaaaacttgcggaaaactctattgggcaggtgaatatgctattttagagccagggcagttagctttgataaaggatattcccatctatatgagggctgagattgctttttctgacagctaccgtatctattcagatatgtttgatttcgcagtggacttaaggcccaatcctgactacagcttgattcaagaaacgattgctttgatgggagacttcctcgctgttcgcggtcagaatttaagacctttttccctaaaaatctgtggcaaaatggaacgagaagggaaaaagtttggtctaggttctagtggcagcgtcgttgtcttggttgtcaaggctttactggctctctataatctttcggttgatcagaatctcttgttcaagctgactagcgctgtcttgctcaagcgaggagacaatggttccatgggcgaccttgcctgtattgtggcagaggatttggttctttaccagtcatttgatcgccagaaggcggctgcttggttagaagaagaaaacttggcgacagttctggagcgtgattggggattttttatctcacaagtgaaaccaactttagaatgtgatttcttagtgggatggaccaaggaagtggctgtatcgagtcacatggtccagcaaatcaagcaaaatatcaatcaaaattttttaagttcctcaaaagaaacggtggtttctttggtcgaagccttggagcaggggaaagccgaaaaagttatcgagcaagtagaagtagccagcaagcttttagaaggcttgagtacagatatttacacgcctttgcttagacagttgaaagaagccagtcaagatttgcaggccgttgccaagagtagtggtgctggtggtggtgactgtggcatcgccctgagttttgatgcgcaatcttctcgaaacactttaaaaaatcgttgggccgatctggggattgagctcttatatcaagaaaggataggacatgacgacaaatcgtaatctagccccgggaggagagaaattatgcaaacggaacacgtcattttattgaatgcacagggagttcccacgggtacgctggaaaagtatgccgcacacacggcagacacccgcttacatctcgcgttctccagttggctgtttaatgccaaaggacaattattagttacccgccgcgcactgagcaaaaaagcatggcctggcgtgtggactaactcggtttgtgggcacccacaactgggagaaagcaacgaagacgcagtgatccgccgttgccgttatgagcttggcgtggaaattacgcctcctgaatctatctatcctgactttcgctaccgcgccaccgatccgagtggcattgtggaaaatgaagtgtgtccggtatttgccgcacgcaccactagtgcgttacagatcaatgatgatgaagtgatggattatcaatggtgtgatttagcagatgtattacacggtattgatgccacgccgtgggcgttcagtccgtggatggtgatgcaggcgacaaatcgcgaagccagaaaacgattatctgcatttacccagcttaaataagcatgcactagagtcgaggaaacagaccatggagtctcgagtgtggaattgtgagcggataacaatttcacacaggaaacagaccatggaattcatgagttttgatattgccaaatacccgaccctggcactggtcgactccacccaggagttacgactgttgccgaaagagagtttaccgaaactctgcgacgaactgcgccgctatttactcgacagcgtgagccgttccagcgggcacttcgcctccgggctgggcacggtcgaactgaccgtggcgctgcactatgtctacaacaccccgtttgaccaattgatttgggatgtggggcatcaggcttatccgcataaaattttgaccggacgccgcgacaaaatcggcaccatccgtcagaaaggcggtctgcacccgttcccgtggcgcggcgaaagcgaatatgacgtattaagcgtcgggcattcatcaacctccatcagtgccggaattggtattgcggttgctgccgaaaaagaaggcaaaaatcgccgcaccgtctgtgtcattggcgatggcgcgattaccgcaggcatggcgtttgaagcgatgaatcacgcgggcgatatccgtcctgatatgctggtgattctcaacgacaatgaaatgtcgatttccgaaaatgtcggcgcgctcaacaaccatctggcacagctgctttccggtaagctttactcttcactgcgcgaaggcgggaaaaaagttttctctggcgtgccgccaattaaagagctgctcaaacgcaccgaagaacatattaaaggcatggtagtgcctggcacgttgtttgaagagctgggctttaactacatcggcccggtggacggtcacgatgtgctggggcttatcaccacgctaaagaacatgcgcgacctgaaaggcccgcagttcctgcatatcatgaccaaaaaaggtcgtggttatgaaccggcagaaaaagacccgatcactttccacgccgtgcctaaatttgatccctccagcggttgtttgccgaaaagtagcggcggtttgccgagctattcaaaaatctttggcgactggttgtgcgaaacggcagcgaaagacaacaagctgatggcgattactccggcgatgcgtgaaggttccggcatggtcgagttttcacgtaaattcccggatcgctacttcgacgtggcaattgccgagcaacacgcggtgacctttgctgcgggtctggcgattggtgggtacaaacccattgtcgcgatttactccactttcctgcaacgcgcctatgatcaggtgctgcatgacgtggcgattcaaaagcttccggtcctgttcgccatcgaccgcgcgggcattgttggtgctgacggtcaaacccatcagggtgcttttgatctctcttacctgcgctgcataccggaaatggtcattatgaccccgagcgatgaaaacgaatgtcgccagatgctctataccggctatcactataacgatggcccgtcagcggtgcgctacccgcgtggcaacgcggtcggcgtggaactgacgccgctggaaaaactaccaattggcaaaggcattgtgaagcgtcgtggcgagaaactggcgatccttaactttggtacgctgatgccagaagcggcgaaagtcgccgaatcgctgaacgccacgctggtcgatatgcgttttgtgaaaccgcttgatgaagcgttaattctggaaatggccgccagccatgaagcgctggtcaccgtagaagaaaacgccattatgggcggcgcaggcagcggcgtgaacgaagtgctgatggcccatcgtaaaccagtacccgtgctgaacattggcctgccggacttctttattccgcaaggaactcaggaagaaatgcgcgccgaactcggcctcgatgccgctggtatggaagccaaaatcaaggcctggctggcataaa

SEQ ID NO: 94 - DNA, 8938 bp, synthetic construct
  misc_feature 1..8938: note = pSNA(-E)-dxsfree
  source 1..8938: mol_type = other DNA
gatctcattaggtagccgtttcaggcacaattgggattgataaaattagtttttttgtgcccccttattatattgatatgacggcactggctgaagccagaaatgtagaccctggaaaatttcatattggtattgggcaagaccaaatggcggtgaacccaatcagccaagatattgtgacatttgcagccaatgccgcagaagcgatcttgaccaaagaagataaagaggccattgatatggtgattgtcgggactgagtccagtatcgatgagtcaaaagcggccgcagttgtcttacatcgtttaatggggattcaacctttcgctcgctctttcgaaatcaaggaagcttgttacggagcaacagcaggcttacagttagctaagaatcacgtagccttacatccagataaaaaagtcttggttgtagcagcagatattgcaaaatatggattaaattctggcggtgagcctacacaaggagctggggcggttgcaatgttagttgctagtgaaccgcgcatcttggctttaaaagaggataatgtgatgctgacgcaagatatctatgacttttggcgtccaacaggccatccgtatcctatggtcgatggtcctttgtcaaacgaaacctacatccaatcttttgcccaagtctgggatgaacataaaaaaagaaccggtcttgattttgcagattatgatgctttagcgttccatattccttacacaaaaatgggcaaaaaagccttattagcaaaaatctccgaccaaactgaagcagaacaggaacgaattttagcccgttatgaagaaagcatcatctatagtcgtcgcgtaggaaacttgtatacgggttcactttatctgggactcatttcccttttagaaaatgcaacgactttaaccgcaggcaatcaaattgggttattcagttatggttctggtgctgtcgctgaatttttcactggtgaattagtagctggttatcaaaatcatttacaaaaagaaactcatttagcactgctagataatcggacagaactttctatcgctgaatatgaagccatgtttgcagaaactttagacacagatattgatcaaacgttagaagatgaattaaaatatagtatttctgctattaataataccgttcgctcttatcgaaactaactgcagcctcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgagcttatcgatgataagctgtcaaacatgagaattacaacttatatcgtatggggctgacttcaggtgctacatttgaagagataaattgcactgaaatctagaaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccaggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagcttttttttaaggcagttattggtgcccttaaacgcctggtgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagagcctgataaaaacggttagcgcttcgttaatacagatgtaggtgttccacagggtagccagcagcatcctgcgatgcagatccggaacataatggtgcagggcgcttgtttcggcgtgggtatggtggcaggccccgtggccgggggactgttgggcgctgccggcacctgtcctacgagttgcatgataaagaagacagtcataagtgcggcgacgatagtcatgccccgcgcccaccggaaggagctaccggacagcggtgcggactgttgtaactcagaataagaaatgaggccgctcatggcgttccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatcctacctgacgctttttatcgcaactctctactgtttctccatacccgtttttttgggctagcaggaggaattcaccatggtaccaatgacaaaaaaagttggtgtcggtcaggcacatagtaagataattttaataggggaacatgcggtcgtttacggttatcctgccatttccctgcctcttttggaggtggaggtgacctgtaaggtagttcctgcagagagtccttggcgcctttatgaggaggataccttgtccatggcggtttatgcctcactggagtatttgaatatcacagaagcctgcattcgttgtgagattgactcggctatccctgagaaacgggggatgggttcgtcagcggctatcagcatagcggccattcgtgcagtatttgactactatcaggctgatctgcctcatgatgtactagaaatcttggtcaatcgagctgaaatgattgcccatatgaatcctagtggtttggatgctaagacctgtcttagtgaccaacctattcgctttatcaagaacgtaggatttacagaacttgagatggatttatccgcctatttggtgattgccgatacgggtgtttatggtcatactcgtgaagccatccaagtggttcaaaataagggcaaggatgccctaccgtttttgcatgccttgggagaattaacccagcaagcagaagttgcgatttcacaaaaagatgctgaaggactgggacaaatcctcagtcaagcgcatttacatttaaaagaaattggagtcagtagccctgaggcagactttttggttgaaacgactcttagccatggtgctctgggtgccaagatgagcggtggtgggctaggaggttgtatcatagccttggtaaccaatttgacacacgcacaagaactagcagaaagattagaagagaaaggagctgttcagacatggatagagagcctgtaacagtacgttcctacgcaaatattgctattatcaaatattggggaaagaaaaaagaaaaagagatggtgcctgctactagcagtatttctctaactttggaaaatatgtatacagagacgaccttgtcgcctttaccagccaatgtaacagctgacgaattttacatcaatggtcagctacaaaatgaggtcgagcatgccaagatgagtaagattattgaccgttatcgtccagctggtgagggctttgtccgtatcgatactcaaaacaatatgcctacggcagcgggcctgtcctcaagttctagtggtttgtccgccctggtcaaggcttgtaatgcttatttcaagcttggattggatagaagtcagttggcacaggaagccaaatttgcctcaggctcttcttctcggagtttttatggaccactaggagcctgggataaggatagtggagaaatttaccctgtagagacagacttgaaactagctatgattatgttggtgctagaggacaagaaaaaaccaatctctagccgtgacgggatgaaactttgtgtggaaacctcgacgacttttgacgactgggttcgtcagtctgagaaggactatcaggatatgctgatttatctcaaggaaaatgattttgccaagattggagaattaacggagaaaaatgctctggctatgcatgctacgacaaagactgctagtccagccttttcttatctgacggatgcctcttatgaggctatggcctttgttcgccagcttcgtgagaaaggagaggcctgctactttaccatggatgctggtcccaatgttaaggtcttctgtcaggagaaagacttggagcatttgtcagaaattttcggtcagcgttatcgcttgattgtgtcaaaaacaaaggatttgagtcaagatgattgctgttaaaacttgcggaaaactctattgggcaggtgaatatgctattttagagccagggcagttagctttgataaaggatattcccatctatatgagggctgagattgctttttctgacagctaccgtatctattcagatatgtttgatttcgcagtggacttaaggcccaatcctgactacagcttgattcaagaaacgattgctttgatgggagacttcctcgctgttcgcggtcagaatttaagacctttttccctaaaaatctgtggcaaaatggaacgagaagggaaaaagtttggtctaggttctagtggcagcgtcgttgtcttggttgtcaaggctttactggctctctataatctttcggttgatcagaatctcttgttcaagctgactagcgctgtcttgctcaagcgaggagacaatggttccatgggcgaccttgcctgtattgtggcagaggatttggttctttaccagtcatttgatcgccagaaggcggctgcttggttagaagaagaaaacttggcgacagttctggagcgtgattggggattttttatctcacaagtgaaaccaactttagaatgtgatttcttagtgggatggaccaaggaagtggctgtatcgagtcacatggtccagcaaatcaagcaaaatatcaatcaaaattttttaagttcctcaaaagaaacggtggtttctttggtcgaagccttggagcaggggaaagccgaaaaagttatcgagcaagtagaagtagccagcaagcttttagaaggcttgagtacagatatttacacgcctttgcttagacagttgaaagaagccagtcaagatttgcaggccgttgccaagagtagtggtgctggtggtggtgactgtggcatcgccctgagttttgatgcgcaatcttctcgaaacactttaaaaaatcgttgggccgatctggggattgagctcttatatcaagaaaggataggacatgacgacaaatcgtaatctagccccgggaggagagaaattatgcaaacggaacacgtcattttattgaatgcacagggagttcccacgggtacgctggaaaagtatgccgcacacacggcagacacccgcttacatctcgcgttctccagttggctgtttaatgccaaaggacaattattagttacccgccgcgcactgagcaaaaaagcatggcctggcgtgtggactaactcggtttgtgggcacccacaactgggagaaagcaacgaagacgcagtgatccgccgttgccgttatgagcttggcgtggaaattacgcctcctgaatctatctatcctgactttcgctaccgcgccaccgatccgagtggcattgtggaaaatgaagtgtgtccggtatttgccgcacgcaccactagtgcgttacagatcaatgatgatgaagtgatggattatcaatggtgtgatttagcagatgtattacacggtattgatgccacgccgtgggcgttcagtccgtggatggtgatgcaggcgacaaatcgcgaagccagaaaacgattatctgcatttacccagcttaaataagcatgcactagagtcgaggaaacagaccatggagtctcgagtgtggaattgtgagcggataacaatttcacacaggaaacagaccatggaattcatgagttttgatattgccaaatacccgaccctggcactggtcgactccacccaggagttacgactgttgccgaaagagagtttaccgaaactctgcgacgaactgcgccgctatttactcgacagcgtgagccgttccagcgggcacttcgcctccgggctgggcacggtcgaactgaccgtggcgctgcactatgtctacaacaccccgtttgaccaattgatttgggatgtggggcatcaggcttatccgcataaaattttgaccggacgccgcgacaaaatcggcaccatccgtcagaaaggcggtctgcacccgttcccgtggcgcggcgaaagcgaatatgacgtattaagcgtcgggcattcatcaacctccatcagtgccggaattggtattgcggttgctgccgaaaaagaaggcaaaaatcgccgcaccgtctgtgtcattggcgatggcgcgattaccgcaggcatggcgtttgaagcgatgaatcacgcgggcgatatccgtcctgatatgctggtgattctcaacgacaatgaaatgtcgatttccgaaaatgtcggcgcgctcaacaaccatctggcacagctgctttccggtaagctttactcttcactgcgcgaaggcgggaaaaaagttttctctggcgtgccgccaattaaagagctgctcaaacgcaccgaagaacatattaaaggcatggtagtgcctggcacgttgtttgaagagctgggctttaactacatcggcccggtggacggtcacgatgtgctggggcttatcaccacgctaaagaacatgcgcgacctgaaaggcccgcagttcctgcatatcatgaccaaaaaaggtcgtggttatgaaccggcagaaaaagacccgatcactttccacgccgtgcctaaatttgatccctccagcggttgtttgccgaaaagtagcggcggtttgccgagctattcaaaaatctttggcgactggttgtgcgaaacggcagcgaaagacaacaagctgatggcgattactccggcgatgcgtgaaggttccggcatggtcgagttttcacgtaaattcccggatcgctacttcgacgtggcaattgccgagcaacacgcggtgacctttgctgcgggtctggcgattggtgggtacaaacccattgtcgcgatttactccactttcctgcaacgcgcctatgatcaggtgctgcatgacgtggcgattcaaaagcttccggtcctgttcgccatcgaccgcgcgggcattgttggtgctgacggtcaaacccatcagggtgcttttgatctctcttacctgcgctgcataccggaaatggtcattatgaccccgagcgatgaaaacgaatgtcgccagatgctctataccggctatcactataacgatggcccgtcagcggtgcgctacccgcgtggcaacgcggtcggcgtggaactgacgccgctggaaaaactaccaattggcaaaggcattgtgaagcgtcgtggcgagaaactggcgatccttaactttggtacgctgatgccagaagcggcgaaagtcgccgaatcgctgaacgccacgctggtcgatatgcgttttgtgaaaccgcttgatgaagcgttaattctggaaatggccgccagccatgaagcgctggtcaccgtagaagaaaacgccattatgggcggcgcaggcagcggcgtgaacgaagtgctgatggcccatcgtaaaccagtacccgtgctgaacattggcctgccggacttctttattccgcaaggaactcaggaagaaatgcgcgccgaactcggcctcgatgccgctggtatggaagccaaaatcaaggcctggctggcataaa

SEQ ID NO: 95 - DNA, 14034 bp, synthetic construct
  misc_feature 1..14034: note = pT-HBSREYbbOdxrfree
  source 1..14034: mol_type = other DNA
ctagcacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcaaagcttgcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctggtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatggaattctacgtactagtgaattcaggaggtaataaatatgggtctgatgctgattgattggtgtgcactggctctggttgttttcattggcctgccgcacggcgcgctggatgctgccatttctttttctatgatctcttctgcaaaacgcattgctcgtctggctggtattctgctgatctatctgctgctggcgaccgcgttcttcctgatctggtatcagctgccagcgtttagcctgctgatcttcctgctgatctccattatccactttggtatggcagacttcaacgcgtccccaagcaaactgaaatggccgcatatcatcgcccacggcggtgttgttactgtttggctgccgctgatccagaaaaacgaagtaactaaactgtttagcatcctgactaacggtccgactccgatcctgtgggacatcctgctgattttcttcctgtgttggtctattggcgtgtgtctgcacacgtacgaaaccctgcgctctaaacattacaacatcgcctttgaactgatcggtctgattttcctggcgtggtatgcgccgcctctggttacgtttgccacttacttctgcttcattcattcccgtcgccacttctcctttgtgtggaagcagctgcaacacatgtcttccaaaaagatgatgattggcagcgcgattatcctgtcctgtacctcttggctgatcggcggtggtatctatttcttcctgaactccaaaatgatcgcctctgaggctgcgctgcagactgtgttcatcggtctggcggcactgaccgtgccgcacatgattctgatcgacttcatcttccgtccgcactcttcccgtatcaaaatcaaaaactaattaattaaaggaggtaataatatgactcataaagcaacggagatcctgacaggtaaagttatgcaaaaatcggtcttaattaccggatgttccagtggaattggcctggaaagcgcgctcgaattaaaacgccagggttttcatgtgctggcaggttgccggaaaccggatgatgttgagcgcatgaacagcatgggatttaccggcgtgttgatcgatctggattcaccagaaagtgttgatcgcgcagccgacgaggtgatcgccctgaccgataattgtctgtatgggatctttaacaatgccggattcggcatgtatggccccctttccaccatcagccgtgcgcagatggaacagcagttttccgccaactttttcggcgcacaccagctcaccatgcgcctgttacccgcgatgttaccgcacggtgaagggcgtattgtgatgacatcatcggtgatgggattaatctccacgccgggtcgtggcgcttacgcggccagtaaatatgcgctggaggcgtggtcagatgcactgcgcatggagctgcgccacagcggaattaaagtcagcctgatcgaacccggtcccattcgtactcgcttcaccgacaacgtcaaccagacgcaaagtgataaaccagtcgaaaatcccggcatcgccgcccgctttacgttgggaccggaagcggtggtggacaaagtacgccatgcttttattagcgagaagccgaagatgcgctatccggtgacgctggtgacctgggcggtaatggtgcttaagcgcctgctgccggggcgcgtgatggacaaaatattgcaggggtaactagtaggaggtaataaatatggtgagtggcagtaaagcgggcgtttcgcctcatcgcgaaatagaagtaatgagacaatccattgacgatcacctggctggcctgttacctgaaaccgacagccaggatatcgtcagccttgcgatgcgtgaaggcgtcatggcacccggtaaacggatccgtccgctgctgatgctgctggccgcccgcgacctccgctaccagggcagtatgcctacgctgctcgatctcgcctgcgccgttgaactgacccataccgcgtcgctgatgctcgacgacatgccctgcatggacaacgccgagctgcgccgcggtcagcccactacccacaaaaaatttggtgagagcgtggcgatccttgcctccgttgggctgctctctaaagcctttggtctgatcgccgccaccggcgatctgccgggggagaggcgtgcccaggcggtcaacgagctctctaccgccgtgggcgtgcagggcctggtactggggcagtttcgcgatcttaacgatgccgccctcgaccgtacccctgacgctatcctcagcaccaaccacctcaagaccggcattctgttcagcgcgatgctgcagatcgtcgccattgcttccgcctcgtcgccgagcacgcgagagacgctgcacgccttcgccctcgacttcggccaggcgtttcaactgctggacgatctgcgtgacgatcacccggaaaccggtaaagatcgcaataaggacgcgggaaaatcgacgctggtcaaccggctgggcgcagacgcggcccggcaaaagctgcgcgagcatattgattccgccgacaaacacctcacttttgcctgtccgcagggcggcgccatccgacagtttatgcatctgtggtttggccatcaccttgccgactggtcaccggtcatgaaaatcgcctgataactcgaggaggtataaaggatgaaaaaaaccgttgtgattggcgcaggctttggtggcctggcgctggcgattcgcctgcaggcggcagggatcccaaccgtactgctggagcagcgggacaagcccggcggtcgggcctacgtctggcatgaccagggctttacctttgacgccgggccgacggtgatcaccgatcctaccgcgcttgaggcgctgttcaccctggccggcaggcgcatggaggattacgtcaggctgctgccggtaaaacccttctaccgactctgctgggagtccgggaagaccctcgactatgctaacgacagcgccgagcttgaggcgcagattacccagttcaacccccgcgacgtcgagggctaccggcgctttctggcttactcccaggcggtattccaggagggatatttgcgcctcggcagcgtgccgttcctctcttttcgcgacatgctgcgcgccgggccgcagctgcttaagctccaggcgtggcagagcgtctaccagtcggtttcgcgctttattgaggatgagcatctgcggcaggccttctcgttccactccctgctggtaggcggcaaccccttcaccacctcgtccatctacaccctgatccacgcccttgagcgggagtggggggtctggttccctgagggcggcaccggggcgctggtgaacggcatggtgaagctgtttaccgatctgggcggggagatcgaactcaacgcccgggtcgaagagctggtggtggccgataaccgcgtaagccaggtccggctggcggatggtcggatctttgacaccgacgccgtagcctcgaacgctgacgtggtgaacacctataaaaagctgctcggccaccatccggtggggcagaagcgggcggcagcgctggagcgcaagagcatgagcaactcgctgtttgtgctctacttcggcctgaaccagcctcattcccagctggcgcaccataccatctgttttggtccccgctaccgggagctgatcgacgagatctttaccggcagcgcgctggcggatgacttctcgctctacctgcactcgccctgcgtgaccgatccctcgctcgcgcctcccggctgcgccagcttctacgtgctggccccggtgccgcatcttggcaacgcgccgctggactgggcgcaggaggggccgaagctgcgcgaccgcatctttgactaccttgaagagcgctatatgcccggcctgcgtagccagctggtgacccagcggatctttaccccggcagacttccacgacacgctggatgcgcatctgggatcggccttctccatcgagccgctgctgacccaaagcgcctggttccgcccgcacaaccgcgacagcgacattgccaacctctacctggtgggcgcaggtactcaccctggggcgggcattcctggcgtagtggcctcggcgaaagccaccgccagcctgatgattgaggatctgcaatgagccaaccgccgctgcttgaccacgccacgcagaccatggccaacggctcgaaaagttttgccaccgctgcgaagctgttcgacccggccacccgccgtagcgtgctgatgctctacacctggtgccgccactgcgatgacgtcattgacgaccagacccacggcttcgccagcgaggccgcggcggaggaggaggccacccagcgcctggcccggctgcgcacgctgaccctggcggcgtttgaaggggccgagatgcaggatccggccttcgctgcctttcaggaggtggcgctgacccacggtattacgccccgcatggcgctcgatcacctcgacggctttgcgatggacgtggctcagacccgctatgtcacctttgaggatacgctgcgctactgctatcacgtggcgggcgtggtgggtctgatgatggccagggtgatgggcgtgcgggatgagcgggtgctggatcgcgcctgcgatctggggctggccttccagctgacgaatatcgcccgggatattattgacgatgcggctattgaccgctgctatctgcccgccgagtggctgcaggatgccgggctgaccccggagaactatgccgcgcgggagaatcgggccgcgctggcgcgggtggcggagcggcttattgatgccgcagagccgtactacatctcctcccaggccgggctacacgatctgccgccgcgctgcgcctgggcgatcgccaccgcccgcagcgtctaccgggagatcggtattaaggtaaaagcggcgggaggcagcgcctgggatcgccgccagcacaccagcaaaggtgaaaaaattgccatgctgatggcggcaccggggcaggttattcgggcgaagacgacgagggtgacgccgcgtccggccggtctttggcagcgtcccgtttagtaatctagaggaggtaataaaatatgcttcgttcgttgctcagaggcctcacgcatatcccccgcgtgaactccgcccagcagcccagctgtgcacacgcgcgactccagtttaagctcaggagcatgcagatgacgctcatgcagcccagcatctcagccaatctgtcgcgcgccgaggaccgcacagaccacatgaggggtgcaagcacctgggcaggcgggcagtcgcaggatgagctgatgctgaaggacgagtgcatcttggtggatgttgaggacaacatcacaggccatgccagcaagctggagtgtcacaagttcctaccacatcagcctgcaggcctgctgcaccgggccttctctgtgttcctgtttgacgatcaggggcgactgctgctgcaacagcgtgcacgctcaaaaatcaccttcccaagtgtgtggacgaacacctgctgcagccaccctttacatgggcagaccccagatgaggtggaccaactaagccaggtggccgacggaacagtacctggcgcaaaggctgctgccatccgcaagttggagcacgagctggggataccagcgcaccagctgccggcaagcgcgtttcgcttcctcacgcgtttgcactactgtgccgcggacgtgcagccagctgcgacacaatcagcgctctggggcgagcacgaaatggactacatcttgttcatccgggccaacgtcaccttggcgcccaaccctgacgaggtggacgaagtcaggtacgtgacgcaagaggagctgcggcagatgatgcagccggacaacgggctgcaatggtcgccgtggtttcgcatcatcgccgcgcgcttccttgagcgttggtgggctgacctggacgcggccctaaacactgacaaacacgaggattggggaacggtgcatcacatcaacgaagcgtgataagcggccgcgctgttgacaattaatcatccggctcgtataatgtgtggaattgtgagcggataacaatttcacacaggaaacagaccatggagttgaaaacagtagttattattgatgcattacgaacaccaattggaaaatataaaggcagcttaagtcaagtaagtgccgtagacttaggaacacatgttacaacacaacttttaaaaagacattccactatttctgaagaaattgatcaagtaatctttggaaatgttttacaagctggaaatggccaaaatcccgcacgacaaatagcaataaacagcggtttatctcatgaaattcccgcaatgacagttaatgaggtctgcggatcaggaatgaaggccgttattttggcgaaacaattgattcaattaggagaagcggaagttttaattgctggcgggattgagaatatgtcccaagcacctaaattacaacgatttaattacgaaacagaaagctatgatgcgcctttttctagtatgatgtacgatgggttaacggatgcctttagtggtcaagcaatgggcttaactgctgaaaatgtggccgaaaagtatcatgtaactagagaagagcaagatcaattttctgtacattcacaattaaaagcagctcaagcacaagcagaagggatattcgctgacgaaatagccccattagaagtatcaggaacgcttgtggagaaagatgaagggattcgccctaattcgagcgttgagaagctaggaacgcttaaaacagtttttaaagaagacggtactgtaacagcagggaatgcatcaaccattaatgatggggcttctgctttgattattgcttcacaagaatatgccgaagcacacggtcttccttatttagctattattcgagacagtgtggaagtcggtattgatccagcctatatgggaatttcgccgattaaagccattcaaaaactgttagcgcgcaatcaacttactacggaagaaattgatctgtatgaaatcaacgaagcatttgcagcaacttcaatcgtggtccaaagagaactggctttaccagaggaaaaggtcaacatttatggtggcggtatttcattaggtcatgcgattggtgccacaggtgctcgtttattaacgagtttaagttatcaattaaatcaaaaagaaaagaaatatggagtggcttctttatgtatcggcggtggcttaggactcgctatgctactagagagacctcagcaaaaaaaaaacagccgattttatcaaatgagtcctgaggaacgcctggcttctcttcttaatgaaggccagatttctgctgatacaaaaaaagaatttgaaaatacggctttatcttcgcagattgccaatcatatgattgaaaatcaaatcagtgaaacagaagtgccgatgggcgttggcttacatttaacagtggacgaaactgattatttggtaccaatggcgacagaagagccctcagtgattgcggctttgagtaatggtgcaaaaatagcacaaggatttaaaacagtgaatcaacaacgtttaatgcgtggacaaatcgttttttacgatgttgcagacgccgagtcattgattgatgaactacaagtaagagaaacggaaatttttcaacaagcagagttaagttatccatctatcgttaaacgcggcggcggcttaagagatttgcaatatcgtgcttttgatgaatcatttgtatctgtcgactttttagtagatgttaaggatgcaatgggggcaaatatcgttaacgctatgttggaaggtgtggccgagttgttccgtgaatggtttgcggagcaaaagattttattcagtattttaagtaattatgccacggagtcggttgttacgatgaaaacggctattccagtttcacgtttaagtaaggggagcaatggccgggaaattgctgaaaaaattgttttagcttcacgctatgcttcattagatccttatcgggcagtcacgcataacaaagggatcatgaatggcattgaagctgtcgttttagctacaggaaatgatacacgcgctgttagcgcttcttgtcatgcttttgcggtgaaggaaggtcgctaccaaggtttgactagttggacgctggatggcgaacaactaattggtgaaatttcagttccgcttgcgttagccacggttggcggtgccacaaaagtcttacctaaatctcaagcagctgctgatttgttagcagtgacggatgcaaaagaactaagtcgagtagtagcggctgttggtttggcacaaaatttagcggcgttacgggccttagtctctgaaggaattcaaaaaggacacatggctctacaagcacgttctttagcgatgacggtcggagctactggtaaagaagttgaggcagtcgctcaacaattaaaacgtcaaaaaacgatgaaccaagaccgagccttggctattttaaatgatttaagaaaacaataaaaaaacagttcagcagaaattattctgctgaactgttttttttcacattaggtagccgtttcagctcgacaggaggagcggctatgcaaccgcattatgatctgattctcgtgggggctggactcgcgaatggccttatcgccctgcgtcttcagcagcagcaacctgatatgcgtattttgcttatcgacgccgcaccccaggcgggcgggaatcatacgtggtcatttcaccacgatgatttgactgagagccaacatcgttggatagctccgctggtggttcatcactggcccgactatcaggtacgctttcccacacgccgtcgtaagctgaacagcggctacttttgtattacttctcagcgtttcgctgaggttttacagcgacagtttggcccgcacttgtggatggataccgcggtcgcagaggttaatgcggaatctgttcggttgaaaaagggtcaggttatcggtgcccgcgcggtgattgacgggcggggttatgcggcaaattcagcactgagcgtgggcttccaggcgtttattggccaggaatggcgattgagccacccgcatggtttatcgtctcccattatcatggatgccacggtcgatcagcaaaatggttatcgcttcgtgtacagcctgccgctctcgccgaccagattgttaattgaagacacgcactatattgataatgcgacattagatcctgaatgcgcgcggcaaaatatttgcgactatgccgcgcaacagggttggcagcttcagacactgctgcgagaagaacagggcgccttacccattactctgtcgggcaatgccgacgcattctggcagcagcgccccctggcctgtagtggattacgtgccggtctgttccatcctaccaccggctattcactgccgctggcggttgccgtggccgaccgcctgagtgcacttgatgtctttacgtcggcctcaattcaccatgccattacgcattttgcccgcgagcgctggcagcagcagggctttttccgcatgctgaatcgcatgctgtttttagccggacccgccgattcacgctggcgggttatgcagcgtttttatggtttacctgaagatttaattgcccgtttttatgcgggaaaactcacgctgaccgatcggctacgtattctgagcggcaagccgcctgttccggtattagcagcattgcaagccattatgacgactcatcgttaactgcaggcatgagtactgtgtggaattgtgagcggataacaatttcacacaggaaacagaccatggaattcgagctcatgaagcaactcaccattctgggctcgaccggctcgattggttgcagcacgctggacgtggtgcgccataatcccgaacacttccgcgtagttgcgctggtggcaggcaaaaatgtcactcgcatggtagaacagtgcctggaattctctccccgctatgccgtaatggacgatgaagcgagtgcgaaacttcttaaaacgatgctacagcaacagggtagccgcaccgaagtcttaagtgggcaacaagccgcttgcgatatggcagcgcttgaggatgttgatcaggtgatggcagccattgttggcgctgctgggctgttacctacgcttgctgcgatccgcgcgggtaaaaccattttgctggccaataaagaatcactggttacctgcggacgtctgtttatggacgccgtaaagcagagcaaagcgcaattgttaccggtcgatagcgaacataacgccatttttcagagtttaccgcaacctatccagcataatctgggatacgctgaccttgagcaaaatggcgtggtgtccattttacttaccgggtctggtggccctttccgtgagacgccattgcgcgatttggcaacaatgacgccggatcaagcctgccgtcatccgaactggtcgatggggcgtaaaatttctgtcgattcggctaccatgatgaacaaaggtctggaatacattgaagcgcgttggctgtttaacgccagcgccagccagatggaagtgctgattcacccgcagtcagtgattcactcaatggtgcgctatcaggacggcagtgttctggcgcagctgggggaaccggatatgcgtacgccaattgcccacaccatggcatggccgaatcgcgtgaactctggcgtgaagccgctcgatttttgcaaactaagtgcgttgacatttgccgcaccggattatgatcgttatccatgcctgaaactggcgatggaggcgttcgaacaaggccaggcagcgacgacagcattgaatgccgcaaacgaaatcaccgttgctgcttttcttgcgcaacaaatccgctttacggatatcgctgcgttgaatttatccgtactggaaaaaatggatatgcgcgaaccacaatgtgtggacgatgtgttatctgttgatgcgaacgcgcgtgaagtcgccagaaaagaggtgatgcgtctcgcaagctgatctag

SEQ ID NO: 96 - DNA, 3355 bp, synthetic construct
  misc_feature 1..3355: note = pEGFP
  source 1..3355: mol_type = other DNA
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaggatccccgggtaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagaattccaactgagcgccggtcgctaccattaccaacttgtctggtgtcaaaaataataggcctactagtcggccgtacgggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcggccttaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 97 - DNA, 4616 bp, synthetic construct
  misc_feature 1..4616: note = pEGFP-dxr
  source 1..4616: mol_type = other DNA
ctagtcggccgtacgggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcggccttaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaggatccccgggtaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagaattccaactgagcgccggtcgctaccattaccaacttgtctggtgtcaaaaataataggactgtgtggaattgtgagcggataacaatttcacacaggaaacagaccatggaattcgagctcatgaagcaactcaccattctgggctcgaccggctcgattggttgcagcacgctggacgtggtgcgccataatcccgaacacttccgcgtagttgcgctggtggcaggcaaaaatgtcactcgcatggtagaacagtgcctggaattctctccccgctatgccgtaatggacgatgaagcgagtgcgaaacttcttaaaacgatgctacagcaacagggtagccgcaccgaagtcttaagtgggcaacaagccgcttgcgatatggcagcgcttgaggatgttgatcaggtgatggcagccattgttggcgctgctgggctgttacctacgcttgctgcgatccgcgcgggtaaaaccattttgctggccaataaagaatcactggttacctgcggacgtctgtttatggacgccgtaaagcagagcaaagcgcaattgttaccggtcgatagcgaacataacgccatttttcagagtttaccgcaacctatccagcataatctgggatacgctgaccttgagcaaaatggcgtggtgtccattttacttaccgggtctggtggccctttccgtgagacgccattgcgcgatttggcaacaatgacgccggatcaagcctgccgtcatccgaactggtcgatggggcgtaaaatttctgtcgattcggctaccatgatgaacaaaggtctggaatacattgaagcgcgttggctgtttaacgccagcgccagccagatggaagtgctgattcacccgcagtcagtgattcactcaatggtgcgctatcaggacggcagtgttctggcgcagctgggggaaccggatatgcgtacgccaattgcccacaccatggcatggccgaatcgcgtgaactctggcgtgaagccgctcgatttttgcaaactaagtgcgttgacatttgccgcaccggattatgatcgttatccatgcctgaaactggcgatggaggcgttcgaacaaggccaggcagcgacgacagcattgaatgccgcaaacgaaatcaccgttgctgcttttcttgcgcaacaaatccgctttacggatatcgctgcgttgaatttatccgtactggaaaaaatggatatgcgcgaaccacaatgtgtggacgatgtgttatctgttgatgcgaacgcgcgtgaagtcgccagaaaagaggtgatgcgtctcgcaagctgatctag

SEQ ID NO: 98 - DNA, 2230 bp, synthetic construct
  misc_feature 1..2230: note = pORI19
  source 1..2230: mol_type = other DNA
ggaggctcaagggagtatgagggaatgaaattccctcatgggtttgattttaaaaattgcttgcaattttgccgagcggtagcgctggaaaatttttgaaaaaaatttggaatttggaaaaaaatggggggaaaggaagcgaattttgcttccgtactacgaccccccattaagtgccgagtgccaatttttgtgccaaaaacgctctatcccaactggctcaagggtttaaggggtttttcaatcgccaacgaatcgccaacgttttcgccaacgttttttataaatctatatttaagtagctttattgttgtttttatgattacaaagtgatacactaactttataaaattatttgattggagttttttaaatggtgatttcagaatcgctagttctagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatggatctcgacccgtgctataattatactaattttataaggaggaaaaaatatgggcatttttagtatttttgtaatcagcacagttcattatcaaccaaacaaaaaataagtggttataatgaatcgttaataagcaaaattcatataaccaaattaaagagggttataatgaacgagaaaaatataaaacacagtcaaaactttattacttcaaaacataatatagataaaataatgacaaatataagattaaatgaacatgataatatctttgaaatcggctcaggaaaaggccattttacccttgaattagtaaagaggtgtaatttcgtaactgccattgaaatagaccataaattatgcaaaactacagaaaataaacttgttgatcacgataatttccaagttttaaacaaggatatattgcagtttaaatttcctaaaaaccaatcctataaaatatatggtaatataccttataacataagtacggatataatacgcaaaattgtttttgatagtatagctaatgagatttatttaatcgtggaatacgggtttgctaaaagattattaaatacaaaacgctcattggcattacttttaatggcagaagttgatatttctatattaagtatggttccaagagaatattttcatcctaaacctaaagtgaatagctcacttatcagattaagtagaaaaaaatcaagaatatcacacaaagataaacaaaagtataattatttcgttatgaaatgggttaacaaagaatacaagaaaatatttacaaaaaatcaatttaacaattccttaaaacatgcaggaattgacgatttaaacaatattagctttgaacaattcttatctcttttcaatagctataaattatttaataagtaagttaagggatgcataaactgcatcccttaacttgtttttcgtgtgcctattttttgtgaatcgggtcgaggcctcggactagcgattttttattaaaacgtctcaaaatcgtttctgagacgttttagcgtttatttcgtttagttatcggcataatcgttaaaacaggcgttatcgtagcgtaaaagcccttgagcgtagcgtggctttgcagcgaagatgttgtctgttagattatgaaagccgatgactgaatgaaataataagcgcagcgcccttctatttcggttgga

SEQ ID NO: 99 - DNA, 4215 bp, synthetic construct
  misc_feature 1..4215: note = pORI19-mvaA
  source 1..4215: mol_type = other DNA
ggaggctcaagggagtatgagggaatgaaattccctcatgggtttgattttaaaaattgcttgcaattttgccgagcggtagcgctggaaaatttttgaaaaaaatttggaatttggaaaaaaatggggggaaaggaagcgaattttgcttccgtactacgaccccccattaagtgccgagtgccaatttttgtgccaaaaacgctctatcccaactggctcaagggtttaaggggtttttcaatcgccaacgaatcgccaacgttttcgccaacgttttttataaatctatatttaagtagctttattgttgtttttatgattacaaagtgatacactaactttataaaattatttgattggagttttttaaatggtgatttcagaatcgctagttctagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtaccagcttcaaccattaatgaagtttgtggttcagggatgaaggcagtaattctagctaagcaacttttacagttgggcgaagcagaagttgttattgctggtggaacggaatcaatgtctaatgcgcctattcttaaaaatcgtaaaactgaagaagaaacattaagcatgttaagtgacggcttaattgatgctttttctggcatttcaatgggaattattggagaaacaattgcggagcagtttgatgttagtcgtgctgaccaagacctattcgcacaaaattctcaagagaaggcagtagcagccagtcaagcaggaatttttaatgacgagatcgtagctcttgggaaacttactgttgatgaaactcctcgaccttcatctagcctagaaaaattggctacattacggacagcctttaaggaaaatggaacagttacagctgggaattcttcaccagttaatgacggagcttctgctttaattttggccactaaagagtatgctgaaaaacatggaatttcttatttagctgaattagttgagagtgcggaagttggtattgaccctgcaattatgggagtatcaccgattgatgcaattcaaaaattagttcaacgctcaggcgttcagctttcagaaatagatctttttgaaattaatgaggcctttgctgcatcaagtattgctgttaatcgtgaattgggcttaaaagattctcaagttaatatttatggaggagcgattgctcttggacatgctattgggtcaagtggtgccaaaatcctgacaactttgggatatgctttgaaacgagaacaaaagcgttatggaattgctagtctttgtattggcggtggtctagggcttgctattttattgaaaaatcctgacttttaaaaattgatggaggaattgcccagtatttctgggcatttttgggaaatatgagaaaaaaattttaactaaaaaatttgagaaaataatagcctgtcaagattttttcttgacagcatgaaattagtctgctataatattaaatataataaaaaagaaataggaaatataaaatgtctgtaaaaattcgtttgactcgtatgggttctaagaaaaaacctttctaccgtattaatgttgctgattcacgtgcaccacgtgatggtaaattcatcgaaacagttggaacttacaatccacttgtaactgaaaaccaagtaactcttaaagaagaacgcgtattggaatggttgagcaacggagcacaaccttcagatacagttcgtaacctcctttcaaaagcgggcgttatgaaaaaattccacgaatcaaaactttctaaataatttttgagataagctgctttttagcagcttttcgtgaatataaaaatcacataaaatggacttgaataactttcaagttagagtagaggtaagaaatgcaaaaagatgtgaaagaactcgttttaactattgtaaaacctttggtgactcagcctgatgaagtttctttggaaattattgaaggtgaggagtttatggaataccatctcaaagttgctgacggagatattggaagaataattggacgtcaaggtcgaattattcaagccattcgtacagttgtatactttgttcctgttgaagggaaaaaagttcgactcctcgtggaccaataataagtaaaaataaaagaactcatcattgatgagttcttttatttttggaaaacggaacgttcttttgagaaataaaatagttatttagaatttccttttattgtccccaaaaatatgctagaatgaaaaaatattgattaataattgtagaaataatcagtgataaatataaattaactgatttttatataggtggataatggataagaaaaaaggaatcttattagtagctttagggacgcctcgttcttgtgagacagaggatgtaagggaatatttaaaagaatttttgggtgatccactcgtcattcatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatggatctcgacccgtgctataattatactaattttataaggaggaaaaaatatgggcatttttagtatttttgtaatcagcacagttcattatcaaccaaacaaaaaataagtggttataatgaatcgttaataagcaaaattcatataaccaaattaaagagggttataatgaacgagaaaaatataaaacacagtcaaaactttattacttcaaaacataatatagataaaataatgacaaatataagattaaatgaacatgataatatctttgaaatcggctcaggaaaaggccattttacccttgaattagtaaagaggtgtaatttcgtaactgccattgaaatagaccataaattatgcaaaactacagaaaataaacttgttgatcacgataatttccaagttttaaacaaggatatattgcagtttaaatttcctaaaaaccaatcctataaaatatatggtaatataccttataacataagtacggatataatacgcaaaattgtttttgatagtatagctaatgagatttatttaatcgtggaatacgggtttgctaaaagattattaaatacaaaacgctcattggcattacttttaatggcagaagttgatatttctatattaagtatggttccaagagaatattttcatcctaaacctaaagtgaatagctcacttatcagattaagtagaaaaaaatcaagaatatcacacaaagataaacaaaagtataattatttcgttatgaaatgggttaacaaagaatacaagaaaatatttacaaaaaatcaatttaacaattccttaaaacatgcaggaattgacgatttaaacaatattagctttgaacaattcttatctcttttcaatagctataaattatttaataagtaagttaagggatgcataaactgcatcccttaacttgtttttcgtgtgcctattttttgtgaatcgggtcgaggcctcggactagcgattttttattaaaacgtctcaaaatcgtttctgagacgttttagcgtttatttcgtttagttatcggcataatcgttaaaacaggcgttatcgtagcgtaaaagcccttgagcgtagcgtggctttgcagcgaagatgttgtctgttagattatgaaagccgatgactgaatgaaataataagcgcagcgcccttctatttcggttgga

SEQ ID NO: 100 - DNA, 5790 bp, synthetic construct
  misc_feature 1..5790: note = pCI372
  source 1..5790: mol_type = other DNA
gtaaatcgctcctttttaggtggcacaaatgtgaggcattttcgctctttccggcgaggctagttacccttaagttattggtatgactggttttaagcgcaaaaaaagttgctttttcgtacctattaatgtatcgttagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatcttgctcttttgtcagagaaatcataactctttttttcgattctgaaatcaccatttaaaaaactccaatcaaataattttataaaattagtgtatcactttgtaatcataaaaacaacaataaagctacttaaatatagatttataaaaaacgttggcgaaaacgttggcgattcgttggcgattgaaaaacccctcaaacccttgagccagttgggatagagcgtttttggcacaaaaattggcactcggcacttaatggggggtcgtagtacggaagcaaaattcgcttcctttccccccatttttttccaaattccaaatttttttcaaaaattttccagcgctaccgctcggcaaaattgcaagcaatttttaaaatcaaacccatgagggaatttcattccctcaaactcccttgagcctcctccaaccgaaatagaaggacgctgcgcttattatttcattcagtcatcggctttcataatctaacagacaacatcttcgctgcaaagccacgctacgctcaagggcttttacgctacgataacgcctgttttaacgattatgccgataactaaacgaaataaacgctaaaacgtctcagaaacgattttgagacgttttaataaaaaatcgacttcgttctttttttacctctcggttatgagttagttcaaattcgttctttttaggttctaaatcgtgtttttcttggaattgtgctgttttatcctttaccttgtctacaaaccccttaaaaacgtttttaaaggcttttaagccgtctgtacgttccttaaggaattcgagctcggtacccggggatcctctagagtcgacctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagatcttttgcccattttatttttataaaatgggcaggtggcgtttgtgtaaagcaaatcgacacaatccaaaggggataaaaggggaaagtgaaacttcccccttttcaagccacattgtaatacaagaacgaagtgttttgtattacaatgtgatagcttgcagtatttatggttttatatggtctattttgttgtgaggattgtaaccgaatagggcgcaatgcttattacaaaatcaatgacaaagggcgattgaggaatgagcgctgaggcattttatctttgagtaagttattgatggatcagaaaaatgtatcacaaattgaaacaaagactcactcatttaagagaagctactatcatgaaattttgttgttgtgataagcaacttctaatacacgatttttagccattacatcactcgtttttagagtgatgtgtaagtgcgcattgcactctttttttacgaaacaagccgaccagcgtttgaaactttttagtttttcatcattctattttaaaacgttctaaaactcgatttaagcgactttaattcgaaactgtctatttgttcaaagggagcattaagaatgcttaaacgagcttttaagggggtttaaattgattttgaattgaatagcttgttgtaagttgtaaaaaaaacaagttaaacaaagtatcagttttccatttaagggttgttagggcttgccctgaccgtctgtaagacgcttgattgcatgatatgagtatttagctagtcaaacagttaaaacagcttatatgagcaattagagggaatccaataaattcctaaaagcggttttgatcttttcttttagcgagtgaacgctgcaagtaaaatgtgagcgttcactcgctcactcctttttttgatgactttgacctttggttttaaatttttgaaaaaaataaaaaataggcgaagcctattatatatttatcttatatattttaatcttttattcttttgcgtcaaaaaaaatcaatattttcaaggctttatagaattatataccaacaaaaaactgtgtatataccaacaaaaaactgtgcatacaccaacaaaaaactgtgcatataccaacttctttgtttgtttcgttggtatataatgatataataaaagcatgaagaatctctctacgaaaagtgtttcttcatgcttatctaaactcactcacaaaggagcagttttctatgtctagtatatcaaaaaatgaacctaatcaaaagcaggtgcaaaccttgaacgaattgtcaaaacgaaaagtagtggaacataattctttaattaccagtattgcgaaaatggataaaacgccactgaaaatgtttgaattagccgtgtcttgtattaataccgaagaaccacccaaagatcatacggtttatctctcaaaagaagaattgtttgccttttttaaggtatctgataatgacaaacatagtcgttttaaacaagcagtagagaatatgcaaaaacaagccttttttcaaattaaagaagaagtaggtaaaggatttaaatttaggagtattgttcccattccatatgtcgagtggacagattatcatgatgacgtaaaaattgaatttcatcgtgaaatcatgccctacttaattaatctaaaacaaaatttcacgcaacatgctttgtctgatattgcagagctgaatagcaaatactctattatcttgtaccgttggttatccatgaattataaccaatacgagcattatagttataagggcggacggagagaagaacaagtggaagcctaccgcaatcctaccatttcaatgcgagaattacgagaaatgacggatacagttgatgaatacccccgctttgatagattagaacatagagttttaaaagaaccaatagaagaaattaacgaaaacacctcttttaacgtgacgtatgacaagataaaaaaaggacgaagcattgattctattgtctttcatatcacgaaaaaacgtcgagcagatgataacagctacaagttagaagataaagattatcaatccgacaaagaggaaaaatcaagaaatgaagctgacttattaaaacaggcaatggaaagcaagtacacacgattattgattgaaaactttctcttatcccctcttgaaatgacggacacggcacttatggcaggtttgcaaaagaacgtctatccgttgtatgacgagttaaaggaattaagaggattgaatggggtcaaagaccacttgtcttatatatctagcaaaaaagaagcctattctaaacgcaatgtagcgaagtatctgaaaaaagcaatcgagcaatatctacctacggttaaaaggcaggacttaaaccatgagtgagaacttaaaaacgattaaagagttggctgatgagttgggcgtatcaaaaaagaaaattcattatcaagtatctaaattagatagtgatttgatacaaaaattagacggcactatatatctagctgatttcactttttgcattctacaaactgcataactcatat

SEQ ID NO: 101 - DNA, 6350 bp, synthetic construct
  misc_feature 1..6350: note = pCIN
  source 1..6350: mol_type = other DNA
gtaaatcgctcctttttaggtggcacaaatgtgaggcattttcgctctttccggcgaggctagttacccttaagttattggtatgactggttttaagcgcaaaaaaagttgctttttcgtacctattaatgtatcgttagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatcttgctcttttgtcagagaaatcataactctttttttcgattctgaaatcaccatttaaaaaactccaatcaaataattttataaaattagtgtatcactttgtaatcataaaaacaacaataaagctacttaaatatagatttataaaaaacgttggcgaaaacgttggcgattcgttggcgattgaaaaacccctcaaacccttgagccagttgggatagagcgtttttggcacaaaaattggcactcggcacttaatggggggtcgtagtacggaagcaaaattcgcttcctttccccccatttttttccaaattccaaatttttttcaaaaattttccagcgctaccgctcggcaaaattgcaagcaatttttaaaatcaaacccatgagggaatttcattccctcaaactcccttgagcctcctccaaccgaaatagaaggacgctgcgcttattatttcattcagtcatcggctttcataatctaacagacaacatcttcgctgcaaagccacgctacgctcaagggcttttacgctacgataacgcctgttttaacgattatgccgataactaaacgaaataaacgctaaaacgtctcagaaacgattttgagacgttttaataaaaaatcgacttcgttctttttttacctctcggttatgagttagttcaaattcgttctttttaggttctaaatcgtgtttttcttggaattgtgctgttttatcctttaccttgtctacaaaccccttaaaaacgtttttaaaggcttttaagccgtctgtacgttccttaagggctagcatagattagtttattcttgacactacaagctaaatgtggtataatcccatagagtgtggaattgtgagcggataacaatttcacacaggaaacagaccatggaattcgagctcggtacccggggatcctctagagtcgacctgcaggcatgcaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttaccggtcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagatcttttgcccattttatttttataaaatgggcaggtggcgtttgtgtaaagcaaatcgacacaatccaaaggggataaaaggggaaagtgaaacttcccccttttcaagccacattgtaatacaagaacgaagtgttttgtattacaatgtgatagcttgcagtatttatggttttatatggtctattttgttgtgaggattgtaaccgaatagggcgcaatgcttattacaaaatcaatgacaaagggcgattgaggaatgagcgctgaggcattttatctttgagtaagttattgatggatcagaaaaatgtatcacaaattgaaacaaagactcactcatttaagagaagctactatcatgaaattttgttgttgtgataagcaacttctaatacacgatttttagccattacatcactcgtttttagagtgatgtgtaagtgcgcattgcactctttttttacgaaacaagccgaccagcgtttgaaactttttagtttttcatcattctattttaaaacgttctaaaactcgatttaagcgactttaattcgaaactgtctatttgttcaaagggagcattaagaatgcttaaacgagcttttaagggggtttaaattgattttgaattgaatagcttgttgtaagttgtaaaaaaaacaagttaaacaaagtatcagttttccatttaagggttgttagggcttgccctgaccgtctgtaagacgcttgattgcatgatatgagtatttagctagtcaaacagttaaaacagcttatatgagcaattagagggaatccaataaattcctaaaagcggttttgatcttttcttttagcgagtgaacgctgcaagtaaaatgtgagcgttcactcgctcactcctttttttgatgactttgacctttggttttaaatttttgaaaaaaataaaaaataggcgaagcctattatatatttatcttatatattttaatcttttattcttttgcgtcaaaaaaaatcaatattttcaaggctttatagaattatataccaacaaaaaactgtgtatataccaacaaaaaactgtgcatacaccaacaaaaaactgtgcatataccaacttctttgtttgtttcgttggtatataatgatataataaaagcatgaagaatctctctacgaaaagtgtttcttcatgcttatctaaactcactcacaaaggagcagttttctatgtctagtatatcaaaaaatgaacctaatcaaaagcaggtgcaaaccttgaacgaattgtcaaaacgaaaagtagtggaacataattctttaattaccagtattgcgaaaatggataaaacgccactgaaaatgtttgaattagccgtgtcttgtattaataccgaagaaccacccaaagatcatacggtttatctctcaaaagaagaattgtttgccttttttaaggtatctgataatgacaaacatagtcgttttaaacaagcagtagagaatatgcaaaaacaagccttttttcaaattaaagaagaagtaggtaaaggatttaaatttaggagtattgttcccattccatatgtcgagtggacagattatcatgatgacgtaaaaattgaatttcatcgtgaaatcatgccctacttaattaatctaaaacaaaatttcacgcaacatgctttgtctgatattgcagagctgaatagcaaatactctattatcttgtaccgttggttatccatgaattataaccaatacgagcattatagttataagggcggacggagagaagaacaagtggaagcctaccgcaatcctaccatttcaatgcgagaattacgagaaatgacggatacagttgatgaatacccccgctttgatagattagaacatagagttttaaaagaaccaatagaagaaattaacgaaaacacctcttttaacgtgacgtatgacaagataaaaaaaggacgaagcattgattctattgtctttcatatcacgaaaaaacgtcgagcagatgataacagctacaagttagaagataaagattatcaatccgacaaagaggaaaaatcaagaaatgaagctgacttattaaaacaggcaatggaaagcaagtacacacgattattgattgaaaactttctcttatcccctcttgaaatgacggacacggcacttatggcaggtttgcaaaagaacgtctatccgttgtatgacgagttaaaggaattaagaggattgaatggggtcaaagaccacttgtcttatatatctagcaaaaaagaagcctattctaaacgcaatgtagcgaagtatctgaaaaaagcaatcgagcaatatctacctacggttaaaaggcaggacttaaaccatgagtgagaacttaaaaacgattaaagagttggctgatgagttgggcgtatcaaaaaagaaaattcattatcaagtatctaaattagatagtgatttgatacaaaaattagacggcactatatatctagctgatttcactttttgcattctacaaactgcataactcatat

SEQ ID NO: 102 - DNA, 7597 bp, synthetic construct
  misc_feature 1..7597: note = pCIN-mvaA
  source 1..7597: mol_type = other DNA
gtaaatcgctcctttttaggtggcacaaatgtgaggcattttcgctctttccggcgaggctagttacccttaagttattggtatgactggttttaagcgcaaaaaaagttgctttttcgtacctattaatgtatcgttagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatcttgctcttttgtcagagaaatcataactctttttttcgattctgaaatcaccatttaaaaaactccaatcaaataattttataaaattagtgtatcactttgtaatcataaaaacaacaataaagctacttaaatatagatttataaaaaacgttggcgaaaacgttggcgattcgttggcgattgaaaaacccctcaaacccttgagccagttgggatagagcgtttttggcacaaaaattggcactcggcacttaatggggggtcgtagtacggaagcaaaattcgcttcctttccccccatttttttccaaattccaaatttttttcaaaaattttccagcgctaccgctcggcaaaattgcaagcaatttttaaaatcaaacccatgagggaatttcattccctcaaactcccttgagcctcctccaaccgaaatagaaggacgctgcgcttattatttcattcagtcatcggctttcataatctaacagacaacatcttcgctgcaaagccacgctacgctcaagggcttttacgctacgataacgcctgttttaacgattatgccgataactaaacgaaataaacgctaaaacgtctcagaaacgattttgagacgttttaataaaaaatcgacttcgttctttttttacctctcggttatgagttagttcaaattcgttctttttaggttctaaatcgtgtttttcttggaattgtgctgttttatcctttaccttgtctacaaaccccttaaaaacgtttttaaaggcttttaagccgtctgtacgttccttaagggctagcatagattagtttattcttgacactacaagctaaatgtggtataatcccatagagtgtggaattgtgagcggataacaatttcacacaggaaacagaccatggaattcgagctcggtacccggggatccaggaggtaataaatatgagaaaaaaattttatcaaatgtcgccacaagagcgattgaattcgttaaacttatccgaaaagagtcaagaaattttaagtgagatggccctagatacaactattttggacaatctcatagaaaatcaaatttctgaatttgaattgccaatgggaattgcccaaaattttgttattaatggacaaagttttctaataccaatggttacagaggaaccgtcagtaattgccgcagcaagtaatggagcaaaaatagctggaaattttgtagctgaaattaaagaacgtttaatgcgtggtcaaattgtattttatgatgttaaaaattcagataagattgcaaatgaaatacttgaaaagcaagaaaaaatctttgaacaagccgaactttcttacccttcaatcgttaaacgaggtggcggtttgagagaggtttctagtcggattttctctagtcaaaagtttttatctgttgatgtcaaggttgatgttaaagacgccatgggtgcaaatattattaattcaatcttagagggaattgctgaactctttcggaggtggtttcctgatgaaaaaattctgtttagtattttatcaaattatgctactgaatcactagtgaaagtgacctgtgaaattccggttgaacgcctttcaaaaaaagcagatggatatgaaattggtcaaaaaattatggctgcttcccaatattcaaaaattgatccttatcgtgctagcacccataacaaagggattatgaacggtatcaatgcggtcattttagctacaggaaatgatactcgggccatctcagctgctattcatgcttatgcggcaaaagatggagcctaccaagggttagccaactgggaacttcaagagaaaatgttagttggcgaactcgaatttcctttaccagtggcaactgtcggagggggagtcaaggttttgccaaaagcacaagcggccatggagattctgggtatcagtgatgctaaggagctcgcaaaagttattgctgcagtgggactagctcaaaacttagccgctttacgtgccctagtttcagaaggaattcaacagggacacatgagtctacaagctcgttctttggctttgagtgtaggagcacaagcggatgaaatcgcgatattaagtcaacaattgcgcaaagaaaaagtgatgaatcaagaagtagcccaaaatttactaaaaaatttgagaaaataatctagagtcgacctgcaggcatgcaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttaccggtcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagatcttttgcccattttatttttataaaatgggcaggtggcgtttgtgtaaagcaaatcgacacaatccaaaggggataaaaggggaaagtgaaacttcccccttttcaagccacattgtaatacaagaacgaagtgttttgtattacaatgtgatagcttgcagtatttatggttttatatggtctattttgttgtgaggattgtaaccgaatagggcgcaatgcttattacaaaatcaatgacaaagggcgattgaggaatgagcgctgaggcattttatctttgagtaagttattgatggatcagaaaaatgtatcacaaattgaaacaaagactcactcatttaagagaagctactatcatgaaattttgttgttgtgataagcaacttctaatacacgatttttagccattacatcactcgtttttagagtgatgtgtaagtgcgcattgcactctttttttacgaaacaagccgaccagcgtttgaaactttttagtttttcatcattctattttaaaacgttctaaaactcgatttaagcgactttaattcgaaactgtctatttgttcaaagggagcattaagaatgcttaaacgagcttttaagggggtttaaattgattttgaattgaatagcttgttgtaagttgtaaaaaaaacaagttaaacaaagtatcagttttccatttaagggttgttagggcttgccctgaccgtctgtaagacgcttgattgcatgatatgagtatttagctagtcaaacagttaaaacagcttatatgagcaattagagggaatccaataaattcctaaaagcggttttgatcttttcttttagcgagtgaacgctgcaagtaaaatgtgagcgttcactcgctcactcctttttttgatgactttgacctttggttttaaatttttgaaaaaaataaaaaataggcgaagcctattatatatttatcttatatattttaatcttttattcttttgcgtcaaaaaaaatcaatattttcaaggctttatagaattatataccaacaaaaaactgtgtatataccaacaaaaaactgtgcatacaccaacaaaaaactgtgcatataccaacttctttgtttgtttcgttggtatataatgatataataaaagcatgaagaatctctctacgaaaagtgtttcttcatgcttatctaaactcactcacaaaggagcagttttctatgtctagtatatcaaaaaatgaacctaatcaaaagcaggtgcaaaccttgaacgaattgtcaaaacgaaaagtagtggaacataattctttaattaccagtattgcgaaaatggataaaacgccactgaaaatgtttgaattagccgtgtcttgtattaataccgaagaaccacccaaagatcatacggtttatctctcaaaagaagaattgtttgccttttttaaggtatctgataatgacaaacatagtcgttttaaacaagcagtagagaatatgcaaaaacaagccttttttcaaattaaagaagaagtaggtaaaggatttaaatttaggagtattgttcccattccatatgtcgagtggacagattatcatgatgacgtaaaaattgaatttcatcgtgaaatcatgccctacttaattaatctaaaacaaaatttcacgcaacatgctttgtctgatattgcagagctgaatagcaaatactctattatcttgtaccgttggttatccatgaattataaccaatacgagcattatagttataagggcggacggagagaagaacaagtggaagcctaccgcaatcctaccatttcaatgcgagaattacgagaaatgacggatacagttgatgaatacccccgctttgatagattagaacatagagttttaaaagaaccaatagaagaaattaacgaaaacacctcttttaacgtgacgtatgacaagataaaaaaaggacgaagcattgattctattgtctttcatatcacgaaaaaacgtcgagcagatgataacagctacaagttagaagataaagattatcaatccgacaaagaggaaaaatcaagaaatgaagctgacttattaaaacaggcaatggaaagcaagtacacacgattattgattgaaaactttctcttatcccctcttgaaatgacggacacggcacttatggcaggtttgcaaaagaacgtctatccgttgtatgacgagttaaaggaattaagaggattgaatggggtcaaagaccacttgtcttatatatctagcaaaaaagaagcctattctaaacgcaatgtagcgaagtatctgaaaaaagcaatcgagcaatatctacctacggttaaaaggcaggacttaaaccatgagtgagaacttaaaaacgattaaagagttggctgatgagttgggcgtatcaaaaaagaaaattcattatcaagtatctaaattagatagtgatttgatacaaaaattagacggcactatatatctagctgatttcactttttgcattctacaaactgcataactcatat

SEQ ID NO: 103 - DNA, 8327 bp, synthetic construct
  misc_feature 1..8327: note = pCIN-mvaA-EGFP
  source 1..8327: mol_type = other DNA
gtaaatcgctcctttttaggtggcacaaatgtgaggcattttcgctctttccggcgaggctagttacccttaagttattggtatgactggttttaagcgcaaaaaaagttgctttttcgtacctattaatgtatcgttagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatcttgctcttttgtcagagaaatcataactctttttttcgattctgaaatcaccatttaaaaaactccaatcaaataattttataaaattagtgtatcactttgtaatcataaaaacaacaataaagctacttaaatatagatttataaaaaacgttggcgaaaacgttggcgattcgttggcgattgaaaaacccctcaaacccttgagccagttgggatagagcgtttttggcacaaaaattggcactcggcacttaatggggggtcgtagtacggaagcaaaattcgcttcctttccccccatttttttccaaattccaaatttttttcaaaaattttccagcgctaccgctcggcaaaattgcaagcaatttttaaaatcaaacccatgagggaatttcattccctcaaactcccttgagcctcctccaaccgaaatagaaggacgctgcgcttattatttcattcagtcatcggctttcataatctaacagacaacatcttcgctgcaaagccacgctacgctcaagggcttttacgctacgataacgcctgttttaacgattatgccgataactaaacgaaataaacgctaaaacgtctcagaaacgattttgagacgttttaataaaaaatcgacttcgttctttttttacctctcggttatgagttagttcaaattcgttctttttaggttctaaatcgtgtttttcttggaattgtgctgttttatcctttaccttgtctacaaaccccttaaaaacgtttttaaaggcttttaagccgtctgtacgttccttaagggctagcatagattagtttattcttgacactacaagctaaatgtggtataatcccatagagtgtggaattgtgagcggataacaatttcacacaggaaacagaccatggaattcgagctcggtacccggggatccaggaggtaataaatatgagaaaaaaattttatcaaatgtcgccacaagagcgattgaattcgttaaacttatccgaaaagagtcaagaaattttaagtgagatggccctagatacaactattttggacaatctcatagaaaatcaaatttctgaatttgaattgccaatgggaattgcccaaaattttgttattaatggacaaagttttctaataccaatggttacagaggaaccgtcagtaattgccgcagcaagtaatggagcaaaaatagctggaaattttgtagctgaaattaaagaacgtttaatgcgtggtcaaattgtattttatgatgttaaaaattcagataagattgcaaatgaaatacttgaaaagcaagaaaaaatctttgaacaagccgaactttcttacccttcaatcgttaaacgaggtggcggtttgagagaggtttctagtcggattttctctagtcaaaagtttttatctgttgatgtcaaggttgatgttaaagacgccatgggtgcaaatattattaattcaatcttagagggaattgctgaactctttcggaggtggtttcctgatgaaaaaattctgtttagtattttatcaaattatgctactgaatcactagtgaaagtgacctgtgaaattccggttgaacgcctttcaaaaaaagcagatggatatgaaattggtcaaaaaattatggctgcttcccaatattcaaaaattgatccttatcgtgctagcacccataacaaagggattatgaacggtatcaatgcggtcattttagctacaggaaatgatactcgggccatctcagctgctattcatgcttatgcggcaaaagatggagcctaccaagggttagccaactgggaacttcaagagaaaatgttagttggcgaactcgaatttcctttaccagtggcaactgtcggagggggagtcaaggttttgccaaaagcacaagcggccatggagattctgggtatcagtgatgctaaggagctcgcaaaagttattgctgcagtgggactagctcaaaacttagccgctttacgtgccctagtttcagaaggaattcaacagggacacatgagtctacaagctcgttctttggctttgagtgtaggagcacaagcggatgaaatcgcgatattaagtcaacaattgcgcaaagaaaaagtgatgaatcaagaagtagcccaaaatttactaaaaaatttgagaaaataatctagagtcgacaggaggtaataaatatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagcatgcaagcttggctgttttggcggatgagagaagattttcggcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctcccccatgcgagagtagggaactgccaggcatcaaataaaacgaaaggctcagtcgaaagactggggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttaccggtcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagatcttttgcccattttatttttataaaatgggcaggtggcgtttgtgtaaagcaaatcgacacaatccaaaggggataaaaggggaaagtgaaacttcccccttttcaagccacattgtaatacaagaacgaagtgttttgtattacaatgtgatagcttgcagtatttatggttttatatggtctattttgttgtgaggattgtaaccgaatagggcgcaatgcttattacaaaatcaatgacaaagggcgattgaggaatgagcgctgaggcattttatctttgagtaagttattgatggatcagaaaaatgtatcacaaattgaaacaaagactcactcatttaagagaagctactatcatgaaattttgttgttgtgataagcaacttctaatacacgatttttagccattacatcactcgtttttagagtgatgtgtaagtgcgcattgcactctttttttacgaaacaagccgaccagcgtttgaaactttttagtttttcatcattctattttaaaacgttctaaaactcgatttaagcgactttaattcgaaactgtctatttgttcaaagggagcattaagaatgcttaaacgagcttttaagggggtttaaattgattttgaattgaatagcttgttgtaagttgtaaaaaaaacaagttaaacaaagtatcagttttccatttaagggttgttagggcttgccctgaccgtctgtaagacgcttgattgcatgatatgagtatttagctagtcaaacagttaaaacagcttatatgagcaattagagggaatccaataaattcctaaaagcggttttgatcttttcttttagcgagtgaacgctgcaagtaaaatgtgagcgttcactcgctcactcctttttttgatgactttgacctttggttttaaatttttgaaaaaaataaaaaataggcgaagcctattatatatttatcttatatattttaatcttttattcttttgcgtcaaaaaaaatcaatattttcaaggctttatagaattatataccaacaaaaaactgtgtatataccaacaaaaaactgtgcatacaccaacaaaaaactgtgcatataccaacttctttgtttgtttcgttggtatataatgatataataaaagcatgaagaatctctctacgaaaagtgtttcttcatgcttatctaaactcactcacaaaggagcagttttctatgtctagtatatcaaaaaatgaacctaatcaaaagcaggtgcaaaccttgaacgaattgtcaaaacgaaaagtagtggaacataattctttaattaccagtattgcgaaaatggataaaacgccactgaaaatgtttgaattagccgtgtcttgtattaataccgaagaaccacccaaagatcatacggtttatctctcaaaagaagaattgtttgccttttttaaggtatctgataatgacaaacatagtcgttttaaacaagcagtagagaatatgcaaaaacaagccttttttcaaattaaagaagaagtaggtaaaggatttaaatttaggagtattgttcccattccatatgtcgagtggacagattatcatgatgacgtaaaaattgaatttcatcgtgaaatcatgccctacttaattaatctaaaacaaaatttcacgcaacatgctttgtctgatattgcagagctgaatagcaaatactctattatcttgtaccgttggttatccatgaattataaccaatacgagcattatagttataagggcggacggagagaagaacaagtggaagcctaccgcaatcctaccatttcaatgcgagaattacgagaaatgacggatacagttgatgaatacccccgctttgatagattagaacatagagttttaaaagaaccaatagaagaaattaacgaaaacacctcttttaacgtgacgtatgacaagataaaaaaaggacgaagcattgattctattgtctttcatatcacgaaaaaacgtcgagcagatgataacagctacaagttagaagataaagattatcaatccgacaaagaggaaaaatcaagaaatgaagctgacttattaaaacaggcaatggaaagcaagtacacacgattattgattgaaaactttctcttatcccctcttgaaatgacggacacggcacttatggcaggtttgcaaaagaacgtctatccgttgtatgacgagttaaaggaattaagaggattgaatggggtcaaagaccacttgtcttatatatctagcaaaaaagaagcctattctaaacgcaatgtagcgaagtatctgaaaaaagcaatcgagcaatatctacctacggttaaaaggcaggacttaaaccatgagtgagaacttaaaaacgattaaagagttggctgatgagttgggcgtatcaaaaaagaaaattcattatcaagtatctaaattagatagtgatttgatacaaaaattagacggcactatatatctagctgatttcactttttgcattctacaaactgcataactcatat

SEQ ID NO: 104 - DNA, 2412 bp, Enterococcus faecalis
  source 1..2412: mol_type = genomic DNA
ttgaaaacagtagttattattgatgcattacgaacaccaattggaaaatataaaggcagcttaagtcaagtaagtgccgtagacttaggaacacatgttacaacacaacttttaaaaagacattccactatttctgaagaaattgatcaagtaatctttggaaatgttttacaagctggaaatggccaaaatcccgcacgacaaatagcaataaacagcggtttatctcatgaaattcccgcaatgacagttaatgaggtctgcggatcaggaatgaaggccgttattttggcgaaacaattgattcaattaggagaagcggaagttttaattgctggcgggattgagaatatgtcccaagcacctaaattacaacgatttaattacgaaacagaaagctatgatgcgcctttttctagtatgatgtacgatgggttaacggatgcctttagtggtcaagcaatgggcttaactgctgaaaatgtggccgaaaagtatcatgtaactagagaagagcaagatcaattttctgtacattcacaattaaaagcagctcaagcacaagcagaagggatattcgctgacgaaatagccccattagaagtatcaggaacgcttgtggagaaagatgaagggattcgccctaattcgagcgttgagaagctaggaacgcttaaaacagtttttaaagaagacggtactgtaacagcagggaatgcatcaaccattaatgatggggcttctgctttgattattgcttcacaagaatatgccgaagcacacggtcttccttatttagctattattcgagacagtgtggaagtcggtattgatccagcctatatgggaatttcgccgattaaagccattcaaaaactgttagcgcgcaatcaacttactacggaagaaattgatctgtatgaaatcaacgaagcatttgcagcaacttcaatcgtggtccaaagagaactggctttaccagaggaaaaggtcaacatttatggtggcggtatttcattaggtcatgcgattggtgccacaggtgctcgtttattaacgagtttaagttatcaattaaatcaaaaagaaaagaaatatggagtggcttctttatgtatcggcggtggcttaggactcgctatgctactagagagacctcagcaaaaaaaaaacagccgattttatcaaatgagtcctgaggaacgcctggcttctcttcttaatgaaggccagatttctgctgatacaaaaaaagaatttgaaaatacggctttatcttcgcagattgccaatcatatgattgaaaatcaaatcagtgaaacagaagtgccgatgggcgttggcttacatttaacagtggacgaaactgattatttggtaccaatggcgacagaagagccctcagtgattgcggctttgagtaatggtgcaaaaatagcacaaggatttaaaacagtgaatcaacaacgtttaatgcgtggacaaatcgttttttacgatgttgcagacgccgagtcattgattgatgaactacaagtaagagaaacggaaatttttcaacaagcagagttaagttatccatctatcgttaaacgcggcggcggcttaagagatttgcaatatcgtgcttttgatgaatcatttgtatctgtcgactttttagtagatgttaaggatgcaatgggggcaaatatcgttaacgctatgttggaaggtgtggccgagttgttccgtgaatggtttgcggagcaaaagattttattcagtattttaagtaattatgccacggagtcggttgttacgatgaaaacggctattccagtttcacgtttaagtaaggggagcaatggccgggaaattgctgaaaaaattgttttagcttcacgctatgcttcattagatccttatcgggcagtcacgcataacaaagggatcatgaatggcattgaagctgtcgttttagctacaggaaatgatacacgcgctgttagcgcttcttgtcatgcttttgcggtgaaggaaggtcgctaccaaggtttgactagttggacgctggatggcgaacaactaattggtgaaatttcagttccgcttgcgttagccacggttggcggtgccacaaaagtcttacctaaatctcaagcagctgctgatttgttagcagtgacggatgcaaaagaactaagtcgagtagtagcggctgttggtttggcacaaaatttagcggcgttacgggccttagtctctgaaggaattcaaaaaggacacatggctctacaagcacgttctttagcgatgacggtcggagctactggtaaagaagttgaggcagtcgctcaacaattaaaacgtcaaaaaacgatgaaccaagaccgagccttggctattttaaatgatttaagaaaacaataa

SEQ ID NO: 105 - DNA, 1149 bp, Enterococcus faecalis
  source 1..1149: mol_type = genomic DNA
atgacaattgggattgataaaattagtttttttgtgcccccttattatattgatatgacggcactggctgaagccagaaatgtagaccctggaaaatttcatattggtattgggcaagaccaaatggcggtgaacccaatcagccaagatattgtgacatttgcagccaatgccgcagaagcgatcttgaccaaagaagataaagaggccattgatatggtgattgtcgggactgagtccagtatcgatgagtcaaaagcggccgcagttgtcttacatcgtttaatggggattcaacctttcgctcgctctttcgaaatcaaggaagcttgttacggagcaacagcaggcttacagttagctaagaatcacgtagccttacatccagataaaaaagtcttggttgtagcagcagatattgcaaaatatggattaaattctggcggtgagcctacacaaggagctggggcggttgcaatgttagttgctagtgaaccgcgcatcttggctttaaaagaggataatgtgatgctgacgcaagatatctatgacttttggcgtccaacaggccatccgtatcctatggtcgatggtcctttgtcaaacgaaacctacatccaatcttttgcccaagtctgggatgaacataaaaaaagaaccggtcttgattttgcagattatgatgctttagcgttccatattccttacacaaaaatgggcaaaaaagccttattagcaaaaatctccgaccaaactgaagcagaacaggaacgaattttagcccgttatgaagaaagcatcatctatagtcgtcgcgtaggaaacttgtatacgggttcactttatctgggactcatttcccttttagaaaatgcaacgactttaaccgcaggcaatcaaattgggttattcagttatggttctggtgctgtcgctgaatttttcactggtgaattagtagctggttatcaaaatcatttacaaaaagaaactcatttagcactgctagataatcggacagaactttctatcgctgaatatgaagccatgtttgcagaaactttagacacagatattgatcaaacgttagaagatgaattaaaatatagtatttctgctattaataataccgttcgctcttatcgaaac

SEQ ID NO: 106 - DNA, 879 bp, Streptococcus pneumoniae
  source 1..879: mol_type = genomic DNA
atgacaaaaaaagttggtgtcggtcaggcacatagtaagataattttaataggggaacatgcggtcgtttacggttatcctgccatttccctgcctcttttggaggtggaggtgacctgtaaggtagttcctgcagagagtccttggcgcctttatgaggaggataccttgtccatggcggtttatgcctcactggagtatttgaatatcacagaagcctgcattcgttgtgagattgactcggctatccctgagaaacgggggatgggttcgtcagcggctatcagcatagcggccattcgtgcagtatttgactactatcaggctgatctgcctcatgatgtactagaaatcttggtcaatcgagctgaaatgattgcccatatgaatcctagtggtttggatgctaagacctgtcttagtgaccaacctattcgctttatcaagaacgtaggatttacagaacttgagatggatttatccgcctatttggtgattgccgatacgggtgtttatggtcatactcgtgaagccatccaagtggttcaaaataagggcaaggatgccctaccgtttttgcatgccttgggagaattaacccagcaagcagaagttgcgatttcacaaaaagatgctgaaggactgggacaaatcctcagtcaagcgcatttacatttaaaagaaattggagtcagtagccctgaggcagactttttggttgaaacgactcttagccatggtgctctgggtgccaagatgagcggtggtgggctaggaggttgtatcatagccttggtaaccaatttgacacacgcacaagaactagcagaaagattagaagagaaaggagctgttcagacatggatagagagcctgtaa

SEQ ID NO: 107 - DNA, 1011 bp, Streptococcus pneumoniae
  source 1..1011: mol_type = genomic DNA
atgattgctgttaaaacttgcggaaaactctattgggcaggtgaatatgctattttagagccagggcagttagctttgataaaggatattcccatctatatgagggctgagattgctttttctgacagctaccgtatctattcagatatgtttgatttcgcagtggacttaaggcccaatcctgactacagcttgattcaagaaacgattgctttgatgggagacttcctcgctgttcgcggtcagaatttaagacctttttccctaaaaatctgtggcaaaatggaacgagaagggaaaaagtttggtctaggttctagtggcagcgtcgttgtcttggttgtcaaggctttactggctctctataatctttcggttgatcagaatctcttgttcaagctgactagcgctgtcttgctcaagcgaggagacaatggttccatgggcgaccttgcctgtattgtggcagaggatttggttctttaccagtcatttgatcgccagaaggcggctgcttggttagaagaagaaaacttggcgacagttctggagcgtgattggggattttttatctcacaagtgaaaccaactttagaatgtgatttcttagtgggatggaccaaggaagtggctgtatcgagtcacatggtccagcaaatcaagcaaaatatcaatcaaaattttttaagttcctcaaaagaaacggtggtttctttggtcgaagccttggagcaggggaaagccgaaaaagttatcgagcaagtagaagtagccagcaagcttttagaaggcttgagtacagatatttacacgcctttgcttagacagttgaaagaagccagtcaagatttgcaggccgttgccaagagtagtggtgctggtggtggtgactgtggcatcgccctgagttttgatgcgcaatcttctcgaaacactttaaaaaatcgttgggccgatctggggattgagctcttatatcaagaaaggataggacatgacgacaaatcgtaa

SEQ ID NO: 108 - DNA, 954 bp, Streptococcus pneumoniae
  source 1..954: mol_type = genomic DNA
atggatagagagcctgtaacagtacgttcctacgcaaatattgctattatcaaatattggggaaagaaaaaagaaaaagagatggtgcctgctactagcagtatttctctaactttggaaaatatgtatacagagacgaccttgtcgcctttaccagccaatgtaacagctgacgaattttacatcaatggtcagctacaaaatgaggtcgagcatgccaagatgagtaagattattgaccgttatcgtccagctggtgagggctttgtccgtatcgatactcaaaacaatatgcctacggcagcgggcctgtcctcaagttctagtggtttgtccgccctggtcaaggcttgtaatgcttatttcaagcttggattggatagaagtcagttggcacaggaagccaaatttgcctcaggctcttcttctcggagtttttatggaccactaggagcctgggataaggatagtggagaaatttaccctgtagagacagacttgaaactagctatgattatgttggtgctagaggacaagaaaaaaccaatctctagccgtgacgggatgaaactttgtgtggaaacctcgacgacttttgacgactgggttcgtcagtctgagaaggactatcaggatatgctgatttatctcaaggaaaatgattttgccaagattggagaattaacggagaaaaatgctctggctatgcatgctacgacaaagactgctagtccagccttttcttatctgacggatgcctcttatgaggctatggcctttgttcgccagcttcgtgagaaaggagaggcctgctactttaccatggatgctggtcccaatgttaaggtcttctgtcaggagaaagacttggagcatttgtcagaaattttcggtcagcgttatcgcttgattgtgtcaaaaacaaaggatttgagtcaagatgattgctgttaa

SEQ ID NO: 109 - DNA, 549 bp, Escherichia coli
  source 1..549: mol_type = genomic DNA
atgcaaacggaacacgtcattttattgaatgcacagggagttcccacgggtacgctggaaaagtatgccgcacacacggcagacacccgcttacatctcgcgttctccagttggctgtttaatgccaaaggacaattattagttacccgccgcgcactgagcaaaaaagcatggcctggcgtgtggactaactcggtttgtgggcacccacaactgggagaaagcaacgaagacgcagtgatccgccgttgccgttatgagcttggcgtggaaattacgcctcctgaatctatctatcctgactttcgctaccgcgccaccgatccgagtggcattgtggaaaatgaagtgtgtccggtatttgccgcacgcaccactagtgcgttacagatcaatgatgatgaagtgatggattatcaatggtgtgatttagcagatgtattacacggtattgatgccacgccgtgggcgttcagtccgtggatggtgatgcaggcgacaaatcgcgaagccagaaaacgattatctgcatttacccagcttaaataa

SEQ ID NO: 110 - DNA, 915 bp, Haematococcus pluvialis
  source 1..915: mol_type = genomic DNA
atgcttcgttcgttgctcagaggcctcacgcatatcccccgcgtgaactccgcccagcagcccagctgtgcacacgcgcgactccagtttaagctcaggagcatgcagatgacgctcatgcagcccagcatctcagccaatctgtcgcgcgccgaggaccgcacagaccacatgaggggtgcaagcacctgggcaggcgggcagtcgcaggatgagctgatgctgaaggacgagtgcatcttggtggatgttgaggacaacatcacaggccatgccagcaagctggagtgtcacaagttcctaccacatcagcctgcaggcctgctgcaccgggccttctctgtgttcctgtttgacgatcaggggcgactgctgctgcaacagcgtgcacgctcaaaaatcaccttcccaagtgtgtggacgaacacctgctgcagccaccctttacatgggcagaccccagatgaggtggaccaactaagccaggtggccgacggaacagtacctggcgcaaaggctgctgccatccgcaagttggagcacgagctggggataccagcgcaccagctgccggcaagcgcgtttcgcttcctcacgcgtttgcactactgtgccgcggacgtgcagccagctgcgacacaatcagcgctctggggcgagcacgaaatggactacatcttgttcatccgggccaacgtcaccttggcgcccaaccctgacgaggtggacgaagtcaggtacgtgacgcaagaggagctgcggcagatgatgcagccggacaacgggctgcaatggtcgccgtggtttcgcatcatcgccgcgcgcttccttgagcgttggtgggctgacctggacgcggccctaaacactgacaaacacgaggattggggaacggtgcatcacatcaacgaagcg

SEQ ID NO: 111 - DNA, 924 bp, Pantoea agglomerans
  source 1..924: mol_type = genomic DNA
atggtgagtggcagtaaagcgggcgtttcgcctcatcgcgaaatagaagtaatgagacaatccattgacgatcacctggctggcctgttacctgaaaccgacagccaggatatcgtcagccttgcgatgcgtgaaggcgtcatggcacccggtaaacggatccgtccgctgctgatgctgctggccgcccgcgacctccgctaccagggcagtatgcctacgctgctcgatctcgcctgcgccgttgaactgacccataccgcgtcgctgatgctcgacgacatgccctgcatggacaacgccgagctgcgccgcggtcagcccactacccacaaaaaatttggtgagagcgtggcgatccttgcctccgttgggctgctctctaaagcctttggtctgatcgccgccaccggcgatctgccgggggagaggcgtgcccaggcggtcaacgagctctctaccgccgtgggcgtgcagggcctggtactggggcagtttcgcgatcttaacgatgccgccctcgaccgtacccctgacgctatcctcagcaccaaccacctcaagaccggcattctgttcagcgcgatgctgcagatcgtcgccattgcttccgcctcgtcgccgagcacgcgagagacgctgcacgccttcgccctcgacttcggccaggcgtttcaactgctggacgatctgcgtgacgatcacccggaaaccggtaaagatcgcaataaggacgcgggaaaatcgacgctggtcaaccggctgggcgcagacgcggcccggcaaaagctgcgcgagcatattgattccgccgacaaacacctcacttttgcctgtccgcagggcggcgccatccgacagtttatgcatctgtggtttggccatcaccttgccgactggtcaccggtcatgaaaatcgcctga

SEQ ID NO: 112 - DNA, 930 bp, Pantoea agglomerans
  source 1..930: mol_type = genomic DNA
atgagccaaccgccgctgcttgaccacgccacgcagaccatggccaacggctcgaaaagttttgccaccgctgcgaagctgttcgacccggccacccgccgtagcgtgctgatgctctacacctggtgccgccactgcgatgacgtcattgacgaccagacccacggcttcgccagcgaggccgcggcggaggaggaggccacccagcgcctggcccggctgcgcacgctgaccctggcggcgtttgaaggggccgagatgcaggatccggccttcgctgcctttcaggaggtggcgctgacccacggtattacgccccgcatggcgctcgatcacctcgacggctttgcgatggacgtggctcagacccgctatgtcacctttgaggatacgctgcgctactgctatcacgtggcgggcgtggtgggtctgatgatggccagggtgatgggcgtgcgggatgagcgggtgctggatcgcgcctgcgatctggggctggccttccagctgacgaatatcgcccgggatattattgacgatgcggctattgaccgctgctatctgcccgccgagtggctgcaggatgccgggctgaccccggagaactatgccgcgcgggagaatcgggccgcgctggcgcgggtggcggagcggcttattgatgccgcagagccgtactacatctcctcccaggccgggctacacgatctgccgccgcgctgcgcctgggcgatcgccaccgcccgcagcgtctaccgggagatcggtattaaggtaaaagcggcgggaggcagcgcctgggatcgccgccagcacaccagcaaaggtgaaaaaattgccatgctgatggcggcaccggggcaggttattcgggcgaagacgacgagggtgacgccgcgtccggccggtctttggcagcgtcccgtttag

SEQ ID NO: 113 - DNA, 1479 bp, Pantoea agglomerans
  source 1..1479: mol_type = genomic DNA
atgaaaaaaaccgttgtgattggcgcaggctttggtggcctggcgctggcgattcgcctgcaggcggcagggatcccaaccgtactgctggagcagcgggacaagcccggcggtcgggcctacgtctggcatgaccagggctttacctttgacgccgggccgacggtgatcaccgatcctaccgcgcttgaggcgctgttcaccctggccggcaggcgcatggaggattacgtcaggctgctgccggtaaaacccttctaccgactctgctgggagtccgggaagaccctcgactatgctaacgacagcgccgagcttgaggcgcagattacccagttcaacccccgcgacgtcgagggctaccggcgctttctggcttactcccaggcggtattccaggagggatatttgcgcctcggcagcgtgccgttcctctcttttcgcgacatgctgcgcgccgggccgcagctgcttaagctccaggcgtggcagagcgtctaccagtcggtttcgcgctttattgaggatgagcatctgcggcaggccttctcgttccactccctgctggtaggcggcaaccccttcaccacctcgtccatctacaccctgatccacgcccttgagcgggagtggggggtctggttccctgagggcggcaccggggcgctggtgaacggcatggtgaagctgtttaccgatctgggcggggagatcgaactcaacgcccgggtcgaagagctggtggtggccgataaccgcgtaagccaggtccggctggcggatggtcggatctttgacaccgacgccgtagcctcgaacgctgacgtggtgaacacctataaaaagctgctcggccaccatccggtggggcagaagcgggcggcagcgctggagcgcaagagcatgagcaactcgctgtttgtgctctacttcggcctgaaccagcctcattcccagctggcgcaccataccatctgttttggtccccgctaccgggagctgatcgacgagatctttaccggcagcgcgctggcggatgacttctcgctctacctgcactcgccctgcgtgaccgatccctcgctcgcgcctcccggctgcgccagcttctacgtgctggccccggtgccgcatcttggcaacgcgccgctggactgggcgcaggaggggccgaagctgcgcgaccgcatctttgactaccttgaagagcgctatatgcccggcctgcgtagccagctggtgacccagcggatctttaccccggcagacttccacgacacgctggatgcgcatctgggatcggccttctccatcgagccgctgctgacccaaagcgcctggttccgcccgcacaaccgcgacagcgacattgccaacctctacctggtgggcgcaggtactcaccctggggcgggcattcctggcgtagtggcctcggcgaaagccaccgccagcctgatgattgaggatctgcaatga

SEQ ID NO: 114 - DNA, 1146 bp, Pantoea ananatis
  source 1..1146: mol_type = genomic DNA
atgcaaccgcattatgatctgattctcgtgggggctggactcgcgaatggccttatcgccctgcgtcttcagcagcagcaacctgatatgcgtattttgcttatcgacgccgcaccccaggcgggcgggaatcatacgtggtcatttcaccacgatgatttgactgagagccaacatcgttggatagctccgctggtggttcatcactggcccgactatcaggtacgctttcccacacgccgtcgtaagctgaacagcggctacttttgtattacttctcagcgtttcgctgaggttttacagcgacagtttggcccgcacttgtggatggataccgcggtcgcagaggttaatgcggaatctgttcggttgaaaaagggtcaggttatcggtgcccgcgcggtgattgacgggcggggttatgcggcaaattcagcactgagcgtgggcttccaggcgtttattggccaggaatggcgattgagccacccgcatggtttatcgtctcccattatcatggatgccacggtcgatcagcaaaatggttatcgcttcgtgtacagcctgccgctctcgccgaccagattgttaattgaagacacgcactatattgataatgcgacattagatcctgaatgcgcgcggcaaaatatttgcgactatgccgcgcaacagggttggcagcttcagacactgctgcgagaagaacagggcgccttacccattactctgtcgggcaatgccgacgcattctggcagcagcgccccctggcctgtagtggattacgtgccggtctgttccatcctaccaccggctattcactgccgctggcggttgccgtggccgaccgcctgagtgcacttgatgtctttacgtcggcctcaattcaccatgccattacgcattttgcccgcgagcgctggcagcagcagggctttttccgcatgctgaatcgcatgctgtttttagccggacccgccgattcacgctggcgggttatgcagcgtttttatggtttacctgaagatttaattgcccgtttttatgcgggaaaactcacgctgaccgatcggctacgtattctgagcggcaagccgcctgttccggtattagcagcattgcaagccattatgacgactcatcgt

SEQ ID NO: 115 - DNA, 828 bp, unidentified
  source 1..828: mol_type = genomic DNA; note = uncultured marine bacterium 66A03
atgggtctgatgctgattgattggtgtgcactggctctggttgttttcattggcctgccgcacggcgcgctggatgctgccatttctttttctatgatctcttctgcaaaacgcattgctcgtctggctggtattctgctgatctatctgctgctggcgaccgcgttcttcctgatctggtatcagctgccagcgtttagcctgctgatcttcctgctgatctccattatccactttggtatggcagacttcaacgcgtccccaagcaaactgaaatggccgcatatcatcgcccacggcggtgttgttactgtttggctgccgctgatccagaaaaacgaagtaactaaactgtttagcatcctgactaacggtccgactccgatcctgtgggacatcctgctgattttcttcctgtgttggtctattggcgtgtgtctgcacacgtacgaaaccctgcgctctaaacattacaacatcgcctttgaactgatcggtctgattttcctggcgtggtatgcgccgcctctggttacgtttgccacttacttctgcttcattcattcccgtcgccacttctcctttgtgtggaagcagctgcaacacatgtcttccaaaaagatgatgattggcagcgcgattatcctgtcctgtacctcttggctgatcggcggtggtatctatttcttcctgaactccaaaatgatcgcctctgaggctgcgctgcagactgtgttcatcggtctggcggcactgaccgtgccgcacatgattctgatcgacttcatcttccgtccgcactcttcccgtatcaaaatcaaaaactaa

SEQ ID NO: 116 - DNA, 810 bp, Escherichia coli
  source 1..810: mol_type = genomic DNA
atgactcataaagcaacggagatcctgacaggtaaagttatgcaaaaatcggtcttaattaccggatgttccagtggaattggcctggaaagcgcgctcgaattaaaacgccagggttttcatgtgctggcaggttgccggaaaccggatgatgttgagcgcatgaacagcatgggatttaccggcgtgttgatcgatctggattcaccagaaagtgttgatcgcgcagccgacgaggtgatcgccctgaccgataattgtctgtatgggatctttaacaatgccggattcggcatgtatggccccctttccaccatcagccgtgcgcagatggaacagcagttttccgccaactttttcggcgcacaccagctcaccatgcgcctgttacccgcgatgttaccgcacggtgaagggcgtattgtgatgacatcatcggtgatgggattaatctccacgccgggtcgtggcgcttacgcggccagtaaatatgcgctggaggcgtggtcagatgcactgcgcatggagctgcgccacagcggaattaaagtcagcctgatcgaacccggtcccattcgtactcgcttcaccgacaacgtcaaccagacgcaaagtgataaaccagtcgaaaatcccggcatcgccgcccgctttacgttgggaccggaagcggtggtggacaaagtacgccatgcttttattagcgagaagccgaagatgcgctatccggtgacgctggtgacctgggcggtaatggtgcttaagcgcctgctgccggggcgcgtgatggacaaaatattgcaggggtaa

SEQ ID NO: 117 - DNA, 1863 bp, Escherichia coli
  source 1..1863: mol_type = genomic DNA
atgagttttgatattgccaaatacccgaccctggcactggtcgactccacccaggagttacgactgttgccgaaagagagtttaccgaaactctgcgacgaactgcgccgctatttactcgacagcgtgagccgttccagcgggcacttcgcctccgggctgggcacggtcgaactgaccgtggcgctgcactatgtctacaacaccccgtttgaccaattgatttgggatgtggggcatcaggcttatccgcataaaattttgaccggacgccgcgacaaaatcggcaccatccgtcagaaaggcggtctgcacccgttcccgtggcgcggcgaaagcgaatatgacgtattaagcgtcgggcattcatcaacctccatcagtgccggaattggtattgcggttgctgccgaaaaagaaggcaaaaatcgccgcaccgtctgtgtcattggcgatggcgcgattaccgcaggcatggcgtttgaagcgatgaatcacgcgggcgatatccgtcctgatatgctggtgattctcaacgacaatgaaatgtcgatttccgaaaatgtcggcgcgctcaacaaccatctggcacagctgctttccggtaagctttactcttcactgcgcgaaggcgggaaaaaagttttctctggcgtgccgccaattaaagagctgctcaaacgcaccgaagaacatattaaaggcatggtagtgcctggcacgttgtttgaagagctgggctttaactacatcggcccggtggacggtcacgatgtgctggggcttatcaccacgctaaagaacatgcgcgacctgaaaggcccgcagttcctgcatatcatgaccaaaaaaggtcgtggttatgaaccggcagaaaaagacccgatcactttccacgccgtgcctaaatttgatccctccagcggttgtttgccgaaaagtagcggcggtttgccgagctattcaaaaatctttggcgactggttgtgcgaaacggcagcgaaagacaacaagctgatggcgattactccggcgatgcgtgaaggttccggcatggtcgagttttcacgtaaattcccggatcgctacttcgacgtggcaattgccgagcaacacgcggtgacctttgctgcgggtctggcgattggtgggtacaaacccattgtcgcgatttactccactttcctgcaacgcgcctatgatcaggtgctgcatgacgtggcgattcaaaagcttccggtcctgttcgccatcgaccgcgcgggcattgttggtgctgacggtcaaacccatcagggtgcttttgatctctcttacctgcgctgcataccggaaatggtcattatgaccccgagcgatgaaaacgaatgtcgccagatgctctataccggctatcactataacgatggcccgtcagcggtgcgctacccgcgtggcaacgcggtcggcgtggaactgacgccgctggaaaaactaccaattggcaaaggcattgtgaagcgtcgtggcgagaaactggcgatccttaactttggtacgctgatgccagaagcggcgaaagtcgccgaatcgctgaacgccacgctggtcgatatgcgttttgtgaaaccgcttgatgaagcgttaattctggaaatggccgccagccatgaagcgctggtcaccgtagaagaaaacgccattatgggcggcgcaggcagcggcgtgaacgaagtgctgatggcccatcgtaaaccagtacccgtgctgaacattggcctgccggacttctttattccgcaaggaactcaggaagaaatgcgcgccgaactcggcctcgatgccgctggtatggaagccaaaatcaaggcctggctggcataa

SEQ ID NO: 118 - DNA, 1197 bp, Escherichia coli
  source 1..1197: mol_type = genomic DNA
atgaagcaactcaccattctgggctcgaccggctcgattggttgcagcacgctggacgtggtgcgccataatcccgaacacttccgcgtagttgcgctggtggcaggcaaaaatgtcactcgcatggtagaacagtgcctggaattctctccccgctatgccgtaatggacgatgaagcgagtgcgaaacttcttaaaacgatgctacagcaacagggtagccgcaccgaagtcttaagtgggcaacaagccgcttgcgatatggcagcgcttgaggatgttgatcaggtgatggcagccattgttggcgctgctgggctgttacctacgcttgctgcgatccgcgcgggtaaaaccattttgctggccaataaagaatcactggttacctgcggacgtctgtttatggacgccgtaaagcagagcaaagcgcaattgttaccggtcgatagcgaacataacgccatttttcagagtttaccgcaacctatccagcataatctgggatacgctgaccttgagcaaaatggcgtggtgtccattttacttaccgggtctggtggccctttccgtgagacgccattgcgcgatttggcaacaatgacgccggatcaagcctgccgtcatccgaactggtcgatggggcgtaaaatttctgtcgattcggctaccatgatgaacaaaggtctggaatacattgaagcgcgttggctgtttaacgccagcgccagccagatggaagtgctgattcacccgcagtcagtgattcactcaatggtgcgctatcaggacggcagtgttctggcgcagctgggggaaccggatatgcgtacgccaattgcccacaccatggcatggccgaatcgcgtgaactctggcgtgaagccgctcgatttttgcaaactaagtgcgttgacatttgccgcaccggattatgatcgttatccatgcctgaaactggcgatggaggcgttcgaacaaggccaggcagcgacgacagcattgaatgccgcaaacgaaatcaccgttgctgcttttcttgcgcaacaaatccgctttacggatatcgctgcgttgaatttatccgtactggaaaaaatggatatgcgcgaaccacaatgtgtggacgatgtgttatctgttgatgcgaacgcgcgtgaagtcgccagaaaagaggtgatgcgtctcgcaagctga

SEQ ID NO: 119 - DNA, 900 bp, Escherichia coli
  source 1..900: mol_type = genomic DNA
atggactttccgcagcaactcgaagcctgcgttaagcaggccaaccaggcgctgagccgttttatcgccccactgccctttcagaacactcccgtggtcgaaaccatgcagtatggcgcattattaggtggtaagcgcctgcgacctttcctggtttatgccaccggtcatatgttcggcgttagcacaaacacgctggacgcacccgctgccgccgttgagtgtatccacgcttactcattaattcatgatgatttaccggcaatggatgatgacgatctgcgtcgcggtttgccaacctgccatgtgaagtttggcgaagcaaacgcgattctcgctggcgacgctttacaaacgctggcgttctcgattttaagcgatgccgatatgccggaagtgtcggaccgcgacagaatttcgatgatttctgaactggcgagcgccagtggtattgccggaatgtgcggtggtcaggcattagatttagacgcggaaggcaaacacgtacctctggacgcgcttgagcgtattcatcgtcataaaaccggcgcattgattcgcgccgccgttcgccttggtgcattaagcgccggagataaaggacgtcgtgctctgccggtactcgacaagtatgcagagagcatcggccttgccttccaggttcaggatgacatcctggatgtggtgggagatactgcaacgttgggaaaacgccagggtgccgaccagcaacttggtaaaagtacctaccctgcacttctgggtcttgagcaagcccggaagaaagcccgggatctgatcgacgatgcccgtcagtcgctgaaacaactggctgaacagtcactcgatacctcggcactggaagcgctagcggactacatcatccagcgtaataaataa

SEQ ID NO: 120 - DNA, 1656 bp, Clausena lansium
  source 1..1656: mol_type = genomic DNA
atgtcaactcaacaagtttcatcagagaacattgttcgtaacgctgcgaatttccatcctaatatatggggaaaccatttcctcacatgtccttctcagacgattgatagttggactcaacagcaccacaaagaactgaaagaagaggtgaggaaaatgatggtgtctgatgcaaataaacctgcccagagattgcgcttgattgatactgtccaaaggctaggtgtggcttaccactttgaaaaggagattgatgatgcattggagaaaataggtcatgacccttttgatgataaagatgatctctacattgtctctctttgttttcgattgctgaggcagcatggaattaagatatcatgtgatgtgtttgagaagtttaaagatgacgatggaaaattcaaggcatcattgatgaatgatgttcaaggcatgctaagtttatatgaggcagcacacctagccattcacggagaagatattttagatgaagcaattgttttcacgaccactcaccttaagtcaacggtatctaattctcctgtaaactctacttttgctgaacaaatacgtcattctctcagagttcctctccgtaaagctgtacctaggttagagtcgaggtatttcttggatatctattcaagagatgatttgcacgataaaactttgctcaatttcgcaaagttagactttaatatactacaagcaatgcaccagaaggaagcaagtgagatgaccaggtggtggagagattttgacttccttaaaaagctgccttatataagagacagagtcgtggagctatatttttggattctggtgggagtgtcttatcagcccaaattcagcactggtagaatttttttgtccaaaataatatgccttgagaccctcgtagatgatacatttgacgcctacggtacttttgacgagctcgcaatctttactgaagcagttacaagatgggaccttggccacagagatgcactaccagaatacatgaaattcattttcaagacactcattgatgtctacagtgaagctgagcaagaactggcaaaggaagggagatcatacagcatacactatgcaatacgatcgttccaagaactagttatgaagtacttctgcgaagccaagtggttaaataaaggttatgttccgagcctggacgattataaatcagtttcattaagaagtatcggttttttaccgatagcggtagcttccttcgttttcatgggtgatattgcaactaaggaggtctttgaatgggaaatgaataaccctaagatcataatagccgcagaaacgattttcagattcctggatgacatagcaggccataggtttgagcaaaagagagaacatagtccatcagctattgaatgctacaagaatcaacatggagtgtctgaggaagaggcagttaaagcgttgtcgttagaagttgctaatagttggaaagatataaatgaggagctgcttctcaacccaatggctattcctttacctctgcttcaggtgattcttgatctctcacgttcggccgattttatgtacggtaatgctcaagatcgcttcacgcattcaacgatgatgaaagaccaagttgatttggtgctgaaggaccccgttaagcttgacgattaa

SEQ ID NO: 121 - DNA, 1719 bp, synthetic construct
  misc_feature 1..1719: note = Matricaria recutita MrBBS
  source 1..1719: mol_type = other DNA
atgtcaaccctgtcagtctccacgccgtccttctcatcgtcgccgctgtcctcagtgaacaaaaatagcacgaaacaacacgttacgcgcaatagtgtgatctttcatgattccatttggggcgaccagttcctggaatacaaagaaaaattcaacgttgcaaccgaaaaacaactgattgaagaactgaaagaagaagtccgcaatgaactgatgatccgtgcgtgcaacgaagccagtcgctatattaaactgatccagctgattgatgtggttgaacgtctgggcctggcctaccactttgaaaaagaaatcgaagaatccctgcaacatatttatgttacctacggtcacaaatggacgaactacaacaacatcgaaagcctgtctctgtggtttcgcctgctgcgtcagaacggttttaatgtcagctctgatatcttcgaaaaccatattgacgaaaaaggcaatttccaagaaagtctgtgcaacgatccgcaaggcatgctggcactgtatgaagcggcctacatgcgcgtggaaggcgaaattatcctggacaaagctctggaatttaccaaactgcacctgggtattatcagcaacgatccgtcttgtgacagttccctgcgtacggaaattaaacaggcgctgaaacagccgctgcgtcgccgtctgccgcgtctggaagcagttcgttatattgctatctaccagcaaaaagcgagtcattccgaagtcctgctgaaactggccaaactggatttcaatgtgctgcaggaaatgcacaaagacgaactgtcacaaatttgtaaatggtggaaagacctggacatccgtaacaaactgccgtatgttcgcgatcgtctgattgaaggctatttttggattctgggtatttacttcgaaccgcagcattcgcgcacccgtatgtttctgatgaaaacgtgcatgtggctgatcgtcctggatgacacctttgataattatggcacgtacgaagaactggaaattttcacccaggcggtggaacgctggagcatcacgtgtctggatgaactgccggaatacatgaaactgatctaccatgaacagttccgtgtgcaccaagaaatggaagaatctctggaaaaagaaggtaaagcataccagatccattacatcaaagaaatggctaaagaaggcacccgctctctgctgctggaagcgaaatggctgaaagaaggttatatgccgacgctggatgaatacctgtcaaactcgctggttacctgcggctatgcactgatgacggctcgctcatacgttgcccgtgatgacggtattgtcaccgaagatgcgtttaaatgggtggccacgcacccgccgatcgttaaagcagcttgtaaaattctgcgtctgatggatgacatcgcgacccataaagaagaacaggaacgcggccacatcgcctcatcgattgaatgctatcgtaaagaaaccggtgcatctgaagaagaagcgtgtatggattttctgaaacaggtcgaagacggctggaaagtgatcaatcaagaatcactgatgccgaccgatgtgccgttcccgctgctgatcccggcgattaacctggcccgcgtttcggacacgctgtataaagataacgacggttacaatcatgctgataaagaagtgattggttacattaaaagtctgtttgtccacccgatgattgtgtga

SEQ ID NO: 122 - DNA, 717 bp, synthetic construct
  misc_feature 1..717: note = EGFP
  source 1..717: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaag

SEQ ID NO: 123 - DNA, 1233 bp, synthetic construct
  misc_feature 1..1233: note = MG1363 (Hydroxymethylglutaryl-CoA reductase)
  source 1..1233: mol_type = other DNA
atgagaaaaaaattttatcaaatgtcgccacaagagcgattgaattcgttaaacttatccgaaaagagtcaagaaattttaagtgagatggccctagatacaactattttggacaatctcatagaaaatcaaatttctgaatttgaattgccaatgggaattgcccaaaattttgttattaatggacaaagttttctaataccaatggttacagaggaaccgtcagtaattgccgcagcaagtaatggagcaaaaatagctggaaattttgtagctgaaattaaagaacgtttaatgcgtggtcaaattgtattttatgatgttaaaaattcagataagattgcaaatgaaatacttgaaaagcaagaaaaaatctttgaacaagccgaactttcttacccttcaatcgttaaacgaggtggcggtttgagagaggtttctagtcggattttctctagtcaaaagtttttatctgttgatgtcaaggttgatgttaaagacgccatgggtgcaaatattattaattcaatcttagagggaattgctgaactctttcggaggtggtttcctgatgaaaaaattctgtttagtattttatcaaattatgctactgaatcactagtgaaagtgacctgtgaaattccggttgaacgcctttcaaaaaaagcagatggatatgaaattggtcaaaaaattatggctgcttcccaatattcaaaaattgatccttatcgtgctagcacccataacaaagggattatgaacggtatcaatgcggtcattttagctacaggaaatgatactcgggccatctcagctgctattcatgcttatgcggcaaaagatggagcctaccaagggttagccaactgggaacttcaagagaaaatgttagttggcgaactcgaatttcctttaccagtggcaactgtcggagggggagtcaaggttttgccaaaagcacaagcggccatggagattctgggtatcagtgatgctaaggagctcgcaaaagttattgctgcagtgggactagctcaaaacttagccgctttacgtgccctagtttcagaaggaattcaacagggacacatgagtctacaagctcgttctttggctttgagtgtaggagcacaagcggatgaaatcgcgatattaagtcaacaattgcgcaaagaaaaagtgatgaatcaagaagtagcccaaaatttactaaaaaatttgagaaaataa

SEQ ID NO: 124 - protein, 803 aa, Enterococcus faecalis
LKTVVIIDALRTPIGKYKGSLSQVSAVDLGTHVTTQLLKRHSTISEEIDQVIFGNVLQAGNGQNPARQIAINSGLSHEIPAMTVNEVCGSGMKAVILAKQLIQLGEAEVLIAGGIENMSQAPKLQRFNYETESYDAPFSSMMYDGLTDAFSGQAMGLTAENVAEKYHVTREEQDQFSVHSQLKAAQAQAEGIFADEIAPLEVSGTLVEKDEGIRPNSSVEKLGTLKTVFKEDGTVTAGNASTINDGASALIIASQEYAEAHGLPYLAIIRDSVEVGIDPAYMGISPIKAIQKLLARNQLTTEEIDLYEINEAFAATSIVVQRELALPEEKVNIYGGGISLGHAIGATGARLLTSLSYQLNQKEKKYGVASLCIGGGLGLAMLLERPQQKKNSRFYQMSPEERLASLLNEGQISADTKKEFENTALSSQIANHMIENQISETEVPMGVGLHLTVDETDYLVPMATEEPSVIAALSNGAKIAQGFKTVNQQRLMRGQIVFYDVADAESLIDELQVRETEIFQQAELSYPSIVKRGGGLRDLQYRAFDESFVSVDFLVDVKDAMGANIVNAMLEGVAELFREWFAEQKILFSILSNYATESVVTMKTAIPVSRLSKGSNGREIAEKIVLASRYASLDPYRAVTHNKGIMNGIEAVVLATGNDTRAVSASCHAFAVKEGRYQGLTSWTLDGEQLIGEISVPLALATVGGATKVLPKSQAAADLLAVTDAKELSRVVAAVGLAQNLAALRALVSEGIQKGHMALQARSLAMTVGATGKEVEAVAQQLKRQKTMNQDRALAILNDLRKQ

SEQ ID NO: 125 - protein, 383 aa, Enterococcus faecalis
MTIGIDKISFFVPPYYIDMTALAEARNVDPGKFHIGIGQDQMAVNPISQDIVTFAANAAEAILTKEDKEAIDMVIVGTESSIDESKAAAVVLHRLMGIQPFARSFEIKEACYGATAGLQLAKNHVALHPDKKVLVVAADIAKYGLNSGGEPTQGAGAVAMLVASEPRILALKEDNVMLTQDIYDFWRPTGHPYPMVDGPLSNETYIQSFAQVWDEHKKRTGLDFADYDALAFHIPYTKMGKKALLAKISDQTEAEQERILARYEESIIYSRRVGNLYTGSLYLGLISLLENATTLTAGNQIGLFSYGSGAVAEFFTGELVAGYQNHLQKETHLALLDNRTELSIAEYEAMFAETLDTDIDQTLEDELKYSISAINNTVRSYRN

SEQ ID NO: 126 - protein, 292 aa, Streptococcus pneumoniae
MTKKVGVGQAHSKIILIGEHAVVYGYPAISLPLLEVEVTCKVVPAESPWRLYEEDTLSMAVYASLEYLNITEACIRCEIDSAIPEKRGMGSSAAISIAAIRAVFDYYQADLPHDVLEILVNRAEMIAHMNPSGLDAKTCLSDQPIRFIKNVGFTELEMDLSAYLVIADTGVYGHTREAIQVVQNKGKDALPFLHALGELTQQAEVAISQKDAEGLGQILSQAHLHLKEIGVSSPEADFLVETTLSHGALGAKMSGGGLGGCIIALVTNLTHAQELAERLEEKGAVQTWIESL

SEQ ID NO: 127 - protein, 336 aa, Streptococcus pneumoniae
  source 1..336: note = mvaK2
MIAVKTCGKLYWAGEYAILEPGQLALIKDIPIYMRAEIAFSDSYRIYSDMFDFAVDLRPNPDYSLIQETIALMGDFLAVRGQNLRPFSLKICGKMEREGKKFGLGSSGSVVVLVVKALLALYNLSVDQNLLFKLTSAVLLKRGDNGSMGDLACIVAEDLVLYQSFDRQKAAAWLEEENLATVLERDWGFFISQVKPTLECDFLVGWTKEVAVSSHMVQQIKQNINQNFLSSSKETVVSLVEALEQGKAEKVIEQVEVASKLLEGLSTDIYTPLLRQLKEASQDLQAVAKSSGAGGGDCGIALSFDAQSSRNTLKNRWADLGIELLYQERIGHDDKS

SEQ ID NO: 128 - protein, 317 aa, Streptococcus pneumoniae
  source 1..317: note = mvaD
MDREPVTVRSYANIAIIKYWGKKKEKEMVPATSSISLTLENMYTETTLSPLPANVTADEFYINGQLQNEVEHAKMSKIIDRYRPAGEGFVRIDTQNNMPTAAGLSSSSSGLSALVKACNAYFKLGLDRSQLAQEAKFASGSSSRSFYGPLGAWDKDSGEIYPVETDLKLAMIMLVLEDKKKPISSRDGMKLCVETSTTFDDWVRQSEKDYQDMLIYLKENDFAKIGELTEKNALAMHATTKTASPAFSYLTDASYEAMAFVRQLREKGEACYFTMDAGPNVKVFCQEKDLEHLSEIFGQRYRLIVSKTKDLSQDDCC

SEQ ID NO: 129 - protein, 182 aa, Escherichia coli
MQTEHVILLNAQGVPTGTLEKYAAHTADTRLHLAFSSWLFNAKGQLLVTRRALSKKAWPGVWTNSVCGHPQLGESNEDAVIRRCRYELGVEITPPESIYPDFRYRATDPSGIVENEVCPVFAARTTSALQINDDEVMDYQWCDLADVLHGIDATPWAFSPWMVMQATNREARKRLSAFTQLK

SEQ ID NO: 130 - protein, 305 aa, Haematococcus pluvialis
MLRSLLRGLTHIPRVNSAQQPSCAHARLQFKLRSMQMTLMQPSISANLSRAEDRTDHMRGASTWAGGQSQDELMLKDECILVDVEDNITGHASKLECHKFLPHQPAGLLHRAFSVFLFDDQGRLLLQQRARSKITFPSVWTNTCCSHPLHGQTPDEVDQLSQVADGTVPGAKAAAIRKLEHELGIPAHQLPASAFRFLTRLHYCAADVQPAATQSALWGEHEMDYILFIRANVTLAPNPDEVDEVRYVTQEELRQMMQPDNGLQWSPWFRIIAARFLERWWADLDAALNTDKHEDWGTVHHINEA

SEQ ID NO: 131 - protein, 309 aa, Pantoea agglomerans
MSQPPLLDHATQTMANGSKSFATAAKLFDPATRRSVLMLYTWCRHCDDVIDDQTHGFASEAAAEEEATQRLARLRTLTLAAFEGAEMQDPAFAAFQEVALTHGITPRMALDHLDGFAMDVAQTRYVTFEDTLRYCYHVAGVVGLMMARVMGVRDERVLDRACDLGLAFQLTNIARDIIDDAAIDRCYLPAEWLQDAGLTPENYAARENRAALARVAERLIDAAEPYYISSQAGLHDLPPRCAWAIATARSVYREIGIKVKAAGGSAWDRRQHTSKGEKIAMLMAAPGQVIRAKTTRVTPRPAGLWQRPV

SEQ ID NO: 132 - protein, 492 aa, Pantoea agglomerans
MKKTVVIGAGFGGLALAIRLQAAGIPTVLLEQRDKPGGRAYVWHDQGFTFDAGPTVITDPTALEALFTLAGRRMEDYVRLLPVKPFYRLCWESGKTLDYANDSAELEAQITQFNPRDVEGYRRFLAYSQAVFQEGYLRLGSVPFLSFRDMLRAGPQLLKLQAWQSVYQSVSRFIEDEHLRQAFSFHSLLVGGNPFTTSSIYTLIHALEREWGVWFPEGGTGALVNGMVKLFTDLGGEIELNARVEELVVADNRVSQVRLADGRIFDTDAVASNADVVNTYKKLLGHHPVGQKRAAALERKSMSNSLFVLYFGLNQPHSQLAHHTICFGPRYRELIDEIFTGSALADDFSLYLHSPCVTDPSLAPPGCASFYVLAPVPHLGNAPLDWAQEGPKLRDRIFDYLEERYMPGLRSQLVTQRIFTPADFHDTLDAHLGSAFSIEPLLTQSAWFRPHNRDSDIANLYLVGAGTHPGAGIPGVVASAKATASLMIEDLQ

SEQ ID NO: 133 - protein, 382 aa, Pantoea ananatis
  source 1..382: note = crtY
MQPHYDLILVGAGLANGLIALRLQQQQPDMRILLIDAAPQAGGNHTWSFHHDDLTESQHRWIAPLVVHHWPDYQVRFPTRRRKLNSGYFCITSQRFAEVLQRQFGPHLWMDTAVAEVNAESVRLKKGQVIGARAVIDGRGYAANSALSVGFQAFIGQEWRLSHPHGLSSPIIMDATVDQQNGYRFVYSLPLSPTRLLIEDTHYIDNATLDPECARQNICDYAAQQGWQLQTLLREEQGALPITLSGNADAFWQQRPLACSGLRAGLFHPTTGYSLPLAVAVADRLSALDVFTSASIHHAITHFARERWQQQGFFRMLNRMLFLAGPADSRWRVMQRFYGLPEDLIARFYAGKLTLTDRLRILSGKPPVPVLAALQAIMTTHR

SEQ ID NO: 134 - protein, 275 aa, unidentified [sequence omitted]

SEQ ID NO: 135 - protein, 269 aa, Escherichia coli
MTHKATEILTGKVMQKSVLITGCSSGIGLESALELKRQGFHVLAGCRKPDDVERMNSMGFTGVLIDLDSPESVDRAADEVIALTDNCLYGIFNNAGFGMYGPLSTISRAQMEQQFSANFFGAHQLTMRLLPAMLPHGEGRIVMTSSVMGLISTPGRGAYAASKYALEAWSDALRMELRHSGIKVSLIEPGPIRTRFTDNVNQTQSDKPVENPGIAARFTLGPEAVVDKVRHAFISEKPKMRYPVTLVTWAVMVLKRLLPGRVMDKILQG

SEQ ID NO: 136 - protein, 620 aa, Escherichia coli
  source 1..620: note = dxs
MSFDIAKYPTLALVDSTQELRLLPKESLPKLCDELRRYLLDSVSRSSGHFASGLGTVELTVALHYVYNTPFDQLIWDVGHQAYPHKILTGRRDKIGTIRQKGGLHPFPWRGESEYDVLSVGHSSTSISAGIGIAVAAEKEGKNRRTVCVIGDGAITAGMAFEAMNHAGDIRPDMLVILNDNEMSISENVGALNNHLAQLLSGKLYSSLREGGKKVFSGVPPIKELLKRTEEHIKGMVVPGTLFEELGFNYIGPVDGHDVLGLITTLKNMRDLKGPQFLHIMTKKGRGYEPAEKDPITFHAVPKFDPSSGCLPKSSGGLPSYSKIFGDWLCETAAKDNKLMAITPAMREGSGMVEFSRKFPDRYFDVAIAEQHAVTFAAGLAIGGYKPIVAIYSTFLQRAYDQVLHDVAIQKLPVLFAIDRAGIVGADGQTHQGAFDLSYLRCIPEMVIMTPSDENECRQMLYTGYHYNDGPSAVRYPRGNAVGVELTPLEKLPIGKGIVKRRGEKLAILNFGTLMPEAAKVAESLNATLVDMRFVKPLDEALILEMAASHEALVTVEENAIMGGAGSGVNEVLMAHRKPVPVLNIGLPDFFIPQGTQEEMRAELGLDAAGMEAKIKAWLA

SEQ ID NO: 137 - protein, 398 aa, Escherichia coli
  source 1..398: note = dxr
MKQLTILGSTGSIGCSTLDVVRHNPEHFRVVALVAGKNVTRMVEQCLEFSPRYAVMDDEASAKLLKTMLQQQGSRTEVLSGQQAACDMAALEDVDQVMAAIVGAAGLLPTLAAIRAGKTILLANKESLVTCGRLFMDAVKQSKAQLLPVDSEHNAIFQSLPQPIQHNLGYADLEQNGVVSILLTGSGGPFRETPLRDLATMTPDQACRHPNWSMGRKISVDSATMMNKGLEYIEARWLFNASASQMEVLIHPQSVIHSMVRYQDGSVLAQLGEPDMRTPIAHTMAWPNRVNSGVKPLDFCKLSALTFAAPDYDRYPCLKLAMEAFEQGQAATTALNAANEITVAAFLAQQIRFTDIAALNLSVLEKMDMREPQCVDDVLSVDANAREVARKEVMRLAS

SEQ ID NO: 138 - protein, 299 aa, Escherichia coli
  source 1..299: note = ispA
MDFPQQLEACVKQANQALSRFIAPLPFQNTPVVETMQYGALLGGKRLRPFLVYATGHMFGVSTNTLDAPAAAVECIHAYSLIHDDLPAMDDDDLRRGLPTCHVKFGEANAILAGDALQTLAFSILSDADMPEVSDRDRISMISELASASGIAGMCGGQALDLDAEGKHVPLDALERIHRHKTGALIRAAVRLGALSAGDKGRRALPVLDKYAESIGLAFQVQDDILDVVGDTATLGKRQGADQQLGKSTYPALLGLEQARKKARDLIDDARQSLKQLAEQSLDTSALEALADYIIQRNK

SEQ ID NO: 139 - protein, 551 aa, Clausena lansium
  source 1..551: note = STS
MSTQQVSSENIVRNAANFHPNIWGNHFLTCPSQTIDSWTQQHHKELKEEVRKMMVSDANKPAQRLRLIDTVQRLGVAYHFEKEIDDALEKIGHDPFDDKDDLYIVSLCFRLLRQHGIKISCDVFEKFKDDDGKFKASLMNDVQGMLSLYEAAHLAIHGEDILDEAIVFTTTHLKSTVSNSPVNSTFAEQIRHSLRVPLRKAVPRLESRYFLDIYSRDDLHDKTLLNFAKLDFNILQAMHQKEASEMTRWWRDFDFLKKLPYIRDRVVELYFWILVGVSYQPKFSTGRIFLSKIICLETLVDDTFDAYGTFDELAIFTEAVTRWDLGHRDALPEYMKFIFKTLIDVYSEAEQELAKEGRSYSIHYAIRSFQELVMKYFCEAKWLNKGYVPSLDDYKSVSLRSIGFLPIAVASFVFMGDIATKEVFEWEMNNPKIIIAAETIFRFLDDIAGHRFEQKREHSPSAIECYKNQHGVSEEEAVKALSLEVANSWKDINEELLLNPMAIPLPLLQVILDLSRSADFMYGNAQDRFTHSTMMKDQVDLVLKDPVKLDD

SEQ ID NO: 140 - protein, 572 aa, synthetic construct
  REGION 1..572: note = Matricaria recutita MrBBS
MSTLSVSTPSFSSSPLSSVNKNSTKQHVTRNSVIFHDSIWGDQFLEYKEKFNVATEKQLIEELKEEVRNELMIRACNEASRYIKLIQLIDVVERLGLAYHFEKEIEESLQHIYVTYGHKWTNYNNIESLSLWFRLLRQNGFNVSSDIFENHIDEKGNFQESLCNDPQGMLALYEAAYMRVEGEIILDKALEFTKLHLGIISNDPSCDSSLRTEIKQALKQPLRRRLPRLEAVRYIAIYQQKASHSEVLLKLAKLDFNVLQEMHKDELSQICKWWKDLDIRNKLPYVRDRLIEGYFWILGIYFEPQHSRTRMFLMKTCMWLIVLDDTFDNYGTYEELEIFTQAVERWSITCLDELPEYMKLIYHEQFRVHQEMEESLEKEGKAYQIHYIKEMAKEGTRSLLLEAKWLKEGYMPTLDEYLSNSLVTCGYALMTARSYVARDDGIVTEDAFKWVATHPPIVKAACKILRLMDDIATHKEEQERGHIASSIECYRKETGASEEEACMDFLKQVEDGWKVINQESLMPTDVPFPLLIPAINLARVSDTLYKDNDGYNHADKEVIGYIKSLFVHPMIV

SEQ ID NO: 141 - protein, 391 aa, synthetic construct
  REGION 1..391: note = EGFP
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKGYPAGWRPLEMVERKKLGVLQPTQQCRLTVHQSKIMVDSPKALKRKAFGDENFSPVPTLSRKSIFEDPPTPASKRSKLSDKVDSAIQTDNLCCGNGGTEISRGSSSSKSSSEASTKACSQKSQSQSVLDMLTSAEPNEQYWQMMAAERKAAL

SEQ ID NO: 142 - protein, 410 aa, Lactococcus lactis
MRKKFYQMSPQERLNSLNLSEKSQEILSEMALDTTILDNLIENQISEFELPMGIAQNFVINGQSFLIPMVTEEPSVIAAASNGAKIAGNFVAEIKERLMRGQIVFYDVKNSDKIANEILEKQEKIFEQAELSYPSIVKRGGGLREVSSRIFSSQKFLSVDVKVDVKDAMGANIINSILEGIAELFRRWFPDEKILFSILSNYATESLVKVTCEIPVERLSKKADGYEIGQKIMAASQYSKIDPYRASTHNKGIMNGINAVILATGNDTRAISAAIHAYAAKDGAYQGLANWELQEKMLVGELEFPLPVATVGGGVKVLPKAQAAMEILGISDAKELAKVIAAVGLAQNLAALRALVSEGIQQGHMSLQARSLALSVGAQADEIAILSQQLRKEKVMNQEVAQNLLKNLRK

SEQ ID NO: 143 - protein, 410 aa, synthetic construct; identical to SEQ ID NO: 142
  REGION 1..410: note = Lactococcus lactis subsp. cremoris MG1363 (Hydroxymethylglutaryl-CoA reductase)